(19) United States
(12) Patent Application Publication
(10) Pub. No.: US 2021/0261553 A1
(43) Pub. Date: Aug. 26, 2021

(54) FUSED TETRAZOLES AS LRRK2 INHIBITORS

(71) Applicant: E-SCAPE Bio, Inc., San Francisco, CA (US)

(72) Inventors: Albert W. Garofalo, South San Francisco, CA (US); Daniele Andreotti, San Giovanni Lupatoto (IT); Silvia Bernardi, Verona (IT); Elena Serra, Verona (IT); Marco Migliore, Bussolengo (IT); Fabio Maria Sabbatini, Verona (IT); Claudia Beato, Castel d'Azzano (IT); Paolo Vincetti, Parma (IT); Federica Budassi, Urbino (IT)

(21) Appl. No.: 17/055,198

(22) PCT Filed: May 14, 2019
(86) PCT No.: PCT/US2019/032163

§ 371 Date: Nov. 13, 2020

Related U.S. Application Data
(60) Provisional application No. 62/776,137, filed on Dec. 6, 2018.
(60) Provisional application No. 62/671,580, filed on May 15, 2018.

(51) Int. Cl. C07D 487/04; C07D 519/00

(52) U.S. Cl. CPC C07D 487/04; C07D 519/00

(57) ABSTRACT

The present invention is directed to fused tetrazoles of formula (IA) which are inhibitors of LRRK2 and are useful in the treatment of CNS disorders.

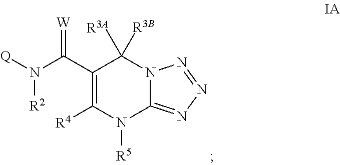

### FIELD OF THE INVENTION

The present invention is directed to aminoindazole compounds which are inhibitors of LRRK2 and are useful in the treatment of CNS disorders.

### BACKGROUND OF THE INVENTION

Parkinson's disease (“PD”) is the most common form of parkinsonism, a movement disorder, and the second most common, age-related neurodegenerative disease estimated to affect 1-2% of the population over age 65. PD is characterized by tremor, rigidity, postural instability, impaired speech, and bradykinesia. It is a chronic, progressive disease with increasing disability and diminished quality of life. In addition to PD, parkinsonism is exhibited in a range of conditions such as progressive supranuclear palsy, corticobasal degeneration, multiple system atrophy, and dementia with Lewy bodies.

Current therapeutic strategies for PD are primarily palliative and focus on reducing the severity of symptoms using supplemental dopaminergic medications. At present, there is no disease-modifying therapy that addresses the underlying neuropathological cause of the disease, thus constituting a significant unmet medical need.

It has long been known that family members of PD patients have an increased risk of developing the disease compared to the general population. Leucine-rich repeat kinase 2 (“LRRK2,” also known as dardarin) is a 286 kDa multi-domain protein that has been linked to PD by genome-wide association studies. LRRK2 expression in the brain is highest in areas impacted by PD (Eur. J. Neurosci. 2006, 23(3):659) and LRRK2 has been found to localize in Lewy Bodies, which are intracellular protein aggregates considered to be a hallmark of the disease. Patients with point mutations in LRRK2 present disease that is indistinguishable from idiopathic patients. While more than 20 LRRK2 mutations have been associated with autosomal-dominantly inherited parkinsonism, the G2019S mutation located within the kinase domain of LRRK2 is by far the most common. This particular mutation is found in >85% of LRRK2-linked PD patients. It has been shown that the G2019S mutation in LRRK2 leads to an enhancement in LRRK2 kinase activity and inhibition of this activity is a therapeutic target for the treatment of PD.

In addition to PD, LRRK2 has been linked to other diseases such as cancer, leprosy, and Crohn's disease (Sci. Signal., 2012, 5(207), pe2). As there are presently limited therapeutic options for treating PD and other disorders associated with aberrant LRRK2 kinase activity, there remains a need for developing LRRK2 inhibitors.

### SUMMARY OF THE INVENTION

The present invention provides a compound of Formula IA:

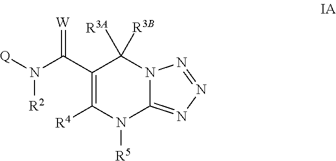

or a pharmaceutically acceptable salt thereof, wherein constituent members are defined herein.

The present invention further provides a pharmaceutical composition comprising a compound of Formula IA, or a pharmaceutically acceptable salt thereof, and at least one pharmaceutically acceptable carrier.

The present invention further provides a method of inhibiting LRRK2 activity, comprising contacting a compound of Formula IA, or a pharmaceutically acceptable salt thereof, with LRRK2.

The present invention further provides a method of treating a disease or disorder associated with elevated expression or activity of LRRK2, or functional variants thereof, comprising administering to a patient in need thereof a therapeutically effective amount of a compound of Formula IA, or a pharmaceutically acceptable salt thereof.

The present invention further provides a method for treating a neurodegenerative disease in a patient, comprising: administering to the patient a therapeutically effective amount of the compound of Formula IA, or a pharmaceutically acceptable salt thereof.

The present invention further provides use of a compound of Formula IA, or a pharmaceutically acceptable salt thereof, in therapy.

The present invention further provide a compound of Formula IA, or a pharmaceutically acceptable salt thereof, for use in the preparation of a medicament for use in therapy.

### DETAILED DESCRIPTION

### Compounds

The present disclosure provides a compound of Formula IA:

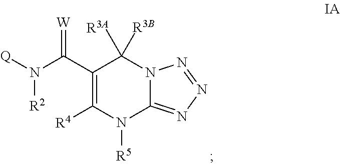

or a pharmaceutically acceptable salt thereof, wherein:

W is O or S;

Q is selected from one of the following:

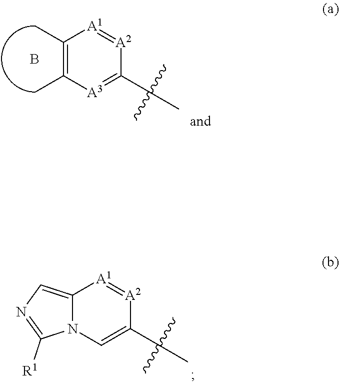

A¹, A², and A³ are each independently selected from N and CR⁶, wherein no more than two of A¹, A², and A³ in (a) are simultaneously N;

ring B is selected from:

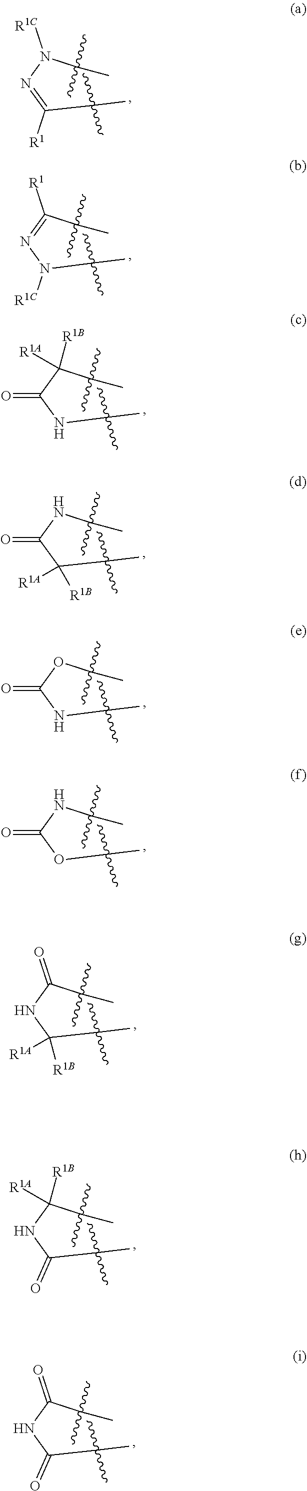

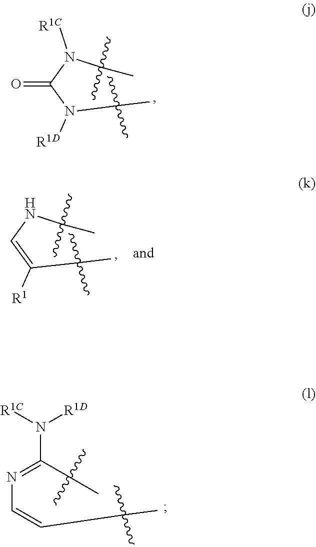

R¹, R^{1A}, and R^{1B} are each independently selected from H, halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}; wherein said C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹ are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, C(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};

or R^{1A} and R^{1B} together form a C₃₋₇ cycloalkyl or 4-10 membered heterocycloalkyl ring, each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};

R^{1C} and R^{1D} are each independently selected from H and C₁₋₃ alkyl;

R² is H or C₁₋₄ alkyl;

R^{3A} and R^{3B} are each independently selected from H, halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹ are each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

or R^{3A} and R^{3B} together form a C₃₋₇ cycloalkyl or 4-10 membered heterocycloalkyl ring, each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

R⁴ is H, C₁₋₄ alkyl, halo, C₁₋₄ haloalkyl, or CN;

R⁵ is H, C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR³², SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2} NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹ are each optionally substituted with 1, 2, 3, 4, or 5 substituents independently selected from Cy³, Cy³-C₁₋₄ alkyl, halo, C₁₋₆ alkyl, C₂₋₆ alkenyl, C₂₋₆ alkynyl, C₁₋₆ haloalkyl, CN, NO₂, OR³², SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, or S(O)₂NR^{c2}R^{d2};

each R⁶ is independently selected from H, halo, C₁₋₆ alkyl, C₁₋₆ haloalkyl, C₆₋₁₀ aryl, C₃₋₇ cycloalkyl, 5-6 membered heteroaryl, 4-7 membered heterocycloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3}, wherein said C₁₋₆ alkyl, C₁₋₆ haloalkyl, C₆₋₁₀ aryl, C₃₋₇ cycloalkyl, 5-6 membered heteroaryl, 4-7 membered heterocycloalkyl of R⁶ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

each Cy¹ is independently selected from C₆₋₁₀ aryl, C₃₋₁₀ cycloalkyl, 5-14 membered heteroaryl, and 4-14 membered heterocycloalkyl, each optionally substituted by 1, 2, 3, 4, or substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};

each Cy² is independently selected from C₆₋₁₀ aryl, C₃₋₁₀ cycloalkyl, 5-14 membered heteroaryl, and 4-14 membered heterocycloalkyl, each optionally substituted by 1, 2, 3, 4, or substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

each Cy³ is independently selected from C₆₋₁₀ aryl, C₃₋₁₀ cycloalkyl, 5-14 membered heteroaryl, and 4-14 membered heterocycloalkyl, each optionally substituted by 1, 2, 3, 4, or substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR³², C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

each R^{a}, R^{b}, R^{c}, R^{d}, R^{a1}, R^{b1}, R^{c1}, R^{d1}, R^{a2}, R^{b2}, R^{c2}, R^{d2}, R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a2}, R^{b2}, R^{c2}, R^{d2}, R^{a3}, R^{b3}, R^{c3}, or R^{d3} is optionally substituted with 1, 2, 3, 4, or 5 substituents independently selected from halo, C₁₋₄ alkyl, C₁₋₄ haloalkyl, C₁₋₆ haloalkyl, C₂₋₆ alkenyl, C₂₋₆ alkynyl, CN, OR³⁴, SR³⁴, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{C4}C(O)OR³⁴, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};

each R^{a4}, R^{b4}, R^{c4}, and R^{d4} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, 4-7 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl are each optionally substituted with 1, 2, or 3 substituents independently selected from OH, CN, amino, halo, C₁₋₆ alkyl, C₁₋₆alkoxy, C₁₋₆ haloalkyl, and C₁₋₆ haloalkoxy; and

each R^{e}, R^{e1}, R^{e2}, R^{e3}, and R^{e4} is independently selected from H, C₁₋₄ alkyl, and CN.

In some embodiments, the compound is other than:

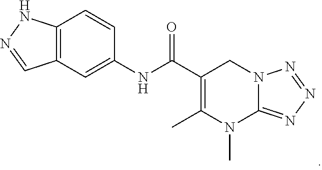 [SMILES: CC1=C(C(=O)Nc2ccc3[nH]ncc3c2)Cn2nnnc2N1C]

In some embodiments, provided herein is a compound of Formula IA, or a pharmaceutically acceptable salt thereof, wherein:

W is O or S;

Q is selected from one of the following:

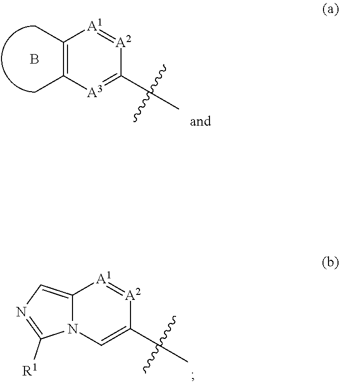

A¹, A², and A³ are each independently selected from N and CR⁶, wherein no more than two of A¹, A², and A³ in (a) are simultaneously N;

ring B is selected from:

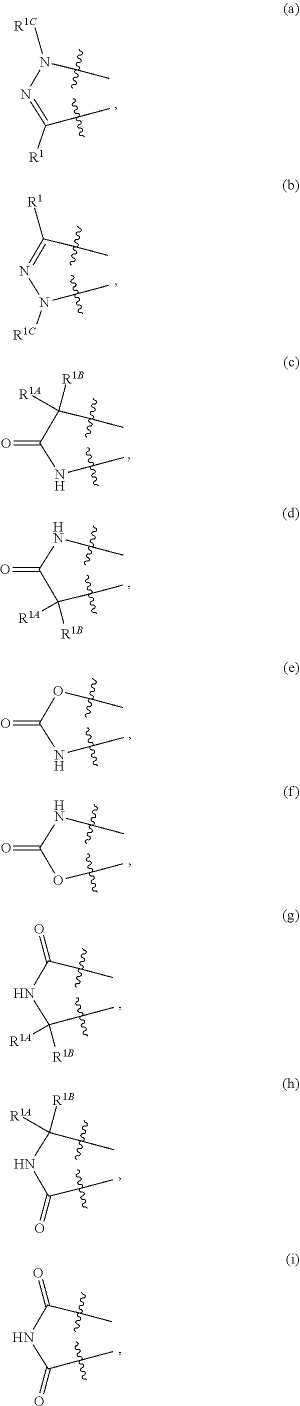

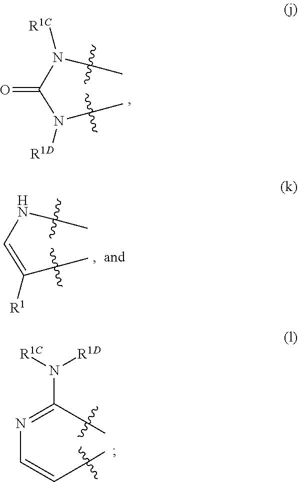

R¹, R^{1A}, and R^{1B} are each independently selected from H, halo, C₁₋₆ alkyl, C₆₋₁₀ aryl, 5-14 membered heteroaryl, C(O)R^{b}, C(O)NR^{c}R^{d}, NR^{c}R^{d}, and NR^{c}C(O)R^{b}; wherein said C₁₋₆ alkyl, C₆₋₁₀ aryl, and 5-14 membered heteroaryl are each optionally substituted with 1, 2, 3, 4, or 5 substituents independently selected from Cy¹, Cy¹-C₁₋₄ alkyl, halo, C₁₋₆ alkyl and S(O)₂NR^{c}R^{d};

R^{1C} and R^{1D} are each independently selected from H and C₁₋₃ alkyl;

R² is H or C₁₋₄ alkyl;

R^{3A} and R^{3B} are each independently selected from H, C₁₋₆ alkyl, C₆₋₁₀ aryl, 5-14 membered heteroaryl, wherein said C₁₋₆ alkyl and 5-14 membered heteroaryl are each optionally substituted with 1, 2, 3, 4, or 5 substituents independently selected from Cy², Cy²-C₁₋₄ alkyl, halo, C₁₋₆ alkyl, and OR^{a1};

or R^{3A} and R^{3B} together form a C₃₋₇ cycloalkyl optionally substituted with 1, 2, 3, 4, or substituents independently selected from Cy², Cy²-C₁₋₄ alkyl, halo, C₁₋₆ alkyl, and OR^{a1};

R⁴ is H or C₁₋₄ alkyl;

R⁵ is H, C₁₋₆ alkyl, C₆₋₁₀ aryl, C₃₋₁₀ cycloalkyl, 5-14 membered heteroaryl, 4-14 membered heterocycloalkyl, 4-10 membered heterocycloalkyl-C₁₋₄ alkyl, CN, NO₂, or C(O)NR^{c2}R^{d2};

R⁶ is H, halo, C₁₋₆ alkyl, C₁₋₆ haloalkyl, OR^{a3}, C(O)NR^{c3}R^{d3}, C(O)OR^{a3}, or NR^{c3}R^{d3};

each Cy¹ is independently selected from 5-14 membered heteroaryl and 4-14 membered heterocycloalkyl;

each Cy² is independently selected from C₆₋₁₀ aryl, C₃₋₁₀ cycloalkyl, 5-14 membered heteroaryl, and 4-14 membered heterocycloalkyl;

each R^{b}, R^{c}, R^{d}, R^{a1}, R^{c2}, R^{d2}, R^{a3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{b}, R^{c}, R^{d}, R^{a1}, R^{c2}, R^{d2}, R^{a3}, R^{c3}, or R^{d3} is optionally substituted with 1, 2, 3, 4, or 5 substituents independently selected from halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};

each R^{a4}, R^{b4}, R^{c4}, and R^{d4} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, 4-7 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl are each optionally substituted with 1, 2, or 3 substituents independently selected from OH, CN, amino, halo, C₁₋₆ alkyl, C₁₋₆alkoxy, C₁₋₆ haloalkyl, and C₁₋₆ haloalkoxy; and

each R^{e4} is independently selected from H, C₁₋₄ alkyl, and CN.

In some embodiments, the compound is other than:

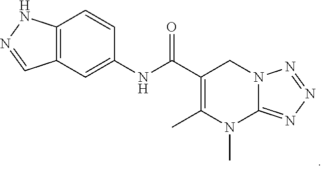 [SMILES: CC1=C(C(=O)Nc2ccc3[nH]ncc3c2)Cn2nnnc2N1C]

The present disclosure also provides a compound of Formula I:

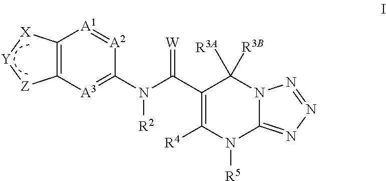

or a pharmaceutically acceptable salt thereof, wherein:

A¹, A², and A³ are each independently selected from N and CR⁶, wherein no more than two of A¹, A², and A³ are simultaneously N;

W is O or S;

the moiety

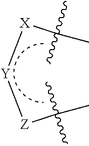

is selected from:

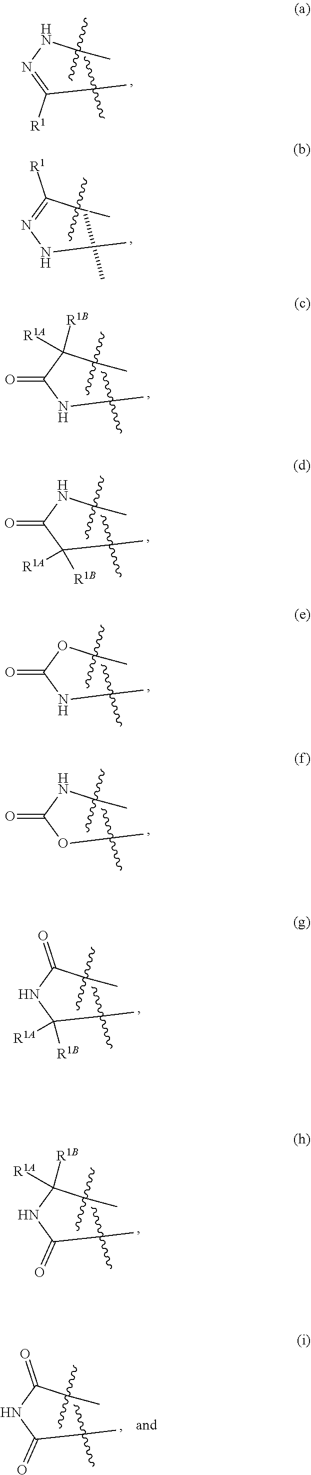

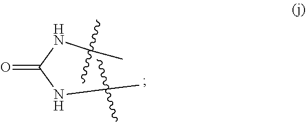 [SMILES: *NC(=O)N*]

R¹, R^{1A}, and R^{1B} are each independently selected from H, halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}; wherein said C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹ are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};

or R^{1A} and R^{1B} together form a C₃₋₇ cycloalkyl or 4-10 membered heterocycloalkyl ring, each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}; R² is H or C₁₋₄ alkyl;

R^{3A} and R^{3B} are each independently selected from H, halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹ are each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

or R^{3A} and R^{3B} together form a C₃₋₇ cycloalkyl or 4-10 membered heterocycloalkyl ring, each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

R⁴ is H, C₁₋₄ alkyl, halo, C₁₋₄ haloalkyl, or CN;

R⁵ is H, C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR³², SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2} NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹ are each optionally substituted with 1, 2, 3, 4, or 5 substituents independently selected from Cy³, Cy³-C₁₋₄ alkyl, halo, C₁₋₆ alkyl, C₂₋₆ alkenyl, C₂₋₆ alkynyl, C₁₋₆ haloalkyl, CN, NO₂, OR³², SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, or S(O)₂NR^{c2}R^{d2};

each R⁶ is independently selected from H, halo, C₁₋₆ alkyl, C₁₋₆ haloalkyl, C₆₋₁₀ aryl, C₃₋₇ cycloalkyl, 5-6 membered heteroaryl, 4-7 membered heterocycloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3}, wherein said C₁₋₆ alkyl, C₁₋₆ haloalkyl, C₆₋₁₀ aryl, C₃₋₇ cycloalkyl, 5-6 membered heteroaryl, 4-7 membered heterocycloalkyl of R⁶ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

each Cy¹ is independently selected from C₆₋₁₀ aryl, C₃₋₁₀ cycloalkyl, 5-14 membered heteroaryl, and 4-14 membered heterocycloalkyl, each optionally substituted by 1, 2, 3, 4, or substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};

each Cy² is independently selected from C₆₋₁₀ aryl, C₃₋₁₀ cycloalkyl, 5-14 membered heteroaryl, and 4-14 membered heterocycloalkyl, each optionally substituted by 1, 2, 3, 4, or substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

each Cy³ is independently selected from C₆₋₁₀ aryl, C₃₋₁₀ cycloalkyl, 5-14 membered heteroaryl, and 4-14 membered heterocycloalkyl, each optionally substituted by 1, 2, 3, 4, or substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR³², C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

each R^{a}, R^{b}, R^{c}, R^{d}, R^{a1}, R^{b1}, R^{c1}, R^{d1}, R^{a2}, R^{b2}, R^{c2}, R^{d2}, R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a2}, R^{b2}, R^{c2}, R^{d2}, R^{a3}, R^{b3}, R^{c3}, or R^{d3} is optionally substituted with 1, 2, 3, 4, or 5 substituents independently selected halo, C₁₋₄ alkyl, C₁₋₄ haloalkyl, C₁₋₆ haloalkyl, C₂₋₆ alkenyl, C₂₋₆ alkynyl, CN, OR³⁴, SR³⁴, C(O)R^{b4}, C(O)NR^{c4}R^{d4}, C(O)OR³⁴, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};

each R^{a4}, R^{b4}, R^{c4}, and R^{d4} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, 4-7 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl are each optionally substituted with 1, 2, or 3 substituents independently selected from OH, CN, amino, halo, C₁₋₆ alkyl, C₁₋₆alkoxy, C₁₋₆ haloalkyl, and C₁₋₆ haloalkoxy; and

each R^{e}, R^{e1}, R^{e2}, R^{e3}, and R^{e4} is independently selected from H, C₁₋₄ alkyl, and CN.

In some embodiments, the compound is other than:

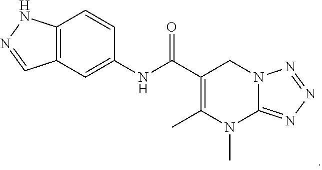 [SMILES: CC1=C(C(=O)Nc2ccc3[nH]ncc3c2)Cn2nnnc2N1C]

In some embodiments, provided herein is a compound of Formula I, or a pharmaceutically acceptable salt thereof, wherein

A¹, A², and A³ are each independently selected from N and CR⁶, wherein no more than two of A¹, A², and A³ are simultaneously N;

W is O or S;

the moiety

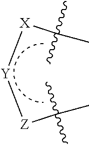

is selected from:

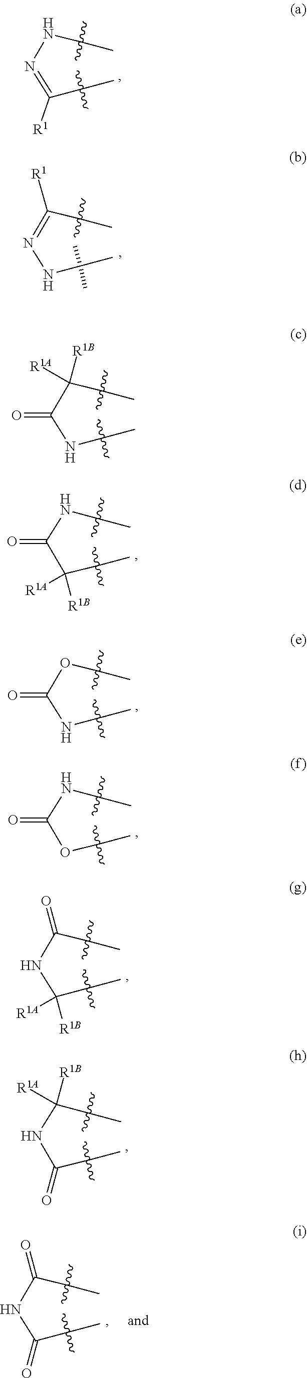

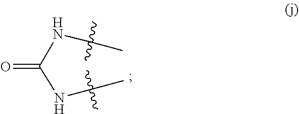 [SMILES: *NC(=O)N*]

R¹, R^{1A}, and R^{1B} are each independently selected from H, halo, C₁₋₆ alkyl, C₆₋₁₀ aryl, 5-14 membered heteroaryl, C(O)R^{b}, C(O)NR^{c}R^{d}, NR^{c}R^{d}, and NR^{c}C(O)R^{b}; wherein said C₁₋₆ alkyl, C₆₋₁₀ aryl, and 5-14 membered heteroaryl are each optionally substituted with 1, 2, 3, 4, or 5 substituents independently selected from Cy¹, Cy¹-C₁₋₄ alkyl, halo, C₁₋₆ alkyl and S(O)₂NR^{c}R^{d};

R² is H or C₁₋₄ alkyl;

R^{3A} and R^{3B} are each independently selected from H, C₁₋₆ alkyl, C₆₋₁₀ aryl, 5-14 membered heteroaryl, wherein said C₁₋₆ alkyl and 5-14 membered heteroaryl are each optionally substituted with 1, 2, 3, 4, or 5 substituents independently selected from Cy², Cy²-C₁₋₄ alkyl, halo, C₁₋₆ alkyl, and OR^{a1};

R⁴ is H or C₁₋₄ alkyl;

R⁵ is H, C₁₋₆ alkyl, C₆₋₁₀ aryl, C₃₋₁₀ cycloalkyl, 5-14 membered heteroaryl, 4-14 membered heterocycloalkyl, 4-10 membered heterocycloalkyl-C₁₋₄ alkyl, CN, NO₂, or C(O)NR^{c2}R^{d2};

R⁶ is H, halo, OR^{a3}, C(O)NR^{c3}R^{d3}, C(O)OR^{a3}, or NR^{c3}R^{d3};

each Cy¹ is independently selected from 5-14 membered heteroaryl and 4-14 membered heterocycloalkyl;

each Cy² is independently selected from C₆₋₁₀ aryl, C₃₋₁₀ cycloalkyl, 5-14 membered heteroaryl, and 4-14 membered heterocycloalkyl;

each R^{b}, R^{c}, R^{d}, R^{a1}, R^{c2}, R^{d2}, R^{a3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{b}, R^{c}, R^{d}, R^{a1}, R^{c2}, R^{d2}, R^{a3}, R^{c3}, or R^{d3} is optionally substituted with 1, 2, 3, 4, or 5 substituents independently selected halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};

each R^{a4}, R^{b4}, R^{c4}, and R^{d4} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, 4-7 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl are each optionally substituted with 1, 2, or 3 substituents independently selected from OH, CN, amino, halo, C₁₋₆ alkyl, C₁₋₆alkoxy, C₁₋₆ haloalkyl, and C₁₋₆ haloalkoxy; and

each R^{e4} is independently selected from H, C₁₋₄ alkyl, and CN;

with the proviso that the compound is other than:

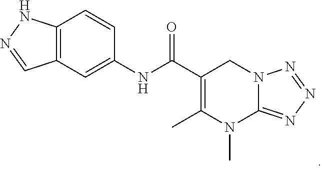 [SMILES: CC1=C(C(=O)Nc2ccc3[nH]ncc3c2)Cn2nnnc2N1C]

In some embodiments, Q is (a) and A¹, A², and A³ are each CR⁶. In some embodiments, Q is (a) and A¹ is N, and A² and A³ are each CR⁶. In some embodiments, Q is (a) and A¹ and A³ are each CR⁶, and A² is N.

In some embodiments, Q is (b) and A¹ and A² are each CR⁶.

In some embodiments, ring B is selected from:

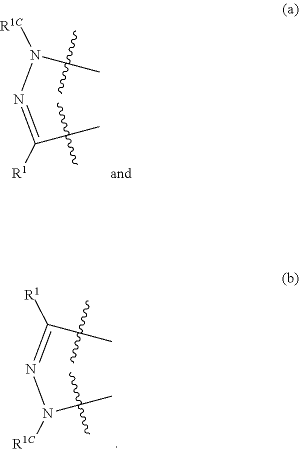

In some embodiments, ring B is selected from:

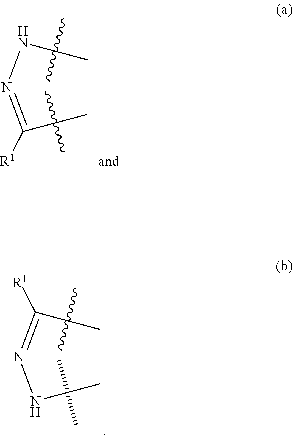

In some embodiments, ring B is

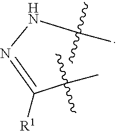

In some embodiments, ring B is H

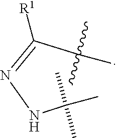

In some embodiments, ring B is selected from:

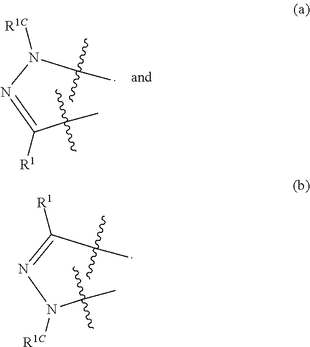

In some embodiments, ring B is selected from:

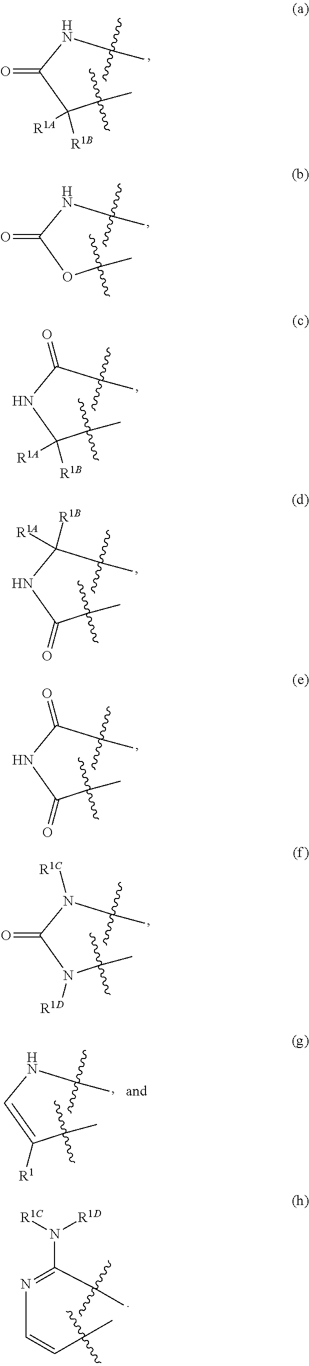

In some embodiments, R^{1A} and R^{1B} are each independently selected from H and C₁₋₆ alkyl. In some embodiments, R^{1A} and R^{1B} are each methyl. In some embodiments, R^{1A} and R^{1B} are each H.

In some embodiments, R^{1C} and R^{1D} are each H. In some embodiments, R^{1C} is C₁₋₃ alkyl. In some embodiments, R^{1C} is methyl. In some embodiments, R^{1C} is H. In some embodiments, R^{1D} is C₁₋₃ alkyl. In some embodiments, R^{1D} is methyl. In some embodiments, R^{1D} is H.

In some embodiments, Q is

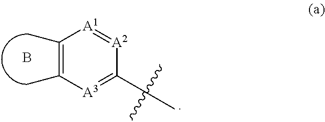

In some embodiments, Q is

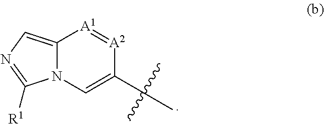

In some embodiments, A¹, A², and A³ are each CR⁶. In some embodiments, A¹ is N, and A² and A³ are each CR⁶. In some embodiments, A¹ and A³ are each CR⁶, and A² is N.

In some embodiments, W is O. In some embodiments, W is S.

In some embodiments, the moiety

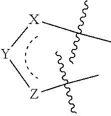

is selected from:

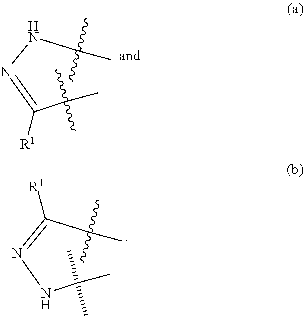

In some embodiments, the moiety

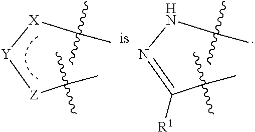

In some embodiments, the moiety

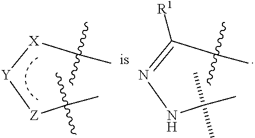

In some embodiments, the moiety

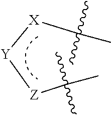

is selected from:

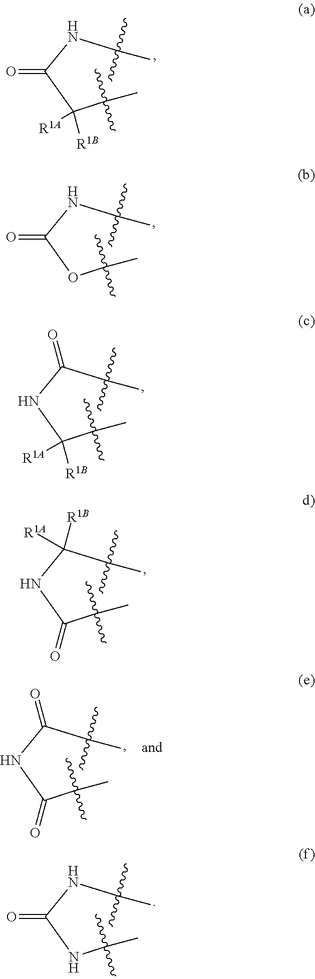

In some embodiments, R¹ is selected from H, halo, C₁₋₆ alkyl, C₆₋₁₀ aryl, 5-14 membered heteroaryl, C(O)R^{b}, C(O)NR^{c}R^{d}, NR^{c}R^{d}, and NR^{c}C(O)R^{b}; wherein said C₁₋₆ alkyl, C₆₋₁₀ aryl, and 5-14 membered heteroaryl are each optionally substituted with 1, 2, 3, 4, or 5 substituents independently selected from Cy¹, Cy¹-C₁₋₄ alkyl, halo, C₁₋₆ alkyl and S(O)₂NR^{c}R^{d}.

In some embodiments, R¹ is selected from H, halo, C₁₋₆ alkyl, C₆₋₁₀ aryl, 4-14 membered heterocycloalkyl, 5-14 membered heteroaryl, C(O)R^{b}, C(O)OR^{a}, C(O)NR^{c}R^{d}, NR^{c}R^{d}, and NR^{c}C(O)R^{b}; wherein said C₁₋₆ alkyl, C₆₋₁₀ aryl, 4-14 membered heterocycloalkyl, and 5-14 membered heteroaryl are each optionally substituted with 1, 2, 3, 4, or 5 substituents independently selected from Cy¹, Cy¹-C₁₋₄ alkyl, halo, C₁₋₆ alkyl and S(O)₂NR^{c}R^{d}.

In some embodiments, R¹ is H.

In some embodiments, R¹ is halo. In some embodiments, R¹ is Br.

In some embodiments, R¹ is C₁₋₆ alkyl. In some embodiments, R¹ is methyl.

In some embodiments, R¹ is methyl or isopropyl.

In some embodiments, R¹ is C₆₋₁₀ aryl, optionally substituted with Cy¹ or SO₂NH₂. In some embodiments, R¹ is phenyl.

In some embodiments, R¹ is 5-10 membered heteroaryl, optionally substituted with Cy¹. In some embodiments, R¹ is pyridinyl or pyrimidinyl. In some embodiments, R¹ is NH₂.

In some embodiments, R¹ is CONH₂.

In some embodiments, R¹ is C(O)OR^{a}.

In some embodiments, R¹ is NR^{c}C(O)R^{b}.

In some embodiments, R¹ is C(O)NR^{c}R^{d}.

In some embodiments, R¹ is pyridinyl, pyrimidinyl, or 1H-benzo[d]imidazolyl, each optionally substituted with Cy¹.

In some embodiments, R¹ is 4-14 membered heterocycloalkyl optionally substituted with 1, 2, 3, 4, or 5 substituents independently selected from Cy¹, Cy¹-C₁₋₄ alkyl, halo, C₁₋₆ alkyl and S(O)₂NR^{c}R^{d}.

In some embodiments, R¹ is pyrrolidinyl.

In some embodiments, R^{a} is independently selected from H, C₁₋₆ alkyl, and C₁₋₆ haloalkyl, wherein said C₁₋₆ alkyl and C₁₋₆ haloalkyl are each optionally substituted with 1, 2, 3, 4, or 5 substituents independently selected from halo, C₁₋₄ alkyl, C₁₋₄ haloalkyl, C₁₋₆ haloalkyl, CN, and OR^{a4}. In some embodiments, R^{a} is C₁₋₆ alkyl. In some embodiments, R^{a} is methyl.

In some embodiments, R^{b} is C₁₋₆ alkyl, C₆₋₁₀ aryl, 5-10 membered heteroaryl, C₃₋₇ cycloalkyl, or C₆₋₁₀ aryl-C₁₋₄ alkyl, each of which is optionally substituted with 1, 2, 3, 4, or substituents independently selected from halo, C₁₋₄ alkyl, C₁₋₄ haloalkyl, C₁₋₆ haloalkyl, CN, OR^{a4}, and NR^{b4}R^{c4}. In some embodiments, R^{b} is H, C₁₋₆ alkyl, 5-10 membered heteroaryl, or C₃₋₇ cycloalkyl. In some embodiments, R^{b} is propyl, furanyl, or cyclopropyl.

In some embodiments, R^{c} is selected from C₁₋₆ alkyl and H. In some embodiments, R^{c} is H.

In some embodiments, R^{d} is 5-10 membered heteroaryl optionally substituted with 1, 2, 3, 4, or 5 substituents independently selected from halo, C₁₋₄ alkyl, C₁₋₄ haloalkyl, C₁₋₆ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, and NR^{c4}R^{d4}.

In some embodiments, R^{d} is 5-10 membered heteroaryl optionally substituted with 1 or 2 substituents independently selected from halo and C₁₋₄ alkyl. In some embodiments, R^{d} is pyridinyl, optionally substituted with methyl.

In some embodiments, R^{b4} and R^{c4} are each independently selected from H, C₁₋₆ alkyl, and C₁₋₆ haloalkyl, wherein said C₁₋₆ alkyl and C₁₋₆ haloalkyl are each optionally substituted with 1, 2, or 3 substituents independently selected from OH, CN, amino, and halo. In some embodiments, R^{b4} and R^{c4} are each independently selected from C₁₋₆ alkyl. In some embodiments, R^{b4} and R^{c4} are each methyl.

In some embodiments, each Cy¹ is independently selected from C₃₋₁₀ cycloalkyl and 4-14 membered heterocycloalkyl, each optionally substituted by 1, 2, 3, 4, or 5 substituents independently selected from halo, C₁₋₆ alkyl, C₁₋₆ haloalkyl, CN, NO₂, OR^{a}, SR^{a}, C(O)R^{b}, and NR^{c}R^{d}. In some embodiments, each Cy¹ is independently selected from 4-14 membered heterocycloalkyl optionally substituted by 1, 2, 3, 4, or 5 substituents independently selected from halo and C₁₋₆ alkyl.

In some embodiments, Cy¹ is morpholinyl.

In some embodiments, Cy¹ is piperidinyl or morpholinyl, each of which is optionally substituted with 1 or 2 substituents independently selected from C₁₋₆ alkyl. In some embodiments, Cy¹ is piperidinyl substituted with 2 methyl groups.

In some embodiments, R^{b} is C₁₋₆ alkyl, C₆₋₁₀ aryl, or 4-10 membered heterocycloalkyl.

In some embodiments, R^{b} is phenyl, morpholino, or methyl.

In some embodiments, R² is H.

In some embodiments, R² is C₁₋₄ alkyl. In some embodiments, R² is methyl.

In some embodiments, R^{3A} and R^{3B} are each independently selected from H, C₁₋₆ alkyl, C₆₋₁₀ aryl, and 5-14 membered heteroaryl, wherein said C₁₋₆ alkyl and 5-14 membered heteroaryl are each optionally substituted with 1, 2, 3, 4, or 5 substituents independently selected from Cy², Cy²-C₁₋₄ alkyl, halo, C₁₋₆ alkyl, and OR^{a1}.

In some embodiments, R^{3A} and R^{3B} are each independently selected from H, methyl, ethyl, isopropyl, phenyl, and OH.

In some embodiments, R^{3A} is C₁₋₆ alkyl optionally substituted with OR^{a1}.

In some embodiments, R^{3A} and R^{3B} are each H.

In some embodiments, R^{3A} is methyl and R^{3B} is H.

In some embodiments, R^{3A} and R^{3B} are each methyl.

In some embodiments, at least one of R^{3A} and R^{3B} is other than H.

In some embodiments, R^{3A} and R^{3B} together form a C₃₋₇ cycloalkyl. In some embodiments, R^{3A} and R^{3B} together form a cyclopentyl group.

In some embodiments, R⁴ is H.

In some embodiments, R⁴ is C₁₋₄ alkyl. In some embodiments, R⁴ is methyl. In some embodiments, R⁴ is ethyl.

In some embodiments, R⁵ is H, C₁₋₆ alkyl, C₆₋₁₀ aryl, C₃₋₁₀ cycloalkyl, 5-14 membered heteroaryl, 4-14 membered heterocycloalkyl, 4-10 membered heterocycloalkyl-C₁₋₄ alkyl, CN, NO₂, or C(O)NR^{c2}R^{d2}.

In some embodiments, R⁵ is H.

In some embodiments, R⁵ is C₁₋₆ alkyl. In some embodiments, R⁵ is methyl. In some embodiments, R⁵ is ethyl.

In some embodiments, R⁵ is C₆₋₁₀ aryl. In some embodiments, R⁵ is phenyl.

In some embodiments, R⁵ is 4-10 membered heterocycloalkyl-C₁₋₄ alkyl. In some embodiments, R⁵ is morpholino-C₁₋₄ alkyl.

In some embodiments, R⁵ is C(O)NR^{c2}R^{d2}.

In some embodiments, R⁵ is H, C₁₋₆ alkyl, C₆₋₁₀ aryl, C₃₋₁₀ cycloalkyl, 5-14 membered heteroaryl, 4-14 membered heterocycloalkyl, 4-10 membered heterocycloalkyl-C₁₋₄ alkyl, CN, NO₂, C(O)NR^{c2}R^{d2}, or C(O)R^{b2}, wherein said C₁₋₆ alkyl, C₆₋₁₀ aryl, C₃₋₁₀ cycloalkyl, 5-14 membered heteroaryl, 4-14 membered heterocycloalkyl, and 4-10 membered heterocycloalkyl-C₁₋₄ alkyl are each optionally substituted with 1, 2, 3, 4, or 5 substituents independently selected from Cy³, Cy³-C₁₋₄ alkyl, halo, C₁₋₆ alkyl, C₁₋₆ haloalkyl, CN, NO₂, OR³², SR³², C(O)R^{b2}, C(O)NR^{c2}R^{d2}, C(O)OR^{a2}, OC(O)R^{b2}, OC(O)NR^{c2}R^{d2}, and NR^{c2}R^{d2}.

In some embodiments, R⁵ is H, C₁₋₆ alkyl, C₆₋₁₀ aryl, C₃₋₁₀ cycloalkyl, 5-14 membered heteroaryl, 4-14 membered heterocycloalkyl, 4-10 membered heterocycloalkyl-C₁₋₄ alkyl, CN, NO₂, C(O)NR^{c2}R^{d2}, or C(O)R^{b2}.

In some embodiments, R^{b2} is H, C₁₋₆ alkyl, or C₁₋₆ haloalkyl. In some embodiments, R^{b2} is C₁₋₆ alkyl. In some embodiments, R^{b2} is methyl.

In some embodiments, R^{c2} and R^{d2} are each independently selected from H and C₁₋₆ alkyl.

In some embodiments, R^{c2} and R^{d2} are each methyl.

In some embodiments, R⁶ is H, halo, OR^{a3}, C(O)NR^{c3}R^{d3}, C(O)OR^{a3}, or NR^{c3}R^{d3}. In some embodiments, R⁶ is H. In some embodiments, R⁶ is halo. In some embodiments, R⁶ is F. In some embodiments, R⁶ is methoxy. In some embodiments, R⁶ is C(O)NR^{c3}R^{d3}. In some embodiments, R⁶ is C(O)OR^{a3}. In some embodiments, R⁶ is NR^{c3}R^{d3}.

In some embodiments, each R⁶ is independently selected from H, halo, OR^{a3}, C(O)NR^{c3}R^{d3}, C(O)OR^{a3}, and NR^{c3}R^{d3}. In some embodiments, each R⁶ is independently selected from H, halo, OR^{a3}, C₁₋₆ alkyl, C₁₋₆ haloalkyl, C(O)NR^{c3}R^{d3}, C(O)OR^{a3}, and NR^{c3}R^{d3}.

In some embodiments, each R⁶ is independently selected from H, F, methyl, methoxy, and CF₃. In some embodiments, each R⁶ is independently selected from H and halo. In some embodiments, each R⁶ is independently selected from H and F. In some embodiments, each R⁶ is independently selected from H and methoxy.

In some embodiments, each R⁶ is independently selected from H, C(O)NR^{c3}R^{d3}, and NR^{c3}R^{d3}.

In some embodiments, R^{c} and R^{d} are each H.

In some embodiments, R^{a3} is H, C₁₋₆ alkyl, or C₁₋₆ haloalkyl. In some embodiments, R^{a3} is C₁₋₆ alkyl.

In some embodiments, R^{a3} is methyl.

In some embodiments, provided herein is a compound having Formula II:

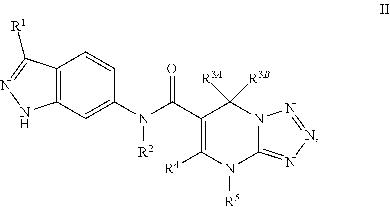

or a pharmaceutically acceptable salt thereof.

In some embodiments, provided herein is a compound having Formula III:

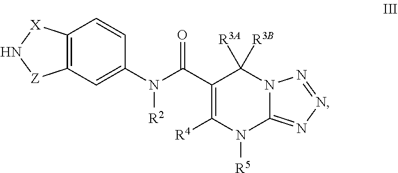

or a pharmaceutically acceptable salt thereof, wherein

X is oxo (═O) or CR^{1A}R^{1B}; and

Z is oxo (═O) or CR^{1A}R^{1B},

wherein if X is CR^{1A}R^{1B} then Z is not CR^{1A}R^{1B}.

In some embodiments, provided herein is a compound having Formula IV:

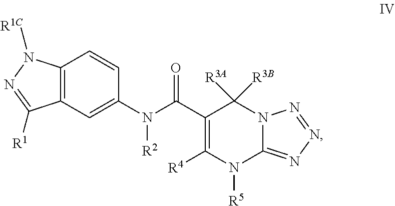

or a pharmaceutically acceptable salt thereof.

In some embodiments, provided herein is a compound selected from:
N-(1H-indazol-5-yl)-4,5,7-trimethyl-4H,7H-[1,2,3,4]tetrazolo[1,5-a]pyrimidine-6-carboxamide;
N-(1H-indazol-5-yl)-4,5,7-trimethyl-4H,7H-[1,2,3,4]tetrazolo[1,5-a]pyrimidine-6-carboxamide;
N-(1H-indazol-6-yl)-4,5-dimethyl-4H,7H-[1,2,3,4]tetrazolo[1,5-a]pyrimidine-6-carboxamide;
N-(1H-Indazol-5-yl)-5-methyl-4-[2-(morpholin-4-yl)ethyl]-4H,7H-[1,2,3,4]tetrazolo[1,5-a]pyrimidine-6-carboxamide;
5-Ethyl-N-(1H-indazol-5-yl)-4-methyl-4H,7H-[1,2,3,4]tetrazolo[1,5-a]pyrimidine-6-carboxamide;
4,5-dimethyl-N-(3-(pyridin-4-yl)-1H-indazol-5-yl)-4,7-dihydrotetrazolo[1,5-a]pyrimidine-6-carboxamide;
4,5-Dimethyl-N-(3-(2-morpholinopyridin-4-yl)-1H-indazol-5-yl)-4,7-dihydrotetrazolo[1,5-a]pyrimidine-6-carboxamide;
7-Ethyl-N-(1H-indazol-5-yl)-4,5-dimethyl-4H,7H-[1,2,3,4]tetrazolo[1,5-a]pyrimidine-6-carboxamide;
4,5-Dimethyl-N-(3-(6-morpholinopyrimidin-4-yl)-1H-indazol-5-yl)-4,7-dihydrotetrazolo[1,5-a]pyrimidine-6-carboxamide;
N-(3-Bromo-1H-indazol-5-yl)-4,5-dimethyl-4,7-dihydrotetrazolo[1,5-a]pyrimidine-6-carboxamide;
4,5-Dimethyl-N-(3-(4-sulfamoylphenyl)-1H-indazol-5-yl)-4,7-dihydrotetrazolo[1,5-a]pyrimidine-6-carboxamide;
N⁶-(1H-indazol-5-yl)-N⁴,N⁴,5-trimethyltetrazolo[1,5-a]pyrimidine-4,6(7H)-dicarboxamide;
N-(1H-indazol-5-yl)-5-methyl-4-phenyl-4,7-dihydrotetrazolo[1,5-a]pyrimidine-6-carboxamide;
N-(4-fluoro-1H-indazol-5-yl)-4,5-dimethyl-4,7-dihydrotetrazolo[1,5-a]pyrimidine-6-carboxamide;
N-(1H-indazol-5-yl)-4,5-dimethyl-4,7-dihydrotetrazolo[1,5-a]pyrimidine-6-carbothioamide;
4,5,7-trimethyl-N-(2H-pyrazolo[3,4-b]pyridin-5-yl)-4,7-dihydrotetrazolo[1,5-a]pyrimidine-6-carboxamide;
N-(6-methoxy-2H-indazol-5-yl)-4,5-dimethyl-4,7-dihydrotetrazolo[1,5-a]pyrimidine-6-carboxamide;
N-(3-carbamoyl-2H-indazol-5-yl)-4,5-dimethyl-4,7-dihydrotetrazolo[1,5-a]pyrimidine-6-carboxamide;
N-(6-fluoro-2H-indazol-5-yl)-4,5-dimethyl-4,7-dihydrotetrazolo[1,5-a]pyrimidine-6-carboxamide;
N-(6-carbamoyl-2H-indazol-5-yl)-4,5-dimethyl-4,7-dihydrotetrazolo[1,5-a]pyrimidine-6-carboxamide;
N-(6-amino-2H-indazol-5-yl)-4,5-dimethyl-4,7-dihydrotetrazolo[1,5-a]pyrimidine-6-carboxamide;
(7R)—N-(3-bromo-2H-indazol-5-yl)-4,5,7-trimethyl-4,7-dihydrotetrazolo[1,5-a]pyrimidine-6-carboxamide;
4,5,7-trimethyl-N-(1H-pyrazolo[3,4-c]pyridin-5-yl)-4,7-dihydrotetrazolo[1,5-a]pyrimidine-6-carboxamide;
4,5,7-trimethyl-N-(3-(2-morpholinopyridin-4-yl)-1H-indazol-5-yl)-4,7-dihydrotetrazolo[1,5-a]pyrimidine-6-carboxamide;
N-(6-amino-2H-indazol-5-yl)-4,5,7-trimethyl-4,7-dihydrotetrazolo[1,5-a]pyrimidine-6-carboxamide;
N-(2H-indazol-5-yl)-4,5,7,7-tetramethyl-4,7-dihydrotetrazolo[1,5-a]pyrimidine-6-carboxamide;
(R)-4,5,7-trimethyl-N-(3-(pyridin-4-yl)-1H-indazol-5-yl)-4,7-dihydrotetrazolo[1,5-a]pyrimidine-6-carboxamide;
N-(3-acetamido-2H-indazol-5-yl)-4,5-dimethyl-4,7-dihydrotetrazolo[1,5-a]pyrimidine-6-carboxamide;
4,5-dimethyl-N-(2-oxoindolin-5-yl)-4,7-dihydrotetrazolo[1,5-a]pyrimidine-6-carboxamide;
4,5-dimethyl-N-(2-oxo-2,3-dihydrobenzo[d]oxazol-6-yl)-4,7-dihydrotetrazolo[1,5-a]pyrimidine-6-carboxamide; and
4,5-dimethyl-N-(2-oxo-2,3-dihydro-1H-benzo[d]imidazol-5-yl)-4,7-dihydrotetrazolo[1,5-a]pyrimidine-6-carboxamide;

or a pharmaceutically acceptable salt thereof.

In some embodiments, provided herein is a compound selected from:
4′,5′-dimethyl-N-(3-methyl-2H-indazol-5-yl)-4′H-spiro[cyclopentane-1,7′-tetrazolo[1,5-a]pyrimidine]-6′-carboxamide;
4,5-dimethyl-N-{3-[3-(morpholin-4-yl)phenyl]-1H-indazol-5-yl}-4H-spiro[[1,2,3,4]tetrazolo[1,5-a]pyrimidine-7,1′-cyclopentane]-6-carboxamide;
(R)—N-(3-(2-((2S,6R)-2,6-dimethylmorpholino)pyridin-4-yl)-1H-indazol-5-yl)-4,5,7-trimethyl-4,7-dihydrotetrazolo[1,5-a]pyrimidine-6-carboxamide;
(R)-4,5,7-trimethyl-N-(3-(pyrrolidin-1-yl)-1H-indazol-5-yl)-4,7-dihydrotetrazolo[1,5-a]pyrimidine-6-carboxamide;
(R)—N-(3-isopropyl-1H-indazol-5-yl)-4,5,7-trimethyl-4,7-dihydrotetrazolo[1,5-a]pyrimidine-6-carboxamide;
trans-(7R)—N-(3-(2-(2,6-dimethylmorpholino)pyridin-4-yl)-1H-indazol-5-yl)-4,5,7-trimethyl-4,7-dihydrotetrazolo[1,5-a]pyrimidine-6-carboxamide;
(7R)—N-(3-{2-[(2S,6S)-2,6-dimethylmorpholin-4-yl]pyridin-4-yl}-1H-indazol-5-yl)-4,5,7-trimethyl-4H,7H-[1,2,3,4]tetrazolo[1,5-a]pyrimidine-6-carboxamide;
(7R)—N-(3-{2-[(2R,6R)-2,6-dimethylmorpholin-4-yl]pyridin-4-yl}-1H-indazol-5-yl)-4,5,7-trimethyl-4H,7H-[1,2,3,4]tetrazolo[1,5-a]pyrimidine-6-carboxamide;
(R)—N-(3-(2-((3R,5S)-3,5-dimethylpiperidin-1-yl)pyridin-4-yl)-1H-indazol-5-yl)-4,5,7-trimethyl-4,7-dihydrotetrazolo[1,5-a]pyrimidine-6-carboxamide;
(R)-4,5,7-trimethyl-N-(3-phenyl-1H-indazol-5-yl)-4,7-dihydrotetrazolo[1,5-a]pyrimidine-6-carboxamide;
(R)—N-(3-(3-((2S,6R)-2,6-dimethylmorpholino)phenyl)-1H-indazol-5-yl)-4,5,7-trimethyl-4,7-dihydrotetrazolo[1,5-a]pyrimidine-6-carboxamide;
4,5,7-trimethyl-N-(3-methyl-1H-indazol-5-yl)-4,7-dihydrotetrazolo[1,5-a]pyrimidine-6-carboxamide;
(R)—N-(1-aminoisoquinolin-6-yl)-4,5,7-trimethyl-4,7-dihydrotetrazolo[1,5-a]pyrimidine-6-carboxamide;
4,5,7,7-tetramethyl-N-(3-(2-morpholinopyridin-4-yl)-1H-indazol-5-yl)-4,7-dihydrotetrazolo[1,5-a]pyrimidine-6-carboxamide;
4,5,7,7-tetramethyl-N-(3-(3-morpholinophenyl)-1H-indazol-5-yl)-4,7-dihydrotetrazolo[1,5-a]pyrimidine-6-carboxamide;
N-(3-(3-((2S,6R)-2,6-dDimethylmorpholino)phenyl)-1H-indazol-5-yl)-4,5,7,7-tetramethyl-4,7-dihydrotetrazolo[1,5-a]pyrimidine-6-carboxamide;
4,5,7,7-tetramethyl-N-(3-phenyl-1H-indazol-5-yl)-4,7-dihydrotetrazolo[1,5-a]pyrimidine-6-carboxamide;
R)-4,5,7-trimethyl-N-(3-methyl-2-oxo-2,3-dihydro-1H-benzo[d]imidazol-5-yl)-4,7-dihydrotetrazolo[1,5-a]pyrimidine-6-carboxamide;
(R)-4,5,7-trimethyl-N-(1-methyl-2-oxo-2,3-dihydro-1H-benzo[d]imidazol-5-yl)-4,7-dihydrotetrazolo[1,5-a]pyrimidine-6-carboxamide;
(R)—N-(3,3-dimethyl-1-oxoisoindolin-5-yl)-4,5,7-trimethyl-4,7-dihydrotetrazolo[1,5-a]pyrimidine-6-carboxamide;
N-(1H-indazol-5-yl)-7-isopropyl-4,5-dimethyl-4,7-dihydrotetrazolo[1,5-a]pyrimidine-6-carboxamide;
4-acetyl-N-(2H-indazol-5-yl)-5-methyl-4,7-dihydrotetrazolo[1,5-a]pyrimidine-6-carboxamide;
N-(3-(2-(4-(dimethylamino)phenyl)acetamido)-2H-indazol-5-yl)-4,5-dimethyl-4,7-dihydrotetrazolo[1,5-a]pyrimidine-6-carboxamide;
N-(4-methoxy-1H-indazol-5-yl)-4,5-dimethyl-4,7-dihydrotetrazolo[1,5-a]pyrimidine-6-carboxamide;
4,5-dimethyl-N-(3-((6-methylpyridin-3-yl)carbamoyl)-2H-indazol-5-yl)-4,7-dihydrotetrazolo[1,5-a]pyrimidine-6-carboxamide;
N-(3-(furan-2-carboxamido)-2H-indazol-5-yl)-4,5-dimethyl-4,7-dihydrotetrazolo[1,5-a]pyrimidine-6-carboxamide;
N-(3-(cyclopropanecarboxamido)-2H-indazol-5-yl)-4,5-dimethyl-4,7-dihydrotetrazolo[1,5-a]pyrimidine-6-carboxamide;
N-(3-butyramido-2H-indazol-5-yl)-4,5-dimethyl-4,7-dihydrotetrazolo[1,5-a]pyrimidine-6-carboxamide;
4,5-dimethyl-N-(3-methyl-2H-indazol-5-yl)-4,7-dihydrotetrazolo[1,5-a]pyrimidine-6-carboxamide;
N-(3-(1H-benzo[d]imidazol-2-yl)-1H-indazol-5-yl)-4,5-dimethyl-4,7-dihydrotetrazolo[1,5-a]pyrimidine-6-carboxamide;
4,5,7,7-tetramethyl-N-(3-methyl-2H-indazol-5-yl)-4,7-dihydrotetrazolo[1,5-a]pyrimidine-6-carboxamide;
methyl 5-(4,5-dimethyl-4,7-dihydrotetrazolo[1,5-a]pyrimidine-6-carboxamido)-2H-indazole-4-carboxylate;
4,5-dimethyl-N-(4-methyl-2H-indazol-5-yl)-4,7-dihydrotetrazolo[1,5-a]pyrimidine-6-carboxamide;
4,5-dimethyl-N-(3-methyl-1H-indol-5-yl)-4,7-dihydrotetrazolo[1,5-a]pyrimidine-6-carboxamide;
4,5-dimethyl-N-(1-methyl-1H-indazol-6-yl)-4,7-dihydrotetrazolo[1,5-a]pyrimidine-6-carboxamide;
4,5-dimethyl-N-(3-methyl-6-(trifluoromethyl)-2H-indazol-5-yl)-4,7-dihydrotetrazolo[1,5-a]pyrimidine-6-carboxamide; and
N-(3-benzamido-2H-indazol-5-yl)-4,5-dimethyl-4,7-dihydrotetrazolo[1,5-a]pyrimidine-6-carboxamide;

or a pharmaceutically acceptable salt thereof.

It is further appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination. Thus, it is contemplated as features described as embodiments of the compounds of Formula IA or Formula I can be combined in any suitable combination.

At various places in the present specification, certain features of the compounds are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

The term “n-membered,” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring and 1,2,3,4-tetrahydro-naphthalene is an example of a 10-membered cycloalkyl group.

At various places in the present specification, variables defining divalent linking groups may be described. It is specifically intended that each linking substituent include both the forward and backward forms of the linking substituent. For example, —NR(CR′R″)ₙ— includes both —NR(CR′R″)ₙ— and —(CR′R″)ₙNR— and is intended to disclose each of the forms individually. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “substituted” means that an atom or group of atoms formally replaces hydrogen as a “substituent” attached to another group. The term “substituted”, unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. The phrase “optionally substituted” means unsubstituted or substituted. The term “substituted” means that a hydrogen atom is removed and replaced by a substituent. A single divalent substituent, e.g., oxo, can replace two hydrogen atoms.

The term “Cₙ₋ₘ” indicates a range which includes the endpoints, wherein n and m are integers and indicate the number of carbons. Examples include C₁₋₄, C₁₋₆ and the like.

The term “alkyl” employed alone or in combination with other terms, refers to a saturated hydrocarbon group that may be straight-chained or branched. The term “Cₙ₋ₘ alkyl”, refers to an alkyl group having n to m carbon atoms. An alkyl group formally corresponds to an alkane with one C—H bond replaced by the point of attachment of the alkyl group to the remainder of the compound. In some embodiments, the alkyl group contains from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkenyl” employed alone or in combination with other terms, refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The term “Cₙ₋ₘ alkenyl” refers to an alkenyl group having n to m carbons. In some embodiments, the alkenyl moiety contains 2 to 6, 2 to 4, or 2 to 3 carbon atoms. Example alkenyl groups include, but are not limited to, ethenyl, n-propenyl, isopropenyl, n-butenyl, sec-butenyl and the like.

The term “alkynyl” employed alone or in combination with other terms, refers to a straight-chain or branched hydrocarbon group corresponding to an alkyl group having one or more triple carbon-carbon bonds. An alkynyl group formally corresponds to an alkyne with one C—H bond replaced by the point of attachment of the alkyl group to the remainder of the compound. The term “Cₙ₋ₘ alkynyl” refers to an alkynyl group having n to m carbons. Example alkynyl groups include, but are not limited to, ethynyl, propyn-1-yl, propyn-2-yl and the like. In some embodiments, the alkynyl moiety contains 2 to 6, 2 to 4, or 2 to 3 carbon atoms.

The term “alkylene”, employed alone or in combination with other terms, refers to a divalent alkyl linking group. An alkylene group formally corresponds to an alkane with two C—H bond replaced by points of attachment of the alkylene group to the remainder of the compound. The term “Cₙ₋ₘ alkylene” refers to an alkylene group having n to m carbon atoms.

Examples of alkylene groups include, but are not limited to, ethan-1,2-diyl, ethan-1,1-diyl, propan-1,3-diyl, propan-1,2-diyl, propan-1,1-diyl, butan-1,4-diyl, butan-1,3-diyl, butan-1,2-diyl, 2-methyl-propan-1,3-diyl and the like.

The term “alkoxy”, employed alone or in combination with other terms, refers to a group of formula —O-alkyl, wherein the alkyl group is as defined above. The term “Cₙ₋ₘ alkoxy” refers to an alkoxy group, the alkyl group of which has n to m carbons. Example alkoxy groups include methoxy, ethoxy, propoxy (e.g., n-propoxy and isopropoxy), t-butoxy and the like. In some embodiments, the alkyl group has 1 to 6, 1 to 4, or 1 to 3 carbon atoms.

The term “Cₙ₋ₘ dialkoxy” refers to a linking group of formula —O—(Cₙ₋ₘ alkyl)-O—, the alkyl group of which has n to m carbons. Example dialkyoxy groups include —OCH₂CH₂O— and OCH₂CH₂CH₂O—. In some embodiments, the two O atoms of a Cₙ₋ₘ dialkoxy group may be attached to the same B atom to form a 5- or 6-membered heterocycloalkyl group.

The term “amino” refers to a group of formula —NH₂.

The term “carbonyl”, employed alone or in combination with other terms, refers to a —C(═O)— group, which also may be written as C(O).

The term “cyano” or “nitrile” refers to a group of formula —C≡N, which also may be written as —CN.

The terms “halo” or “halogen”, used alone or in combination with other terms, refers to fluoro, chloro, bromo and iodo. In some embodiments, “halo” refers to a halogen atom selected from F, Cl, or Br. In some embodiments, halo groups are F.

The term “haloalkyl” as used herein refers to an alkyl group in which one or more of the hydrogen atoms has been replaced by a halogen atom. The term “Cₙ₋ₘ haloalkyl” refers to a Cₙ₋ₘ alkyl group having n to m carbon atoms and from at least one up to {2(n to m)+1} halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CH₂F, CCl₃, CHCl₂, C₂Cl₅ and the like. In some embodiments, the haloalkyl group is a fluoroalkyl group.

The term “haloalkoxy”, employed alone or in combination with other terms, refers to a group of formula —O-haloalkyl, wherein the haloalkyl group is as defined above. The term “Cₙ₋ₘ haloalkoxy” refers to a haloalkoxy group, the haloalkyl group of which has n to m carbons. Example haloalkoxy groups include trifluoromethoxy and the like. In some embodiments, the haloalkoxy group has 1 to 6, 1 to 4, or 1 to 3 carbon atoms.

The term “oxo” refers to an oxygen atom as a divalent substituent, forming a carbonyl group when attached to carbon, or attached to a heteroatom forming a sulfoxide or sulfone group, or an N-oxide group. In some embodiments, heterocyclic groups may be optionally substituted by 1 or 2 oxo (═O) substituents.

The term “sulfido” refers to a sulfur atom as a divalent substituent, forming a thiocarbonyl group (C═S) when attached to carbon.

The term “oxidized” in reference to a ring-forming N atom refers to a ring-forming N-oxide.

The term “oxidized” in reference to a ring-forming S atom refers to a ring-forming sulfonyl or ring-forming sulfinyl.

The term “aromatic” refers to a carbocycle or heterocycle having one or more polyunsaturated rings having aromatic character (i.e., having (4n+2) delocalized π (pi) electrons where n is an integer).

The term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms.

Aryl groups include, e.g., phenyl, naphthyl, and the like. In some embodiments, aryl groups have from 6 to about 10 carbon atoms. In some embodiments aryl groups have 6 carbon atoms. In some embodiments aryl groups have 10 carbon atoms. In some embodiments, the aryl group is phenyl.

The term “heteroaryl” or “heteroaromatic,” employed alone or in combination with other terms, refers to a monocyclic or polycyclic aromatic heterocycle having at least one heteroatom ring member selected from sulfur, oxygen and nitrogen. In some embodiments, the heteroaryl ring has 1, 2, 3 or 4 heteroatom ring members independently selected from nitrogen, sulfur and oxygen. In some embodiments, any ring-forming N in a heteroaryl moiety can be an N-oxide. In some embodiments, the heteroaryl has 5-14 ring atoms including carbon atoms and 1, 2, 3 or 4 heteroatom ring members independently selected from nitrogen, sulfur and oxygen. In some embodiments, the heteroaryl has 5-10 ring atoms including carbon atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. In other embodiments, the heteroaryl is an eight-membered, nine-membered or ten-membered fused bicyclic heteroaryl ring. Example heteroaryl groups include, but are not limited to, pyridinyl (pyridyl), pyrimidinyl, pyrazinyl, pyridazinyl, and the like.

A five-membered heteroaryl ring is a heteroaryl group having five ring atoms wherein one or more (e.g., 1, 2 or 3) ring atoms are independently selected from N, O and S.

Exemplary five-membered ring heteroaryls include thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl and 1,3,4-oxadiazolyl.

A six-membered heteroaryl ring is a heteroaryl group having six ring atoms wherein one or more (e.g., 1, 2 or 3) ring atoms are independently selected from N, O and S. Exemplary six-membered ring heteroaryls are pyridyl, pyrazinyl, pyrimidinyl, triazinyl, isoindolyl, and pyridazinyl.

The term “cycloalkyl,” employed alone or in combination with other terms, refers to a non-aromatic hydrocarbon ring system (monocyclic, bicyclic or polycyclic), including cyclized alkyl and alkenyl groups. The term “Cₙ₋ₘ cycloalkyl” refers to a cycloalkyl that has n to m ring member carbon atoms. Cycloalkyl groups can include mono- or polycyclic (e.g., having 2, 3 or 4 fused rings) groups and spirocycles. Cycloalkyl groups can have 3, 4, 5, 6 or 7 ring-forming carbons (C₃₋₇). In some embodiments, the cycloalkyl group has 3 to 6 ring members, 3 to 5 ring members, or 3 to 4 ring members. In some embodiments, the cycloalkyl group is monocyclic. In some embodiments, the cycloalkyl group is monocyclic or bicyclic.

In some embodiments, the cycloalkyl group is a C₃₋₆ monocyclic cycloalkyl group. Ring-forming carbon atoms of a cycloalkyl group can be optionally oxidized to form an oxo or sulfido group. Cycloalkyl groups also include cycloalkylidenes. In some embodiments, cycloalkyl is cyclopropyl, cyclobutyl, cyclopentyl or cyclohexyl. Also included in the definition of cycloalkyl are moieties that have one or more aromatic rings fused (i.e., having a bond in common with) to the cycloalkyl ring, e.g., benzo or thienyl derivatives of cyclopentane, cyclohexane and the like. A cycloalkyl group containing a fused aromatic ring can be attached through any ring-forming atom including a ring-forming atom of the fused aromatic ring. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, bicyclo[1.1.1]pentanyl, bicyclo[2.1.1]hexanyl, and the like. In some embodiments, the cycloalkyl group is cyclopropyl, cyclobutyl, cyclopentyl, or cyclohexyl.

The term “heterocycloalkyl,” employed alone or in combination with other terms, refers to a non-aromatic ring or ring system, which may optionally contain one or more alkenylene groups as part of the ring structure, which has at least one heteroatom ring member independently selected from nitrogen, sulfur, oxygen and phosphorus, and which has 4-10 ring members, 4-7 ring members, or 4-6 ring members. Included within the term “heterocycloalkyl” are monocyclic 4-, 5-, 6- and 7-membered heterocycloalkyl groups. Heterocycloalkyl groups can include mono- or bicyclic (e.g., having two fused or bridged rings) or spirocyclic ring systems. In some embodiments, the heterocycloalkyl group is a monocyclic group having 1, 2 or 3 heteroatoms independently selected from nitrogen, sulfur and oxygen. Ring-forming carbon atoms and heteroatoms of a heterocycloalkyl group can be optionally oxidized to form an oxo or sulfido group or other oxidized linkage (e.g., C(O), S(O), C(S) or S(O)₂, N-oxide etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the heterocycloalkyl ring, e.g., benzo or thienyl derivatives of piperidine, morpholine, azepine, etc. A heterocycloalkyl group containing a fused aromatic ring can be attached through any ring-forming atom including a ring-forming atom of the fused aromatic ring.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, di acetyl tartaric acid, dibenzoyltartaric acid, mandelic acid, malic acid, lactic acid or the various optically active camphorsulfonic acids such as (3-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

In some embodiments, the compounds of the invention have the (R)-configuration. In other embodiments, the compounds have the (R)-configuration. In compounds with more than one chiral centers, each of the chiral centers in the compound may be independently (R) or (5), unless otherwise indicated.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, e.g., 1H- and 3H-imidazole, 1H-, 2H- and 4H-1,2,4-triazole, 1H- and 2H-isoindole and 1H- and 2H-pyrazole. Tautomeric forms can be in equilibrium or sterically locked into one form by appropriate substitution.

Compounds of the invention can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can used in various studies such as NMR spectroscopy, metabolism experiments, and/or assays.

Substitution with heavier isotopes such as deuterium, may afford certain therapeutic advantages resulting from greater metabolic stability, for example, increased in vivo half-life or reduced dosage requirements, and hence may be preferred in some circumstances. (A. Kerekes et. al. J. Med Chem. 2011, 54, 201-210; R. Xu et. al. J. Label Compd Radiopharm. 2015, 55, 308-312).

The term, “compound,” as used herein is meant to include all stereoisomers, geometric isomers, tautomers and isotopes of the structures depicted. The term is also meant to refer to compounds of the inventions, regardless of how they are prepared, e.g., synthetically, through biological process (e.g., metabolism or enzyme conversion), or a combination thereof.

All compounds, and pharmaceutically acceptable salts thereof, can be found together with other substances such as water and solvents (e.g., hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds of the invention, or salts thereof, are substantially isolated. By “substantially isolated” is meant that the compound is at least partially or substantially separated from the environment in which it was formed or detected. Partial separation can include, e.g., a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature,” as used herein, are understood in the art, and refer generally to a temperature, e.g., a reaction temperature, that is about the temperature of the room in which the reaction is carried out, e.g., a temperature from about 20° C. to about 30° C.

The present invention also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol or butanol) or acetonitrile (MeCN) are preferred. Lists of suitable salts are found in Remington's Pharmaceutical Sciences, 17^{th} Ed., (Mack Publishing Company, Easton, 1985), p. 1418, Berge et al., J. Pharm. Sci., 1977, 66(1), 1-19 and in Stahl et al., Hand book of Pharmaceutical Salts; Properties, Selection, and Use, (Wiley, 2002). In some embodiments, the compounds described herein include the N-oxide forms.

### Synthesis

Compounds of the invention, including salts thereof, can be prepared using known organic synthesis techniques and can be synthesized according to any of numerous possible synthetic routes, such as those in the Schemes below.

The reactions for preparing compounds of the invention can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry; Reactions, Mechanisms, and Structure, 6^{th} Ed. (Wiley, 2007); Peturssion et al., “Protecting Groups in Carbohydrate Chemistry,” J. Chem. Educ., 1997, 77(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high performance liquid chromatography (HPLC) or thin layer chromatography (TLC).

The Schemes below provide general guidance in connection with preparing the compounds of the invention. One skilled in the art would understand that the preparations shown in the Schemes can be modified or optimized using general knowledge of organic chemistry to prepare various compounds of the invention.

Compounds of Formula IA can be prepared, e.g., using a process as illustrated in the schemes below.

Compounds of Formula (1-3) and (1-4) with a variety of substitution such as those described herein can be prepared using a process as illustrated in Scheme 1. In the process depicted in Scheme 1, an appropriately substituted amine is coupled with an appropriately substituted carboxylic acid using a peptide coupling reagent (e.g., 1-[bis(dimethylamino)methylene]-1H-1,2,3-triazolo[4,5-b]pyridinium 3-oxid hexafluorophosphate (“HATU”) or 1-ethyl-3-(3-dimethylaminopropyl)carbodiimide) in the presence of a base (e.g., triethylamine or Hunig's base) to provide a compound of Formula (1-3). Compounds of Formula (1-3) can be converted to compounds of Formula (1-4) using an appropriate thiation reagent (e.g., Lawesson's reagent or P₄S₁₀).

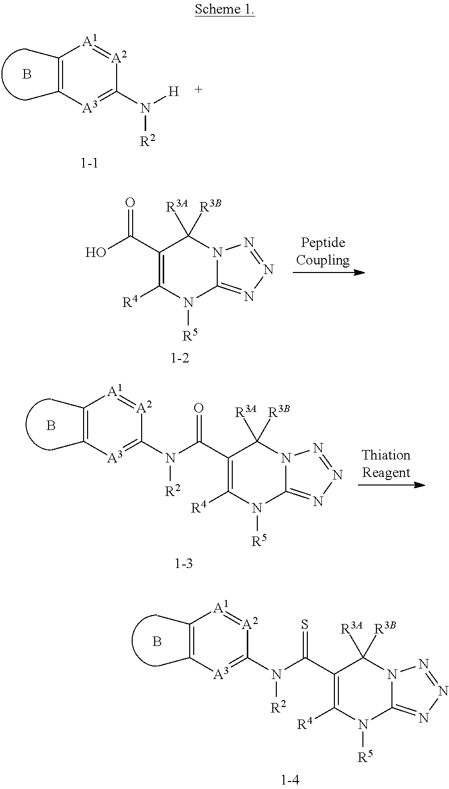

Compound of Formula (1-2) can be prepared using a process as illustrated in Scheme 2. In the process depicted in Scheme 2, an appropriately substituted amine is treated with a halide (R⁵X; X=I, Cl, or Br) in the presence of a base (e.g., Cs₂CO₃) to provide a compound of Formula (2-2). A compound of Formula (2-2) can be saponified with a base (e.g., LiOH or NaOH) to provide a compound of Formula (1-2).

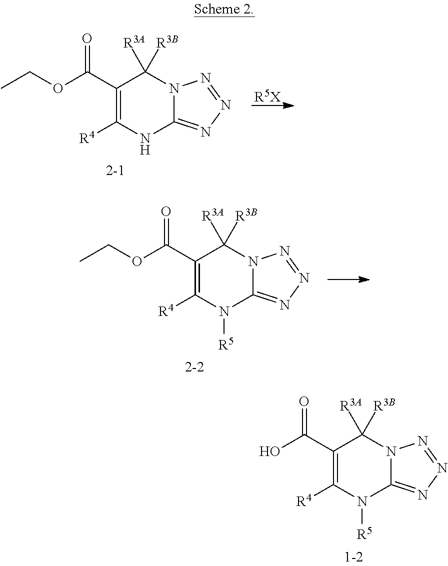

### LRRK2

Over-activation of LRRK2 kinase activity, e.g., in kinase mutant G2019S, is a mechanism in alpha-synuclein related neurodegeneration, and is implicated in diseases that are characterized by the formation of Lewy bodies. Compounds as described herein, e.g., compounds of Formula IA or Formula I, exhibit inhibitory activity against LRRK2 kinase, including LRRK2 mutant kinase, such as mutant G2019S. Kinase activity can be determined using a kinase assay, which typically employs a kinase substrate and a phosphate group donor, such as ATP (or a derivative thereof). An exemplary kinase assay is described in Example A.

The present disclosure provides methods of modulating (e.g., inhibiting) LRRK2 activity, by contacting LRRK2 with a compound of the invention, or a pharmaceutically acceptable salt thereof. In some embodiments, the contacting can be administering to a patient a compound provided herein, or a pharmaceutically acceptable salt thereof. In certain embodiments, the compounds of the present disclosure, or pharmaceutically acceptable salts thereof, are useful for therapeutic administration to treat neurodegenerative disease. For example, a method of treating a disease or disorder associated with inhibition of LRRK2 interaction can include administering to a patient in need thereof a therapeutically effective amount of a compound provided herein, or a pharmaceutically acceptable salt thereof. The compounds of the present disclosure can be used alone, in combination with other agents or therapies or as an adjuvant or neoadjuvant for the treatment of diseases or disorders, including neurodegenerative diseases. For the uses described herein, any of the compounds of the disclosure, including any of the embodiments thereof, may be used.

Compounds and compositions as described herein, e.g., compounds of Formula IA or Formula I are useful in the treatment and/or prevention of LRRK2 kinase mediated disorders, including LRRK2 kinase mutant mediated diseases. LRRK2 kinase mutant G2019S mediated diseases include, but are not limited to, neurological diseases such as Parkinson's disease and other Lewy body diseases such as Parkinson disease with dementia, Parkinson's disease at risk syndrome, dementia with Lewy bodies (e.g., diffuse Lewy body disease (DLBD), Lewy body dementia, Lewy body disease, cortical Lewy body disease or senile dementia of Lewy type), Lewy body variant of Alzheimer's disease (i.e., diffuse Lewy body type of Alzheimer's disease), combined Parkinson's disease and Alzheimer's disease, as well as diseases associated with glial cortical inclusions, such as syndromes identified as multiple system atrophy, including striatonigral degeneration, olivopontocerebellar atrophy, and Shy-Drager syndrome, or other diseases associated with Parkinsonism, such as Hallervorden-Spatz syndrome (also referred to as Hallervorden-Spatz disease), fronto-temporal dementia, Sandhoff disease, progressive supranuclear palsy, corticobasal degeneration, autonomic dysfunctions (e.g., postural or orthostatic hypotension), cerebellar dysfunctions, ataxia, movement disorders, cognitive deterioration, sleep disorders, hearing disorders, tremors, rigidity (e.g., joint stiffness, increased muscle tone), bradykinesia, akinesia and postural instability (failure of postural reflexes, along other disease related factors such as orthostatic hypotension or cognitive and sensory changes, which lead to impaired balance and falls); cancers, including melanoma, acute myelogenous leukemia, breast carcinoma, lung adenocarincoma, prostate adenocarcinoma, renal cell carcinoma, and papillary thyroid carcinoma; autoimmune diseases such as Inflammatory Bowel Disease (e.g. Crohn's disease and ulcerative colitis); and leprosy.

In some embodiments, a method of treating a disease is provided comprising administering to a patient in need thereof a therapeutically effective amount of a compound selected from the group consisting of Formula IA or Formula I, or a pharmaceutically acceptable salt thereof, wherein the disease is selected from the group consisting of Parkinson's disease, Parkinson disease with dementia, Parkinson's disease at risk syndrome, dementia with Lewy bodies, Lewy body variant of Alzheimer's disease, combined Parkinson's disease and Alzheimer's disease, multiple system atrophy, striatonigral degeneration, olivopontocerebellar atrophy, Shy-Drager syndrome, Hallervorden-Spatz syndrome, fronto-temporal dementia, Sandhoff disease, progressive supranuclear palsy, corticobasal degeneration, postural hypotension, orthostatic hypotension, cerebellar dysfunctions, ataxia, movement disorders, cognitive deterioration, sleep disorders, hearing disorders, tremors, rigidity, bradykinesia, akinesia, postural instability, melanoma, acute myelogenous leukemia, breast carcinoma, lung adenocarincoma, prostate adenocarcinoma, renal cell carcinoma, papillary thyroid carcinoma, Crohn's disease, ulcerative colitis, and leprosy.

In some embodiments, a method of treating a neurological disease is provided comprising administering to a patient in need thereof a therapeutically effective amount of a compound selected from the group consisting of Formula IA or Formula I, or a pharmaceutically acceptable salt thereof, wherein the neurological disease is selected from the group consisting of Parkinson's disease, Parkinson disease with dementia, Parkinson's disease at risk syndrome, dementia with Lewy bodies, Lewy body variant of Alzheimer's disease, combined Parkinson's disease and Alzheimer's disease, multiple system atrophy, striatonigral degeneration, olivopontocerebellar atrophy, Shy-Drager syndrome, Hallervorden-Spatz syndrome, fronto-temporal dementia, Sandhoff disease, progressive supranuclear palsy, corticobasal degeneration, postural hypotension, orthostatic hypotension, cerebellar dysfunctions, ataxia, movement disorders, cognitive deterioration, sleep disorders, hearing disorders, tremors, rigidity, bradykinesia, akinesia, and postural instability.

In some embodiments, a method of treating a neurological disease is provided comprising administering to a patient in need thereof a therapeutically effective amount of a compound selected from the group consisting of Formula IA or Formula I, or a pharmaceutically salt thereof, wherein the neurological disease is selected from the group consisting of Parkinson's disease, Parkinson disease with dementia, Parkinson's disease at risk syndrome, dementia with Lewy bodies, Lewy body variant of Alzheimer's disease, combined Parkinson's disease and Alzheimer's disease, multiple system atrophy, striatonigral degeneration, olivopontocerebellar atrophy, and Shy-Drager syndrome.

In some embodiments, a method of treating Parkinson's disease is provided comprising administering to a patient in need thereof a therapeutically effective amount of a compound selected from the group consisting of Formula IA or Formula I, or a pharmaceutically acceptable salt thereof.

In some embodiments, a method of treating a cancer is provided comprising administering to a patient in need thereof a therapeutically effective amount of a compound selected from the group consisting of Formula IA or Formula I, or a pharmaceutically acceptable salt thereof, wherein the cancer is selected from the group consisting of melanoma, acute myelogenous leukemia, breast carcinoma, lung adenocarincoma, prostate adenocarcinoma, renal cell carcinoma, and papillary thyroid carcinoma.

In some embodiments, a method of treating an autoimmune disease is provided comprising administering to a patient in need thereof a therapeutically effective amount of a compound selected from the group consisting of Formula IA or Formula I, or a pharmaceutically acceptable salt thereof, wherein the autoimmune disease is selected from the group consisting of Crohn's disease and ulcerative colitis.

In some embodiments, a method of treating leprosy is provided comprising administering to a patient in need thereof a therapeutically effective amount of a compound selected from the group consisting of Formula IA or Formula I, or a pharmaceutically acceptable salt thereof, or a composition comprising such compound or salt thereof.

In some embodiments, the compounds as described herein, e.g., compounds of Formula IA or Formula I, are inhibitors of LRRK2 kinase activity. In some embodiments, the compounds as described herein, e.g. compounds of Formula IA or Formula I, are inhibitors of LRRK2 mutant kinase activity. In some embodiments, the compounds as described herein, e.g. compounds of Formula IA or Formula I, are inhibitors of LRRK2 mutant G2019S kinase activity.

Compounds as described herein, e.g., compounds of Formula IA or Formula I, exhibit cellular biological activities, including but not limited to reduction in phosphorylation of ser910 or ser935 in HEK-293 cells transfected with either wild-type LRRK2 or LRRK2 G2019S mutant.

In some embodiments, compounds of Formula IA or Formula I are selective LRRK2 G2019S mutant inhibitors as compared to wild-type LRRK2.

As used herein, the term “contacting” refers to the bringing together of the indicated moieties in an in vitro system or an in vivo system such that they are in sufficient physical proximity to interact.

The terms “individual” or “patient,” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

As used herein, the term “selective” or “selectivity” as it relates to kinase activity, means that a compound as described herein, e.g. a compound of Formula IA or Formula I, is a more potent inhibitor of a particular kinase, such as LRRK2 kinase, when compared to another kinase. While LRRK2 has other enzymatic activities, it is understood that when inhibitory activity or selectivity of LRRK2, or any mutation thereof, is mentioned, it is the LRRK2 kinase activity that is being referred to, unless clearly stated otherwise. As such, selectivity of LRRK2 relative to another kinase indicates a comparison of the IC₅₀ of a compound on the kinase activity of LRRK2 to the IC₅₀ of the compound on the kinase activity of another kinase. For example, a compound that is 10 fold selective for LRRK2 kinase activity relative to another kinase activity will have a ratio of IC₅₀(other kinase)÷IC₅₀(LRRK2)=10 (or a ratio of IC₅₀(LRRK2)÷IC₅₀(other kinase)=0.1).

In some embodiments, a compound as described herein, e.g., a compound of Formula IA or Formula I, is selective for a LRRK2 mutant over wild type LRRK2. Selectivity of LRRK2 mutants relative to wild type LRRK2 indicates a comparison of the IC₅₀ of a compound on the kinase activity of the mutant LRRK2 to the IC₅₀ of the compound on the kinase activity of wild type LRRK2. For example, a compound that is 10 fold selective for LRRK2 mutant kinase activity relative to wild type LRKK2 kinase activity will have a ratio of IC₅₀(wild type LRRK2)÷IC₅₀(mutant LRRK2)=10. In some embodiments, a compound provided herein is greater than 1 fold selective, greater than 2 fold selective, greater than 5 fold selective, greater than 10 fold selective, greater than 25 fold selective, or greater than 50 fold selective for LRRK2 mutant kinase over wild type LRRK2. In some embodiments, the LRRK2 mutant is LRRK2 G2019S.

The term “LRRK2-mediated condition”, “Leucine-rich repeat kinase 2 mediated disorder” or any other variation thereof, as used herein means any disease or other condition in which LRRK2, including any mutations thereof, is known to play a role, or a disease state that is associated with elevated activity or expression of LRRK2, including any mutations thereof. For example, a “LRRK2-mediated condition” may be relieved by inhibiting LRRK2 kinase activity. Such conditions include certain neurodegenerative diseases, such as Lewy body diseases, including, but not limited to, Parkinson's disease, Lewy body variant of Alzheimer's disease, combined Parkinson's disease and Alzheimer's disease, dementia with Lewy bodies, diffuse Lewy body disease, as well as any syndrome identified as multiple system atrophy; certain cancers, such as melanoma, papillary renal cell carcinoma and papillary thyroid carcinoma; certain autoimmune diseases, such as Inflammatory Bowel Disease (e.g. Crohn's disease and ulcerative colitis); and leprosy.

The term “neurodegenerative diseases” includes any disease or condition characterized by problems with movements, such as ataxia, and conditions affecting cognitive abilities (e.g., memory) as well as conditions generally related to all types of dementia. “Neurodegenerative diseases” may be associated with impairment or loss of cognitive abilities, potential loss of cognitive abilities and/or impairment or loss of brain cells. Exemplary “neurodegenerative diseases” include Alzheimer's disease, Parkinson's disease, amyotrophic lateral sclerosis (ALS), Down syndrome, dementia, multi-infarct dementia, mild cognitive impairment (MCI), epilepsy, seizures, Huntington's disease, neurodegeneration induced by viral infection (e.g. AIDS, encephalopathies), traumatic brain injuries, as well as ischemia and stroke.

“Neurodegenerative diseases” also includes any undesirable condition associated with the disease. For instance, a method of treating a neurodegenerative disease includes methods of treating or preventing loss of neuronal function characteristic of neurodegenerative disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Combination Therapies

One or more additional pharmaceutical agents or treatment methods can be used in combination with a compound of Formula IA or Formula I for treatment of LRRK2-associated diseases, disorders, or conditions, or diseases or conditions as described herein. The agents can be combined with the present compounds in a single dosage form, or the agents can be administered simultaneously or sequentially as separate dosage forms. In some embodiments, the additional pharmaceutical agent is a dopamine precursor, including, for example, levodopa, melevodopa, and etilevodopa. In some embodiments, the additional pharmaceutical agent is a dopamine agonist, including, for example, pramipexole, ropinorole, apomorphine, rotigotine, bromocriptine, cabergoline, and pergolide. In some embodiments, the additional pharmaceutical agent is a monamine oxidase B (“MAO B”) inhibitor, including, for example, selegiline and rasagiline. In some embodiments, the additional pharmaceutical agent is a catechol O-methyltransferase (“COMT”) inhibitor, including, for example, tolcapone and entacapone. In some embodiments, the additional pharmaceutical agent is an anticholinergic agent including, for example, benztropine, trihexyphenidyl, procyclidine, and biperiden. In some embodiments, the additional pharmaceutical agent is a glutamate (“NMDA”) blocking drug, including, for example, amantadine. In some embodiments, the additional pharmaceutical agent is an adenosine A2a antagonist, including, for example, istradefylline and preladenant. In some embodiments, the additional pharmaceutical agent is a 5-HT1a antagonist, including, for example, piclozotan and pardoprunox. In some embodiments, the additional pharmaceutical agent is an alpha 2 antagonist, including, for example, atipamezole and fipamezole.

### Formulations, Dosage Forms, and Administration

When employed as pharmaceuticals, the compounds of the present disclosure can be administered in the form of pharmaceutical compositions. Thus the present disclosure provides a composition comprising a compound of Formula IA or Formula I or any of the formulas as described herein, a compound as recited in any of the claims and described herein, or a pharmaceutically acceptable salt thereof, or any of the embodiments thereof, and at least one pharmaceutically acceptable carrier. These compositions can be prepared in a manner well known in the pharmaceutical arts, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, the compound of the present disclosure or a pharmaceutically acceptable salt thereof, in combination with one or more pharmaceutically acceptable carriers. In some embodiments, the composition is suitable for topical administration. In making the compositions of the invention, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions and sterile packaged powders.

In some embodiments, the composition is a sustained release composition comprising at least one compound described herein, or a pharmaceutically acceptable salt thereof, and at least one pharmaceutically acceptable carrier or excipient

The compositions can be formulated in a unit dosage form, each dosage containing from about 5 to about 1,000 mg (1 g). The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms and the like.

The therapeutic dosage of a compound of the present invention can vary according to, e.g., the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers.

### EXAMPLES

Experimental procedures for compounds of the invention are provided below. Where the preparation of starting materials is not described, these are commercially available, known in the literature, or readily obtainable by those skilled in the art using standard procedures. Where it is stated that compounds were prepared analogously to earlier examples or intermediates, it will be appreciated by the skilled person that the reaction time, number of equivalents of reagents and temperature can be modified for each specific reaction and that it may be necessary or desirable to employ different work-up or purification techniques. Where reactions are carried out using microwave irradiation, the microwave used is a Biotage Initiator. The actual power supplied varies during the course of the reaction in order to maintain a constant temperature.

All solvents used were commercially available and were used without further purification. Reactions were typically run using anhydrous solvents under an inert atmosphere of nitrogen.

### Liquid Chromatography-Mass Spectrometry Method A

Total ion current (TIC) and DAD UV chromatographic traces together with MS and UV spectra associated with the peaks were taken on a UPLC/MS Acquity™ system equipped with PDA detector and coupled to a Waters single quadrupole mass spectrometer operating in alternated positive and negative electrospray ionization mode. [LC/MS-ES (+/−): analyses performed using an Acquity UPLC™ CSH, C18 column (50×2.1 mm, 1.7 μm particle size), column temperature 40° C., mobile phase: A—water+0.1% HCOOH/B—CH₃CN+0.1% HCOOH, flow rate: 1.0 mL/min, runtime=2.0 min, gradient: t=0 min 3% B, t=1.5 min 99.9% B, t=1.9 min 99.9% B, t=2.0 min 3% B, stop time 2.0 min. Positive ES 100-1000, Negative ES 100-1000, UV detection DAD 210-350 nm.

### Liquid Chromatography-Mass Spectrometry Method B

Total ion current (TIC) and DAD UV chromatographic traces together with MS and UV spectra associated with the peaks were taken on a UPLC/MS Acquity™ system equipped with PDA detector and coupled to a Waters single quadrupole mass spectrometer operating in alternated positive and negative electrospray ionization mode. [LC/MS-ES (+/−): analyses performed using an Acquity UPLC™ BEH, C18 column (50×2.1 mm, 1.7 μm particle size), column temperature 40° C., mobile phase: A—0.1% v/v aqueous ammonia solution pH 10/B—CH₃CN, flow rate: 1.0 mL/min, runtime=2.0 min, gradient: t=0 min 3% B, t=1.5 min 99.9% B, t=1.9 min 99.9% B, t=2.0 min 3% B, stop time 2.0 min. Positive ES 100-1000, Negative ES 100-1000, UV detection DAD 210-350 nm.

### Other Analytical Methods

¹H Nuclear magnetic resonance (NMR) spectroscopy was carried out using one of the following instruments: a Bruker Avance 400 instrument equipped with probe DUAL 400 MHz SI, a Bruker Avance 400 instrument equipped with probe 6 SI 400 MHz 5 mm ¹H-¹³C ID, a Bruker Avance III 400 instrument with nanobay equipped with probe Broadband BBFO 5 mm direct, a 400 MHz Agilent Direct Drive instrument with ID AUTO-X PFG probe, all operating at 400 MHz, or an Agilent VNMRS500 Direct Drive instrument equipped with a 5 mm Triple Resonance ¹H{¹³C/^{l5}N} cryoprobe operating at 500 MHz. The spectra were acquired in the stated solvent at around room temperature unless otherwise stated. In all cases, NMR data were consistent with the proposed structures. Characteristic chemical shifts (δ) are given in parts-per-million using conventional abbreviations for designation of major peaks: e.g. s, singlet; d, doublet; t, triplet; q, quartet; dd, doublet of doublets; dt, doublet of triplets; br, broad.

Where thin layer chromatography (TLC) has been used it refers to silica gel TLC using silica gel F254 (Merck) plates, Rf is the distance travelled by the compound divided by the distance travelled by the solvent on a TLC plate. Column chromatography was performed using an automatic flash chromatography (Biotage SP1 or Isolera) system over Biotage silica gel cartridges (KP-Sil or KP-NH) or in the case of reverse phase chromatography over Biotage C18 cartridges (KP-C18).

### Intermediate 1. Ethyl 5-methyl-4H,7H-[1,2,3,4]tetrazolo[1,5-a]pyrimidine-6-carboxylate

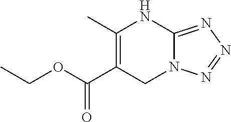

To a mixture of 5-aminotetrazole monohydrate (606 mg, 5.88 mmol), formaldehyde aqueous solution (36.5-38% in H₂O; 477 mg, 5.88 mmol) and ethyl acetate (742 μL, 5.88 mmol) in ethyl alcohol (1.5 mL) was added a catalytic amount of acetic acid (84 μL, 1.47 mmol). The mixture was then heated under microwave irradiation at 120° C. for 10 min. Volatiles were removed under reduced pressure and then the residue was purified on Biotage (C18 30 g cartridge, reverse phase, H₂O/CH₃CN as eluent, 95:5 to 60:40) to afford the title compound as a white solid (765 mg, 3.66 mmol, 62% yield). ¹H NMR (400 MHz, DMSO-d₆) δ 10.89 (s, 1H) 5.10 (d, J=0.66 Hz, 2H) 4.14 (q, J=7.04 Hz, 2H) 2.35 (s, 3H) 1.25 (t, J=7.04 Hz, 3H). MS-ESI (m/z) calcd for C₈H₁₂N₅O₂ [M+H]+: 210.09. Found 210.16.

### Intermediate 2. Ethyl 4,5-dimethyl-4H,7H-[1,2,3,4]tetrazolo[1,5-a]pyrimidine-6-carboxylate

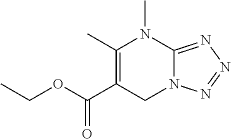 [SMILES: CCOC(=O)C1=C(C)N(C)c2nnnn2C1]

To a solution of Intermediate 1 (200 mg, 0.96 mmol) in DMF (4 mL) was added Mel (119 μL, 1.91 mmol) and Cs₂CO₃ (405 mg, 1.24 mmol) and the mixture was stirred at 50° C. for 1 h. Cooled H₂O (15 mL) was added and the mixture was extracted with EtOAc (15 mL). The organic layer was separated, dried over Na₂SO₄, filtered and concentrated. The residue was purified on Biotage (10 g cartridge, normal phase, cyclohexane/EtOAc as eluent, 100:0 to 20:80) to afford the title compound as a colorless oil (95 mg, 0.426 mmol 44% yield). MS-ESI (m/z) calculated for C₉H₁₃N₅O₂ [M+H]+: 224.11. Found 224.19.

### Intermediate 3. 4,5-Dimethyl-4H,7H-[1,2,3,4]tetrazolo[1,5-a]pyrimidine-6-carboxylic Acid

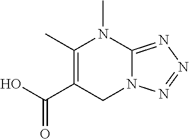

LiOH (54 mg, 1.28 mmol) was added to a solution of Intermediate 2 (95 mg, 0.43 mmol) in an EtOH/THF/H₂O mixture (4:1:0.6, 4.15 mL). The mixture was stirred at 55° C. for 1 h. Subsequently, the mixture was acidified with 1M HCl and extracted with DCM (10 mL, 3×). The pH of the aqueous layer was brought to pH=7 with 1M NaOH and extracted with DCM (10 mL). The combined organic layers were concentrated in vacuo to obtain the title compound (110 mg crude, 0.43 mmol theoretical) as a white solid, which was used without further purification. MS-ESI (m/z) calcd for C₉H₁₃N₅O₂ [M+H]+: 196.08. Found 196.12.

### Intermediate 4. 4,5,7-Trimethyl-4,7-dihydrotetrazolo[1,5-a]pyrimidine-6-carboxylic Acid

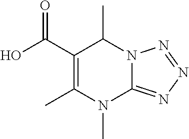

### Step 1. Ethyl 5,7-dimethyl-4,7-dihydrotetrazolo[1,5-a]pyrimidine-6-carboxylate

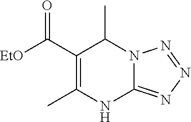 [SMILES: CCOC(=O)C1=C(C)Nc2nnnn2C1C]

A mixture of 5-aminotetrazole monohydrate (7.22 g, 70.00 mmol), ethyl acetoacetate (8.85 mL, 70.00 mmol) and acetaldehyde (5.89 mL, 105.00 mmol) in water (300 mL) was heated at reflux for 9 hours. Heating was switched off and the suspension was stirred at room temperature for 15 hours. The solid formed was filtered to obtain ethyl 5,7-dimethyl-4H,7H-[1,2,3,4]tetrazolo[1,5-a]pyrimidine-6-carboxylate (9.56, 61%) as a white solid. MS-ESI (m/z) calcd for C₉H₁₄N₁₅O₂ [M+H]+: 224.11. Found 224.0.

### Step 2. Ethyl 4,5,7-trimethyl-4,7-dihydrotetrazolo[1,5-a]pyrimidine-6-carboxylate

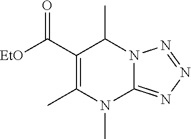 [SMILES: CCOC(=O)C1=C(C)N(C)c2nnnn2C1C]

To a suspension of ethyl 5,7-dimethyl-4,7-dihydrotetrazolo[1,5-a]pyrimidine-6-carboxylate (9.56 g, 43 mmol) in CH₃CN (250 mL) was added Mel (2.92 mL, 47 mmol) and Cs₂CO₃ (15.35 g, 47 mmol) and the mixture was stirred at 50° C. for 1 hour. The solvent was evaporated and water was added. The mixture was then stirred for 2 hours and DCM was added; the biphasic solution was stirred for 10 minutes. The two phases were separated and the organic layer was kept while DCM was added to the water layer and the biphasic solution was stirred for 10 minutes. The two phases were separated, the water layer was discarded while the organic layer was combined with the previous one, passed through a phase separator and evaporated to obtain ethyl 4,5,7-trimethyl-4,7-dihydrotetrazolo[1,5-a]pyrimidine-6-carboxylate (10.16 g, 100%) as a clear oil. MS-ESI (m/z) calcd for C₁₀H₁₆N₅O₂ [M+H]+: 238.12. Found 238.0.

### Step 3. 4,5,7-trimethyl-4,7-dihydrotetrazolo[1,5-a]pyrimidine-6-carboxylic Acid

To a solution of ethyl 4,5,7-trimethyl-4,7-dihydrotetrazolo[1,5-a]pyrimidine-6-carboxylate (10.16 g, 43 mmol) in THF (80 mL) was added a suspension of LiOH (3.08 g, 128 mmol) in water (25 mL) and the mixture was stirred at 55° C. for 24 hours. The THF was evaporated and the slurry was diluted with water, then concentrated HCl was added dropwise at 0° C. until pH 1 and the mixture was stirred at 0° C. for 30 minutes. The solid formed was filtered under vacuum to obtain 4,5,7-trimethyl-4,7-dihydrotetrazolo[1,5-a]pyrimidine-6-carboxylic acid (Intermediate 4; 7.84 g, 87%) as a white solid. ¹H NMR (400 MHz, DMSO-d₆) δ 12.75 (br s, 1H), 5.67 (s, 1H), 3.47 (s, 3H), 3.3 (br s, 3H), 1.47 (s, 3H). MS-ESI (m/z) calcd for C₈H₁₂N₅O₂ [M+H]+: 210.09. Found 210.0.

### Separation of Enantiomers of 4,5,7-Trimethyl-4,7-dihydrotetrazolo[1,5-a]pyrimidine-6-carboxylic Acid

Racemic 4,5,7-trimethyl-4,7-dihydrotetrazolo[1,5-a]pyrimidine-6-carboxylic (Intermediate 4) was subjected to semi-preparative chiral HPLC. Column: Chiralpak AS-H (25×2.0 cm), 5 μm. Mobile phase: n-hexane/(EtOH+0.1% formic acid) 85/15% v/v. Flow rate (mL/min): 17 mL/min. DAD detection: 220 nm. Loop: 1000 μL. Total amount: 850 mg. Solubilization: 850 mg in 62 mL (42 mL EtOH+0.1% formic acid and 20 mL of hexafluoro-2-propanol)=13.7 mg/mL. Injection: 13.7 mg.

### First Eluting Enantiomer (Intermediate 4a)

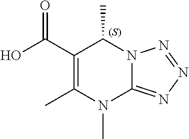

(7S)-4,5,7-Trim ethyl-4,7-dihydrotetrazolo[1,5-a]pyrimidine-6-carboxylic acid (340 mg, 1.625 mmol, 40% yield, white solid). MS-ESI (m/z) calcd for C₈H₁₂N₅O₂ [M+H]+: 210.09. Found 209.9. Analytical chiral HPLC (e.e.=100%, 11.4 min).

### Second Eluting Enantiomer (Intermediate 4b)

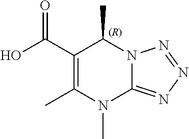

(7R)-4,5,7-Trimethyl-4,7-dihydrotetrazolo[1,5-a]pyrimidine-6-carboxylic acid (335 mg, 1.603 mmol, 39% yield, white solid). MS-ESI (m/z) calcd for C₈H₁₂N₅O₂ [M+H]+: 210.09. Found 209.9. Analytical chiral HPLC (e.e.=100%, 15.0 min).

### Intermediate 5. 3-phenyl-1H-indazol-5-amine

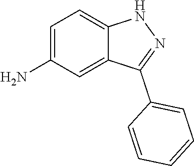 [SMILES: Nc1ccc2[nH]nc(-c3ccccc3)c2c1]

### Step 1. 3-Bromo-1H-indazol-5-amine

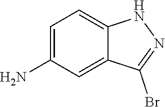 [SMILES: Nc1ccc2[nH]nc(Br)c2c1]

A mixture of 3-bromo-5-nitro-1H-indazole (10 g, 41.32 mmol), ammonium chloride (2.43 g, 45.45 mmol) and iron powder (9.23 g, 165.28 mmol) in EtOH/H₂O (1:1, 200 mL) was stirred at 80° C. for 1 hr. The solids were removed by filtration through a Celite pad and the cake was washed with EtOH. Volatiles were removed under vacuum and the recovered material was re-dissolved in EtOAc. Water was added and the phases were separated. The aqueous layer was extracted with EtOAc (2×) and the combined organic layers washed with water (1×), dried over anhydrous Na₂SO₄ and the solvent was removed under reduced pressure to afford the desired product (8.5 g, 40.0 mmol, 97% yield) as a light brown solid. ¹H NMR (400 MHz, DMSO-d₆) δ 12.90 (s, 1H), 7.27 (d, J=9.0 Hz, 1H), 6.86 (dd, J=2.0, 8.8 Hz, 1H), 6.55 (d, J=1.8 Hz, 1H), 5.01 (s, 2H). MS-ESI (m/z) calcd for C₇H₇BrN₃ [M+H]+: 212.0. Found 212.0.

### Step 2. 3-Phenyl-1H-indazol-5-amine

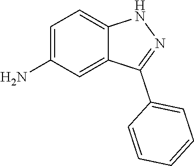 [SMILES: Nc1ccc2[nH]nc(-c3ccccc3)c2c1]

Phenylboronic acid (1.0 g, 8.20 mmol) and 3-bromo-1H-indazol-5-amine (1.16 g, 5.47 mmol) were dissolved in a mixture of DMF (10 mL) and 8.5 mL of an aqueous 2M Na₂CO₃ solution. The mixture was purged with nitrogen for 5 min, and then Pd(PPh₃)₄ (320 mg, 0.27 mmol) was added. The reaction mixture was stirred at 120° C. for 3 hrs. The mixture was then partitioned between water and EtOAc. The phases were separated and the aqueous layer was extracted with EtOAc (2×). The combined organic layers were washed with water (1×), dried over anhydrous Na₂SO₄ and the solvent was removed under reduced pressure. The crude material was purified by flash chromatography on a 55 g NH-silica gel column (cyclohexane/EtOAc, 1:0 to 1:1 as eluent) to afford the impure product which was further purified by reverse phase flash chromatography on a 55 g C18 column (eluting with a gradient of acetonitrile in water from 5% to 20% containing 0.1% formic acid) to afford the desired product, Intermediate 5 (435 mg, 2.08 mmol, 25% yield) as a brownish solid. ¹H NMR (400 MHz, DMSO-d₆) δ 12.78 (s, 1H), 7.90 (d, J=7.5 Hz, 2H), 7.48 (t, J=7.6 Hz, 2H), 7.32 (dd, J=20.1, 8.0 Hz, 2H), 7.12 (s, 1H), 6.83 (d, J=8.7 Hz, 1H). MS-ESI (m/z) calcd for C₁₃H₁₂N₃ [M+H]+: 210.1. Found 210.1.

### Example 1. N-(1H-Indazol-5-yl)-4,5-dimethyl-4H,7H-[1,2,3,4]tetrazolo[1,5-a]pyrimidine-6-carboxamide

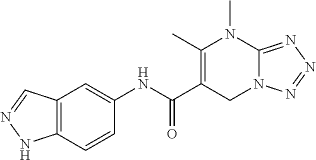 [SMILES: CC1=C(C(=O)Nc2ccc3[nH]ncc3c2)Cn2nnnc2N1C]

Intermediate 3 (110 mg, 0.43 mmol theoretical) was dissolved in DMF (3 mL). TEA (119 μL, 0.85 mmol), 1H-indazol-5-amine (113 mg, 0.85 mmol) and HATU (194 mg, 0.51 mmol) were added at 0° C. and the reaction mixture was stirred at 0° C. for 2 h. The solvent was evaporated, and the residue was taken up in CH₃CN (1 mL, with 0.1% formic acid) and then purified on Biotage (C18 12 g cartridge, reverse phase, water/formic acid 0.1% and ACN/formic acid 0.1% as eluent, 98:2 to 1:9) to give a purple solid (22.3 mg, 0.072 mmol) which was in turn purified on Biotage (10 g cartridge, normal phase, EtOAc/MeOH as eluent, 10:0 to 9:1) to afford the title compound as a pale, pink solid (4.1 mg, 0.013 mmol). ¹H NMR (400 MHz, DMSO-d₆) δ 12.99 (br s, 1H), 9.99 (s, 1H), 8.14 (s, 1H), 8.04 (s, 1H), 7.44-7.55 (m, 2H), 5.28 (s, 2H), 3.43 (s, 3H), 2.25 (s, 3H). MS-ESI (m/z) calcd for C₁₄H₁₅N₈O [M+H]+: 311.13. Found 311.03.

### Example 2. N-(1H-indazol-5-yl)-4,5,7-trimethyl-4H,7H-[1,2,3,4]tetrazolo[1,5-a]pyrimidine-6-carboxamide

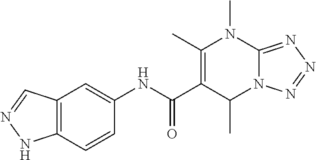 [SMILES: CC1=C(C(=O)Nc2ccc3[nH]ncc3c2)C(C)n2nnnc2N1C]

To a solution of Intermediate 4 (70 mg, 0.34 mmol) and 1H-indazol-5-amine (89 mg, 0.67 mmol) in dry DMF (2 mL) at 0° C., was added HATU (153 mg, 0.402 mmol) and TEA (94 μL, 0.669 mmol) and the resulting mixture was stirred for 2 h at room temperature. H₂O (15 mL) was added and the mixture was extracted with EtOAc (15 mL). The organic layer was separated, concentrated and the residue was purified on Biotage (C18 12 g cartridge, reverse phase, H₂O/formic acid 0.1% and ACN/formic acid 0.1% as eluent, 98:2 to 1:9) to give a solid which was triturated with MeOH to afford the title compound (21 mg, 0.065 mmol, 19% yield), as a pale purple solid. ¹H NMR (400 MHz, DMSO-d₆) δ 13.00 (br s, 1H), 10.16 (s, 1H), 8.16 (s, 1H), 8.04 (s, 1H), 7.44-7.54 (m, 2H), 5.73 (q, J=5.9 Hz, 1H), 3.43 (s, 3H), 2.18 (s, 3H), 1.56 (d, J=6.3 Hz, 3H). MS-ESI (m/z) calcd for C₁₇H₁₂N₄O [M+H]+: 325.14. Found 325.25.

Enantiomers of the title compound were separated using semipreparative chiral HPLC: (Column: Whelk O1 (R,R) (25×2.0 cm), 10 μm; mobile phase: n-hexane/EtOH 40/60% v/v; flow rate (mL/min): 17 mL/min. DAD detection: 220 nm; loop: 3000 μL. total amount: 13 mg; solubilization: 13 mg in 3 mL hexafluoro-2-propanol/EtOH 1/1=4.3 mg/mL; injection: 13 mg/injection). Analytic chiral HPLC: (column: Whelk O1 (R,R) (25×0.46 cm), 10 μm; mobile phase: n-hexane/EtOH 40/60% v/v; flow rate (mL/min): 1.0 ml/min. DAD detection: 220 nm; loop: 25 μL).

### Example 2a; Enantiomer 1 (First Eluting Enantiomer)

100% Pure, e.e.=100%, 1.7 mg, white solid. Analytic chiral HPLC: 17.7 min. Semi-preparative chiral HPLC: 20.7 min. ¹H NMR (400 MHz, DMSO-d₆) δ 12.99 (br s, 1H), 10.17 (s, 1H), 8.16 (s, 1H), 8.05 (s, 1H), 7.43-7.57 (m, 2H), 5.74 (q, J=6.0 Hz, 1H), 3.43 (s, 3H), 2.19 (s, 3H), 1.57 (d, J=6.5 Hz, 3H). MS-ESI (m/z) calcd for C₁₇H₁₂N₄O [M+H]+: 325.14. Found 325.06.

### Example 2b; Enantiomer 2, Second Eluting Enantiomer

99% pure, e.e.=100%, 1.4 mg, white solid. Analytic chiral HPLC: 22.5 min. Semi-preparative chiral HPLC: 27.5 min. ¹H NMR (400 MHz, DMSO-d₆) δ 12.99 (br s, 1H), 10.17 (br s, 1H), 8.16 (s, 1H), 8.05 (s, 1H), 7.29-7.63 (m, 2H), 5.74 (q, J=6.0 Hz, 1H), 3.43 (s, 3H), 2.19 (s, 3H), 1.57 (d, J=6.2 Hz, 3H). MS-ESI (m/z) calcd for C₁₇H₁₂N₄O [M+H]+: 325.14. Found 325.06.

### Example 3. N-(1H-indazol-6-yl)-4,5-dimethyl-4H,7H-[1,2,3,4]tetrazolo[1,5-a]pyrimidine-6-carboxamide

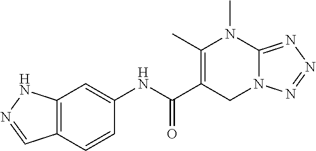 [SMILES: CC1=C(C(=O)Nc2ccc3cn[nH]c3c2)Cn2nnnc2N1C]

Intermediate 3 (70 mg, 0.36 mmol) and 1H-indazol-6-amine (96 mg, 0.72 mmol) were dissolved in dry DMF (2 mL). The solution was cooled to 0° C. with an ice water bath. Triethylamine (0.1 mL, 0.72 mmol) and HATU (164 mg, 0.43 mmol) were then added. The mixture was stirred at 0° C. for 30 min and then at room temperature overnight. The solution was loaded directly onto a 12 g Biotage C18 column and purified by reverse phase chromatography, using a 5-35% gradient of ACN in H₂O containing 0.1% formic acid. The purest fractions were combined and evaporated under reduced pressure to afford the title compound (15 mg, 0.048 mmol, 13% yield) as a white solid. [M+H]^{+ 1}H NMR (400 MHz, DMSO-d6) δ 12.92 (s, 1H), 10.11 (s, 1H), 8.16 (s, 1H), 7.98 (s, 1H), 7.69 (d, J=8.8 Hz, 1H), 7.19 (dd, J=1.7, 8.7 Hz, 1H), 5.29 (s, 2H), 3.44 (s, 3H), 2.25 (s, 3H). MS-ESI (m/z) calcd for C₁₄H₁₅N₈O [M+H]+: 311.13. Found 311.24.

### Example 4. N-(1H-Indazol-5-yl)-5-methyl-4-[2-(morpholin-4-yl)ethyl]-4H,7H-[1,2,3,4]tetrazolo[1,5-a]pyrimidine-6-carboxamide

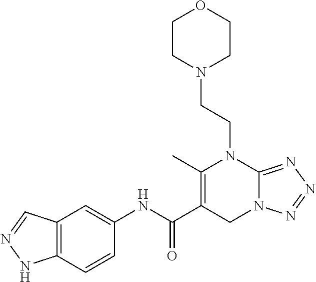 [SMILES: CC1=C(C(=O)Nc2ccc3[nH]ncc3c2)Cn2nnnc2N1CCN1CCOCC1]

### Step 1. Ethyl 5-methyl-4-[2-(morpholin-4-yl)ethyl]-4H,7H-[1,2,3,4]tetrazolo[1,5-a]pyrimidine-6-carboxylate

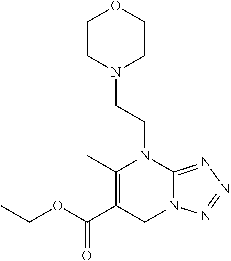 [SMILES: CCOC(=O)C1=C(C)N(CCN2CCOCC2)c2nnnn2C1]

To a solution of Intermediate 1 (120 mg, 0.57 mmol) in dry DMF (3 mL) was added 4-(2-chloroethyl)morpholine hydrochloride (139 mg, 0.75 mmol) and Cs₂CO₃ (561 mg, 1.72 mmol) and the mixture was stirred at 50° C. for 2 h. H₂O (15 mL) was added followed by EtOAc (15 mL). The organic layer was separated, washed with brine, dried over Na₂SO₄, filtered and concentrated. The residue was purified on Biotage (C18 12 g cartridge, reverse phase, H₂O/ACN as eluent, 98:2 to 20:80) to afford the title compound as a colorless oil (37 mg, 0.115 mmol, 20% yield). NMR (400 MHz, DMSO-d₆) δ 5.13 (d, J=0.9 Hz, 2H), 4.16 (q, J=7.0 Hz, 2H), 4.03 (t, J=6.7 Hz, 2H), 3.42-3.60 (m, 4H), 2.52-2.61 (m, 6H), 2.40-2.47 (m, 3H), 1.25 (t, J=7.2 Hz, 3H). MS-ESI (m/z) calcd for C₁₄H₂₃N₅O₃ [M+H]+: 323.18. Found 323.25.

### Step 2. 5-Methyl-4-[2-(morpholin-4-yl)ethyl]-4H,7H-[1,2,3,4]tetrazolo[1,5-a]pyrimidine-6-carboxylic Acid

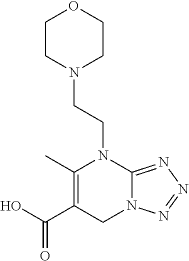 [SMILES: CC1=C(C(=O)O)Cn2nnnc2N1CCN1CCOCC1]

LiOH (14.5 mg, 0.34 mmol) was added to a solution of ethyl 5-methyl-4-[2-(morpholin-4-yl)ethyl]-4H,7H-[1,2,3,4]tetrazolo[1,5-a]pyrimidine-6-carboxylate (37 mg, 0.115 mmol) in an EtOH/THF/H₂O mixture (4:1:0.6, 4.15 mL). The mixture was stirred at 55° C. for 4 h. Subsequently, HCl 1M was added until pH 7 and the mixture was concentrated to obtain the title compound (40 mg, 0.115 mmol theoretical) as crude, which was used in the next step without any other purification. MS-ESI (m/z) calcd for C₁₂H₁₉N₆O₃ [M+H]+: 295.14. Found 295.28.

### Step 3. N-(1H-Indazol-5-yl)-5-methyl-4-[2-(morpholin-4-yl)ethyl]-4H,7H-[1,2,3,4]tetrazolo[1,5-a]pyrimidine-6-carboxamide

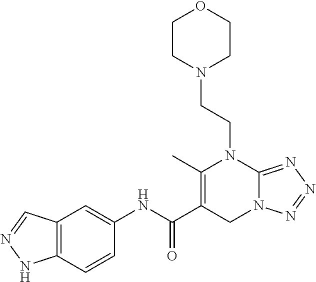 [SMILES: CC1=C(C(=O)Nc2ccc3[nH]ncc3c2)Cn2nnnc2N1CCN1CCOCC1]

5-Methyl-4-[2-(morpholin-4-yl)ethyl]-4H,7H-[1,2,3,4]tetrazolo[1,5-a]pyrimidine-6-carboxylic acid (40 mg crude, 0.115 mmol theoretical) was dissolved in DMF (1.5 mL). TEA (32 μL, 0.23 mmol), 1H-indazol-5-amine (18 mg, 0.14 mmol) and HATU (52 mg, 0.14 mmol) were added at 0° C. and the reaction mixture was stirred for 3 h. A UPLC check of the mixture showed that the reaction was not complete. The temperature was brought to 40° C. and HATU (52 mg, 0.14 mmol) and 1H-indazol-5-amine (18 mg, 0.14 mmol) were added. The reaction was stirred at 40° C. for 3 h. H₂O (15 mL) was added followed by EtOAc (15 mL). The organic layer was separated, concentrated in vacuo and the residue purified on Biotage (NH₂ 11 g cartridge, normal phase, EtOAc/MeOH as eluent, 10:0 to 9:1) to give a solid which was further purified on preparative TLC (NH₂ TLC, normal phase, EtOAc/MeOH as eluent, 10:0 to 9:1) to afford the title compound (4.5 mg, 0.011 mmol, 10% yield), as a white solid. NMR (400 MHz, DMSO-d₆) δ 12.91 (br s, 1H), 10.02 (br s, 1H), 8.14 (s, 1H), 8.04 (s, 1H), 7.41-7.57 (m, 2H), 5.27 (s, 2H), 3.97 (t, J=6.7 Hz, 2H), 3.52 (t, J=4.4 Hz, 4H), 2.56 (t, J=6.7 Hz, 2H), 2.41-2.48 (m, 4H), 2.26 (s, 3H). MS-ESI (m/z) calcd for C₁₉H₂₃N₉O₂ [M+H]+: 410.20. Found 410.32.

### Example 5. 5-Ethyl-N-(1H-indazol-5-yl)-4-methyl-4H,7H-[1,2,3,4]tetrazolo[1,5-a]pyrimidine-6-carboxamide

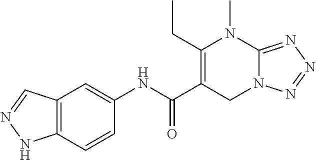 [SMILES: CCC1=C(C(=O)Nc2ccc3[nH]ncc3c2)Cn2nnnc2N1C]

### Step 1. Ethyl 5-ethyl-4H,7H-[1,2,3,4]tetrazolo[1,5-a]pyrimidine-6-carboxylate

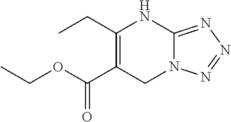 [SMILES: CCOC(=O)C1=C(CC)Nc2nnnn2C1]

To a mixture of 5-aminotetrazole monohydrate (1.03 g, 10.00 mmol), formaldehyde aqueous solution (36.5-38%) (0.73 ml; 10.00 mmol) and ethyl propionylacetate (1.43 mL, 10.00 mmol) in EtOH (3.0 mL) was added acetic acid (140 μL, 2.50 mmol). The mixture was then heated under microwave irradiation (time: 10 min, pre-stirring: 20 sec, temp: 120° C., abs lev: very high, vial: 20 mL). The solvent was evaporated and the residue was purified by column chromatography (C₁₈, ACN in H₂O+0.1% formic acid 0% to 40% in 6 CV, 40% for 5 CV) to obtain the title compound as a white solid (142 mg, 0.63 mmol, 6% yield). ¹H NMR (400 MHz, DMSO-d₆) δ 10.87 (s, 1H), 5.10 (s, 2H), 4.14 (q, J=7.1 Hz, 2H), 2.76 (q, J=7.5 Hz, 2H), 1.24 (t, J=7.1 Hz, 3H), 1.15 (t, J=7.4 Hz, 3H). MS-ESI (m/z) calcd for C₉H₁₄N₅O₂ [M+H]+: 224.11. Found 223.99.

### Step 2. Ethyl 5-ethyl-4-methyl-4H,7H-[1,2,3,4]tetrazolo[1,5-a]pyrimidine-6-carboxylate

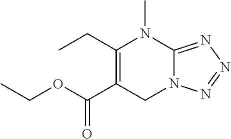 [SMILES: CCOC(=O)C1=C(CC)N(C)c2nnnn2C1]

To a solution of ethyl 5-ethyl-4H,7H-[1,2,3,4]tetrazolo[1,5-a]pyrimidine-6-carboxylate (140 mg, 0.63 mmol) in CH₃CN (5 mL) was added Mel (43 μL, 0.69 mmol) and Cs₂CO₃ (226 mg, 0.69 mmol), and the mixture was stirred at 50° C. for 1 h. The solvent was evaporated and H₂O was added, and the mixture was stirred for 1 h, and then filtered under vacuum to obtain the title compound (75 mg, 50%) as a white solid. ¹H NMR (400 MHz, DMSO-d₆) δ 5.11 (s, 2H), 4.15 (q, J=7.1 Hz, 2H), 3.49 (s, 3H), 2.98 (q, J=7.4 Hz, 2H), 1.25 (t, J=7.1 Hz, 3H), 1.14 (t, J=7.4 Hz, 3H). MS-ESI (m/z) calcd for C₁₀H₁₆N₅O₂ [M+H]+: 238.12. Found 238.02.

### Step 3. 5-Ethyl-4-methyl-4H,7H-[1,2,3,4]tetrazolo[1,5-a]pyrimidine-6-carboxylic Acid

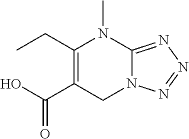

To a solution of ethyl 5-ethyl-4-methyl-4H,7H-[1,2,3,4]tetrazolo[1,5-a]pyrimidine-6-carboxylate (70 mg, 0.30 mmol) in THF (2 mL) was added a solution of LiOH (63 mg, 0.90 mmol) in H₂O (2 mL). The mixture was stirred at 50° C. for 15 h. The THF was evaporated and the water solution was acidified with concentrated HCl, then extracted with EtOAc, dried over Na₂SO₄ and evaporated to obtain the title compound (34 mg, 0.16 mmol, 54% yield) as a white solid. ¹H NMR (400 MHz, DMSO-d₆) δ 12.53 (br s, 1H), 5.07 (s, 3H), 3.47 (s, 3H), 3.00 (q, J=7.4 Hz, 2H), 1.13 (t, J=7.4 Hz, 3H). MS-ESI (m/z) calcd for C₈H₁₂N₅O₂ [M+H]+: 210.09. Found 210.15.

### Step 4. 5-Ethyl-N-(1H-indazol-5-yl)-4-methyl-4H,7H-[1,2,3,4]tetrazolo[1,5-a]pyrimidine-6-carboxamide

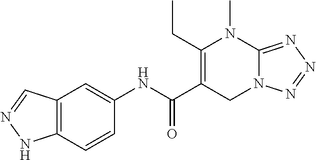 [SMILES: CCC1=C(C(=O)Nc2ccc3[nH]ncc3c2)Cn2nnnc2N1C]

5-Ethyl-4-methyl-4H,7H-[1,2,3,4]tetrazolo[1,5-a]pyrimidine-6-carboxylic acid (34 mg, 0.16 mmol) and 1H-indazol-5-amine (43 mg, 0.32 mmol) were added in dry DMF (1.5 mL) at 0° C., and mixed with HATU (74 mg, 0.2 mmol) and triethylamine (45 μl, 0.32 mmol). The reaction mixture was stirred for 30 min at 0° C. At this point the reaction mixture was concentrated in vacuo, the residue was taken up in CH₃CN (with 0.1% TFA) and then purified by flash chromatography on C₁₈ column (Water/ACN+0.1% formic acid 98:2→10:90) to obtain the target compound as a red solid. The compound was re-purified by silica gel chromatography (from 100% EtOAc to 80/20 EtOAc/MeOH in 12CV) to give the title compound (32 mg, 0.10 mmol, 62% yield) as white solid. ¹H NMR (400 MHz, DMSO-d₆) δ 13.00 (br s, 1H), 9.97 (s, 1H), 8.15 (s, 1H), 8.04 (s, 1H), 7.33-7.70 (m, 2H), 5.31 (s, 2H), 3.43 (s, 3H), 2.63 (q, J=7.6 Hz, 2H), 1.17 (t, J=7.5 Hz, 3H). MS-ESI (m/z) calcd for C₁₅H₁₇N₈O [M+H]+: 325.14. Found 325.26.

### Example 6. 4,5-dimethyl-N-(3-(pyridin-4-yl)-1H-indazol-5-yl)-4,7-dihydrotetrazolo[1,5-a]pyrimidine-6-carboxamide

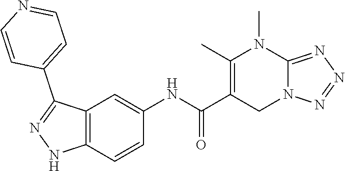 [SMILES: CC1=C(C(=O)Nc2ccc3[nH]nc(-c4ccncc4)c3c2)Cn2nnnc2N1C]

### Step 1. 5-Nitro-3-(pyridin-4-yl)-1H-indazole

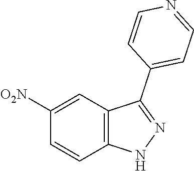 [SMILES: O=[N+]([O-])c1ccc2[nH]nc(-c3ccncc3)c2c1]

A mixture of 3-bromo-5-nitro-1H-indazole (450 mg, 1.86 mmol), 4-pyridylboronic acid (274.25 mg, 2.23 mmol), KOAc (547 mg, 5.58 mmol), Pd(Amphos)Cl₂ (132 mg, 185.93 μmol, 132 μL) in EtOH (6 mL) and H₂O (1.5 mL) was degassed and purged with N₂ (3×); then the mixture was stirred at 100° C. for 16 h under N₂ atmosphere. LC-MS showed 3-bromo-5-nitro-1H-indazole was consumed completely and one peak with desired mass was detected. The reaction mixture was concentrated to give a residue. The residue was diluted with 2N HCl (40 mL) and EtOAc (20 mL). A yellow solid formed which was collected and dried under vacuum to afford the title compound (350 mg, crude).

### Step 2. 3-(Pyridin-4-yl)-1H-indazol-5-amine

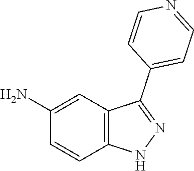 [SMILES: Nc1ccc2[nH]nc(-c3ccncc3)c2c1]

To a solution of 5-nitro-3-(pyridin-4-yl)-1H-indazole (350 mg, 1.46 mmol) in EtOH (4 mL) and H₂O (1 mL) was added Zn (476 mg, 7.29 mmol) and NH₄Cl (390 mg, 7.29 mmol). The mixture was stirred at 80° C. for 12 hrs. LC-MS showed 5-nitro-3-(pyridin-4-yl)-1H-indazole was consumed completely and one main peak with desired mass was detected. The reaction mixture was filtered, the cake was collected and redissolved in DMF (10 mL). The mixture was filtered and the filtrate was concentrated to give the title compound (220 mg, crude) as a yellow gum.

### Step 3. 4,5-dimethyl-N-(3-(pyridin-4-yl)-1H-indazol-5-yl)-4,7-dihydrotetrazolo[1,5-a]pyrimidine-6-carboxamide

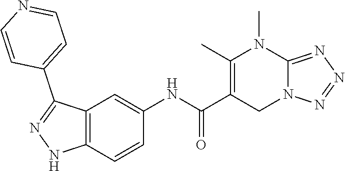 [SMILES: CC1=C(C(=O)Nc2ccc3[nH]nc(-c4ccncc4)c3c2)Cn2nnnc2N1C]

To a solution of Intermediate 3 (100 mg, 512 umol) in DCM (5 mL) was added a >50 wt % solution of propylphosphonic anhydride solution in ethyl acetate (489 mg, 768.53 umol, 457 uL, 50% purity) in EtOAc and TEA (155.53 mg, 1.54 mmol), then 3-(pyridin-4-yl)-1H-indazol-5-amine (118.49 mg, 563.59 umol) was added. The mixture was stirred at 15° C. for 12 hrs. LC-MS showed Intermediate 3 was consumed completely and one main peak with desired mass was detected. The reaction mixture was concentrated and purified by prep-HPLC (neutral condition) and further purified by prep-HPLC (TFA condition) to afford the title compound (14 mg, 25.92 μmol, 5% yield, TFA salt) as a light yellow solid. ¹H NMR (DMSO-d6, 400 MHz) δ 13.99 (s, 1H), 10.16 (s, 1H), 8.88 (d, J=6.0 Hz, 2H), 8.68 (s, 1H), 8.26 (d, J=6.0 Hz, 2H), 7.59-7.76 (m, 2H), 5.32 (s, 2H), 3.46 (s, 3H), 2.29 (s, 3H). MS-ESI (m/z) calcd for C₁₉H₁₈N₉O [M+H]+: 388.2. Found: 388.1.

### Example 7. 4,5-Dimethyl-N-(3-(2-morpholinopyridin-4-yl)-1H-indazol-5-yl)-4,7-dihydrotetrazolo[1,5-a]pyrimidine-6-carboxamide

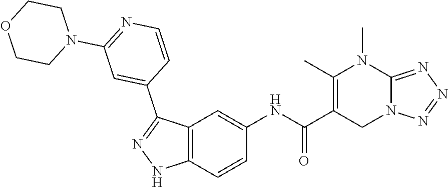 [SMILES: CC1=C(C(=O)Nc2ccc3[nH]nc(-c4ccnc(N5CCOCC5)c4)c3c2)Cn2nnnc2N1C]

### Step 1. 4-(4-Bromo-2-pyridinyl)-morpholine

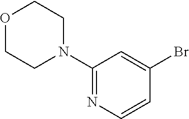 [SMILES: Brc1ccnc(N2CCOCC2)c1]

To a solution of 4-bromo-2-fluoropyridine (3 g, 17.05 mmol) in DMSO (40 mL) was added K₂CO₃ (7.07 g, 51.14 mmol) and morpholine (2.23 g, 25.57 mmol, 2.25 mL). The mixture was stirred at 100° C. for 12 hrs. LC-MS showed 4-bromo-2-fluoropyridine was consumed completely and the desired mass was detected. The residue was diluted with H₂O (50 mL) and extracted with EtOAc (25 mL×4). The combined organic layers were washed with brine (40 mL), dried over Na₂SO₄, filtered and concentrated under reduced pressure to give a residue. The residue was purified by column chromatography (SiO₂, Petroleum ether/EtOAc=1:0 to 100:1) to give the title compound (3.5 g, 14.40 mmol, 84% yield) as a white solid.

### Step 2. 4-(4-(4,4,5,5-Tetramethyl-1,3,2-dioxaborolan-2-yl)pyridin-2-yl)morpholine

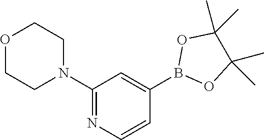 [SMILES: CC1(C)OB(c2ccnc(N3CCOCC3)c2)OC1(C)C]

A mixture of 4-(4-bromo-2-pyridinyl)-morpholine (1.5 g, 6.17 mmol), bis(pinacolato)diboron (1.88 g, 7.40 mmol), KOAc (1.51 g, 15.43 mmol), and Pd(dppf)Cl₂ (451 mg, 617.03 μmol) in dioxane (15 mL) was degassed and purged with N₂ (3×); then the mixture was stirred at 80° C. for 12 h under N₂ atmosphere. LC-MS showed 4-(4-bromo-2-pyridinyl)-morpholine was completely consumed and the desired mass was detected. The mixture was filtered and concentrated under reduced pressure to give the title compound (1.28 g, crude) as a black oil.

### Step 3. 4-(4-(5-nitro-1H-indazol-3-yl)pyridin-2-yl)morpholine

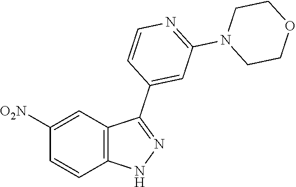 [SMILES: O=[N+]([O-])c1ccc2[nH]nc(-c3ccnc(N4CCOCC4)c3)c2c1]

A mixture of 4-(4-(4,4,5,5-tetramethyl-1,3,2-dioxaborolan-2-yl)pyridin-2-yl)morpholine (1.28 g, 6.15 mmol), 3-bromo-5-nitro-1H-indazole (1.79 g, 7.38 mmol), AcOK (1.81 g, 18.46 mmol), Pd(Amphos)Cl₂ (436 mg, 615.32 μmol) in EtOH (20 mL) and H₂O (5 mL) was degassed and purged with N₂ (3×); then the mixture was stirred at 100° C. for 12 h under N₂ atmosphere. LC-MS showed desired mass was detected. The reaction mixture was concentrated under reduced pressure to remove solvent. To the reaction mixture was added HCl (2N, 10 mL) to adjust to pH=4. EtOAc (40 mL) was added and the solid precipitated. The mixture was filtered and the cake was dried to give the title compound (1.2 g, crude) as a gray solid.

### Step 4. 3-(2-Morpholinopyridin-4-yl)-1H-indazol-5-amine

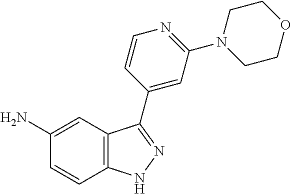 [SMILES: Nc1ccc2[nH]nc(-c3ccnc(N4CCOCC4)c3)c2c1]

To a solution of 4-(4-(5-nitro-1H-indazol-3-yl)pyridin-2-yl)morpholine (1.2 g, 3.69 mmol) in EtOH (10 mL) and H₂O (2.5 mL) was added Zn (1.21 g, 18.44 mmol) and NH₄Cl (986.56 mg, 18.44 mmol). The mixture was stirred at 80° C. for 16 hr. LC-MS showed 4-(4-(5-nitro-1H-indazol-3-yl)pyridin-2-yl)morpholine was consumed completely and one main peak with desired mass was detected. The reaction mixture was diluted with H₂O (50 mL), filtered and the cake was collected. The cake was then redissolved in DMF (20 mL); the resulting mixture was filtered and the filtrate was concentrated under reduced pressure to give the title compound (560 mg, crude) as a brown solid.

### Step 5. 4,5-Dimethyl-N-(3-(2-morpholinopyridin-4-yl)-1H-indazol-5-yl)-4,7-dihydrotetrazolo[1,5-a]pyrimidine-6-carboxamide

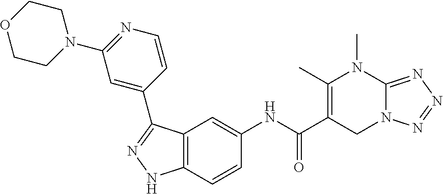 [SMILES: CC1=C(C(=O)Nc2ccc3[nH]nc(-c4ccnc(N5CCOCC5)c4)c3c2)Cn2nnnc2N1C]

To a solution of Intermediate 3 (100 mg, 512.35 μmol) in DCM (3 mL) was added propylphosphonic anhydride solution (“T₃P,” 424 mg, 666.06 μmol, 50% purity in EtOAc) and TEA (156 mg, 1.54 mmol) and 3-(2-morpholinopyridin-4-yl)-1H-indazol-5-amine (182 mg, 614.82 mmol). The mixture was stirred at 15° C. for 12 h. LC-MS showed Intermediate 3 was consumed completely and the desired mass was detected. The residue was purified by prep-HPLC (neutral condition) to afford the title compound (41 mg, 79.34 μmol, 15% yield) as a white solid. NMR (DMSO-d6, 400 MHz) δ 13.44 (s, 1H), 10.06 (s, 1H), 8.52 (s, 1H), 8.28 (d, J=5.1 Hz, 1H), 7.58-7.66 (m, 2H), 7.28 (s, 1H), 7.22 (d, J=5.3 Hz, 1H), 5.30 (s, 2H), 3.72-3.77 (m, 4H), 3.50-3.55 (m, 4H), 3.44 (s, 3H), 2.27 (s, 3H). MS-ESI (m/z) calcd for C₂₃H₂₅N₁₀O₂ [M+H]+: 473.2. Found: 473.3.

### Example 8. 7-Ethyl-N-(1H-indazol-5-yl)-4,5-dimethyl-4H,7H-[1,2,3,4]tetrazolo[1,5-a]pyrimidine-6-carboxamide

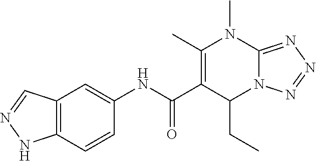 [SMILES: CCC1C(C(=O)Nc2ccc3[nH]ncc3c2)=C(C)N(C)c2nnnn21]

### Step 1. Ethyl 7-ethyl-5-methyl-4,7-dihydrotetrazolo[1,5-a]pyrimidine-6-carboxylate

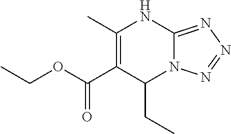 [SMILES: CCOC(=O)C1=C(C)Nc2nnnn2C1CC]

A mixture of 5-aminotetrazole monohydrate (800 mg; 7.8 mmol), propionaldehyde (453 mg; 7.8 mmol) and ethyl acetoacetate (0.98 mL; 7.8 mmol) in H₂O (45 mL) was heated at reflux for 1 h. The reaction mixture was cooled to room temperature. A further amount of propionaldehyde was added dropwise (3.9 mmol, 226 mg) and the reaction mixture was stirred for 1 h. The reaction mixture was cooled to room temperature and water was partially evaporated to a volume of about 3 mL. A white solid formed and was recovered by filtration through a glass frit, washing with cold water. The solid was dried to give the title compound as a white solid (675 mg, but presence of about 500 mol % of aminotetrazole). This product was used as such in the next step. ¹H NMR (DMSO-d6, 400 MHz) δ 11.02 (br s, 1H), 5.67 (t, J=4.1 Hz, 1H), 4.07-4.26 (m, 2H), 2.33-2.42 (m, 3H), 1.73-1.99 (m, 2H), 1.25 (t, J=7.2 Hz, 3H), 0.66 (t, J=7.4 Hz, 3H). MS-ESI (m/z) calcd for C₁₀H₁₆N₅O₂ [M+H]+: 238.12. Found 238.21.

### Step 2. Ethyl 7-ethyl-4,5-dimethyl-4,7-dihydrotetrazolo[1,5-a]pyrimidine-6-carboxylate

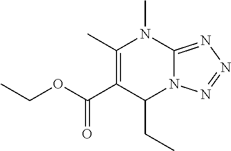 [SMILES: CCOC(=O)C1=C(C)N(C)c2nnnn2C1CC]

To a solution of ethyl 7-ethyl-4,5-dimethyl-4H,7H-[1,2,3,4]tetrazolo[1,5-a]pyrimidine-6-carboxylate (675 mg but containing only about 250 mg of desired starting material, 1.05 mmol) in DMF (15 mL) was added Mel (390 μL, 6.3 mmol) and Cs₂CO₃ (2200 mg, 6.3 mmol) and the mixture was stirred at 50° C. for 15 h. The solvent was evaporated and H₂O (20 mL) was added followed by EtOAc (20 mL). The organic layer was separated, dried over Na₂SO₄, filtered and concentrated to afford the title compound (145 mg, 0.58 mmol, 55% yield). ¹H NMR (400 MHz, CDCl₃) δ 5.79 (t, J=4.39 Hz, 1H), 4.19-4.33 (m, 2H), 3.53-3.61 (m, 3H), 2.56-2.65 (m, 3H), 1.95-2.05 (m, 1H), 1.78 (dqd, J=14.6, 7.4, 5.1 Hz, 1H), 1.29-1.39 (m, 3H), 1.68 (s, 1H), 0.70-0.85 (m, 3H). MS-ESI (m/z) calcd for C₁₁H₁₈N₅O₂ [M+H]+: 252.14. Found 252.22.

### Step 3. 7-Ethyl-4,5-dimethyl-4H,7H-[1,2,3,4]tetrazolo[1,5-a]pyrimidine-6-carboxylic Acid

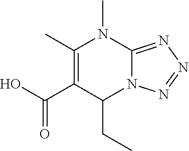 [SMILES: CCC1C(C(=O)O)=C(C)N(C)c2nnnn21]

To a solution of ethyl 7-ethyl-4,5-dimethyl-4,7-dihydrotetrazolo[1,5-a]pyrimidine-6-carboxylate (145 mg, 0.58 mmol) in THF (2 mL) was added a solution of LiOH (72 mg, 1.73 mmol) in H₂O (2 mL). The mixture was stirred at 50° C. for 15 h. The THF was evaporated and the aqueous solution was acidified with 1M HCl, then extracted with EtOAc, dried over Na₂SO₄, filtered and evaporated to obtain the title compound (118 mg, 0.53 mmol, 91% yield) as a colorless oil. ¹H NMR (400 MHz, CDCl₃) δ 5.80 (dd, J=5.0, 4.0 Hz, 1H), 3.58-3.67 (m, 3H), 2.63-2.71 (m, 3H), 2.06-2.11 (m, 1H), 1.84-1.95 (m, 1H), 0.77-0.85 (m, 3H). MS-ESI (m/z) calcd for C₉H₁₄N₅O₂ [M+H]+: 224.11. Found 224.36.

### Step 4. 7-Ethyl-N-(1H-indazol-5-yl)-4,5-dimethyl-4H,7H-[1,2,3,4]tetrazolo[1,5-a]pyrimidine-6-carboxamide

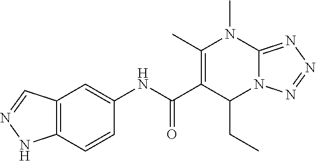 [SMILES: CCC1C(C(=O)Nc2ccc3[nH]ncc3c2)=C(C)N(C)c2nnnn21]

7-Ethyl-4,5-dimethyl-4H,7H-[1,2,3,4]tetrazolo[1,5-a]pyrimidine-6-carboxylic acid (118 mg, 0.53 mmol) was dissolved in DMF (2 mL). TEA (0.148 mL, 1.06 mmol), 1H-indazol-5-amine (105.9 mg, 0.79 mmol) and HATU (201.4 mg, 0.53 mmol) were added and the reaction mixture was stirred at room temperature for 1 h. The solvent was evaporated and EtOAc (20 mL) was added followed by H₂O (10 mL). The organic layer was separated, dried over Na₂SO₄, filtered and concentrated to obtain the crude product which was purified on Biotage (C18 25 g cartridge, reverse phase, water/formic acid 0.1% and ACN/formic acid 0.1% as eluent, 10:0 to 2:8) to give a light purple solid (84 mg) which was in turn purified on Biotage (25 g cartridge, normal phase, EtOAc/MeOH 10:0 to 8:2 as eluent) to afford the title compound (racemic mixture), as a white solid (50 mg, 0.148 mmol, 28% yield). ¹H NMR (400 MHz, DMSO-d₆) δ 13.00 (br. s, 1H), 10.12 (s, 1H), 8.15 (s, 1H), 8.04 (s, 1H), 7.42-7.56 (m, 2H), 5.77 (br s, 1H), 3.44 (s, 3H), 2.23 (d, J=0.7 Hz, 3H), 1.97-2.12 (m, 1H), 1.69-1.86 (m, 1H), 0.78 (t, J=7.5 Hz, 3H). MS-ESI (m/z) calcd for C₁₆H₁₉N₈O [M+H]+: 339.16. Found 339.3.

Enantiomers of the title compound were separated using semi-preparative chiral HPLC (Column: Chiralpak AS-H (25×2.0 cm), 5 μm; mobile phase: n-hexane/EtOH 75/25% v/v; flow rate (mL/min): 17; DAD detection: 220 nm; loop: 1000 μL; total amount: 46 mg; solubilization: 46 mg in 4.0 mL EtOH/MeOH 1/1=11.5 mg/mL; injection: 11.5 mg/injection). Analytical chiral HPLC (column: Chiralpak AS-H (25×0.46 cm), 5 μm; mobile phase: n-hexane/EtOH 75/25% v/v; flow rate (mL/min): 1.0; DAD: 220 nm; loop: 15 μL).

### Example 8a; Enantiomer 1, First Eluting Enantiomer

0.3% a/a by UV (7.0 min). 98% pure, e.e.=100%, 14.2 mg, white solid. Analytic chiral HPLC: 7.0 min. Semi-preparative chiral HPLC: 7.4 min. ¹H NMR (400 MHz, DMSO-d₆) δ 13.00 (br. s., 1H), 10.12 (s, 1H), 8.15 (s, 1H), 8.04 (s, 1H), 7.42-7.56 (m, 2H), 5.77 (br s., 1H), 3.44 (s, 3H), 2.23 (d, J=0.7 Hz, 3H), 1.97-2.12 (m, 1H), 1.69-1.86 (m, 1H), 0.78 (t, J=7.5 Hz, 3H). MS-ESI (m/z) calcd for C₁₆H₁₉N₈O [M+H]+: 339.16. Found 339.3.

### Example 8b; Enantiomer 2, Second Eluting Enantiomer

99.7% a/a by UV (11.7 min). 98% pure, e.e.=99.4%, 12 mg, white solid. Analytic chiral HPLC: 17.7 min. Semi-preparative chiral HPLC: 12.7 min. ¹H NMR (400 MHz, DMSO-d₆) δ 13.00 (br. s, 1H), 10.12 (s, 1H), 8.15 (s, 1H), 8.05 (s, 1H), 7.38-7.59 (m, 2H), 5.77 (br s, 1H), 3.43 (s, 3H), 2.23 (s, 3H), 1.99-2.12 (m, 1H), 1.69-1.86 (m, 1H), 0.78 (t, J=7.4 Hz, 3H). MS-ESI (m/z) calcd for C₁₆H₁₉N₈O [M+H]+: 339.16. Found 339.3.

### Example 9. 4,5-Dimethyl-N-(3-(6-morpholinopyrimidin-4-yl)-1H-indazol-5-yl)-4,7-dihydrotetrazolo[1,5-a]pyrimidine-6-carboxamide

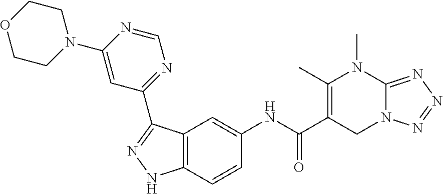 [SMILES: CC1=C(C(=O)Nc2ccc3[nH]nc(-c4cc(N5CCOCC5)ncn4)c3c2)Cn2nnnc2N1C]

### Step 1. 3-Bromo-5-nitro-1-((2-(trimethylsilyl)ethoxy)methyl)-1H-indazole

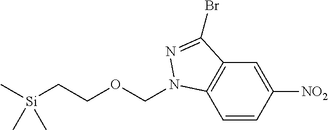 [SMILES: C[Si](C)(C)CCOCn1nc(Br)c2cc([N+](=O)[O-])ccc21]

To a solution of 3-bromo-5-nitro-1H-indazole (1 g, 4.13 mmol) in DMF (10 mL) was added NaH (248 mg, 6.20 mmol, 60% purity). The mixture was stirred at 20° C. for 0.5 hr. 2-(trimethylsilyl)ethoxymethyl chloride (895.50 mg, 5.37 mmol, 951 μL) was then added to the reaction mixture and the mixture was stirred at 20° C. for 2 h. LC-MS showed 3-bromo-5-nitro-1H-indazole was consumed completely and the desired mass was detected. The reaction mixture was quenched by addition of H₂O 15 mL, and then extracted with EtOAC (5 mL×3). The combined organic layers were dried over Na₂SO₄, filtered and concentrated under reduced pressure to give a residue. The residue was purified by flash silica gel chromatography (ISCO®; 12 g SepaFlash® Silica Flash Column, eluent of 08% EtOAc/Petroleum ether gradient at 50 mL/min) to afford the title compound (1.50 g, 3.88 mmol, 94% yield) as a yellow solid.

### Step 2. 4-(6-Chloropyrimidin-4-yl)morpholine

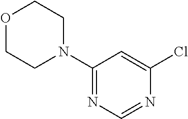 [SMILES: Clc1cc(N2CCOCC2)ncn1]

A mixture of 4,6-dichloropyrimidine (5 g, 33.56 mmol), morpholine (2.92 g, 33.56 mmol, 2.95 mL) and TEA (3.74 g, 36.92 mmol, 5.14 mL) in EtOH (50 mL) was degassed and purged with N₂ (3×) and then the mixture was stirred at 20° C. for 16 hrs under N₂ atmosphere. LC-MS showed 4,6-dichloropyrimidine was consumed completely and the desired mass was detected. The reaction mixture was concentrated under reduced pressure to give a residue. EtOAc (150 mL) was added to the residue and the resulting mixture was filtered. The cake was collected and purified by flash silica gel chromatography (ISCO®; 20 g SepaFlash® Silica Flash Column, Eluent of 0-60% EtOAc/petroleum ether gradient at 50 mL/min) to afford the title compound (1.08 g, 4.49 mmol, 13% yield) as a white solid.

### Step 3. 4-(6-(Trimethylstannyl)pyrimidin-4-yl)morpholine

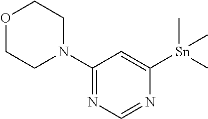 [SMILES: [CH3][Sn]([CH3])([CH3])[c]1cc(N2CCOCC2)ncn1]

A mixture of 4-(6-chloropyrimidin-4-yl)morpholine (800 mg, 4.01 mmol), trimethyl(trimethylstannyl)stannane (1.47 g, 4.49 mmol, 931 μL), Pd(PPh₃)₄ (185.23 mg, 160.29 umol), LiCl (204 mg, 4.81 mmol, 98 μL) and 2,6-ditert-butyl-4-methyl-phenol (18 mg, 80.15 μmol) in dioxane (10 mL) was degassed and purged with N₂ (3×) and the mixture was stirred at 100° C. for 2 h under N₂ atmosphere. LC-MS showed ˜55% of 4-(6-chloropyrimidin-4-yl)morpholine remained. The reaction mixture was then stirred at 100° C. for another 12 h. LC-MS showed 4-(6-chloropyrimidin-4-yl)morpholine was consumed completely and the desired mass was detected. The title compound (1.3 g, crude, theoretical amount) was used into next step directly as a black solution.

### Step 4. 4-(6-(5-Nitro-1-((2-(trimethylsilyl)ethoxy)methyl)-1H-indazol-3-yl)pyrimidin-4-yl)morpholine

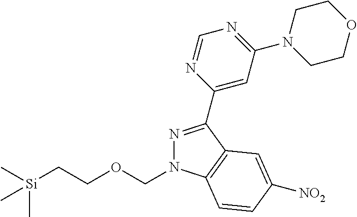 [SMILES: C[Si](C)(C)CCOCn1nc(-c2cc(N3CCOCC3)ncn2)c2cc([N+](=O)[O-])ccc21]

A mixture of 4-(6-(trimethylstannyl)pyrimidin-4-yl)morpholine (1.3 g, 3.96 mmol), 3-bromo-5-nitro-1-((2-(trimethylsilyl)ethoxy)methyl)-1H-indazole (from Step 1; 1.48 g, 3.96 mmol) and Pd(PPh₃)₄ (46 mg, 39.63 μmol) in dioxane (15 mL) was degassed and purged with N₂ (3×) and the mixture was stirred at 100° C. for 14 hrs under N₂ atmosphere. LC-MS showed 25% of 4-(6-(trimethylstannyl)pyrimidin-4-yl)morpholine remained and 13.9% desired mass was detected. The reaction mixture was concentrated under reduced pressure to remove solvent. The residue was purified by flash silica gel chromatography (ISCO®; 20 g SepaFlash® Silica Flash Column, Eluent of 0-23% EtOAc/Petroleum ether gradient @ 50 mL/min) to afford the title compound (440 mg, 693.24 μmol, 17% yield) as a yellow solid.

### Step 5. 4-(6-(5-Nitro-1H-indazol-3-yl)pyrimidin-4-yl)morpholine

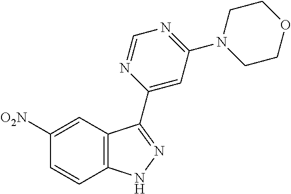 [SMILES: O=[N+]([O-])c1ccc2[nH]nc(-c3cc(N4CCOCC4)ncn3)c2c1]

A mixture of 4-(6-(5-nitro-1-((2-(trimethylsilyl)ethoxy)methyl)-1H-indazol-3-yl)pyrimidin-4-yl)morpholine (429 mg, 939.62 umol), TBAF (1 M in THF, 9.40 mL) and ethane-1,2-diamine (282 mg, 4.70 mmol, 314 μL) in THF (5 mL) was degassed and purged with N₂ (3×) and the mixture was stirred at 50° C. for 12 h under N₂ atmosphere. LC-MS showed 4-(6-(5-Nitro-1-((2-(trimethylsilyl)ethoxy)methyl)-1H-indazol-3-yl)pyrimidin-4-yl)morpholine was consumed completely and one main peak with the desired mass was detected. The reaction mixture was diluted with H₂O (20 mL) and extracted with EtOAc (10 mL×3). The combined organic layers were dried over Na₂SO₄, filtered and concentrated under reduced pressure to give the title compound (433 mg, 732.58 umol, 78% yield) as yellow solid, which was used in the next step without further purification.

### Step 6. 3-(6-Morpholinopyrimidin-4-yl)-1H-indazol-5-amine

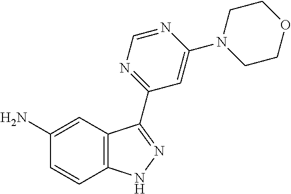 [SMILES: Nc1ccc2[nH]nc(-c3cc(N4CCOCC4)ncn3)c2c1]

A mixture of 4-(6-(5-nitro-1H-indazol-3-yl)pyrimidin-4-yl)morpholine (200 mg, 612.92 umol), Zn (200 mg, 3.06 mmol) and NH₄Cl (164 mg, 3.06 mmol, 107 uL) in EtOH (4 mL) and H₂O (1 mL) was degassed and purged with N₂ (3×) and the mixture was stirred at 80° C. for 12 h under an N₂ atmosphere. The resulting reaction mixture was filtered and the cake was collected. The cake was washed with DMF (20 mL), the mixture was filtered and filtrate was concentrated under reduced pressure to give the title compound (352 mg, crude) as black brown oil, which was used in the next step without further purification.

### Step 7. 4,5-Dimethyl-N-(3-(6-morpholinopyrimidin-4-yl)-1H-indazol-5-yl)-4,7-dihydrotetrazolo[1,5-a]pyrimidine-6-carboxamide

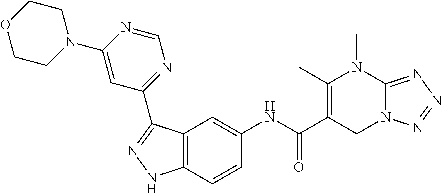 [SMILES: CC1=C(C(=O)Nc2ccc3[nH]nc(-c4cc(N5CCOCC5)ncn4)c3c2)Cn2nnnc2N1C]

A mixture of 3-(6-morpholinopyrimidin-4-yl)-1H-indazol-5-amine (150.31 mg, 507.23 μmol), Intermediate 3 (66 mg, 338.15 μmol), TEA (103 mg, 1.01 mmol, 141 μL) and T₃P (258 mg, 405.78 μmol, 241 μL, 50% purity in EtOAc) in DCM (2 mL) was degassed and purged with N₂ (3×), and then the mixture was stirred at 20° C. for 12 h under N₂ atmosphere. LC-MS showed 3-(6-morpholinopyrimidin-4-yl)-1H-indazol-5-amine was consumed completely and the desired mass was detected. The reaction mixture was concentrated under reduced pressure to remove solvent. The residue was purified by prep-HPLC (TFA condition) to afford the title compound (11.47 mg, 17.03 umol, 5% yield, TFA salt) as a red solid. ¹H NMR (DMSO-d6, 400 MHz) δ 13.81 (s, 1H), 10.12 (s, 1H), 8.74-8.69 (m, 2H), 7.74-7.68 (m, 1H), 7.66-7.60 (m, 1H), 7.44-7.40 (m, 1H), 5.30 (s, 2H), 3.76 (d, J=8.6 Hz, 8H), 3.43 (s, 3H), 2.27 (s, 3H). MS-ESI (m/z) calcd for C₂₂H₂₃N₁₁O₂ [M+H]+: 474.2. Found 474.2.

### Example 10. N-(3-Bromo-1H-indazol-5-yl)-4,5-dimethyl-4,7-dihydrotetrazolo[1,5-a]pyrimidine-6-carboxamide

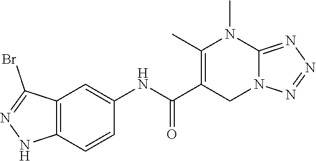

To a solution of 3-bromo-1H-indazol-5-amine (30 mg, 141.48 μmol) and Intermediate 3 (28 mg, 141.48 μmol) in EtOAc (2 mL) was added T₃P (270 mg, 424.43 μmol, 252 μL, 50% purity in EtOAc) and TEA (57 mg, 565.91 μmol, 79 μL). The mixture was stirred at 60° C. for 12 h. LC-MS showed 3-bromo-1H-indazol-5-amine was consumed completely and one main peak with the desired MS was detected. The reaction mixture was concentrated under reduced pressure to remove solvent. The residue was purified by prep-HPLC (TFA condition) to afford the title compound (9 mg, 21.88 μmol, 15% yield, TFA salt) as a yellow solid. ¹H NMR (DMSO-d6, 400 MHz) S 13.18-13.54 (m, 1H), 10.09 (s, 1H), 8.08 (s, 1H), 7.51-7.59 (m, 2H), 5.29 (s, 2H), 3.43 (s, 3H), 2.26 (s, 3H). MS-ESI (m/z) calcd for C₁₄H₁₄BrN₈O [M+H]+: 389.04. Found: 389.0.

### Example 11. 4,5-Dimethyl-N-(3-(4-sulfamoylphenyl)-1H-indazol-5-yl)-4,7-dihydrotetrazolo[1,5-a]pyrimidine-6-carboxamide

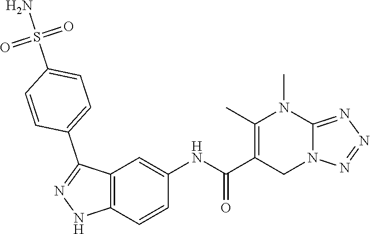 [SMILES: CC1=C(C(=O)Nc2ccc3[nH]nc(-c4ccc(S(N)(=O)=O)cc4)c3c2)Cn2nnnc2N1C]

To a solution of N-(3-bromo-1H-indazol-5-yl)-4,5-dimethyl-4,7-dihydrotetrazolo[1,5-a]pyrimidine-6-carboxamide (from Example 10, 50 mg, 128.47 μmol) and (4-sulfamoylphenyl)boronic acid (25.82 mg, 128.47 μmol) in 2 mL of DMF and 0.5 mL of H₂O was added tetrakis(triphenylphosphine)palladium(O) (14.84 mg, 12.85 μmol) and Na₂CO₃ (41 mg, 385.40 μmol). The mixture was stirred at 100° C. for 12 h under N₂ atmosphere. LC-MS showed N-(3-bromo-1H-indazol-5-yl)-4,5-dimethyl-4,7-dihydrotetrazolo[1,5-a]pyrimidine-6-carboxamide was consumed completely and one main peak with desired MS was detected. The reaction mixture was filtered and the filtrate was concentrated to give a residue. The residue was purified by prep-HPLC (TFA condition) to afford the title compound (12 mg, 20.62 μmol, 16% yield, TFA salt) as a white solid. NMR (DMSO-d6, 400 MHz) S 13.44 (s, 1H), 10.06 (s, 1H), 8.50 (s, 1H), 8.10 (m, J=8.6 Hz, 2H), 7.97 (m, J=8.6 Hz, 2H), 7.61 (s, 2H), 7.42 (s, 2H), 5.30 (s, 2H), 3.44 (s, 3H), 2.25-2.29 (m, 3H). MS-ESI (m/z) calcd for C₂₀H₂₀N₉O₃S [M+H]+: 466.13. Found: 466.1.

### Example 12. N⁶-(1H-indazol-5-yl)-N⁴,N⁴,5-trimethyltetrazolo[1,5-a]pyrimidine-4,6(7H)-dicarboxamide

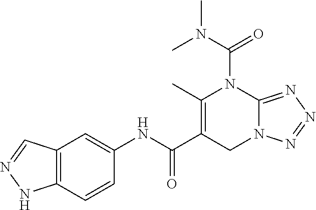 [SMILES: CC1=C(C(=O)Nc2ccc3[nH]ncc3c2)Cn2nnnc2N1C(=O)N(C)C]

### Step 1. Ethyl 4-(dimethylcarbamoyl)-5-methyl-4,7-dihydrotetrazolo[1,5-a]pyrimidine-6-carboxylate

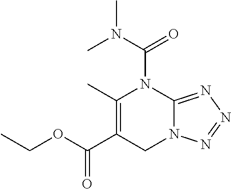 [SMILES: CCOC(=O)C1=C(C)N(C(=O)N(C)C)c2nnnn2C1]

To a solution of Intermediate 1 (0.4 g, 1.91 mmol) in toluene (15 mL) was added triphosgene (567 mg, 1.91 mmol) and DIEA (1.24 g, 9.56 mmol, 1.67 mL). The mixture was stirred at 20° C. for 2 h followed by addition of N-methylmethanamine (467.74 mg, 5.74 mmol, 526 μL, HCl). The mixture was stirred at 20° C. for 14 h. LC-MS showed the desired m/z was detected. The reaction mixture was diluted with MeOH (20 mL) and concentrated under reduced pressure to give a residue. The residue was purified by column chromatography (SiO₂, Petroleum ether/EtOAc=1:0 to 1:1) to give the title compound (180 mg, 406.78 μmol, 21% yield) as a yellow solid.

### Step 2. 4-(Dimethylcarbamoyl)-5-methyl-4,7-dihydrotetrazolo[1,5-a]pyrimidine-6-carboxylic Acid

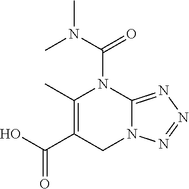 [SMILES: CC1=C(C(=O)O)Cn2nnnc2N1C(=O)N(C)C]

To a solution of ethyl 4-(dimethylcarbamoyl)-5-methyl-4,7-dihydrotetrazolo[1,5-a]pyrimidine-6-carboxylate (180 mgs, 406.78 μmol) in EtOH (4 mL) and H₂O (4 mL) was added LiOH.H₂O (83 mg, 2.03 mmol). The mixture was stirred at 15° C. for 12 h. LC-MS showed the desired m/z was detected. The reaction mixture was concentrated under reduced pressure to give a residue. The residue was diluted with H₂O (20 mL) and extracted with 1N HCl to pH=3 and extracted with EtOAc (10 mL×3). The organic layers were dried over Na₂SO₄, filtered and concentrated under reduced pressure to give the title compound (90 mg, 320.49 μmol, 79% yield) as a yellow solid, which was used into the next step without further purification.

### Step 3. N⁶-(1H-indazol-5-yl)-N⁴,N⁴,5-trimethyltetrazolo[1,5-a]pyrimidine-4,6(7H)-dicarboxamide

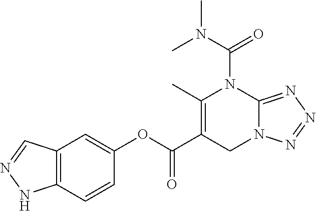 [SMILES: CC1=C(C(=O)Oc2ccc3[nH]ncc3c2)Cn2nnnc2N1C(=O)N(C)C]

To a solution of 4-(dimethylcarbamoyl)-5-methyl-4,7-dihydrotetrazolo[1,5-a]pyrimidine-6-carboxylic acid (85 mg, 336.99 μmol) and 3-bromo-1H-indazol-5-amine (45 mg, 336.99 μmol) in DCM (5 mL) was added T₃P/EtOAc (322 mg, 505.49 μmol, 301 μL, 50% purity) and TEA (102 mg, 1.01 mmol, 141 μL). The mixture was stirred at 15° C. for 2 h. LC-MS showed the desired m/z was detected. The reaction mixture was concentrated under reduced pressure to give a residue. The residue was purified by prep-HPLC (TFA condition) to afford the title compound (38 mg, 77.48 μmol, 23% yield, TFA salt) as a light pink solid. NMR (CD₃OD, 400 MHz) δ 8.14 (s, 1H), 8.05 (s, 1H), 7.58-7.49 (m, 2H), 5.32 (s, 2H), 3.17 (s, 3H), 3.14 (s, 3H), 2.24 (s, 3H). MS-ESI (m/z) calcd for C₁₆H₁₈N₉O₂ [M+H]+: 368.15. Found: 368.2.

### Example 13. N-(1H-indazol-5-yl)-5-methyl-4-phenyl-4,7-dihydrotetrazolo[1,5-a]pyrimidine-6-carboxamide

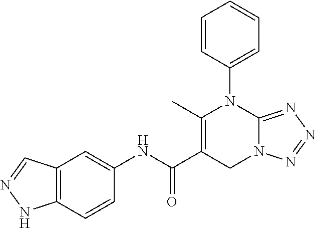 [SMILES: CC1=C(C(=O)Nc2ccc3[nH]ncc3c2)Cn2nnnc2N1c1ccccc1]

### Step 1. Ethyl 5-methyl-4-phenyl-4,7-dihydrotetrazolo[1,5-a]pyrimidine-6-carboxylate

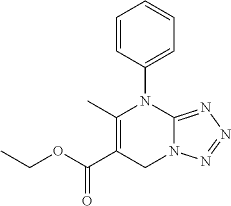 [SMILES: CCOC(=O)C1=C(C)N(c2ccccc2)c2nnnn2C1]

To a solution of Intermediate 1 (1 g, 4.78 mmol) and phenylboronic acid (1.75 g, 14.34 mmol) in DCM (20 mL) was added Cu(OAc)₂, (2.60 g, 14.34 mmol), TEA (1.45 g, 14.34 mmol, 2.00 mL), pyridine (3.02 g, 38.24 mmol, 3.09 mL) and 4 Å MS (100 mg, 4.78 mmol). The mixture was stirred at 25° C. for 12 h under O₂ (15 PSI) atmosphere. TLC indicated that the reaction was complete. The reaction mixture was filtered and the filtrate was concentrated under reduced pressure to give a residue. The residue was purified by column chromatography (SiO₂, Petroleum ether/EtOAc=1:0 to 1:1) to afford the title compound (150 mg, 394.32 μmol, 8% yield) as a brown oil.

### Step 2. 5-Methyl-4-phenyl-4,7-dihydrotetrazolo[1,5-a]pyrimidine-6-carboxylic Acid

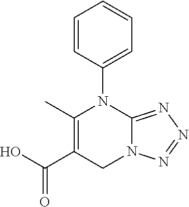 [SMILES: CC1=C(C(=O)O)Cn2nnnc2N1c1ccccc1]

To a solution of ethyl 5-methyl-4-phenyl-4,7-dihydrotetrazolo[1,5-a]pyrimidine-6-carboxylate (150 mg, 525.76 μmol) in EtOH (2 mL) and H₂O (2 mL) was added LiOH.H₂O (132 mg, 3.15 mmol) and the mixture was stirred at 20° C. for 12 h. LC-MS showed ethyl 5-methyl-4-phenyl-4,7-dihydrotetrazolo[1,5-a]pyrimidine-6-carboxylate was consumed completely and one main peak with the desired MS was detected. The reaction mixture was concentrated under reduced pressure to remove EtOH. The residue was diluted with H₂O (10 mL) and extracted with EtOAc (3 mL). The organic layer was discarded and the aqueous phase was treated with 1 M HCl to adjust the pH to 1-2 and then extracted with EtOAc (3 mL×3). The organic layer was washed with brine (10 mL), dried over Na₂SO₄, filtered and concentrated under reduced pressure to afford the title compound (90 mg, crude) as a brown solid.

### Step 3. N-(1H-indazol-5-yl)-5-methyl-4-phenyl-4,7-dihydrotetrazolo[1,5-a]pyrimidine-6-carboxamide

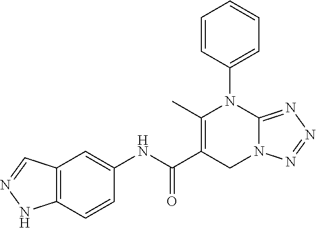 [SMILES: CC1=C(C(=O)Nc2ccc3[nH]ncc3c2)Cn2nnnc2N1c1ccccc1]

To a solution of 5-methyl-4-phenyl-4,7-dihydrotetrazolo[1,5-a]pyrimidine-6-carboxylic acid (130 mg, 505.35 μmol) and 1H-indazol-5-amine (81 mg, 606.42 μmol) in EtOAc (2 mL) was added T₃P (964.76 mg, 1.52 mmol, 902 μL, 50% purity in EtOAc) and TEA (204.55 mg, 2.02 mmol, 281.36 μL). The mixture was stirred at 60° C. for 12 h. LC-MS showed 5-methyl-4-phenyl-4,7-dihydrotetrazolo[1,5-a]pyrimidine-6-carboxylic acid was consumed completely and one peak with the desired MS was detected. The reaction mixture was concentrated under reduced pressure to remove solvent. The residue was purified by prep-HPLC (TFA condition) to afford the title compound (31 mg, 80.20 μmol, 16% yield, TFA salt) as a red gum. ¹H NMR (DMSO-d6, 400 MHz) δ 10.07 (s, 1H), 8.16 (s, 1H), 8.05 (s, 1H), 7.43-7.61 (m, 7H) 5.41 (s, 2H), 1.88 (s, 3H). MS-ESI (m/z) calcd for C₁₉H₁₈N₈O [M+H]+: 373.14. Found: 373.1.

### Example 14. N-(4-fluoro-1H-indazol-5-yl)-4,5-dimethyl-4,7-dihydrotetrazolo[1,5-a]pyrimidine-6-carboxamide

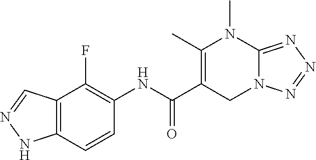 [SMILES: CC1=C(C(=O)Nc2ccc3[nH]ncc3c2F)Cn2nnnc2N1C]

To a solution of Intermediate 3 (123 mg, 635.17 μmol) in DCM (3 mL) was added 4-fluoro-1H-indazol-5-amine (80 mg, 529.31 μmol) and T₃P (505 mg, 793.96 μmol 472.10 μL, 50% purity in EtOAc) and TEA (161 mg, 1.59 mmol). The mixture was stirred at 20° C. for 12 h. LC-MS showed Intermediate 3 was consumed completely and one main peak with desired mass was detected. The reaction mixture was concentrated under reduced pressure to give a residue. The residue was purified by prep-HPLC (TFA condition) to afford the title compound (30 mg, 62.02 μmol, 12% yield, TFA salt) as a white solid. ¹H NMR (DMSO-d6, 400 MHz) δ 13.39 (s, 1H), 9.68 (s, 1H), 8.19 (s, 1H), 7.37 (m, 1H), 5.28 (s, 2H), 3.43 (s, 3H), 2.32 (s, 3H). MS-ESI (m/z) calcd for C₁₄H₁₄FN₈O [M+H]+: 329.12. Found: 329.1.

### Example 15. N-(1H-indazol-5-yl)-4,5-dimethyl-4,7-dihydrotetrazolo[1,5-a]pyrimidine-6-carbothioamide

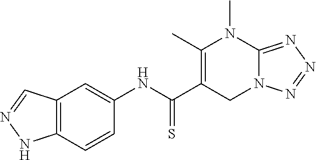 [SMILES: CC1=C(C(=S)Nc2ccc3[nH]ncc3c2)Cn2nnnc2N1C]

Lawesson's reagent (782 mg, 1.93. mmol) was added to a solution of N-(1H-indazol-5-yl)-4,5-dimethyl-4H,7H-[1,2,3,4]tetrazolo[1,5-a]pyrimidine-6-carboxamide (from Example 1, 300 mg, 0.95 mmol) in anhydrous dioxane (8 mL). The solution was stirred and heated at 100° C. for 2 h. An additional portion of Lawesson's reagent (782 mg, 1.93. mmol) was added and the mixture was left stirring at 100° C. for 18 h. The solvent was removed to afford the crude product which was purified by preparative HPLC (Method A) to afford the title compound (108 mg, 80% by UPLC). 30 mg of this crude material was taken up in DMSO and further purified by flash chromatography on a C18 column (100% H₂O+0.1% formic acid to 50/50 water+0.1% formic acid/ACN+0.1% formic acid) to give the title compound (10 mg, 0.3 mmol). ¹H NMR (400 MHz, DMSO-d6) δ 13.17 (br s, 1H), 11.80 (s, 1H), 8.38 (s, 1H), 8.13 (s, 1H), 7.49-7.66 (m, 2H), 5.30 (s, 2H), 3.45 (s, 3H), 2.18 (s, 3H). MS-ESI (m/z) calcd for C₁₄H₁₅N₈O [M+H]+: 327.11. Found 327.2.

### Example 16. 4,5,7-trimethyl-N-(2H-pyrazolo[3,4-b]pyridin-5-yl)-4,7-dihydrotetrazolo[1,5-a]pyrimidine-6-carboxamide

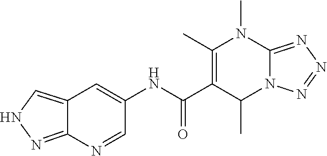

Intermediate 4 (50 mg, 0.239 mmol), HATU (90.9 mg, 0.239 mmol) and TEA (24.19 mg, 0.239 mmol, 33 μL) were stirred at room temperature in DMF (1 mL) for 5 min. 1H-pyrazolo[3,4-b]pyridin-5-amine (32.05 mg, 0.239 mmol) was added to reaction mixture and stirred at room temperature overnight. An additional equivalent of 1H-pyrazolo[3,4-b]pyridin-5-amine (32.05 mg, 0.239 mmol) was added and the reaction mixture was stirred at room temperature for 48 hrs. EtOAc and H₂O were added, the phases were separated and the organic layer was washed with H₂O (5×), brine, dried over Na₂SO₄, filtered and evaporated under reduced pressure. The material was purified by reverse phase chromatography to afford the title compound (50 mg, 0.15 mmol). ¹H NMR (400 MHz, DMSO-d₆) δ 13.60 (br s, 1H), 10.38 (s, 1H), 8.62 (d, J=2.42 Hz, 1H), 8.56 (d, J=2.20 Hz, 1H), 8.14 (s, 1H), 5.78 (d, J=6.16 Hz, 1H), 3.45 (s, 3H), 2.22 (s, 3H), 1.57 (d, J=6.38 Hz, 3H). MS-ESI (m/z) calcd for C₁₄H₁₆N₉O [M+H]+: 326.14. Found 326.24.

### Example 17. N-(6-methoxy-2H-indazol-5-yl)-4,5-dimethyl-4,7-dihydrotetrazolo[1,5-a]pyrimidine-6-carboxamide

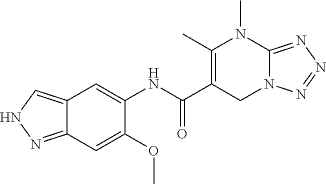 [SMILES: COc1cc2n[nH]cc2cc1NC(=O)C1=C(C)N(C)c2nnnn2C1]

A mixture of 6-methoxy-1H-indazol-5-amine (70 mg, 429 μmol), Intermediate 3 (100 mg, 512 μmol), T₃P (407.55 mg, 640 μmol, 50% purity in EtOAc) and TEA (130 mg, 1.28 mmol) in DCM (2 mL) was degassed and purged with N₂ (3×). The mixture was then stirred at 15° C. for 12 hrs under N₂ atmosphere. LC-MS showed 6-methoxy-1H-indazol-5-amine was consumed completely and the desired mass was detected. The reaction mixture was concentrated under reduced pressure and purified by prep-HPLC (TFA condition) to afford the title compound (62 mg, 120 μmol, 28% yield, 88% purity, TFA salt) as a white solid. ¹H NMR (400 MHz, DMSO-d₆) δ 9.11 (s, 1H), 8.09 (s, 1H), 7.95 (s, 1H), 7.02 (s, 1H), 5.24 (s, 2H), 3.89 (s, 3H), 3.42 (s, 3H), 2.29 (s, 3H). MS-ESI (m/z) calcd for C₁₅H₁₇N₈O₂ [M+H]+: 341.14. Found 341.1.

### Example 18. N-(3-carbamoyl-2H-indazol-5-yl)-4,5-dimethyl-4,7-dihydrotetrazolo[1,5-a]pyrimidine-6-carboxamide

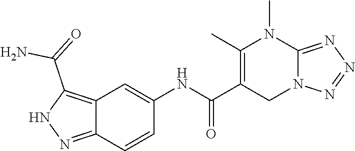

### Step 1. 5-nitro-1H-indazole-3-carboxamide

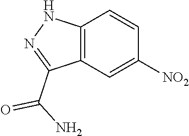 [SMILES: NC(=O)c1n[nH]c2ccc([N+](=O)[O-])cc12]

To a solution of 5-nitro-1H-indazole-3-carboxylic acid (300 mg, 1.45 mmol) in THF (10 mL) was added CDI (258.3 mg, 1.59 mmol) and the reaction mixture was stirred at 15° C. for 1.5 h. NH₃.H₂O (1.02 g, 7.24 mmol, 1.12 mL, 25% purity) was added and the reaction mixture was stirred at 15° C. for 15 min. LC-MS showed the reaction was complete. The reaction mixture was concentrated, dissolved in EtOAc (50 mL), washed with a 0.1 N HCl solution (30 mL), saturated NaHCO₃ (30 mL) and brine (30 mL). The organic layer was separated, dried and evaporated under vacuum to afford the title compound (200 mg, 882.82 μmol, 61% yield, 91% purity) as a light yellow solid.

### Step 2. 5-amino-1H-indazole-3-carboxamide

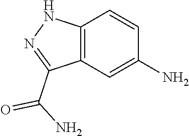 [SMILES: NC(=O)c1n[nH]c2ccc(N)cc12]

To a solution of 5-nitro-1H-indazole-3-carboxamide (180 mg, 794.54 μmol) in EtOH (0.5 mL) was added NH₄Cl (212.50 mg, 3.97 mmol) and Fe (221.9 mg, 3.97 mmol) and the reaction mixture was stirred at 80° C. for 1 h. LC-MS showed the reaction was complete. The reaction mixture was filtered and the filtrate concentrated to afford the title compound (140 mg, 723.14 μmol, 91% yield, 91% purity) as a brown solid.

### Step 3. N-(3-carbamoyl-1H-indazol-5-yl)-4,5-dimethyl-4,7-dihydrotetrazolo[1,5-a]pyrimidine-6-carboxamide

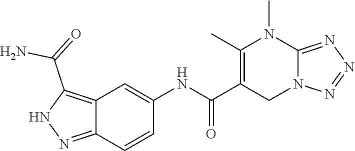 [SMILES: CC1=C(C(=O)Nc2ccc3n[nH]c(C(N)=O)c3c2)Cn2nnnc2N1C]

To a solution of 5-amino-1H-indazole-3-carboxamide (120 mg, 681.14 μmol) in DCM (4 mL) was added T₃P/EtOAc (650.18 mg, 1.02 mmol, 607 μL, 50% purity) and 5-methyl-4,7-dihydrotetrazolo[1,5-a]pyrimidine-6-carboxylic acid (133 mg, 681.14 μmol). The reaction mixture was stirred at 15° C. for 30 min. LC-MS showed the reaction was complete. The reaction mixture was concentrated and the residue was purified by prep-HPLC (TFA condition) to afford the title compound (23 mgs, 48.30 μmol, 7% yield, 97% purity, TFA salt) as a white solid. ¹H NMR (400 MHz, DMSO-d₆) δ 13.48 (s, 1H), 10.02 (s, 1H), 8.50 (s, 1H), 7.75-7.61 (m, 2H), 7.56 (d, J=8.8 Hz, 1H), 7.32 (s, 1H), 5.29 (s, 2H), 3.43 (s, 3H), 2.26 (s, 3H). MS-ESI (m/z) calcd for C₁₅H₁₆N₉O₂ [M+H]+: 354.13. Found 354.1.

### Example 19. N-(6-fluoro-2H-indazol-5-yl)-4,5-dimethyl-4,7-dihydrotetrazolo[1,5-a]pyrimidine-6-carboxamide

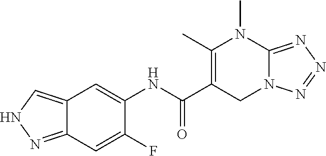 [SMILES: CC1=C(C(=O)Nc2cc3c[nH]nc3cc2F)Cn2nnnc2N1C]

To a solution of Intermediate 3 (70 mg, 359 μmol) and 6-fluoro-1H-indazol-5-amine (54 mg, 359 μmol) in DCM (3 mL) was added TEA (181 mg, 1.79 mmol, 250 μL) and T₃P (342 mg, 538 μmol, 320 μL, 50% purity in EtOAc). The mixture was stirred at 20° C. for 4 h. LC-MS showed Intermediate 3 was consumed completely and one peak with desired mass was detected. The mixture was concentrated and purified by prep-HPLC (neutral condition) to afford the title compound (11 mg, 32 μmol, 9% yield, 100% purity) as a white solid. ¹H NMR (400 MHz, DMSO-d₆) δ 13.14 (s, 1H), 9.68 (s, 1H), 8.10 (s, 1H), 7.98 (d, J=7.21 Hz, 1H), 7.44 (d, J=10.51 Hz, 1H), 5.28 (s, 2H) 3.44 (s, 3H), 2.32 (s, 3H). MS-ESI (m/z) calcd for C₁₄H₁₄FN₈O [M+H]+: 329.12. Found 329.1.

### Example 20. N-(6-carbamoyl-2H-indazol-5-yl)-4,5-dimethyl-4,7-dihydrotetrazolo[1,5-a]pyrimidine-6-carboxamide

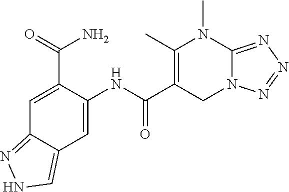 [SMILES: CC1=C(C(=O)Nc2cc3c[nH]nc3cc2C(N)=O)Cn2nnnc2N1C]

### Step 1. Methyl 5-nitro-1H-indazole-6-carboxylate

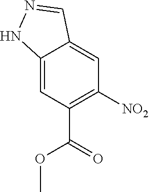 [SMILES: COC(=O)c1cc2[nH]ncc2cc1[N+](=O)[O-]]

H₂SO₄ (3.68 g, 36.77 mmol, 2 mL, 98% purity) was added dropwise into HNO₃ (1.40 g, 14.44 mmol, 1 mL, 65% purity) under 0° C. for 10 min. Methyl 1H-indazole-6-carboxylate (1 g, 5.68 mmol) was then taken into H₂SO₄ (25 mL, 98% purity), and added dropwise to the mixture of H₂SO₄ and HNO₃ prepared before at 0° C. The mixture was stirred at 15° C. for 20 min then warmed to 5° C. and stirred for 2 h. LC-MS showed methyl 1H-indazole-6-carboxylate was consumed completely and the desired mass was detected. The reaction mixture was added to ice, filtered and the filtrate cake was collected and concentrated under reduced pressure to afford the title compound (1.18 g crude) as a light yellow solid.

### Step 2. 5-Nitro-1H-indazole-6-carboxylic Acid

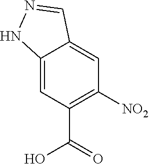 [SMILES: O=C(O)c1cc2[nH]ncc2cc1[N+](=O)[O-]]

To a solution of methyl 5-nitro-1H-indazole-6-carboxylate (300 mg, 1.36 mmol) in MeOH (3 mL) was added NaOH (2 M, 1.36 mL). The mixture was stirred at 20° C. for 1 h. TLC indicated 5-nitro-1H-indazole-6-carboxylic acid was consumed and a new more polar compound was present. The reaction mixture was concentrated under reduced pressure to remove MeOH. The pH of the residue was adjusted to pH 5 with 5M HCl and filtered. The filter cake was washed with H₂O until neutral and dried under reduced pressure to afford the title compound (627 mg crude) as a light yellow solid.

### Step 3. 5-Nitro-1H-indazole-6-carboxamide

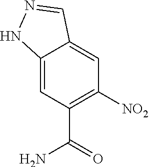 [SMILES: NC(=O)c1cc2[nH]ncc2cc1[N+](=O)[O-]]

To a solution of 5-nitro-1H-indazole-6-carboxamide (627 mg, 3.03 mmol) in THF (30 mL) was added CDI (589 mg, 3.63 mmol). The reaction mixture was stirred at 20° C. for 1.5 h then NH₃.H₂O (4.24 g, 30.27 mmol, 4.66 mL, 25% purity) was added to the mixture. The reaction mixture was stirred at 20° C. for 15 h. LC-MS showed 5-nitro-1H-indazole-6-carboxamide was consumed and a peak with the desired mass was detected. The reaction mixture was concentrated under reduced pressure to remove THF. The pH of the mixture was adjusted to pH 14 with a 2M NaOH solution and extracted with EtOAc (30 mL×3). The combined organic layers were washed with brine (30 mL). The organic phase was dried over anhydrous sodium sulfate, filtered and concentrated under reduced pressure to afford the title compound (570 mg crude) as a light yellow solid.

### Step 4. 5-Amino-1H-indazole-6-carboxamide

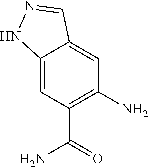 [SMILES: NC(=O)c1cc2[nH]ncc2cc1N]

To a solution of 5-amino-1H-indazole-6-carboxamide (200 mg, 913 μmol) in H₂O (2.5 mL) and ethanol (2.5 mL) was added Fe (270.88 mg, 4.85 mmol) and NH₄Cl (259.47 mg, 4.85 mmol). The mixture was stirred at 80° C. for 1 h. LC-MS showed 5-amino-1H-indazole-6-carboxamide was consumed completely and one main peak with the desired mass was detected. The reaction mixture was concentrated under reduced pressure to remove EtOH. The pH of the mixture was adjusted to pH 11 with 2 M NaOH and extracted with EtOAc (30 mL×3). The combined organic layers were washed with brine (30 mL). The organic phase was dried over anhydrous sodium sulfate, filtered and concentrated under reduced pressure to afford the title compound (148 mg crude) as a yellow solid.

### Step 5. N-(6-carbamoyl-1H-indazol-5-yl)-4,5-dimethyl-4,7-dihydrotetrazolo[1,5-a]pyrimidine-6-carboxamide

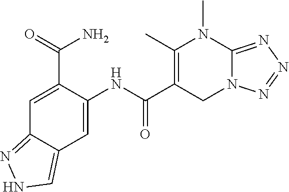 [SMILES: CC1=C(C(=O)Nc2cc3c[nH]nc3cc2C(N)=O)Cn2nnnc2N1C]

To a solution of 5-amino-1H-indazole-6-carboxamide (90 mg, 511 μmol) and Intermediate 3 (120 mg, 613 μmol) in pyridine (5 mL) was added EDCI (147 mg, 766 μmol). The mixture was stirred at 20° C. for 12 h. LC-MS showed the starting material was consumed completely and one main peak with desired mass was detected. The reaction mixture was concentrated under reduced pressure. The residue was dissolved in 2 mL DMF and purified by prep-HPLC (basic condition) to afford the title compound (12 mg, 32 μmol, 92% purity) as a yellow solid. NMR (400 MHz, DMSO-d₆) δ 13.41 (s, 1H), 11.41 (s, 1H), 8.76 (s, 1H), 8.50 (s, 1H), 8.14 (s, 1H), 8.00 (s, 1H), 7.83 (s, 1H), 7.79-7.86 (m, 1H), 5.26 (s, 2H), 3.44 (s, 3H), 2.40 (s, 3H). MS-ESI (m/z) calcd for C₁₅H₁₆N₉O₂ [M+H]+: 354.13. Found 354.1.

### Example 21. N-(6-amino-2H-indazol-5-yl)-4,5-dimethyl-4,7-dihydrotetrazolo[1,5-a]pyrimidine-6-carboxamide

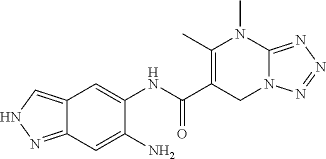 [SMILES: CC1=C(C(=O)Nc2cc3c[nH]nc3cc2N)Cn2nnnc2N1C]

4,5-dimethyl-4H,7H-[1,2,3,4]tetrazolo[1,5-a]pyrimidine-6-carboxylic acid (65 mg, 0.333 mmol), HATU (152 mg, 0.399 mmol) and TEA (40.46 mg, 0.399 mmol, 56 μL) were stirred at room temperature in DMF (3 mL) for 5 min. 1H-indazole-5,6-diamine (49 mg, 0.333 mmol) was added and the reaction was stirred a room temperature overnight. 1H-indazole-5,6-diamine (24.66 mg, 0.166 mmol) was added and the reaction mixture was stirred at room temperature for 6 h. H₂O was slowly added and the precipitate was filtered. The crude material was purified by reverse phase flash chromatography (H₂O/ACN from 10/0 to 7/3) to afford the title compound (30 mg, 0.092 mmol). ¹H NMR (400 MHz, DMSO-d₆) δ 12.37 (s, 1H), 9.17 (s, 1H), 7.79 (s, 1H), 7.51 (s, 1H), 6.74 (s, 1H), 5.33 (br. s., 2H), 5.06 (br. s., 2H), 3.43 (s, 3H), 2.32 (s, 3H). MS-ESI (m/z) calcd for C₁₄H₁₆N₉O [M+H]+: 326.14. Found 326.07.

### Example 22. (7R)—N-(3-bromo-2H-indazol-5-yl)-4,5,7-trimethyl-4,7-dihydrotetrazolo[1,5-a]pyrimidine-6-carboxamide

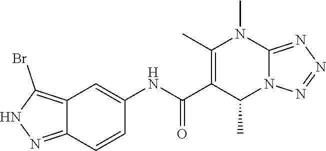 [SMILES: CC1=C(C(=O)Nc2ccc3n[nH]c(Br)c3c2)[C@@H](C)n2nnnc2N1C]

Intermediate 4b (35 mg, 0.17 mmol) and 3-bromo-1H-indazol-5-amine (71 mg, 0.33 mmol) were dissolved in dry DMF (2 mL). TEA (0.05 mL, 0.33 mmol) and HATU (76 mg, 0.20 mmol) were added. The mixture was stirred at room temperature overnight. EtOAc (20 mL) and H₂O (30 mL) were added, the organic layer was separated, dried over sodium sulphate, filtered and concentrated to give a crude product (120 mg) which was purified by prep-HPLC (Method A) to afford the title compound (32.5 mg, 0.08 mmol) as a white solid. 5 ¹H NMR (400 MHz, DMSO-d₆) δ 13.32 (br s, 1H), 10.20-10.39 (m, 1H), 8.10 (s, 1H), 7.45-7.66 (m, 2H), 5.69-5.86 (m, 1H), 3.44 (s, 3H), 2.20 (s, 3H), 1.56 (d, J=6.38 Hz, 3H). MS-ESI (m/z) calcd for C₁₅H₁₆BrN₈O [M+H]+: 403.06. Found 405.23.

### Example 23. 4,5,7-trimethyl-N-(1H-pyrazolo[3,4-c]pyridin-5-yl)-4,7-dihydrotetrazolo[1,5-a]pyrimidine-6-carboxamide

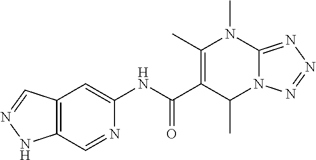 [SMILES: CC1=C(C(=O)Nc2cc3cn[nH]c3cn2)C(C)n2nnnc2N1C]

Intermediate 4 (100 mg, 0.478 mmol), 1H-pyrazolo[4,3-b]pyridin-5-amine (64 mg, 0.478 mmol) and TEA (47.89 mg, 0.478 mmol, 66 μL) were stirred at 0° C. in DMF (3 mL). T₃P (152 mg, 0.478 mmol, 50% wt in EtOAc) was added to the reaction mixture and the reaction was stirred at room temperature overnight. Another equivalent of T₃P was added (152 mg, 0.478 mmol) and stirred at room temperature for 40 h. H₂O and ETOAc were added to the reaction mixture. The phases were separated and the organic layer was washed with H₂O x 3, saturated aq. NaHCO₃×3, brine and dried over Na₂SO₄. The reaction was filtered and concentrated under reduced pressure. The final product was purified by prep-HPLC (Method B) to afford the compound as a racemic mixture (4.1 mg, 0.012 mmol). ¹H NMR (400 MHz, DMSO-d₆) δ 13.57 (br s, 1H), 10.60 (s, 1H), 8.82-8.87 (m, 1H), 8.44 (d, J=1.25 Hz, 1H), 8.22 (s, 1H), 5.75-5.83 (m, 1H), 3.39-3.45 (m, 3H), 2.18 (d, J=1.00 Hz, 3H), 1.55 (d, J=6.27 Hz, 3H). MS-ESI (m/z) calcd for C₁₄H₁₆N₉O [M+H]+: 326.14. Found 326.26.

### Example 24. 4,5,7-trimethyl-N-(3-(2-morpholinopyridin-4-yl)-1H-indazol-5-yl)-4,7-dihydrotetrazolo[1,5-a]pyrimidine-6-carboxamide

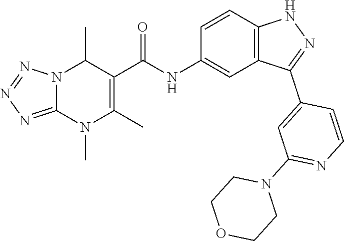 [SMILES: CC1=C(C(=O)Nc2ccc3[nH]nc(-c4ccnc(N5CCOCC5)c4)c3c2)C(C)n2nnnc2N1C]

### Step 1. 3-Bromo-5-nitro-1H-indazole

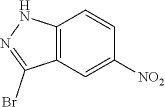 [SMILES: O=[N+]([O-])c1ccc2[nH]nc(Br)c2c1]

To a suspension of 5-nitro-1H-indazole (1.0 g, 6.13 mmol) in 2.0 M NaOH aqueous solution (25 mL) at ambient temperature, was added dropwise a solution of Br₂ (0.31 mL, 6.13 mmol) in 2.0 M NaOH aqueous solution (10 mL). The mixture was stirred for 3 h at room temperature. To the reaction mixture was added aq. Na₂S₂O₃ saturated solution (15 mL), followed by 2 M HCl aqueous solution (until acidic pH). The precipitate was collected by filtration and washed with water to afford the title compound (1.38 g, 5.70 mmol, 93% yield) as a yellow solid. ¹H NMR (400 MHz, DMSO-d₆) δ 14.10 (br s, 1H), 8.51 (d, J=2.2 Hz, 1H), 8.28 (dd, J=2.1, 9.1 Hz, 1H), 7.80 (d, J=9.2 Hz, 1H). MS-ESI (m/z) calcd for C₇H₅BrN₃O₂ [M+H]+: 241.95. Found 242.05/244.07.

### Step 2. 3-Bromo-5-nitro-1-{[2-(trimethylsilyl)ethoxy]methyl}-1H-indazole

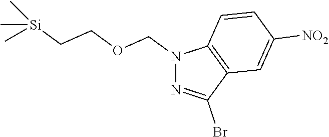 [SMILES: C[Si](C)(C)CCOCn1nc(Br)c2cc([N+](=O)[O-])ccc21]

To a solution of 3-bromo-5-nitro-1H-indazole (400 mg, 1.65 mmol) in DMF (7 mL) at 0° C. was added NaH (60% w/w, 79 mg, 1.98 mmol) and the mixture was stirred for 15 min. To the reaction mixture was then added SEM-Cl (0.35 mL, 1.98 mmol) and the reaction was warmed to room temperature and stirred for 2 hrs. The reaction was carefully quenched with an aqueous solution of NH₄Cl and the mixture was extracted with EtOAc (2×). The combined organic extracts were concentrated to dryness under reduced pressure and purified by flash chromatography on a 25 g silica gel column, using as eluent a gradient of EtOAc in cyclohexane from 0 to 40% to provide the title compound (372 mg, 1.0 mmol, 60% yield) as a white solid. NMR (400 MHz, Chloroform-d) δ 8.66 (d, J=1.8 Hz, 1H), 8.38 (dd, J=2.0, 9.2 Hz, 1H), 7.68 (d, J=9.2 Hz, 1H), 5.76 (s, 2H), 3.68-3.52 (m, 2H), 0.95-0.87 (m, 2H), −0.03 (s, 9H). MS-ESI (m/z) calcd for C₁₃H₁₉BrN₃O₃Si [M+H]+: 372.03. Found 372.16/374.13.

### Step 3. 5-nitro-3-(4,4,5,5-tetramethyl-1,3,2-dioxaborolan-2-yl)-7-{[2-(trimethylsilyl)ethoxy]methyl}-1H-indazole

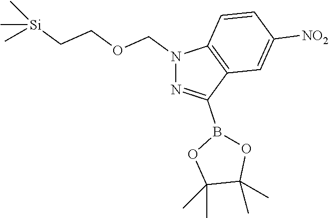 [SMILES: CC1(C)OB(c2nn(COCC[Si](C)(C)C)c3ccc([N+](=O)[O-])cc23)OC1(C)C]

3-Bromo-5-nitro-1-{[2-(trimethylsilyl)ethoxy]methyl}-1H-indazole (372 mg, 1.0 mmol), bis(pinacolato)diboron (279 mg, 1.1 mmol) and KOAc (294 mg, 3.0 mmol) were suspended in 1,4-dioxane (3 mL). The mixture was purged with N₂ for 5 min, and then Pd(dppf)Cl₂ (36 mg, 0.05 mmol) was added. The resulting mixture was heated to 100° C. for 1 h under nitrogen atmosphere. The crude was portioned between H₂O and EtOAc. The phases were separated, the aqueous layer was extracted with EtOAc (2×) and the combined organic layers washed with H₂O (1×), dried over anhydrous Na₂SO₄ and the solvent was removed under reduced pressure. Compound 3 was isolated as a brown oil and used as such in the subsequent reaction.

### Step 4. 4-(4-bromopyridin-2-yl)morpholine

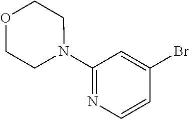 [SMILES: Brc1ccnc(N2CCOCC2)c1]

4-Bromo-2-fluoropyridine (1.0 g, 5.68 mmol) was dissolved in 5 mL DMF and morpholine (0.60 mL, 6.82 mmol) and Cs₂CO₃ (3.70 g, 11.36 mmol) were added at room temperature. The mixture was stirred in a sealed vial at 100° C. overnight. The mixture was portioned between H₂O and EtOAc. The phases were separated, the aqueous layer was extracted with EtOAc (2×) and the combined organic layers washed with brine (1×), dried over anhydrous Na₂SO₄ and evaporated to dryness. The crude material was purified via column chromatography on a 50 g silica gel column using as eluent a gradient of EtOAc in cyclohexane from 0 to 50%. The desired fractions were collected together to afford the title compound (1.24 g, 5.10 mmol, 90% yield) as a white solid. ¹H NMR (400 MHz, DMSO-d₆) δ 8.01 (d, J=5.3 Hz, 1H), 7.06 (d, J=1.3 Hz, 1H), 6.88 (dd, J=1.5, 5.3 Hz, 1H), 3.73-3.62 (m, 4H), 3.54-3.42 (m, 4H). MS-ESI (m/z) calcd for C₉H₁₂BrN₂O [M+H]+: 243.01. Found 243.09/245.10.

### Step 5. 3-[2-(morpholin-4-yl)pyridin-4-yl]-5-nitro-1-{[2-(trimethylsilyl)ethoxy]methyl}-1H-indazole

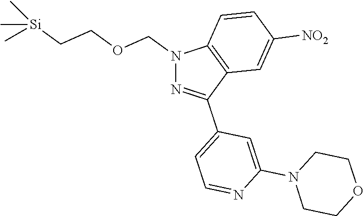 [SMILES: C[Si](C)(C)CCOCn1nc(-c2ccnc(N3CCOCC3)c2)c2cc([N+](=O)[O-])ccc21]

5-Nitro-3-(4,4,5,5-tetramethyl-1,3,2-dioxaborolan-2-yl)-1-{[2-(trimethylsilyl) ethoxy]methyl}-1H-indazole (crude, 1.0 mmol), 4-(4-bromopyridin-2-yl)morpholine (292 mg, 1.2 mmol) and Cs₂CO₃ (977 mg, 3.0 mmol) were suspended in THF (5 mL) and H₂O (1 mL). The mixture was purged with N₂ for 5 min, and then Pd(dppf)Cl₂ (73 mg, 0.1 mmol) was added. The reaction mixture was stirred at 100° C. for 1 h under nitrogen atmosphere. The mixture was portioned between H₂O and EtOAc. The phases were separated, the aqueous layer was extracted with EtOAc (2×) and the combined organic layers washed with H₂O (1×), dried over anhydrous Na₂SO₄ and the solvent was removed under reduced pressure. The obtained crude was purified by flash chromatography on a 25 g silica gel column, eluting with a gradient of EtOAc in cyclohexane from 0 to 30%. Compound 5 (200 mg, 0.44 mmol, 44% yield over 2 steps) was obtained as a yellow oil. ¹H NMR (400 MHz, Chloroform-d) δ 8.99 (d, J=2.0 Hz, 1H), 8.44-8.35 (m, 2H), 7.74 (d, J=9.2 Hz, 1H), 7.24 (dd, J=1.3, 5.1 Hz, 1H), 7.21 (s, 1H), 5.85 (s, 2H), 3.94-3.88 (m, 4H), 3.70-3.61 (m, 6H), 0.99-0.88 (m, 2H), −0.03 (s, 9H). MS-ESI (m/z) calcd for C₂₂H₃₀N₅O₄Si [M+H]+: 456.20. Found 456.36.

### Step 6. 3-[2-(morpholin-4-yl)pyridin-4-yl]-1-{[2-(trimethylsilyl)ethoxy]methyl}-1H-indazol-5-amine

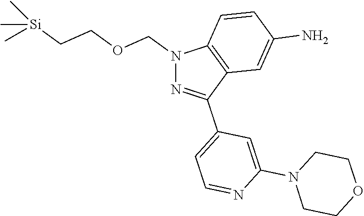 [SMILES: C[Si](C)(C)CCOCn1nc(-c2ccnc(N3CCOCC3)c2)c2cc(N)ccc21]

A mixture of 3-[2-(morpholin-4-yl)pyridin-4-yl]-5-nitro-1-{[2-(trimethylsilyl)ethoxy]methyl}-1H-indazole (200 mg, 0.44 mmol), ammonium chloride (26 mg, 0.48 mmol) and iron powder (98 mg, 1.76 mmol) in EtOH/H₂O (1:1) was stirred at 80° C. for 30 min. The solids were filtered off over a celite pad and the cake was washed with EtOH. Volatiles were removed under vacuum and redissolved in EtOAc. H₂O was added, the two phases were separated, the aqueous layer was extracted with EtOAc (2×). The combined organic layers washed with H₂O (1×), dried over anhydrous Na₂SO₄ and the solvent was removed under reduced pressure to afford the title compound (150 mg, 0.035 mmol, 80% yield). NMR (400 MHz, Chloroform-d) δ 8.33 (d, J=5.1 Hz, 1H), 7.47 (d, J=8.8 Hz, 1H), 7.26-7.18 (m, 3H), 6.96 (dd, J=2.0, 8.8 Hz, 1H), 5.74 (s, 2H), 3.94-3.87 (m, 4H), 3.77 (br. s., 2H), 3.66-3.60 (m, 6H), 0.99-0.85 (m, 2H), −0.04 (s, 9H). MS-ESI (m/z) calcd for C₂₂H₃₂N₅O₂Si [M+H]+: 426.22. Found 426.35.

### Step 7. (7R)-4,5,7-trimethyl-N-{3-[2-(morpholin-4-yl)pyridin-4-yl]-1-{[2-(trimethylsilyl)ethoxy]methyl}-1H-indazol-5-yl}-4H,7H-[1,2,3,4]tetrazolo[1,5-a]pyrimidine-6-carboxamide

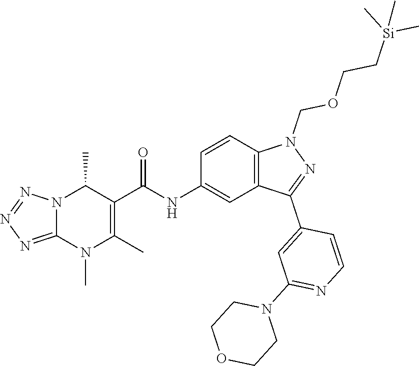 [SMILES: CC1=C(C(=O)Nc2ccc3c(c2)c(-c2ccnc(N4CCOCC4)c2)nn3COCC[Si](C)(C)C)[C@@H](C)n2nnnc2N1C]

Intermediate 4b (35 mg, 0.17 mmol) and 3-[2-(morpholin-4-yl)pyridin-4-yl]-1-{[2-(trimethylsilyl)ethoxy]methyl}-1H-indazol-5-amine (71 mg, 0.17 mmol) were dissolved in dry DMF (2 mL). The solution was cooled to 0° C. with an ice-H₂O bath and TEA (0.05 mL, 0.33 mmol) and HATU (76 mg, 0.20 mmol) were added. The mixture was stirred at 0° C. for 5 min, at room temperature overnight, heated at 50° C. for 2 hrs and then at 70° C. for additional 2 hrs. The mixture was portioned between H₂O and EtOAc, the aqueous phase was extracted with EtOAc (2×) and the combined organic layers were washed with H₂O (1×), dried over anhydrous Na₂SO₄, filtered and evaporated to dryness. The crude material was purified by normal phase column chromatography on a 10 g silica gel column, using as eluent a gradient of EtOAc in cyclohexane from 50 to 100% and then on a 11 g NH-column using as eluent a gradient of EtOAc in cyclohexane form 0 to 80%. The purest fractions were collected together and evaporated to dryness to give the title compound (35 mg, 0.057 mmol, 33% yield). ¹H NMR (400 MHz, Chloroform-d) δ 8.52 (s, 1H), 8.36 (d, J=5.1 Hz, 1H), 8.28 (s, 1H), 7.69-7.57 (m, 2H), 7.28-7.26 (m, 1H), 5.81 (s, 2H), 5.61 (q, J=5.9 Hz, 1H), 3.93-3.83 (m, 4H), 3.70-3.56 (m, 6H), 3.49 (s, 3H), 2.31 (s, 3H), 1.74 (d, J=6.4 Hz, 3H), 1.02-0.86 (m, 2H), 0.00-−0.07 (m, 9H). MS-ESI (m/z) calcd for C₃₀H₄₁N₁₀O₃Si [M+H]+: 617.31. Found 617.38.

### Step 8. (7S)-4,5,7-trimethyl-N-{3-[2-(morpholin-4-yl)pyridin-4-yl]-1H-indazol-5-yl}4H,7H-[1,2,3,4]tetrazolo[1,5-a]pyrimidine-6-carboxamide and (7R)-4,5,7-trimethyl-N-{3-[2-(morpholin-4-yl)pyridin-4-yl]-1H-indazol-5-yl}-4H,7H-[1,2,3,4]tetrazolo[1,5-a]pyrimidine-6-carboxamide

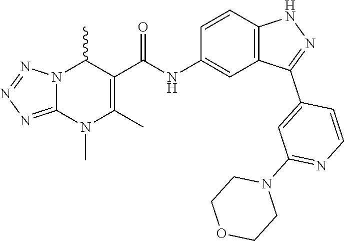 [SMILES: CC1=C(C(=O)Nc2ccc3[nH]nc(-c4ccnc(N5CCOCC5)c4)c3c2)C(C)n2nnnc2N1C]

To a solution of (7R)-4,5,7-trimethyl-N-{3-[2-(morpholin-4-yl)pyridin-4-yl]-1-{[2-(trimethylsilyl)ethoxy]methyl}-1H-indazol-5-yl}-4H,7H-[1,2,3,4]tetrazolo[1,5-a]pyrimidine-6-carboxamide (35 mg, 0.06 mmol) in THF (2 mL) was added a solution of HCl 4M in dioxane (0.5 mL). The reaction mixture was stirred at room temperature for 1 h. UPLC check showed that the starting material started to degradate. H₂O and EtOAc were added to the reaction mixture, the phases were separated, the aqueous layer was extracted with EtOAc (2×), and the combined organic layers washed with H₂O (1×), dried over anhydrous Na₂SO₄ and evaporated to dryness. The residue was re-dissolved in THF (2 mL), and TBAF (1M in THF, 1 mL). The reaction mixture was stirred at room temperature over 72 hrs and then at 70° C. for 3 hrs. After cooling to room temperature, the mixture was diluted with EtOAc, washed with H₂O, and concentrated in vacuo. The crude material was purified by reverse phase column chromatography, on a 12 g C18 column, using as eluent a gradient of ACN in water from 5 to 25%, in presence of 0.1% formic acid. The title compound (5 mg) was obtained. Chiral QC showed it was a racemic mixture. The material was submitted to preparative chiral HPLC separation. (Column: Chiralpak AD-H (25×2.0 cm), 5μ; mobile phase: n-Hexane/(EtOH/MeOH 1/1) 70/30% v/v; flow rate (mL/min): 18 ml/min; DAD detection: 220 nm; loop: 500 μL; total amount: 3 mg; solubilization: 3 mg in 1 ml EtOH=3 mg/ml; injection: 1.5 mg/injection). Analytical chiral HPLC (column: Chiralpak AD-H (25×0.46 cm), 5 mm; mobile phase: n-Hexane/(EtOH/MeOH 1/1) 70/30% v/v; flow rate (mL/min): 1.0; DAD: 220 nm; loop: 35 mL). The two enantiomers were collected separately.

### Example 24a; Enantiomer 1, First Eluting Enantiomer

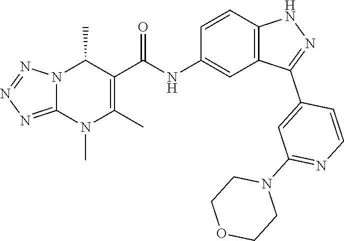 [SMILES: CC1=C(C(=O)Nc2ccc3[nH]nc(-c4ccnc(N5CCOCC5)c4)c3c2)[C@@H](C)n2nnnc2N1C]

(7S)-4,5,7-trimethyl-N-{3-[2-(morpholin-4-yl)pyridin-4-yl]-1H-indazol-5-yl}-4H,7H-[1,2,3,4]tetrazolo[1,5-a]pyrimidine-6-carboxamide (1.0 mg, 0.002 mmol, 3% yield, 100% e.e., white solid). Analytic chiral HPLC: 7.8 min. Semi-preparative chiral HPLC: 6.9 min. ¹H NMR (400 MHz, Methanol-d₄) δ 8.57 (s, 1H), 8.27 (d, J=5.28 Hz, 1H), 7.55-7.66 (m, 2H), 7.39 (s, 1H), 7.31-7.36 (m, 1H), 5.76 (q, J=6.24 Hz, 1H), 3.82-3.93 (m, 4H), 3.57-3.65 (m, 4H), 3.52 (s, 3H), 2.30 (d, J=1.10 Hz, 3H), 1.70 (d, J=6.38 Hz, 3H). MS-ESI (m/z) calcd for C₂₄H₂₇N₁₀O2 [M+H]+: 487.22. Found 487.76.

### Example 24b; Enantiomer 2, Second Eluting Enantiomer

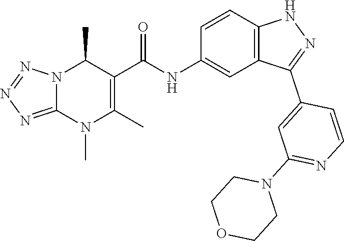 [SMILES: CC1=C(C(=O)Nc2ccc3[nH]nc(-c4ccnc(N5CCOCC5)c4)c3c2)[C@H](C)n2nnnc2N1C]

(7R)-4,5,7-trimethyl-N-{3-[2-(morpholin-4-yl)pyridin-4-yl]-1H-indazol-5-yl}-4H,7H-[1,2,3,4]tetrazolo[1,5-a]pyrimidine-6-carboxamide (1.5 mg, 0.003 mmol, 5% yield, 100% e.e., white solid). Analytic chiral HPLC: 10.4 min. Semi-preparative chiral HPLC: 8.8 min. ¹H NMR (400 MHz, Methanol-d₄) δ 8.57 (s, 1H), 8.27 (d, J=5.28 Hz, 1H), 7.55-7.70 (m, 2H), 7.39 (s, 1H), 7.34 (d, J=5.28 Hz, 1H), 5.76 (q, J=6.24 Hz, 1H), 3.82-3.91 (m, 4H), 3.57-3.66 (m, 4H), 3.52 (s, 3H), 2.30 (d, J=1.10 Hz, 3H), 1.70 (d, J=6.38 Hz, 3H). MS-ESI (m/z) calcd for C₂₄H₂₇N₁₀O2 [M+H]+: 487.22. Found 487.85.

### Example 25. N-(6-amino-2H-indazol-5-yl)-4,5,7-trimethyl-4,7-dihydrotetrazolo[1,5-a]pyrimidine-6-carboxamide

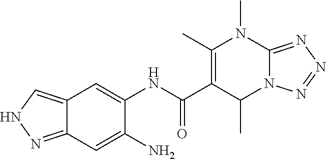 [SMILES: CC1=C(C(=O)Nc2cc3c[nH]nc3cc2N)C(C)n2nnnc2N1C]

Intermediate 4 (325.9 mg, 1.558 mmol), HATU (711 mg, 1.87 mmol) and TEA (157.6 mg, 1.87 mmol, 217 μL) were stirred at room temperature in DMF (4 mL) for 5 min. 1H-indazole-5,6-diamine (300 mg, 2.02 mmol) was added to reaction mixture and the reaction was stirred a room temperature overnight. H₂O was slowly added and the solid obtained was stirred at room temperature for 30 min. The reaction mixture was filtered, washed with H₂O and Et₂O. The solid obtained was further washed with MeOH (1 mL) and Et₂O to afford the title compound (414 mg, 1.22 mmol). ¹H NMR (400 MHz, DMSO-d₆) δ 12.40 (s, 1H), 9.45 (s, 1H), 7.80 (s, 1H), 7.57 (s, 1H), 6.77 (s, 1H), 5.76 (q, J=5.92 Hz, 1H), 4.99 (br s, 2H), 3.42 (s, 3H), 2.24 (s, 3H), 1.59 (d, J=6.36 Hz, 3H). MS-ESI (m/z) calcd for C₁₅H₁₈N₉O [M+H]+: 340.16. Found 340.08.

### Example 26. N-(2H-indazol-5-yl)-4,5,7,7-tetramethyl-4,7-dihydrotetrazolo[1,5-a]pyrimidine-6-carboxamide

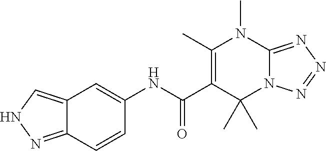 [SMILES: CC1=C(C(=O)Nc2ccc3n[nH]cc3c2)C(C)(C)n2nnnc2N1C]

### Step 1. Methyl 2-acetyl-3-methylbut-2-enoate

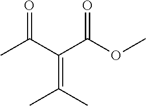

To a mixture of ZnCl₂ (0.35 g, 2.58 mmol), methyl acetoacetate (1.85 mL, 17.2 mmol) and acetone (1.9 mL, 25.8 mmol) was added acetic anhydride (2.2 mL, 23 mmol). The reaction medium was then heated to 50° C. for 48 h then diluted with DCM (100 mL) and washed with H₂O (30 mL). The organic phase was dried over Na₂SO₄, filtered and concentrated under reduced pressure. The residue obtained was purified on SP1 (100 g, silica cartridge, cyclohexane/EtOAc 10:0 to 85:15 as eluent) to give the desired product, methyl 2-acetyl-3-methylbut-2-enoate (550 mg, 3.5 mmol) as a yellow oil. ¹H NMR (400 MHz, DMSO-d₆) δ 3.73-3.83 (m, 3H), 2.23-2.34 (m, 3H), 2.06-2.16 (m, 3H), 1.90-1.98 (m, 3H). MS-ESI (m/z) calcd for C₈H₁₃O₃ [M+H]+: 157.08. Found 157.12.

### Step 2. Methyl 5,7,7-trimethyl-4H,7H-[1,2,3,4]tetrazolo[1,5-a]pyrimidine-6-carboxylate

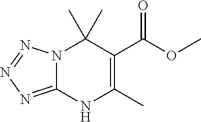 [SMILES: COC(=O)C1=C(C)Nc2nnnn2C1(C)C]

A mixture of 5-aminotetrazole monohydrate (66 mg, 0.64 mmol) and methyl 2-acetyl-3-methylbut-2-enoate (100 mg, 0.64 mmol) was heated in EtOH (5 mL) in the presence of molecular sieves for 4 h at reflux. The reaction was cooled to room temperature, filtered and concentrated to afford the title compound (80 mg, 0.36 mmol, yield 56%) as a white solid. ¹H NMR (400 MHz, DMSO-d₆) δ 10.70 (s, 1H), 3.79-3.88 (m, 3H), 2.38-2.48 (m, 3H), 1.94-2.02 (m, 6H). MS-ESI (m/z) calcd for C₉H₁₄N₅O₂ [M+H]+: 224.11. Found 224.26.

### Step 3. Methyl 4,5,7,7-tetramethyl-4H,7H-[1,2,3,4]tetrazolo[1,5-a]pyrimidine-6-carboxylate

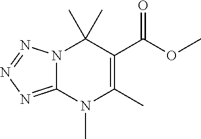 [SMILES: COC(=O)C1=C(C)N(C)c2nnnn2C1(C)C]

To a solution of methyl 5,7,7-trimethyl-4H,7H-[1,2,3,4]tetrazolo[1,5-a]pyrimidine-6-carboxylate (73 mg, 0.33 mmol) in DMF (5 mL) was added Mel (0.121 mL, 1.95 mmol) and Cs₂CO₃ (699 mg, 1.95 mmol) and the mixture was stirred at 50° C. for 0.5 h. The solvent was evaporated and H₂O was added (20 mL) followed by EtOAc (20 mL). The organic layer was separated, dried over sodium sulphate, filtered and concentrated to afford the title compound (75 mg, 0.31 mmol) as a white solid. NMR (400 MHz, DMSO-d₆) δ 4.84 (s, 1H), 3.76-3.88 (m, 3H), 3.47-3.58 (m, 3H), 2.21-2.32 (m, 3H), 1.78-1.92 (m, 6H). MS-ESI (m/z) calcd for C₁₀H₁₆N₅O₂ [M+H]+: 238.12. Found 238.2.

### Step 4. Tetramethyl-4H,7H-[1,2,3,4]tetrazolo[1,5-a]pyrimidine-6-carboxylic Acid

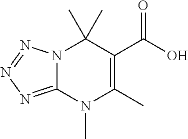 [SMILES: CC1=C(C(=O)O)C(C)(C)n2nnnc2N1C]

To a solution of methyl 4,5,7,7-tetramethyl-4H,7H-[1,2,3,4]tetrazolo[1,5-a]pyrimidine-6-carboxylate (75 mg, 0.31 mmol) in THF (2 mL) was added a solution of LiOH (39 mg, 0.93 mmol) in H₂O (2 mL). The mixture was stirred at 50° C. for 15 h. THF was evaporated and the H₂O solution was acidified with 1M HCl, extracted with EtOAc, dried over Na₂SO₄, filtered and evaporated to obtain the title compound (118 mg) which was used without further purification. ¹H NMR (400 MHz, DMSO-d₆) δ 3.34-3.39 (m, 3H), 1.90 (s, 3H), 1.64-1.71 (m, 6H). MS-ESI (m/z) calcd for C₉H₁₄N₅O₂ [M+H]+: 224.11. Found 224.18.

### Step 5. Tert-butyl 5-{4,5,7,7-tetramethyl-4H,7H-[1,2,3,4]tetrazolo[1,5-a]pyrimidine-6-amido}-1H-indazole-1-carboxylate

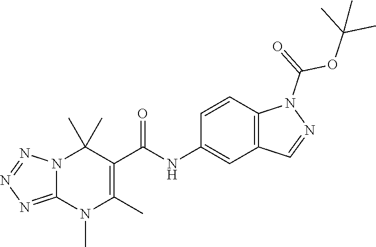 [SMILES: CC1=C(C(=O)Nc2ccc3c(cnn3C(=O)OC(C)(C)C)c2)C(C)(C)n2nnnc2N1C]

Tetramethyl-4H,7H-[1,2,3,4]tetrazolo[1,5-a]pyrimidine-6-carboxylic acid (60 mg, 0.27 mmol) was dissolved in DMF (2 mL). TEA (0.075 mL, 0.54 mmol), tert-butyl 5-amino-1H-indazole-1-carboxylate (53.8 mg, 0.4 mmol) and HATU (103 mg, 0.27 mmol) were added and the reaction mixture was stirred at room temperature for 1 h. The solvent was evaporated, EtOAc (20 mL) was added followed by H₂O (10 mL). The organic layer was separated, dried over Na₂SO₄, filtered and concentrated to obtain the title compound (120 mg crude). The crude material was purified via prep HPLC (Method B) to afford the title compound (7.5 mg, 0.017 mmol) as a white solid. ¹H NMR (400 MHz, DMSO-d₆) δ 8.44 (br s, 1H), 8.04-8.22 (m, 3H), 7.45-7.57 (m, 1H), 3.52 (s, 3H), 1.90-1.99 (m, 3H), 1.72-1.77 (m, 9H), 1.59 (s, 6H). MS-ESI (m/z) calcd for C₂₁H₂₇N₈O₃ [M+H]+: 439.21. Found 439.8.

### Step 6. N-(1H-Indazol-5-yl)-4,5,7,7-tetramethyl-4H,7H-[1,2,3,4]tetrazolo[1,5-a]pyrimidine-6-carboxamide

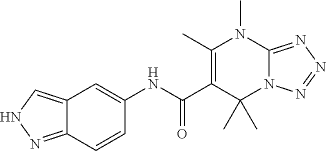 [SMILES: CC1=C(C(=O)Nc2ccc3n[nH]cc3c2)C(C)(C)n2nnnc2N1C]

Tert-butyl 5-{4,5,7,7-tetramethyl-4H,7H-[1,2,3,4]tetrazolo[1,5-a]pyrimidine-6-amido}-1H-indazole-1-carboxylate (7.5 mg, 0.017 mmol) was dissolved in DCM (2 mL). TFA (0.5 mL) was added dropwise at 0° C. and the reaction mixture was stirred at room temperature for 3 h. The solvent was evaporated under reduced pressure. The reaction material was purified via prep-HPLC (Method A) to afford the title compound (2.1 mg, 0.006 mmol) as a white solid. ¹H NMR (400 MHz, DMSO-d₆) δ 8.15 (d, J=1.10 Hz, 1H), 8.06 (s, 1H), 7.47-7.63 (m, 2H), 3.46-3.54 (m, 3H), 2.23 (s, 3H), 1.90 (s, 6H). MS-ESI (m/z) calcd for C₁₆H₁₉N₈O [M+H]+: 339.16. Found 339.8.

### Example 27. (R)-4,5,7-trimethyl-N-(3-(pyridin-4-yl)-1H-indazol-5-yl)-4,7-dihydrotetrazolo[1,5-a]pyrimidine-6-carboxamide

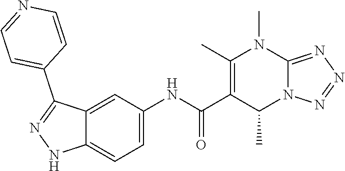 [SMILES: CC1=C(C(=O)Nc2ccc3[nH]nc(-c4ccncc4)c3c2)[C@@H](C)n2nnnc2N1C]

(7R)—N-(3-bromo-2H-indazol-5-yl)-4,5,7-trimethyl-4,7-dihydrotetrazolo[1,5-a]pyrimidine-6-carboxamide (from Example 22; 50 mg, 0.12 mmol), (pyridin-4-yl)boronic acid (31 mg, 0.25 mmol) and Na₂CO₃ (39 mg, 0.37 mmol) were suspended in DMF (2 mL) and H₂O (0.5 mL). The mixture was purged with N₂ for 5 min, and then Pd(PPh₃)₄ (7 mg, 0.006 mmol) was added. The reaction mixture was stirred at 100° C. for 6 h under nitrogen atmosphere and then it was irradiated with MW at 100° C. for 30 min. The mixture was portioned between H₂O and EtOAc. The phases were separated, the aqueous layer was extracted with EtOAc (2×) and the combined organic layers washed with H₂O (1×), dried over anhydrous Na₂SO₄ and the solvent was removed under reduced pressure. The crude material was purified by flash chromatography on a 10 g silica gel column, eluting with a gradient of EtOAc in cyclohexane from 0 to 100%, followed by a gradient of MeOH in EtOAc from 0 to 10%. The purest fractions were collected and purified again via reverse phase column chromatography using as eluent a gradient of ACN in H₂O from 0 to 25% in presence of 0.1% formic acid. The title compound (5 mg, 0.012 mmol, 10% yield) was recovered as a yellow solid. ¹H NMR (400 MHz, DMSO-d₆) δ 13.58 (br s, 1H), 10.30 (s, 1H), 8.67-8.78 (m, 2H), 8.59 (s, 1H), 7.87-7.97 (m, 2H), 7.64 (s, 2H), 5.62-5.95 (m, 1H), 3.45 (s, 3H), 2.22 (s, 3H), 1.58 (d, J=6.38 Hz, 3H). MS-ESI (m/z) calcd for C₂₀H₂₀N₉O [M+H]+: 402.17. Found 402.19.

### Example 28. N-(3-acetamido-2H-indazol-5-yl)-4,5-dimethyl-4,7-dihydrotetrazolo[1,5-a]pyrimidine-6-carboxamide

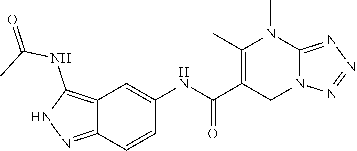 [SMILES: CC(=O)Nc1[nH]nc2ccc(NC(=O)C3=C(C)N(C)c4nnnn4C3)cc12]

### Step 1. 4,5-dimethyl-4,7-dihydrotetrazolo[1,5-a]pyrimidine-6-carbonyl Chloride

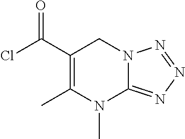

To a solution of Intermediate 3 (100 mg, 512.35 μmol) in DCM (2 mL) were added (COCl)₂ (98 mg, 769 μmol, 67 μL) and DMF (374 μg, 5.12 μmol) (one drop). The mixture was stirred at 25° C. for 30 min. TLC showed the reaction was complete. The reaction mixture was concentrated to afford the title compound (110 mg crude) as a yellow solid.

### Step 2. N-(5-Nitro-1H-indazol-3-yl)acetamide

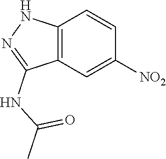 [SMILES: CC(=O)Nc1n[nH]c2ccc([N+](=O)[O-])cc12]

To a solution of 5-nitro-1H-indazol-3-amine (400 mg, 2.25 mmol) in pyridine (6 mL) was added a solution of acetyl chloride (185.07 mg, 2.36 mmol, 168.24 μL) in ACN (2 mL) at 0° C. The reaction mixture was stirred at 0° C. for 1 h. LC-MS showed formation of desired product. The mixture was concentrated under vacuum and washed with MeOH (5 mL), filtered and concentrated under vacuum to afford the title compound (328 mg, 1.42 mmol, 63% yield, 96% purity) as an orange solid.

### Step 3. N-(5-amino-1H-indazol-3-yl)acetamide

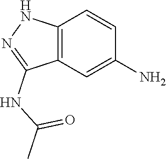

To a solution of N-(5-nitro-1H-indazol-3-yl)acetamide (200 mg, 908.33 μmol) in EtOH (4 mL) and H₂O (1 mL) was added Fe (253.63 mg, 4.54 mmol) and NH₄Cl (242.94 mg, 4.54 mmol). The mixture was stirred at 80° C. for 12 h. LC-MS showed the reaction was complete. The reaction mixture was filtered and the filtrate was concentrated under vacuum to afford the title compound (208 mg crude) as a gray solid.

### Step 4. N-(3-acetamido-1H-indazol-5-yl)-4,5-dimethyl-4,7-dihydrotetrazolo[1,5-a]pyrimidine-6-carboxamide

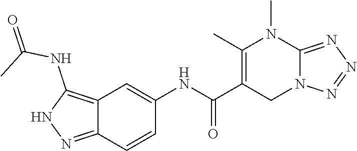 [SMILES: CC(=O)Nc1[nH]nc2ccc(NC(=O)C3=C(C)N(C)c4nnnn4C3)cc12]

To a solution of N-(5-amino-1H-indazol-3-yl)acetamide (70 mg, 368.03 μmol) in pyridine (2 mL) was added a solution of 4,5-dimethyl-4,7-dihydrotetrazolo[1,5-a]pyrimidine-6-carbonyl chloride (Step 1; 110 mg, 514.92 μmol) in ACN (1 mL) at 0° C. The mixture was stirred at 25° C. for 12 h. The mixture was concentrated under vacuum and the residue was purified by prep-HPLC (TFA condition) to afford the product at 87% purity, it was repurified by prep-HPLC (basic condition) to afford the title compound (1.14 mg, 2.95 μmol, 1% yield, 95% purity) as a brown liquid. ¹H NMR (400 MHz, DMSO-d₆) δ 12.60 (s, 1H), 10.26 (s, 1H), 9.94 (s, 1H), 8.02 (s, 1H), 7.53 (d, J=8.80 Hz, 1H), 7.40 (d, J=8.93 Hz, 1H), 5.28 (s, 2H), 3.43 (s, 3H), 2.25 (s, 3H), 2.10 (s, 3H). MS-ESI (m/z) calcd for C₁₆H₁₈N₉O₂ [M+H]+: 368.15. Found 368.1.

### Example 29. 4,5-dimethyl-N-(2-oxoindolin-5-yl)-4,7-dihydrotetrazolo[1,5-a]pyrimidine-6-carboxamide

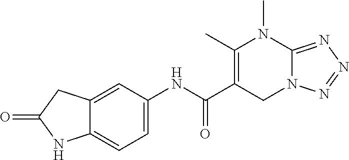 [SMILES: CC1=C(C(=O)Nc2ccc3c(c2)CC(=O)N3)Cn2nnnc2N1C]

To a solution of 5-aminoindolin-2-one (73 mg, 492 μmol) in DCM (2 mL) was added Intermediate 3 (80 mg, 410 μmol), T₃P/EtOAc (783 mg, 1.23 mmol, 731 μL, 50% purity) and TEA (166 mg, 1.64 mmol, 228 μL). The reaction mixture was then stirred at 25° C. for 12 h. LC-MS showed the reaction was complete. The reaction mixture was concentrated, washed with MeOH (3 mL), filtered and concentrated under vacuum to afford the title compound (54 mg, 153 μmol, 37% yield, 92% purity) as a white solid. ¹H NMR (400 MHz, DMSO-d₆) δ 10.32 (m, 1H), 9.83 (s, 1H), 7.53 (s, 1H), 7.37 (d, J=8.38 Hz, 1H), 6.75 (d, J=8.38 Hz, 1H), 5.22 (s, 2H), 3.47 (s, 2H), 3.40 (s, 3H), 2.20 (s, 3H). MS-ESI (m/z) calcd for C₁₅H₁₆N₇O₂ [M+H]+: 326.13. Found 326.2.

### Example 30. 4,5-dimethyl-N-(2-oxo-2,3-dihydrobenzo[d]oxazol-6-yl)-4,7-dihydrotetrazolo[1,5-a]pyrimidine-6-carboxamide

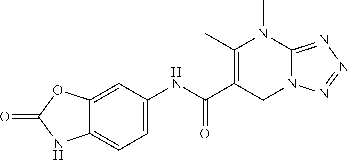 [SMILES: CC1=C(C(=O)Nc2ccc3[nH]c(=O)oc3c2)Cn2nnnc2N1C]

To a solution of 6-aminobenzo[d]oxazol-2(3H)-one (77 mg, 512 μmol) in DCM (4 mL) was added Intermediate 3 (0.1 g, 512 μmol), T₃P/EtOAc (489 mg, 783 μmol, 50% purity) and TEA (156 mg, 1.54 mmol). The reaction mixture was stirred at 15° C. for 13 h. LC-MS showed the reaction was complete. The reaction mixture was concentrated and purified by prep-HPLC (TFA condition) to afford the title compound (54 mg, 153 μmol, 37% yield, 92% purity) as a white solid. NMR (400 MHz, DMSO-d₆) δ 11.58 (s, 1H), 10.01 (s, 1H), 7.71 (d, J=1.5 Hz, 1H), 7.30 (dd, J=1.8, 8.4 Hz, 1H), 7.05 (d, J=8.4 Hz, 1H), 5.25 (s, 2H), 3.42 (s, 3H), 2.23 (s, 3H). MS-ESI (m/z) calcd for C₁₄H₁₄N₇O₃ [M+H]+: 328.11. Found 328.1.

### Example 31. 4,5-dimethyl-N-(2-oxo-2,3-dihydro-1H-benzo[d]imidazol-5-yl)-4,7-dihydrotetrazolo[1,5-a]pyrimidine-6-carboxamide

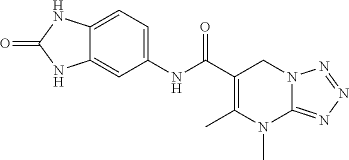 [SMILES: CC1=C(C(=O)Nc2ccc3[nH]c(=O)[nH]c3c2)Cn2nnnc2N1C]

To a solution of 5-amino-1,3-dihydro-2H-benzo[d]imidazol-2-one (76 mg, 512 μmol) in DCM (1 mL) was added Intermediate 3 (100 mg, 512 μmol) and T₃P/EtOAc (489 mg, 768 μmol, 457 μL, 50% purity). The reaction mixture was then stirred at 20° C. for 30 minutes. TEA (153 mg, 1.54 mmol, 214 μL) was added and the reaction mixture was stirred at 20° C. for 12 hrs. LC-MS showed the reaction was complete. The reaction mixture was washed with acetonitrile (1 mL), saturated NaHCO₃ (1 mL) and H₂O (5 mL), then concentrated to afford the title compound (28 mg, 83 μmol, 16% yield, 96% purity) as a white solid. ¹H NMR (400 MHz, DMSO-d₆) δ 10.52 (m, 3H), 7.46 (s, 1H), 7.08 (d, J=7.5 Hz, 1H), 6.84 (d, J=7.9 Hz, 1H), 5.24 (s, 2H), 3.41 (s, 3H), 2.21 (s, 3H). MS-ESI (m/z) calcd for C₁₄H₁₅N₈O₂ [M+H]+: 327.12. Found 327.1.

### Example 32. 4′,5′-Dimethyl-N-(3-methyl-2H-indazol-5-yl)-4′H-spiro[cyclopentane-1,7′-tetrazolo[1,5-a]pyrimidine]-6′-carboxamide

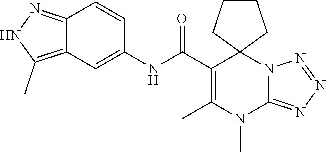 [SMILES: CC1=C(C(=O)Nc2ccc3n[nH]c(C)c3c2)C2(CCCC2)n2nnnc2N1C]

### Step 1. Methyl 2-cyclopentylidene-3-oxobutanoate

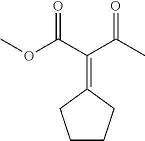

To a mixture of zinc chloride (0.7 g, 5.17 mmol), 3-oxobutanoic acid methyl ester (3.7 mL, 34.45 mmol) and cyclopentanone (4.58 mL, 51.67 mmol) was added methyl acetoacetate (4.21 mL, 44.78 mmol) and the reaction mixture was stirred at 50° C. for 30 hrs. The reaction was cooled to rt, diluted with water (20 mL) and DCM (100 mL), the organic layer was separated, dried over sodium sulfate, filtered and purified on Biotage SP1 (340 g silica gel column) using cyclohexane/EtOAc 10:0 to 6:4 as eluent to recover the desired product, (0.580 g, 3.18 mmol, 9.24% yield) as a yellow oil. ¹H NMR (400 MHz, CDCl₃) δ 3.83 (s, 1H), 3.81 (s, 3H), 2.57-2.78 (m, 4H), 2.30-2.33 (m, 3H), 1.72-1.77 (m, 4H). MS-ESI (m/z) calcd for C₁₀H₁₄O₃ [M+H]+: 183.1. Found 183.1.

### Step 2. Methyl 5′-methyl-4H-spiro[cyclopentane-1,7′-tetrazolo[1,5-a]pyrimidine]-6′-carboxylate

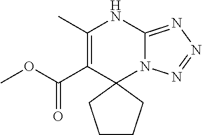 [SMILES: COC(=O)C1=C(C)Nc2nnnn2C12CCCC2]

A mixture of 2H-tetrazol-5-amine hydrate (328.1 mg, 3.18 mmol) and methyl 2-cyclopentylidene-3-oxobutanoate (580 mg, 3.18 mmol) in EtOH (25 mL) was heated at reflux for 15 hrs in the presence of molecular sieves. The reaction was cooled to rt, diluted with water (50 mL) and EtOAc (100 mL), the organic layer was separated, dried over sodium sulfate, filtered and concentrated to afford the desired product (480 mg, 1.93 mmol, 60.5% yield) as a brown solid. ¹H NMR (400 MHz, CD₃OD) δ 3.75-3.84 (m, 3H), 2.36-2.44 (m, 2H), 2.25-2.32 (m, 3H), 2.07-2.23 (m, 4H), 1.90-2.00 (m, 2H). MS-ESI (m/z) calcd for C₁₁H₁₅N₅O₂ [M+H]+: 250.1. Found 250.4.

### Step 3. Methyl 4′,5′-dimethyl-4H-spiro[cyclopentane-1,7′-tetrazolo[1,5-a]pyrimidine]-6′-carboxylate

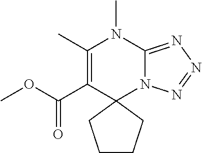 [SMILES: COC(=O)C1=C(C)N(C)c2nnnn2C12CCCC2]

Methyl 5-methylspiro[4H-tetrazolo[1,5-a]pyrimidine-7,1′-cyclopentane]-6-carboxylate (1250 mg, 5.01 mmol) was dissolved in DMF (15 mL) and Cs₂CO₃ (3288 mg, 10.03 mmol) was added portionwise. Iodomethane (0.47 mL, 7.52 mmol) was then added and the reaction mixture was stirred for 3 hrs at 50° C. The reaction was cooled to rt, diluted with water (50 mL) and EtOAc (100 mL). The organic layer was separated, dried over sodium sulfate, filtered and concentrated. The crude was purified on Biotage SP1 (50 g silica gel cartridge, cyclohexane:EtOAc 9:1 to 1:1 as eluent) to afford the desired product, (950 mg, 3.61 mmol, 71.95% yield) as a beige oil. ¹H NMR (400 MHz, CDCl₃) δ 3.81-3.87 (m, 3H), 3.51-3.57 (m, 3H), 2.20-2.37 (m, 7H), 2.05-2.20 (m, 2H), 1.82-1.94 (m, 2H). MS-ESI (m/z) calcd for C₁₂H₁₈N₅O₂ [M+H]+: 264.2. Found 264.2.

### Step 4. 4′,5′-Dimethyl-N-(3-methyl-2H-indazol-5-yl)-4′H-spiro[cyclopentane-1,7′-tetrazolo[1,5-a]pyrimidine]-6′-carboxamide

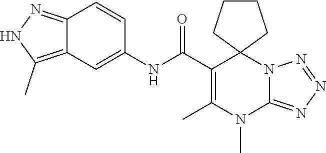 [SMILES: CC1=C(C(=O)Nc2ccc3n[nH]c(C)c3c2)C2(CCCC2)n2nnnc2N1C]

Methyl 4′,5′-dimethylspiro[cyclopentane-1,7′-tetrazolo[1,5-a]pyrimidine]-6′-carboxylate (50 mg, 0.190 mmol) and 3-methyl-1H-indazol-5-amine (36.33 mg, 0.250 mmol) were dissolved in toluene (5 mL). Trimethylaluminum (0.19 mL, 0.380 mmol) 2M in toluene was added dropwise. The reaction was heated at 120° C. for 3 hrs. The reaction was cooled to rt and a further amount of trimethylaluminum (0.19 mL, 0.380 mmol) was added. The reaction was then heated at 120° C. for 15 hrs. The reaction was cooled to rt and diluted with water (20 mL) and EtOAc (50 mL). The organic layer was separated, dried over sodium sulfate, filtered and concentrated to afford crude material (130 mg) which was purified by prep HPLC (Method A), to give the desired product, (11 mg, 0.03 mmol, 15.31% yield) as a light grey solid. NMR (400 MHz, acetone-d₆) δ 11.79 (br. s., 1H), 9.49 (br. s., 1H), 8.24 (d, J=1.54 Hz, 1H), 7.53-7.57 (m, 1H), 7.47-7.51 (m, 1H), 3.50 (s, 3H), 2.52-2.59 (m, 5H), 2.27 (s, 3H), 2.23 (dt, J=12.71, 6.30 Hz, 2H), 1.97-2.04 (m, 2H), 1.80-1.88 (m, 2H). MS-ESI (m/z) calcd for C₁₉H₂₂N₈O [M+H]+: 379.2. Found 379.2.

### Example 33. 4,5-Dimethyl-N-{3-[3-(morpholin-4-yl)phenyl]-1H-indazol-5-yl}-4H-spiro[[1,2,3,4]tetrazolo[1,5-a]pyrimidine-7,1′-cyclopentane]-6-carboxamide

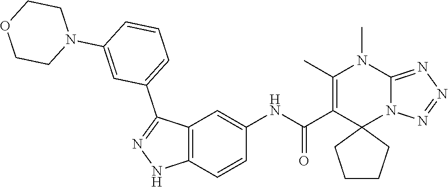 [SMILES: CC1=C(C(=O)Nc2ccc3[nH]nc(-c4cccc(N5CCOCC5)c4)c3c2)C2(CCCC2)n2nnnc2N1C]

Methyl 4′,5′-dimethylspiro[cyclopentane-1,7′-tetrazolo[1,5-a]pyrimidine]-6′-carboxylate (50 mg, 0.190 mmol) and 3-(3-morpholin-4-ylphenyl)-1H-indazol-5-amine (72.67 mg, 0.250 mmol) were dissolved in toluene (5 mL). Trimethylaluminum (0.19 mL, 0.380 mmol) 2M sol. in toluene was added dropwise. The reaction was heated at 120° C. for 3 hrs. The reaction was cooled to rt and a further amount of trimethylaluminum (0.19 mL, 0.380 mmol) was added. The reaction was heated at 120° C. for 15 hrs. The reaction was cooled to rt, diluted with water (20 mL) and EtOAc (50 mL), the organic layer was separated, dried over sodium sulfate, filtered and concentrated to afford crude material (150 mg) which was purified by prep HPLC (Method A), to afford the desired compound (22 mg, 0.04 mmol, 22.04% yield) as a white solid. ¹H NMR (400 MHz, DMSO-d₆) δ 13.15 (s, 1H), 10.39 (s, 1H), 8.52 (s, 1H), 7.53-7.61 (m, 2H), 7.34-7.49 (m, 3H), 7.03 (d, J=7.70 Hz, 1H), 3.75-3.85 (m, 4H), 3.45 (s, 3H), 3.20-3.25 (m, 4H), 2.36-2.45 (m, 2H), 2.14-2.29 (m, 5H), 1.87-2.01 (m, 2H), 1.74 (br. s., 2H). MS-ESI (m/z) calcd for C₂₈H₃₂N₉O₂ [M+H]+: 526.3. Found 526.3.

### Example 34. (R)—N-(3-(2-((2S,6R)-2,6-Dimethylmorpholino)pyridin-4-yl)-1H-indazol-5-yl)-4,5,7-trimethyl-4,7-dihydrotetrazolo[1,5-a]pyrimidine-6-carboxamide

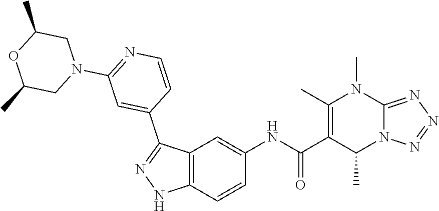 [SMILES: CC1=C(C(=O)Nc2ccc3[nH]nc(-c4ccnc(N5C[C@@H](C)O[C@@H](C)C5)c4)c3c2)[C@@H](C)n2nnnc2N1C]

### Step 1. (2S,6R)-4-(4-Bromopyridin-2-yl)-2,6-dimethylmorpholine

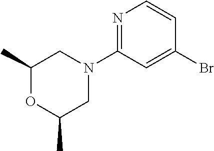

4-Bromo-2-fluoropyridine (1.17 mL, 11.4 mmol, 1 eq) was dissolved in 10 mL of DMF and 2,6-cis-dimethylmorpholine (1.68 mL, 13.6 mmol, 1.2 eq) and Cs₂CO₃ (7.40 g, 22.7 mmol, 2 eq) were added at rt. The mixture was stirred in a sealed vial at 100° C. overnight. Then the mixture was partitioned between water and EtOAc. The phases were separated, the aqueous layer was extracted with EtOAc (2×) and the combined organic layers were washed with brine (1×), dried over anhydrous Na₂SO₄ and evaporated to dryness. The crude material was purified by column chromatography on a 50 g silica gel column, using a 0-30% gradient of EtOAc in cyclohexane as eluent to afford (2R,6S)-4-(4-bromopyridin-2-yl)-2,6-dimethylmorpholine (2.55 g, 9.40 mmol, 82% yield) as a colorless oil. ¹H NMR (400 MHz, CDCl₃) δ 8.04-7.98 (m, 1H), 6.79 (dd, 7=1.2, 2.8 Hz, 2H), 4.04 (dd, 7=2.1, 13.1 Hz, 2H), 3.82-3.64 (m, 2H), 2.56 (dd, 7=10.6, 12.8 Hz, 2H), 1.29 (d, 7=6.4 Hz, 6H). MS-ESI (m/z) calcd for CᵤH₁₆BrN₂O [M+H]+: 271.0. Found 271.2.

### Step 2. (2S,6R)-2,6-Dimethyl-4-(4-(4,4,5,5-tetramethyl-1,3,2-dioxaborolan-2-yl)pyridin-2-yl)morpholine

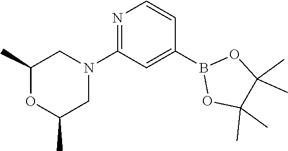

(2R,6S)-4-(4-Bromopyridin-2-yl)-2,6-dimethylmorpholine (2.40 g, 8.85 mmol, 1.0 eq), bis(pinacolato)diboron (2.47 g, 9.74 mmol, 1.1 eq) and KOAc (2.60 g, 26.55 mmol, 3.0 eq) were suspended in 1,4-dioxane (40 mL). The mixture was purged with N₂ for 5 min, and then Pd(dppf)Cl₂ (324 mg, 0.44 mmol, 0.05 eq) was added. The resulting mixture was heated to 100° C. for 1 h under a nitrogen atmosphere. The mixture was partitioned between water and EtOAc. The phases were separated; the organic layer was washed with water (1×), dried over anhydrous Na₂SO₄ and the solvent was removed under reduced pressure to give the crude product that was used without further purification. MS-ESI (m/z) calcd for C₁₇H₂₈BN₂O₃ [M+H]+: 319.2. Found 319.2.

### Step 3. 3-(2-((2S,6R)-2,6-Dimethylmorpholino)pyridin-4-yl)-1H-indazol-5-amine

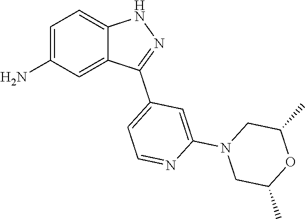 [SMILES: C[C@@H]1CN(c2cc(-c3n[nH]c4ccc(N)cc34)ccn2)C[C@H](C)O1]

(2R,6S)-2,6-Dimethyl-4-[4-(tetramethyl-1,3,2-dioxaborolan-2-yl)pyridin-2-yl]morpholine (crude, 8.85 mmol theoretical, 1 eq) and 3-bromo-1H-indazol-5-amine (2.25 g, 10.62 mmol, 1.2 eq) were dissolved in DMF (40 mL) and 11 mL of an aqueous 2M Na₂CO₃ solution were added. The mixture was purged with N₂ for 5 min, and then Pd(PPh₃)₄ (511 mg, 0.443 mmol, 0.05 eq) was added. The reaction mixture was stirred at 100° C. overnight under nitrogen atmosphere. The mixture was then partitioned between water and EtOAc. The phases were separated, the aqueous layer was extracted with EtOAc (2×) and the combined organic layers were washed with water (1×), dried over anhydrous Na₂SO₄ and the solvent was removed under reduced pressure. The crude material was purified by flash chromatography on a 110 g NH-silica gel column, eluting with a 0-10% gradient of MeOH in EtOAc followed by reverse phase flash chromatography on a 120 g C18 column eluting with a 0-35% gradient of CH₃CN in water containing 0.1% NH₃. 3-{2-[(2R,6S)-2,6-dimethylmorpholin-4-yl]pyridin-4-yl}-1H-indazol-5-amine (894 mg, 2.76 mmol, 31% yield over two steps) was obtained as a yellow solid. ¹H NMR (400 MHz, DMSO-d₆) δ 13.00 (s, 1H), 8.19 (d, J=5.2 Hz, 1H), 7.33 (d, J=8.8 Hz, 1H), 7.24-7.16 (m, 2H), 7.12 (s, 1H), 6.85 (d, J=8.7 Hz, 1H), 4.98 (s, 2H), 4.18 (dd, J=12.7, 2.3 Hz, 2H), 3.73-3.60 (m, 2H), 2.47-2.40 (m, 2H), 1.20 (d, J=6.2 Hz, 6H). MS-ESI (m/z) calcd for C₁₈N₂₂N₅O [M+H]+: 324.2. Found 324.2.

### Step 4. (R)—N-(3-(2-((2S,6R)-2,6-Dimethylmorpholino)pyridin-4-yl)-1H-indazol-5-yl)-4,5,7-trimethyl-4,7-dihydrotetrazolo[1,5-a]pyrimidine-6-carboxamide

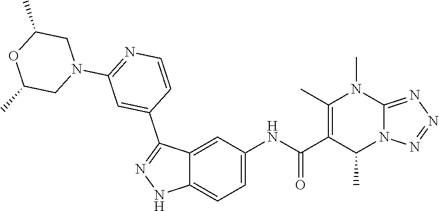 [SMILES: CC1=C(C(=O)Nc2ccc3[nH]nc(-c4ccnc(N5C[C@@H](C)O[C@@H](C)C5)c4)c3c2)[C@@H](C)n2nnnc2N1C]

(7R)-4,5,7-Trimethyl-4H,7H-[1,2,3,4]tetrazolo[1,5-a]pyrimidine-6-carboxylic acid (97 mg, 0.46 mmol, 1 eq) and 3-{2-[(2R,6S)-2,6-dimethylmorpholin-4-yl]pyridin-4-yl}-1H-indazol-5-amine (150 mg, 0.46 mmol, 1 eq) were dissolved in dry DMF (3 mL). Then the solution was cooled to 0° C. with an ice-water bath and TEA (0.13 mL, 0.92 mmol, 2 eq) and HATU (211 mg, 0.56 mmol, 1.2 eq) were added. The mixture was stirred at 0° C. for 5 min and then at room temperature over the weekend. The crude material was loaded directly onto a 12 g C18 column and purified by reverse phase chromatography using a 5-30% gradient of CH₃CN in H₂O containing 0.1% formic acid. The purest fractions were combined, evaporated to dryness and purified again by column chromatography on an 11 g NH-silica gel column, using a 0-10% gradient of MeOH in EtOAc as eluent. The product (40 mg, 0.078 mmol, 17% yield) was obtained pure as a white solid. ¹H NMR (400 MHz, DMSO-d₆) δ 13.43 (s, 1H), 10.27 (s, 1H), 8.65 (s, 1H), 8.27 (d, J=5.28 Hz, 1H), 7.53-7.65 (m, 2H), 7.31 (s, 1H), 7.21 (d, J=5.28 Hz, 1H), 5.77 (q, J=6.53 Hz, 1H), 4.23 (d, J=12.76 Hz, 2H), 3.60-3.78 (m, 2H), 3.44 (s, 3H), 2.47 (m, signal under DMSO, 2H), 2.21 (s, 3H), 1.57 (d, J=6.38 Hz, 3H), 1.21 (d, 0.7=6.16 Hz, 6H). MS-ESI (m/z) calcd for C₂₆H₃₁N₁₀O2 [M+H]+: 515.3. Found 515.3.

### Example 35. (R)-4,5,7-trimethyl-N-(3-(pyrrolidin-1-yl)-1H-indazol-5-yl)-4,7-dihydrotetrazolo[1,5-a]pyrimidine-6-carboxamide

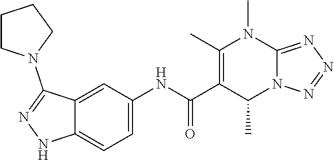 [SMILES: CC1=C(C(=O)Nc2ccc3[nH]nc(N4CCCC4)c3c2)[C@@H](C)n2nnnc2N1C]

### Step 1. 5-Nitro-3-(pyrrolidin-1-yl)-1H-indazole

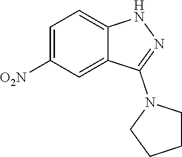 [SMILES: O=[N+]([O-])c1ccc2[nH]nc(N3CCCC3)c2c1]

3-Bromo-5-nitro-1H-indazole (500 mg, 2.06 mmol, 1 eq) was dissolved in pyrrolidine (3.5 mL). The mixture was stirred in a sealed tube at 120° C. for 16 h and then at 150° C. for 24 h. The mixture was cooled to room temperature and partitioned between EtOAc and water. The 2 phases were separated, the aqueous layer was extracted with EtOAc (1×) and then the combined organic phases were washed with water (1×), dried over anhydrous Na₂SO₄ and evaporated to dryness. The crude product was purified by flash chromatography, first on a 50 g silica gel column, using as eluent a gradient of EtOAc in cyclohexane from 0 to 100% and then by reverse phase column chromatography on a 30 g C18 column, using as eluent a gradient of CH₃CN in H₂O from 5 to 100% containing 0.1% formic acid. The target compound (250 mg, 1.08 mmol, 52% yield) was obtained as an orange solid. ¹H NMR (400 MHz, DMSO-d6) δ ppm 12.48 (br. s., 1H), 8.71 (d, J=2.0 Hz, 1H), 8.08 (dd, 0.7=1.5, 9.0 Hz, 1H), 7.40 (d, J=92 Hz, 1H), 3.69-3.53 (m, 4H), 2.00 (td, J=3.4, 6.5 Hz, 4H). MS-ESI (m/z) calcd for C₁₁H₁₃N₄O₂ [M+H]+: 233.1. Found 233.3.

### Step 2. 3-(Pyrrolidin-1-yl)-1H-indazol-5-amine

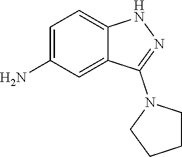 [SMILES: Nc1ccc2[nH]nc(N3CCCC3)c2c1]

5-Nitro-3-(pyrrolidin-1-yl)-1H-indazole (250 mg, 1.08 mmol, 1 eq) was dissolved in EtOH (15 mL) and 10% Pd/C (1 spatula tip) was added. The mixture was left to react under H₂ (1 atm) at room temperature for 2 h. The catalyst was then filtered off and the filter washed with EtOH. Volatiles were removed under vacuum to afford the product (210 mg, 1.04 mmol, 96% yield) as a brown solid. LC-MS: m/z=203.13 [M+H]+, 0.51 min. ¹H NMR (400 MHz, DMSO-d₆) δ 11.11 (br. s., 1H), 7.02 (d, J=8.6 Hz, 1H), 6.91 (d, 0.7=1.5 Hz, 1H), 6.70 (dd, 0.7=2.1, 8.7 Hz, 1H), 4.57 (br. s., 2H), 3.53-3.38 (m, 4H), 1.97-1.86 (m, 4H). MS-ESI (m/z) calcd for C₁₁H₁₅N₄ [M+H]+: 203.1. Found 203.1.

### Step 3. (R)-4,5,7-Trimethyl-N-(3-(pyrrolidin-1-yl)-1H-indazol-5-yl)-4,7-dihydrotetrazolo[1,5-a]pyrimidine-6-carboxamide

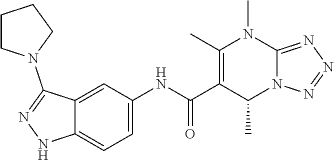 [SMILES: CC1=C(C(=O)Nc2ccc3[nH]nc(N4CCCC4)c3c2)[C@@H](C)n2nnnc2N1C]

(7R)-4,5,7-Trimethyl-4H,7H-[1,2,3,4]tetrazolo[1,5-a]pyrimidine-6-carboxylic acid (50 mg, 0.24 mmol, 1 eq) and 3-(pyrrolidin-1-yl)-1H-indazol-5-amine (58 mg, 0.29 mmol, 1.2 eq) were dissolved in dry DMF (2 mL). Then the solution was cooled to 0° C. with an ice-water bath and TEA (0.07 mL, 0.48 mmol, 2 eq.) and HATU (110 mg, 0.29 mmol, 1.2 eq) were added. The mixture was stirred at 0° C. for 5 min and then at room temperature overnight. The crude material was purified by reverse phase chromatography on a 12 g C18 column using a 5-35% gradient of CH₃CN in H₂O containing 0.1% formic acid as eluent. The purest fractions were combined and evaporated to dryness to afford the product (49 mg, 0.12 mmol, 52% yield) as a white solid. ¹H NMR (400 MHz, DMSO-d₆) δ 11.68 (br. s., 1H), 10.06 (s, 1H), 8.24 (s, 1H), 7.45 (d, 7=7.70 Hz, 1H), 7.27 (d, 7=8.80 Hz, 1H), 5.68-5.78 (m, 1H), 3.50-3.54 (m, 4H), 3.43 (s, 3H), 2.19 (s, 3H), 1.89-2.04 (m, 4H), 1.56 (d, 7=6.38 Hz, 3H). MS-ESI (m/z) calcd for C₁₉H₂₄N₉O [M+H]+: 394.2. Found 394.4.

### Example 36. (R)—N-(3-Isopropyl-1H-indazol-5-yl)-4,5,7-trimethyl-4,7-dihydrotetrazolo[1,5-a]pyrimidine-6-carboxamide

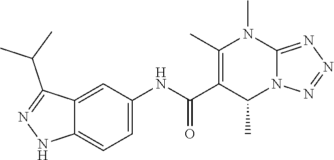 [SMILES: CC1=C(C(=O)Nc2ccc3[nH]nc(C(C)C)c3c2)[C@@H](C)n2nnnc2N1C]

### Step 1. 3-Bromo-1-(4-methoxybenzyl)-5-nitro-1H-indazole

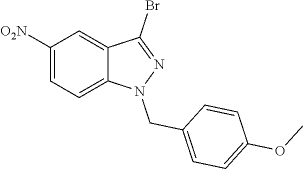 [SMILES: COc1ccc(Cn2nc(Br)c3cc([N+](=O)[O-])ccc32)cc1]

A mixture of 3-bromo-5-nitro-1H-indazole (1.0 g, 4.13 mmol, 1 eq) and K₂CO₃ (1.71 g, 12.39 mmol, 3 eq) in DMF (5 mL) was stirred at rt for 30 min, then 4-methoxybenzyl chloride (1.20 mL, 8.26 mmol, 2 eq) was added. The reaction mixture was stirred at rt overnight. The mixture was partitioned between water and EtOAc. The phases were separated; the aqueous layer was extracted with EtOAc (2×) and the combined organic layers were washed with water (1×), dried over anhydrous Na₂SO₄ and the solvent was removed under reduced pressure. The crude material was purified by flash chromatography on a 50 g silica gel column, eluting with a 0-20% gradient of EtOAc in cyclohexane. The product (1.38 g, 3.81 mmol, 92% yield) was obtained as an off-white solid. ¹H NMR (400 MHz, DMSO-d₆) δ=8.48 (d, J=22 Hz, 1H), 8.32 (dd, 7=2.2, 9.2 Hz, 1H), 8.08 (d, 7=9.5 Hz, 1H), 7.33-7.22 (m, 1H), 6.96-6.83 (m, 1H), 5.68 (s, 2H), 3.71 (s, 3H). MS-ESI (m/z) calcd for C₁₅H₁₃BrN₃O₃ [M+H]+: 362.0. Found 362.2.

### Step 2. I-(4-Methoxybenzyl)-5-nitro-3-(prop-1-en-2-yl)-1H-indazole

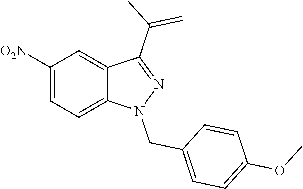 [SMILES: C=C(C)c1nn(Cc2ccc(OC)cc2)c2ccc([N+](=O)[O-])cc12]

4,4,5,5-Tetramethyl-2-(prop-1-en-2-yl)-1,3,2-dioxaborolane (0.17 mL, 0.92 mmol, 1 eq) and 3-bromo-1-(4-methoxybenzyl)-5-nitro-1H-indazole (400 mg, 1.10 mmol, 1.2 eq) were dissolved in THF/H₂O (5 mL/1 mL) and K₂CO₃ (381 mg, 2.76 mmol, 3 eq.) was added. The mixture was purged with N₂ for 5 min, and then Pd(dppf)Cl₂ (34 mg, 0.046 mmol, 0.05 eq.) was added. The reaction mixture was stirred under a nitrogen atmosphere at 100° C. for 3 h and left at 80° C. overnight. The mixture was partitioned between water and EtOAc, the aqueous phase was extracted with EtOAc (2×) and the combined organic layers were washed with water (1×), dried over anhydrous Na₂SO₄, filtered and evaporated to dryness. The crude material was purified by column chromatography on a 25 g silica gel column using a 0-30% gradient of MeOH in EtOAc as eluent. The product (320 mg, 0.99 mmol, 90% yield) was obtained as a yellow solid. NMR (400 MHz, DMSO-d₆) δ=8.81 (d, J=2.0 Hz, 1H), 8.25 (dd, J=2.2, 9.2 Hz, 1H), 7.96 (d, J=9.2 Hz, 1H), 7.25 (d, J=8.8 Hz, 2H), 6.96-6.80 (m, 2H), 5.86 (s, 1H), 5.67 (s, 2H), 5.51 (s, 1H), 3.70 (s, 3H), 2.26 (s, 3H). MS-ESI (m/z) calcd for C₁₈H₁₈N₃O₃ [M+H]+: 324.1. Found 324.2.

### Step 3. 3-Isopropyl-1-(4-methoxybenzyl)-1H-indazol-5-amine

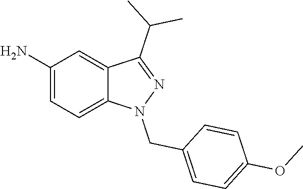 [SMILES: COc1ccc(Cn2nc(C(C)C)c3cc(N)ccc32)cc1]

1-(4-Methoxybenzyl)-5-nitro-3-(prop-1-en-2-yl)-1H-indazole (320 mg, 0.99 mmol, 1 eq) was dissolved in EtOH (8 mL) and EtOAc (8 mL) and Pd/C (1 spatula tip) was added. The mixture was left to react under H₂ (1 atm.) at room temperature overnight. The catalyst was then filtered off and the filter washed with EtOH. Volatiles were removed under vacuum to afford the title compound (280 mg, 0.95 mmol, 96% yield) as a yellow oil. ¹H NMR (400 MHz, METHANOL-d₄) δ=7.22 (d, J=8.8 Hz, 1H), 7.13-7.05 (m, 3H), 6.97-6.88 (m, 1H), 6.86-6.78 (m, 2H), 5.42 (s, 2H), 3.75 (s, 3H), 3.37-3.30 (m, 1H, signal under solvent), 1.44 (d, J=7.0 Hz, 6H). MS-ESI (m/z) calcd for C₁₈H₂₂N₃O [M+H]+: 296.2. Found 296.3.

### Step 4. 3-Isopropyl-1H-indazol-5-amine

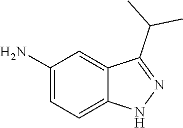

3-Isopropyl-1-(4-methoxybenzyl)-1H-indazol-5-amine (280 mg, 0.95 mmol, 1 eq) was dissolved in TFA (3 mL) and the mixture was left to react at 70° C. for 24 h and then irradiated under MW at 90° C. (3×, 1 h). The mixture was concentrated in vacuo, and the residue was dissolved in MeOH (3 mL) and treated with Na₂CO₃ (2 mL of 2M aqueous solution) at 45° C. for 16 h. The solvents were removed under reduced pressure and the residue was dissolved in EtOAc and water. The phases were separated, the aqueous layer was extracted with EtOAc (2×) and the combined organic extracts washed with water (1×), dried over anhydrous Na₂SO₄ and evaporated to dryness. The residue was purified by flash chromatography on a 28 g NH-silica gel column, using as eluent a gradient of EtOAc in cyclohexane from 50 to 100%. 3-(propan-2-yl)-1H-indazol-5-amine (110 mg, 0.62 mmol, 66% yield) was recovered as an off-white solid. LC-MS: m/z=176.14 [M+H]+, 0.55 min. ¹H NMR (400 MHz, DMSO-d6) δ=12.05 (s, 1H), 7.16 (d, J=8.6 Hz, 1H), 6.78 (s, 1H), 6.74 (dd, 0.7=1.9, 8.7 Hz, 1H), 4.69 (s, 2H), 3.20 (td, 0.7=6.9, 13.9 Hz, 1H), 1.33 (d, J=6.8 Hz, 6H). MS-ESI (m/z) calcd for C₁₀H₁₄N₃ [M+H]+: 176.1. Found 176.1.

### Step 5. (R)—N-(3-Isopropyl-1H-indazol-5-yl)-4,5,7-trimethyl-4,7-dihydrotetrazolo[1,5-a]pyrimidine-6-carboxamide

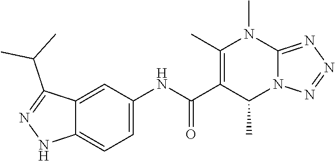

(7R)-4,5,7-Trimethyl-4H,7H-[1,2,3,4]tetrazolo[1,5-a]pyrimidine-6-carboxylic acid (50 mg, 0.24 mmol, 1 eq) and 3-isopropyl-1H-indazol-5-amine (51 mg, 0.29 mmol, 1.2 eq) were dissolved in dry DMF (2 mL). The solution was cooled to 0° C. with an ice-water bath and TEA (0.07 mL, 0.48 mmol, 2 eq.) and HATU (110 mg, 0.29 mmol, 1.2 eq) were added. The mixture was stirred at 0° C. for 5 min, at room temperature overnight, and then heated at 70° C. for 2 h. The mixture was loaded directly onto a 12 g C18 cartridge and purified by reverse phase chromatography using a 5-35% gradient of CH₃CN in H₂O containing 0.1% formic acid. The purest fractions were combined and evaporated to dryness to afford the target product (27 mg, 0.074 mmol, 31% yield) as a light yellow solid. ¹H NMR (400 MHz, DMSO-d₆) δ ppm 12.54 (s, 1H), 10.13 (s, 1H), 8.17 (s, 1H), 7.33-7.53 (m, 2H), 5.74 (d, J=6.38 Hz, 1H), 3.44 (s, 3H), 3.32 (m, 1H, peak under H₂O solvent), 2.20 (s, 3H), 1.56 (d, J=6.38 Hz, 3H), 1.37 (d, J=6.82 Hz, 6H). MS-ESI (m/z) calcd for C₁₈₁H₂₃N₈O [M+H]+: 367.2. Found 367.2.

### Example 37. trans-(7R)—N-(3-(2-(2,6-Dimethylmorpholino)pyridin-4-yl)-1H-indazol-5-yl)-4,5,7-trimethyl-4,7-dihydrotetrazolo[1,5-a]pyrimidine-6-carboxamide

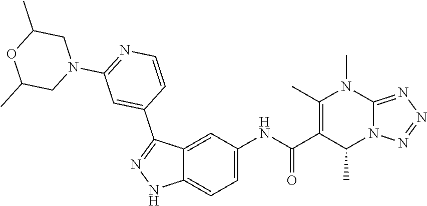

### Step 1. trans-4-(4-Bromopyridin-2-yl)-2,6-dimethylmorpholine

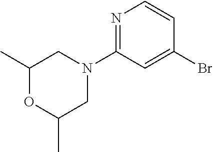

4-Bromo-2-fluoropyridine (1.0 g, 5.68 mmol, 1 eq) was dissolved in 5 mL of DMF. Morpholine (0.785 g, 6.82 mmol, 1.2 eq) and Cs₂CO₃ (3.70 g, 11.36 mmol, 2 eq) were then added at rt. The mixture was stirred in a sealed vial at 100° C. overnight. Then the mixture was partitioned between water and EtOAc. The phases were separated and the aqueous layer was extracted with EtOAc (2×). The combined organic layers were washed with brine (1×), dried over anhydrous Na₂SO₄ and evaporated to dryness. The crude material was purified by column chromatography on a 50 g silica gel column using a 0-50% gradient of EtOAc in cyclohexane as eluent. Product-containing fractions were combined and the solvent was removed to afford the product (1.32 g, 4.87 mmol, 86% yield) as a colourless oil. ¹H NMR (400 MHz, DMSO-d₆) δ 7.96 (d, J=5.3 Hz, 1H), 7.04 (d, J=1.5 Hz, 1H), 6.85-6.77 (m, 1H), 3.99 (pd, J=6.4, 3.3 Hz, 2H), 3.60 (dd, J=12.9, 3.4 Hz, 2H), 3.23 (dd, J=12.8, 6.4 Hz, 2H), 1.13 (d, J=6.4 Hz, 6H). MS-ESI (m/z) calcd for CᵤH₁₆BrN₂O [M+H]+: 271.0. Found 271.1.

### Step 2. trans-2,6-Dimethyl-4-(4-(4,4,5,5-tetramethyl-1,3,2-dioxaborolan-2-yl)pyridin-2-yl)morpholine

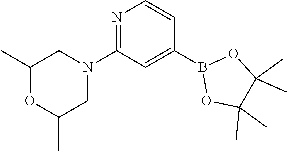

trans-4-(4-Bromopyridin-2-yl)-2,6-dimethylmorpholine (1.32 g, 4.87 mmol, 1.0 eq), bis(pinacolato)diboron (1.36 g, 5.35 mmol, 1.1 eq) and KOAc (1.43 g, 14.61 mmol, 3.0 eq) were suspended in 1,4-dioxane (20 mL). The mixture was purged with N₂ for 5 min and then Pd(dppf)Cl₂ (178 mg, 0.24 mmol, 0.05 eq) was added. The resulting mixture was heated to 100° C. for 1 h under a nitrogen atmosphere. The crude material was partitioned between water and EtOAc and the phases were separated. The aqueous layer was extracted with EtOAc (2×) and the combined organic layers were washed with water (1×), dried over anhydrous Na₂SO₄ and the solvent was removed under reduced pressure to afford the title compound (4.87 mmol theoretical) as a brown oil which was used without further purification. ¹H NMR (400 MHz, DMSO-d₆) δ 8.13 (dd, J=0.9, 4.8 Hz, 1H), 6.94 (s, 1H), 6.82 (d, J=4.8 Hz, 1H), 4.02 (dt, J=3.6, 6.4 Hz, 2H), 3.57-3.51 (m, 2H), 3.19 (dd, J=6.4, 12.5 Hz, 2H), 1.30 (s, 12H), 1.17 (d, J=3.5 Hz, 6H). MS-ESI (m/z) calcd for C₁₁H₁₈BN₂O₃ (boronic acid) [M+H]+: 236.1. Found 236.1.

### Step 3. 3-(2-(2,6-Dimethylmorpholino)pyridin-4-yl)-1H-indazol-5-amine

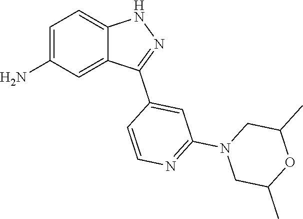

trans-2,6-Di methyl-4-(4-(4,4,5,5-tetramethyl-1,3,2-dioxaborolan-2-yl)pyridin-2-yl)morpholine (crude, 4.87 mmol theoretical, 1 eq) and 3-bromo-1H-indazol-5-amine (1.24 g, 5.84 mmol, 1.2 eq) were dissolved in DMF (20 mL) and 6 mL of a 2M Na₂CO₃ aqueous solution. The mixture was purged with N₂ for 5 min, and then Pd(PPh₃)₄ (281 mg, 0.24 mmol, 0.05 eq) was added. The reaction mixture was stirred at 100° C. overnight under nitrogen atmosphere. The mixture was partitioned between water and EtOAc. The phases were separated, the aqueous layer was extracted with EtOAc (2×) and the combined organic layers washed with water (1×), dried over anhydrous Na₂SO₄ and the solvent was removed under reduced pressure. The crude material was purified first by flash chromatography on a 110 g NH-silica gel column, eluting with a 5-100% gradient of EtOAc in cyclohexane, and then by reverse phase flash chromatography on a 60 g C18 column eluting with a 5-45% gradient of CH₃CN in water containing 0.1% NH₃ to afford the product as a yellow solid. (467 mg, 1.44 mmol, 30% yield over two steps). ¹H NMR (400 MHz, DMSO-d₆) δ 13.00 (s, 1H), 8.18 (d, J=5.2 Hz, 1H), 7.33 (d, J=8.8 Hz, 1H), 7.20-7.14 (m, 2H), 7.12 (d, J=1.9 Hz, 1H), 6.84 (dd, J=8.8, 1.9 Hz, 1H), 4.98 (s, 2H), 4.11-4.03 (m, 2H), 3.66 (dd, J=12.6, 3.4 Hz, 2H), 3.26 (dd, J=12.5, 6.3 Hz, 2H), 1.20 (d, J=6.4 Hz, 6H). MS-ESI (m/z) calcd for C₁₈H₂₂N₅O [M+H]+: 324.2. Found 324.2.

### Step 4. trans-(7R)—N-(3-(2-(2,6-Dimethylmorpholino)pyridin-4-yl)-1H-indazol-5-yl)-4,5,7-trimethyl-4,7-dihydrotetrazolo[1,5-a]pyrimidine-6-carboxamide

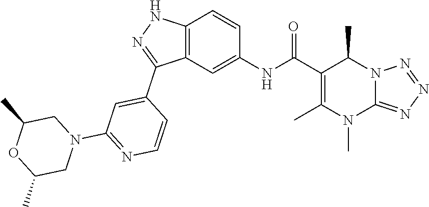

(7R)-4,5,7-Trimethyl-4H,7H-[1,2,3,4]tetrazolo[1,5-a]pyrimidine-6-carboxylic acid (50 mg, 0.24 mmol, 1 eq) and trans-2,6-dimethylmorpholin-4-yl]pyridin-4-yl}-1H-indazol-5-amine (78 mg, 0.24 mmol, 1 eq) were dissolved in dry DMF (2 mL). Then the solution was cooled to 0° C. with an ice-water bath and TEA (0.07 mL, 0.48 mmol, 2 eq) and HATH (109 mg, 0.29 mmol, 1.2 eq) were added. The mixture was stirred at 0° C. for 5 min, then at room temperature overnight and finally heated to 60° C. for 2 h. The mixture was partitioned between water and EtOAc, the aqueous phase was extracted with EtOAc (2×) and the combined organic layers were washed with water (1×), dried over anhydrous Na₂SO₄, filtered and evaporated to dryness. The crude material was purified by column chromatography on a 28 g NH-silica gel column using a 0-10% gradient of MeOH in EtOAc as eluent. The product-containing fractions were combined, evaporated to dryness and purified again by reverse phase chromatography on a 12 g C18 column using a 5-35% gradient of CH₃CN in H₂O containing 0.1% NH₃ as eluent. The title compound (36 mg, 0.07 mmol, 29% yield) was obtained pure as a white solid. ¹H NMR (400 MHz, DMSO-d₆) δ 13.43 (br. s., 1H), 10.27 (s, 1H), 8.62 (s, 1H), 8.25 (d, J=5.28 Hz, 1H), 7.50-7.69 (m, 2H), 7.27 (s, 1H), 7.18 (d, J=5.28 Hz, 1H), 5.67-5.88 (m, 1H), 4.08 (td, J=6.27, 3.30 Hz, 2H), 3.69 (dd, J=12.65, 3.19 Hz, 2H), 3.44 (s, 3H), 3.32-3.28 (m, 2H), 2.21 (s, 3H), 1.57 (d, J=6.38 Hz, 3H), 1.21 (d, J=6.38 Hz, 6H). MS-ESI (m/z) calcd for C₂₆H₃₁N₁₀O₂ [M+H]+: 515.3. Found 515.3.

### Separation of Enantiomers of trans-(7R)—N-(3-(2-(2,6-Dimethylmorpholino)pyridin-4-yl)-1H-indazol-5-yl)-4,5,7-trimethyl-4,7-dihydrotetrazolo[1,5-a]pyrimidine-6-carboxamide

### Semipreparative Chiral SFC:

Column: Chiralpak AS-H (25×2.0 cm), 5 μm. Modifier: (Methanol+0.1% Isopropylamine) 20%. Flow rate (mL/min): 45 mL/min. Pressure: 120 bar. Temperature: 38° C. UV detection: 220 nm. Loop: 800 μL. Total amount: 25 mg. Sample preparation: 25 mg in 3 mL (EtOH/MeOH 1/1)=8.3 mg/mL. Injection: 6.6 mg/injection.

### Analytic Chiral HPLC:

Column: Chiralpak AS-H (25×0.46 cm), 5 μm. Modifier: (Methanol+0.1% Isopropylamine) 20%. Flow rate (mL/min): 2.5 mL/min. Pressure: 120 bar. Temperature: 38° C. UV detection: 220 nm. Loop: 25 μL.

### First Eluting Diastereoisomer

### Example 37a. (7R)—N-(3-{2-[(2S,6S)-2,6-dimethylmorpholin-4-yl]pyridin-4-yl}-1H-indazol-5-yl)-4,5,7-trimethyl-4H,7H-[1,2,3,4]tetrazolo[1,5-a]pyrimidine-6-carboxamide

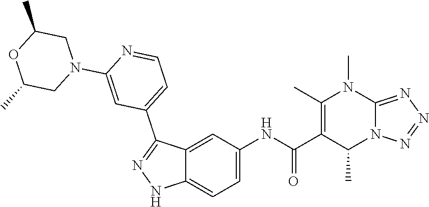

(7.9 mg, 0.015 mmol). ¹H NMR (400 MHz, DMSO-d₆) δ 13.42 (s, 1H), 10.26 (s, 1H), 8.61 (s, 1H), 8.24 (d, J=5.2 Hz, 1H), 7.64-7.52 (m, 2H), 7.26 (s, 1H), 7.17 (d, J=5.2 Hz, 1H), 5.76 (q, J=6.3 Hz, 1H), 4.07 (pd, J=6.3, 3.2 Hz, 2H), 3.68 (dd, J=12.6, 3.4 Hz, 2H), 3.44 (s, 3H), 3.30-3.27 (m, 2H), 2.20 (s, 3H), 1.57 (d, J=6.3 Hz, 3H), 1.20 (d, J=6.4 Hz, 6H). Analytical chiral-HPLC (e.e.=100%, 9.9 min). Single diastereoisomer of unknown absolute configuration on the trans morpholine. Stereochemistry on the trans morpholine arbitrarily assigned. MS-ESI (m/z) calcd for C₂₆H₃₁N₁₀O₂ [M+H]+: 515.3. Found 515.3.

### Second Eluting Diastereoisomer

### Example 37b. (7R)—N-(3-{2-[(2R,6R)-2,6-dimethylmorpholin-4-yl]pyridin-4-yl}-1H-indazol-5-yl)-4,5,7-trimethyl-4H,7H-[1,2,3,4]tetrazolo[1,5-a]pyrimidine-6-carboxamide

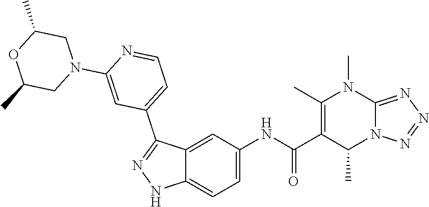

(8.9 mg, 0.017 mmol). ¹H NMR (400 MHz, DMSO-d₆) δ 13.41 (s, 1H), 10.26 (s, 1H), 8.62 (s, 1H), 8.24 (d, J=5.1 Hz, 1H), 7.64-7.51 (m, 2H), 7.26 (s, 1H), 7.17 (d, J=5.2 Hz, 1H), 5.76 (q, J=6.2 Hz, 1H), 4.07 (pd, J=6.4, 3.2 Hz, 2H), 3.68 (dd, J=12.7, 3.4 Hz, 2H), 3.44 (s, 3H), 3.30-3.27 (m, 2H), 2.20 (s, 3H), 1.57 (d, J=6.3 Hz, 3H), 1.21 (d, J=6.4 Hz, 6H). Analytical chiral-HPLC (e.e.=99.6%, 11.1 min). Single diastereoisomer of unknown absolute configuration on the trans morpholine. Stereochemistry on the trans morpholine arbitrarily assigned. MS-ESI (m/z) calcd for C₂₆H₃₁N₁₀O₂ [M+H]+: 515.3. Found 515.3.

### Example 38. (R)—N-(3-(2-((3R,5S)-3,5-Dimethylpiperidin-1-yl)pyridin-4-yl)-1H-indazol-5-yl)-4,5,7-trimethyl-4,7-dihydrotetrazolo[1,5-a]pyrimidine-6-carboxamide

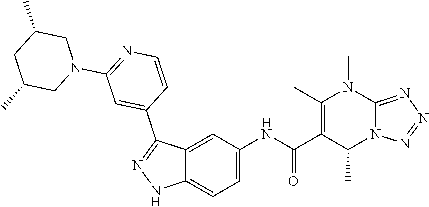

### Step 1. 4-Bromo-2-(63R,5S)-3,5-dimethylpiperidin-1-yl)pyridine

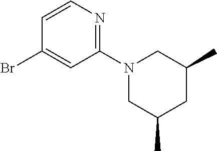

4-Bromo-2-fluoropyridine (0.45 mL, 4.42 mmol, 1 eq) was dissolved in 5 mL DMF and cis-3,5-dimethylpiperidine (0.5 g, 4.42 mmol, 1 eq) and Cs₂CO₃ (2.88 g, 8.84 mmol, 2 eq) were added at room temperature. The mixture was stirred in a sealed vial at 100° C. overnight. Then the mixture was partitioned between water and EtOAc. The phases were separated, the aqueous layer was extracted with EtOAc (2×) and the combined organic layers washed with brine (1×), dried over anhydrous Na₂SO₄ and evaporated to dryness. The crude material was purified by column chromatography on a 50 g silica gel column, using a 0-30% gradient of EtOAc in cyclohexane as eluent. Product-containing fractions were combined to afford the product (1.0 g, 3.71 mmol, 84% yield) as a colorless oil. ¹H NMR (400 MHz, CDCl₃) δ 7.98 (d, J=53 Hz, 1H), 6.81 (d, J=1.3 Hz, 1H), 6.69 (dd, J=1.3, 5.3 Hz, 1H), 4.33-4.16 (m, 2H), 2.39-2.24 (m, 2H), 1.87-1.82 (m, 1H), 1.77-1.62 (m, 2H), 0.97 (d, J=6.6, 6H), 0.81 (q, J=12.0 Hz, 1H). MS-ESI (m/z) calcd for C₁₂H₁₈BrN₂ [M+H]+: 269.1. Found 269.2.

### Step 2. 2-((3R,5S)-3,5-Dimethylpiperidin-1-yl)-4-(4,4,5,5-tetramethyl-1,3,2-dioxaborolan-2-yl)pyridine

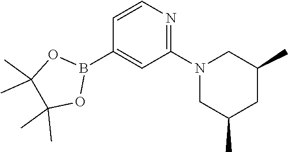

4-Bromo-2-[(3R,5S)-3,5-dimethylpiperidin-1-yl]pyridine (1.0 g, 3.71 mmol, 1 eq), bis(pinacolato)diboron (1.04 g, 4.09 mmol, 1.1 eq) and KOAc (1.09 g, 11.13 mmol, 3 eq.) were suspended in 1,4-dioxane (15 mL). The mixture was purged with N₂ for 5 min, and then Pd(dppf)Cl₂ (135 mg, 0.18 mmol, 0.05 eq.) was added. The resulting mixture was heated to 100° C. for 16 h under a nitrogen atmosphere. The crude material was partitioned between water and EtOAc. The phases were separated, the aqueous layer was extracted with EtOAc (2×) and the combined organic layers were washed with water (1×), dried over anhydrous Na₂SO₄ and the solvent was removed under reduced pressure to afford the product as a brown oil which was used without further purification. LC-MS: m/z=235.26 as boronic acid [M+H]+, 0.53 min. MS-ESI (m/z) calcd for C₁₂H₂₀BN₂O₂ (boronic acid) [M+H]+: 235.2.2. Found 235.3.

### Step 3. 3-(2-((3R,5S)-3,5-Dimethylpiperidin-1-yl)pyridin-4-yl)-1H-indazol-5-amine

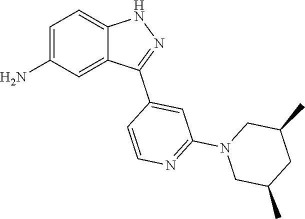

2-[(3R,5S)-3,5-Dimethylpiperidin-1-yl]-4-(4,4,5,5-tetramethyl-1,3,2-dioxaborolan-2-yl)pyridine (crude, 3.71 mmol theoretical, 1 eq) and 3-bromo-1H-indazol-5-amine (865 mg, 4.08 mmol, 1.1 eq) were dissolved in DMF (15 mL) and 4 mL of an aqueous 2M Na₂CO₃ solution. The mixture was purged with N₂ for 5 min, and then Pd(PPh₃)₄ (214 mg, 0.18 mmol, 0.05 eq) was added. The reaction mixture was stirred at 100° C. overnight under a nitrogen atmosphere. The mixture was partitioned between water and EtOAc. The phases were separated and the aqueous layer was extracted with EtOAc (2×). The combined organic layers were washed with water (1×), dried over anhydrous Na₂SO₄ and the solvent was removed under reduced pressure. The crude material was purified first by flash chromatography on a 55 g NH-silica gel column, eluting with a 50-100% gradient of EtOAc in cyclohexane, and then by reverse phase flash chromatography on a 28 g C18 column eluting with a 0-50% gradient of acetonitrile in water containing 0.1% NH₃ to afford the product (140 mg, 0.43 mmol, 11% yield over two steps). ¹H NMR (400 MHz, DMSO-d₆) δ 12.97 (s, 1H), 8.17 (d, J=5.3 Hz, 1H), 7.33 (d, 7=8.8 Hz, 1H), 7.20 (s, 1H), 7.13-7.06 (m, 2H), 6.85 (dd, 7=1.8, 8.8 Hz, 1H), 4.98 (s, 2H), 4.34 (d, 7=9.7 Hz, 2H), 2.33 (t, 7=12.1 Hz, 2H), 1.81 (d, 7=12.5 Hz, 1H), 1.73-1.55 (m, 2H), 0.94 (d, 7=6.6 Hz, 6H), 0.81 (q, 7=12.1 Hz, 1H). MS-ESI (m/z) calcd for C₁₉H₂₄N₅ [M+H]+: 322.2. Found 322.2.

### Step 4. (R)—N-(3-(2-((3R,5S)-3,5-Dimethylpiperidin-1-yl)pyridin-4-yl)-1H-indazol-5-yl)-4,5,7-trimethyl-4,7-dihydrotetrazolo[1,5-a]pyrimidine-6-carboxamide

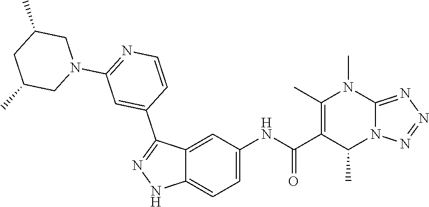

(7R)-4,5,7-Trimethyl-4H,7H-[1,2,3,4]tetrazolo[1,5-a]pyrimidine-6-carboxylic acid (50 mg, 0.24 mmol, 1 eq) and 3-{2-[(3R,5S)-3,5-dimethylpiperidin-1-yl]pyridin-4-yl}-1H-indazol-5-amine (77 mg, 0.24 mmol, 1 eq) were dissolved in dry DMF (2 mL). The solution was cooled to 0° C. with an ice-water bath and TEA (0.07 mL, 0.48 mmol, 2 eq) and HATU (109 mg, 0.29 mmol, 1.2 eq) were added. The mixture was stirred at 0° C. for 5 min, at room temperature overnight, and then heated to 60° C. for 2 h. The mixture was partitioned between water and EtOAc. The aqueous phase was extracted with EtOAc (2×) and the combined organic layers were washed with water (1×), dried over anhydrous Na₂SO₄, filtered and evaporated to dryness. The crude material was purified by column chromatography on an 11 g NH-silica gel column, using as eluent a 0-10% gradient of MeOH in EtOAc. Product-containing fractions were combined, evaporated to dryness and further purified by preparative HPLC (Method A). The target product (21 mg, 0.041 mmol, 17% yield) was obtained as a white solid (2% w/w formic acid by NMR). ¹H NMR (400 MHz, DMSO-d₆) δ 13.42 (br. s., 1H), 10.28 (s, 1H), 8.67 (s, 1H), 8.22 (d, J=5.06 Hz, 1H), 7.57-7.66 (m, 1H), 7.50-7.56 (m, 1H), 7.28 (s, 1H), 7.12 (d, J=5.06 Hz, 1H), 5.65-5.86 (m, 1H), 4.38 (d, J=12.98 Hz, 2H), 3.44 (s, 3H), 2.32-2.41 (m, 2H), 2.21 (s, 3H), 1.82 (d, J=12.76 Hz, 1H), 1.60-1.72 (m, 2H), 1.57 (d, J=6.38 Hz, 3H), 0.95 (d, J=6.60 Hz, 6H), 0.83 (q, J=12.18 Hz, 1H). MS-ESI (m/z) calcd for C₂₇H₃₃N₁₀O [M+H]+: 513.3. Found 513.9.

### Example 39. (R)-4,5,7-Trimethyl-N-(3-phenyl-1H-indazol-5-yl)-4,7-dihydrotetrazolo[1,5-a]pyrimidine-6-carboxamide

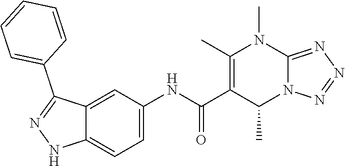

(7R)-4,5,7-Trimethyl-4H,7H-[1,2,3,4]tetrazolo[1,5-a]pyrimidine-6-carboxylic acid (50 mg, 0.24 mmol, 1 eq) and 3-phenyl-1H-indazol-5-amine (Intermediate 5; 50 mg, 0.24 mmol, 1 eq) were dissolved in dry DMF (2 mL). Then the solution was cooled to 0° C. with an ice-water bath and TEA (0.07 mL, 0.48 mmol, 2 eq) and HATU (109 mg, 0.29 mmol, 1.2 eq) were added. The mixture was stirred at 0° C. for 5 min and then at rt overnight. The mixture was partitioned between water and EtOAc, the aqueous phase was extracted with EtOAc (2×) and the combined organic layers were washed with water (1×), dried over anhydrous Na₂SO₄, filtered and evaporated to dryness. The crude material was purified first by column chromatography on a 28 g NH-silica gel column, using a 0-10% gradient of MeOH in EtOAc, then by reverse phase chromatography on a 12 g C18 column using a 5-45% gradient of CH₃CN in H₂O containing 0.1% formic acid. The product (18 mg, 0.045 mmol, 19% yield) was obtained as a white solid. ¹H NMR (400 MHz, DMSO-d₆) δ 13.22 (s, 1H), 10.24 (s, 1H), 8.49 (s, 1H), 7.94 (d, J=126 Hz, 2H), 7.50-7.65 (m, 4H), 7.37-7.46 (m, 1H), 5.76 (m, J=6.38 Hz, 1H), 3.44 (s, 3H), 2.21 (s, 3H), 1.57 (d, J=6.38 Hz, 3H). MS-ESI (m/z) calcd for C₂₁H₂₁N₈O [M+H]+: 401.2. Found 401.4.

### Example 40. (R)—N-(3-(3-((2S,6R)-2,6-Dimethylmorpholino)phenyl)-1H-indazol-5-yl)-4,5,7-trimethyl-4,7-dihydrotetrazolo[1,5-a]pyrimidine-6-carboxamide

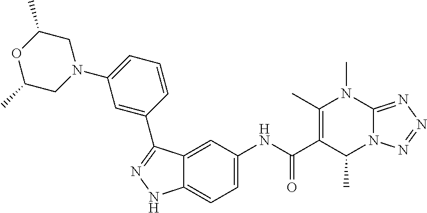 [SMILES: CC1=C(C(=O)Nc2ccc3[nH]nc(-c4cccc(N5C[C@@H](C)O[C@@H](C)C5)c4)c3c2)[C@@H](C)n2nnnc2N1C]

### Step 1. (2S,6R)-4-(3-Bromophenyl)-2,6-dimethylmorpholine

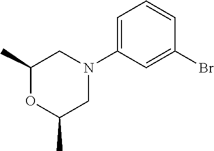

A mixture of 1,3-dibromobenzene (2.56 mL, 21.20 mmol, 1 eq), (2R,6S)-2,6-dimethylmorpholine (2.61 mL, 21.20 mmol, 1 eq), NaO-t-Bu (2.44 g, 25.44 mmol, 1.2 eq), rac-BINAP (0.99 g, 1.59 mmol, 0.075 eq) and Pd₂(dba)₃ (0.485 g, 0.53 mmol, 0.025 eq) in toluene (20 mL) was heated at 80° C. overnight under a nitrogen atmosphere. After cooling to rt, the mixture was diluted with DCM and filtered. The filtrate was washed with water (1×) and the organic layer was evaporated under reduced pressure. The crude material was then purified by column chromatography on a silica gel cartridge, using a 0-10% gradient of EtOAc in cyclohexane. The product (3.35 g, 12.40 mmol, 58% yield) was obtained as a yellow oil. ¹H NMR (400 MHz, DMSO-d₆) δ 7.18-7.11 (m, 1H), 7.08 (t, J=2.2 Hz, 1H), 6.92 (td, J=9.2, 8.7, 2.1 Hz, 2H), 3.70-3.57 (m, 4H), 2.31-2.20 (m, 2H), 1.14 (d, J=6.1 Hz, 6H). MS-ESI (m/z) calcd for C₁₂H₁₇BrNO [M+H]+: 270.0. Found 270.2.

### Step 2. (2S,6R)-2,6-Dimethyl-4-(3-(4,4,5,5-tetramethyl-1,3,2-dioxaborolan-2-yl)phenyl)morpholine

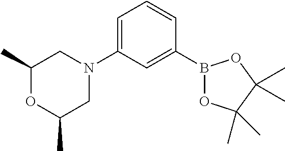

(2R,6S)-4-(3-Bromophenyl)-2,6-dimethylmorpholine (3.35 g, 12.40 mmol, 1.0 eq), bis(pinacolato)diboron (3.46 g, 13.64 mmol, 1.1 eq) and KOAc (3.65 g, 37.2 mmol, 3.0 eq) were suspended in 1,4-dioxane (60 mL). The mixture was purged with N₂ for 5 min, and then Pd(dppf)Cl₂ (454 mg, 0.62 mmol, 0.05 eq) was added. The resulting mixture was heated to 100° C. for 1 h under a nitrogen atmosphere. The crude material was partitioned between water and EtOAc. The phases were separated, the aqueous layer was extracted with EtOAc (2×) and the combined organic layers washed with water (1×), dried over anhydrous Na₂SO₄ and the solvent was removed under reduced pressure to afford the product (12.40 mmol theoretical) as a brown oil which was used without further purification. ¹H NMR (400 MHz, DMSO-d₆) δ 7.28-7.21 (m, 1H), 7.19-7.06 (m, 3H), 3.76-3.64 (m, 2H), 3.55 (d, 7=10.3 Hz, 2H), 2.28-2.18 (m, 2H), 1.29 (s, 12H), 1.16 (d, 7=3.1 Hz, 6H). MS-ESI (m/z) calcd for C₁₈H₂₉BNO₃ [M+H]+: 318.2. Found 318.4.

### Step 3. 3-(3-((2S,6R)-2,6-Dimethylmorpholino)phenyl)-1H-indazol-5-amine

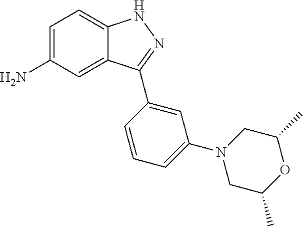 [SMILES: C[C@@H]1CN(c2cccc(-c3n[nH]c4ccc(N)cc34)c2)C[C@H](C)O1]

(2R,6S)-2,6-dimethyl-4-[3-(tetramethyl-1,3,2-dioxaborolan-2-yl)phenyl]morpholine (crude, 12.40 mmol theoretical, 1 eq) and 3-bromo-1H-indazol-5-amine (3.15 g, 14.88 mmol, 1.2 eq) were dissolved in 60 mL of DMF and 16 mL of a 2M aqueous Na₂CO₃ solution. The mixture was purged with N₂ for 5 min, and then Pd(PPh₃)₄ (716 mg, 0.62 mmol, 0.05 eq) was added. The reaction mixture was stirred at 100° C. overnight under nitrogen atmosphere. The mixture was partitioned between water and EtOAc. The phases were separated; the aqueous layer was extracted with EtOAc (2×) and the combined organic layers washed with water (1×), dried over anhydrous Na₂SO₄ and the solvent removed under reduced pressure. The crude material was purified first by flash chromatography on a 110 g NH-silica gel column, eluting with a 30-100% gradient of EtOAc in cyclohexane followed by reverse phase flash chromatography on a 120 g C18 column eluting with a 0-45% gradient of acetonitrile in water containing 0.1% NH₃ to afford the product (909 mg, 2.82 mmol, 23% yield over two steps). NMR (400 MHz, DMSO-d₆) δ 12.69 (s, 1H), 7.40 (d, J=2.4 Hz, 1H), 7.36-7.24 (m, 3H), 7.08 (d, J=1.9 Hz, 1H), 6.95 (dt, J=6.2, 2.7 Hz, 1H), 6.81 (dd, J=8.8, 1.9 Hz, 1H), 4.89 (s, 2H), 3.74 (dqd, 7=12.4, 6.1, 2.2 Hz, 2H), 3.63 (dd, 7=12.1, 2.3 Hz, 2H), 2.33 (t, J=11.1 Hz, 2H), 1.19 (d, J=6.2 Hz, 6H). MS-ESI (m/z) calcd for C₁₉H₂₃N₄O [M+H]+: 323.2. Found 323.2.

### Step 4. (R)—N-(3-(3-((2S,6R)-2,6-Dimethylmorpholino)phenyl)-1H-indazol-5-yl)-4,5,7-trimethyl-4,7-dihydrotetrazolo[1,5-a]pyrimidine-6-carboxamide

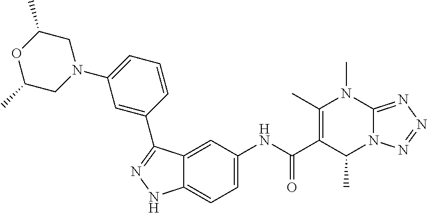 [SMILES: CC1=C(C(=O)Nc2ccc3[nH]nc(-c4cccc(N5C[C@@H](C)O[C@@H](C)C5)c4)c3c2)[C@@H](C)n2nnnc2N1C]

(7R)-4,5,7-trimethyl-4H,7H-[1,2,3,4]tetrazolo[1,5-a]pyrimidine-6-carboxylic acid (50 mg, 0.24 mmol, 1 eq) and 3-{3-[(2R,6S)-2,6-dimethylmorpholin-4-yl]phenyl}-1H-indazol-5-amine (77.4 mg, 0.24 mmol, 1 eq) were dissolved in dry DMF (3 mL). Then the solution was cooled to 0° C. with an ice-water bath and TEA (0.07 mL, 0.48 mmol, 2 eq) and HATU (109 mg, 0.29 mmol, 1.2 eq) were added. The mixture was stirred at 0° C. for 5 min and then at room temperature overnight. The mixture was partitioned between water and EtOAc, the aqueous phase was extracted with EtOAc (2×) and the combined organic layers were washed with water (1×), dried over anhydrous Na₂SO₄, filtered and evaporated to dryness. The crude material was purified by reverse phase column chromatography on a 30 g C18 column, using a 0-45% gradient of CH₃CN in H₂O containing 0.1% HCOOH. The product containing fractions were combined and evaporated to dryness to afford the product (28.5 mg, 0.06 mmol, 23% yield). ¹H NMR (400 MHz, DMSO-d₆) δ 13.14 (s, 1H), 10.23 (s, 1H), 8.59 (s, 1H), 7.59-7.44 (m, 3H), 7.42-7.31 (m, 2H), 7.02 (dd, J=8.1, 2.2 Hz, 1H), 5.75 (q, J=6.2 Hz, 1H), 3.81-3.63 (m, 4H), 3.43 (s, 3H), 2.40-2.30 (m, 2H), 2.20 (s, 3H), 1.56 (d, J=6.2 Hz, 3H), 1.19 (d, J=6.2 Hz, 6H). MS-ESI (m/z) calcd for C₂₇H₃₂N₉O₂ [M+H]+: 514.3. Found 514.5.

### Example 41. 4,5,7-Trimethyl-N-(3-methyl-1H-indazol-5-yl)-4,7-dihydrotetrazolo[1,5-a]pyrimidine-6-carboxamide

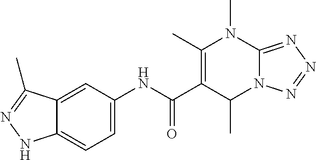 [SMILES: CC1=C(C(=O)Nc2ccc3[nH]nc(C)c3c2)C(C)n2nnnc2N1C]

Racemic 4,5,7-trimethyl-4H,7H-[1,2,3,4]tetrazolo[1,5-a]pyrimidine-6-carboxylic acid (1.0 g, 4.78 mmol, 1 eq) and 3-methyl-1H-indazol-5-amine (1.41 g, 9.56 mmol, 2 eq) were dissolved in dry DMF (20 mL). The solution was cooled to 0° C. with an ice-water bath and TEA (1.33 mL, 9.56 mmol, 2 eq) and HATU (2.18 g, 5.75 mmol, 1.2 eq) were added. The mixture was stirred at 0° C. for 5 min and then at room temperature for 72 hr. The reaction was partitioned between water and EtOAc and the phases were separated. The aqueous layer was extracted with EtOAc (2×) and the combined organic phases washed with water (1×), dried over anhydrous Na₂SO₄ and evaporated to dryness. The crude material was dissolved in DMSO and purified by column chromatography on a 110 g C18 column using a 5-50% gradient of CH₃CN in H₂O containing 0.1% formic acid. The target compound (592 mg, 1.75 mmol, 37% yield) was obtained as a light pink solid. ¹H NMR (400 MHz, DMSO-d₆) δ 12.57 (s, 1H), 10.14 (s, 1H), 8.11 (s, 1H), 7.43 (d, J=1.00 Hz, 2H), 5.74 (q, J=5.94 Hz, 1H), 3.43 (s, 3H), 2.47 (s, 3H), 2.20 (d, J=1.00 Hz, 3H), 1.57 (d, J=6.53 Hz, 3H). MS-ESI (m/z) calcd for C₁₆H₁₉N₈O [M+H]+: 339.2. Found 339.4.

### Example 42. (R)—N-(1-Aminoisoquinolin-6-yl)-4,5,7-trimethyl-4,7-dihydrotetrazolo[1,5-a]pyrimidine-6-carboxamide

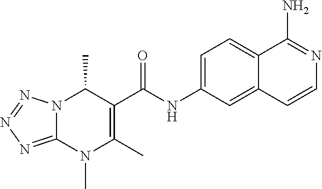 [SMILES: CC1=C(C(=O)Nc2ccc3c(N)nccc3c2)[C@@H](C)n2nnnc2N1C]

### Step 1. tert-butyl N-[(tert-butoxy)carbonyl]-N-(6-nitroisoquinolin-1-yl)carbamate

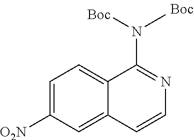 [SMILES: CN(C(=O)OC(C)(C)C)c1nccc2cc([N+](=O)[O-])ccc12]

A suspension of 6-nitroisoquinolin-1-amine (110 mg, 0.54 mmol, 1 eq), di-tert-butyl dicarbonate (335 mg, 1.53 mmol, 2.6 eq) and DMAP (3.5 mg, catalytic) in CH₃CN (3.0 mL) was stirred at 70° C. for 1 h. After that time, volatiles were removed under reduced pressure and the residue was purified by flash chromatography on a 25 g silica gel column, using as eluent a gradient of EtOAc in cyclohexane from 0 to 20%. Product-containing fractions were combined to afford tert-butyl N-[(tert-butoxy)carbonyl]-N-(6-nitroisoquinolin-1-yl)carbamate (160 mg, 0.410 mmol, 70% yield) as a pale yellow solid. ¹H NMR (400 MHz, CDCl₃) δ 8.84 (d, J=2.0 Hz, 1H), 8.64 (d, J=5.5 Hz, 1H), 8.41 (dd, J=22, 9.0 Hz, 1H), 8.17 (d, J=9.2 Hz, 1H), 7.88 (d, J=5.7 Hz, 1H), 1.36 (s, 18H). MS-ESI (m/z) calcd for C₁₉H₂₄N₃O₆ [M+H]+: 390.2. Found 390.2.

### Step 2. tert-butyl N-(6-aminoisoquinolin-1-yl)-N-[(tert-butoxy)carbonyl]carbamate

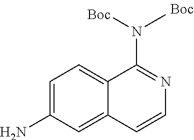 [SMILES: CN(C(=O)OC(C)(C)C)c1nccc2cc(N)ccc12]

tert-Butyl N-[(tert-butoxy)carbonyl]-N-(6-nitroisoquinolin-1-yl)carbamate (160 mg, 0.41 mmol, 1 eq) was dissolved in EtOH (5.0 mL) and Pd/C 10% (50 mg) was added. The mixture was left to react under H₂ (1 atm) at room temperature for 90 minutes. The catalyst was then filtered off and the filter washed with EtOH. The filtrate was recovered and dried under reduced pressure to afford tert-butyl N-(6-aminoisoquinolin-1-yl)-N-[(tert-butoxy)carbonyl]carbamate (143 mg, 0.40 mmol, 97% yield) as an off-white solid. ¹H NMR (400 MHz, DMSO-d₆) δ 8.04 (d, 7=5.7 Hz, 1H), 7.50 (d, 7=9.0 Hz, 1H), 7.38 (d, 7=5.7 Hz, 1H), 7.05 (dd, 7=2.1, 8.9 Hz, 1H), 6.78 (d, 7=2.0 Hz, 1H), 6.06 (s, 2H), 1.31 (s, 18H). MS-ESI (m/z) calcd for C₁₉H₂₆N₃O₄ [M+H]+: 360.2. Found 360.4.

### Step 3. tert-butyl N-[(tert-butoxy)carbonyl]-N-{6-[(7R)-4,5,7-trimethyl-4H,7H-[1,2,3,4]tetrazolo[1,5-a]pyrimidine-6-amido]isoquinolin-1-yl}carbamate

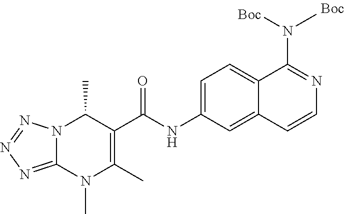 [SMILES: CC1=C(C(=O)Nc2ccc3c(N(C)C(=O)OC(C)(C)C)nccc3c2)[C@@H](C)n2nnnc2N1C]

(7R)-4,5,7-Trimethyl-4H,7H-[1,2,3,4]tetrazolo[1,5-a]pyrimidine-6-carboxylic acid (55 mg, 0.26 mmol, 1 eq) and tert-butyl N-(6-aminoisoquinolin-1-yl)-N-[(tert-butoxy)carbonyl]carbamate (114 mg, 0.32 mmol, 1.2 eq) were dissolved in pyridine (0.5 mL). Then EDCI (61 mg, 0.32 mmol, 1.2 eq) and DMAP (3 mg, 0.025 mmol, 0.1 eq) were added. The resulting solution was stirred at 70° C. for 16 h. The mixture was diluted with EtOAc and washed with water (3×) and brine (1×). The orange organic layer was dried over anhydrous Na₂SO₄ and evaporated to dryness under reduced pressure. The crude material was purified first by reverse phase column chromatography on a 12 g C18 column, using as eluent a gradient of CH₃CN in H₂O from 0 to 60% in presence of 0.1% HCOOH, then by normal phase column chromatography on an 11 g NH-silica gel column, eluting with a gradient of EtOAc in cyclohexane from 50 to 100%. tert-butyl-N-[(tert-butoxy)carbonyl]-N-{6-[(7R)-4,5,7-trimethyl-4H,7H-[1,2,3,4]tetrazolo[1,5-a]pyrimidine-6-amido]isoquinolin-1-yl}carbamate was obtained as a white solid (43 mg, 0.078 mmol 29% yield). ¹H NMR (400 MHz, DMSO-d₆) δ 10.63 (s, 1H), 8.49 (d, J=1.8 Hz, 1H), 8.33 (d, J=5.7 Hz, 1H), 7.91-7.77 (m, 3H), 5.81 (q, J=6.2 Hz, 1H), 3.45 (s, 3H), 2.22 (s, 3H), 1.57 (d, J=6.4 Hz, 3H), 1.31 (s, 18H). MS-ESI (m/z) calcd for C₂₇H₃₅N₈O₅ [M+H]+: 551.3. Found 551.3.

### Step 4. (R)—N-(1-Aminoisoquinolin-6-yl)-4,5,7-trimethyl-4,7-dihydrotetrazolo[1,5-a]pyrimidine-6-carboxamide

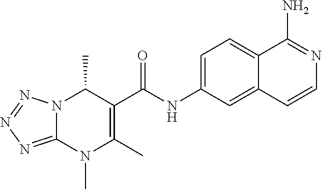 [SMILES: CC1=C(C(=O)Nc2ccc3c(N)nccc3c2)[C@@H](C)n2nnnc2N1C]

tert-Butyl-N-[(tert-butoxy)carbonyl]-N-{6-[(7R)-4,5,7-trimethyl-4H,7H-[1,2,3,4]tetrazolo[1,5-a]pyrimidine-6-amido]isoquinolin-1-yl}carbamate (40 mg, 0.073 mmol) was dissolved in DCM (3 mL), then TFA (1 mL) was added to the solution that was stirred for 1.5 h at room temperature. Volatiles were removed under reduced pressure and the crude material was purified by reverse phase column chromatography on a 12 g C18 column, using as eluent a gradient of CH₃CN in H₂O from 5 to 50% in presence of 0.1% HCOOH to afford a white solid. This was further purified by SCX (500 mg), washing with MeOH and eluting with NH₃ 1 M in MeOH, to afford the product as a white solid (13 mg, 0.037 mmol, 51% yield). LC-MS: m/z=351.23 [M+H]+, 0.46 min. ¹H NMR (400 MHz, DMSO-d₆) δ 10.39 (s, 1H), 8.07-8.18 (m, 2H), 7.75 (d, J=5.94 Hz, 1H), 7.55 (dd, J=9.02, 1.98 Hz, 1H), 6.83 (d, J=5.72 Hz, 1H), 6.67 (s, 2H), 5.78 (q, J=6.16 Hz, 1H), 3.45 (s, 3H), 2.20 (d, J=0.88 Hz, 3H), 1.56 (d, J=6.38 Hz, 3H). MS-ESI (m/z) calcd for C₁₇H₁₉N₈O [M+H]+: 351.2. Found 351.2.

### Example 43. 4,5,7,7-Tetramethyl-N-(3-(2-morpholinopyridin-4-yl)-1H-indazol-5-yl)-4,7-dihydrotetrazolo[1,5-a]pyrimidine-6-carboxamide

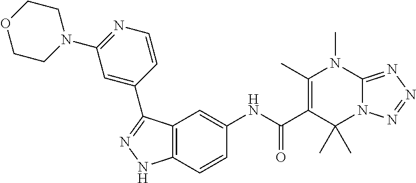 [SMILES: CC1=C(C(=O)Nc2ccc3[nH]nc(-c4ccnc(N5CCOCC5)c4)c3c2)C(C)(C)n2nnnc2N1C]

Tetramethyl-4H,7H-[1,2,3,4]tetrazolo[1,5-a]pyrimidine-6-carboxylic acid (210 mg, 21% pure by NMR, 0.31 mmol theoretical) and 3-[2-(morpholin-4-yl)pyridin-4-yl]-1H-indazol-5-amine (109 mg, 0.37 mmol) were dissolved in dry DMF (2.5 mL). Then the solution was cooled to 0° C. with an ice-water bath and TEA (87 μL, 0.62 mmol) and HATU (143 mg, 0.38 mmol) were sequentially added. The mixture was stirred at 0° C. for 5 min and then at room temperature for 18 hrs. The mixture was partitioned between EtOAc (20 mL) and water (30 mL). The organic layer was separated and the aqueous phase was extracted (2×20 mL) with EtOAc. The combined organic layers were collected, dried over sodium sulfate, filtered and concentrated under reduced pressure. The crude material was purified by column chromatography on an NH-silica gel column (EtOAc/MeOH, 10:0→9:1, as eluent) to afford a not pure fraction which was further purified by chiral semi-preparative HPLC to afford the desired product (10.2 mg, 0.02 mmol, 6.5% yield) as a white solid. Semi-preparative chiral HPLC column: Chiralcel OD-H (25×2.0 cm), 5μ. Mobile phase: (MeOH+0.1% isopropylamine) 25% v/v. Flow rate (ml/min): 45 ml/min. DAD detection: 220 nm. Loop: 600 μL. Total amount: 40 mg. Solubilization: 40 mg in 1.5 ml EtOH/MeOH 1/1=26.7 mg/mL. Injection: 16 mg/injection. ¹H NMR (400 MHz, DMSO-d₆) δ 13.45 (br. s., 1H), 10.34 (s, 1H), 8.56 (s, 1H), 8.30 (d, J=5.06 Hz, 1H), 7.61 (d, J=1.10 Hz, 2H), 7.29 (s, 1H), 7.23 (dd, J=5.28, 1.10 Hz, 1H), 3.68-3.82 (m, 4H), 3.50-3.61 (m, 4H), 3.45 (s, 3H), 2.14 (s, 3H), 1.79 (s, 6H). MS-ESI (m/z) calcd for C₂₅H₂₉N₁₀O₂ [M+H]+: 501.2. Found 501.3.

### Example 44. 4,5,7,7-Tetramethyl-N-(3-(3-morpholinophenyl)-1H-indazol-5-yl)-4,7-dihydrotetrazolo[1,5-a]pyrimidine-6-carboxamide

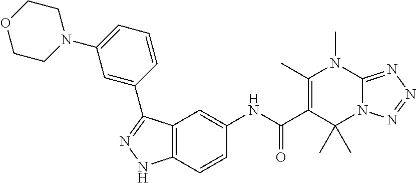 [SMILES: CC1=C(C(=O)Nc2ccc3[nH]nc(-c4cccc(N5CCOCC5)c4)c3c2)C(C)(C)n2nnnc2N1C]

To a solution of methyl 4,5,7,7-tetramethyl-4H,7H-[1,2,3,4]tetrazolo[1,5-a]pyrimidine-6-carboxylate (70 mg, 0.3 mmol) and 3-[3-(morpholin-4-yl)phenyl]-1H-indazol-5-amine (113 mg, 0.38 mmol) in dry toluene (3 mL), was added trimethylaluminum (2 M solution in toluene, 0.44 mL, 0.89 mmol). The reaction mixture was stirred at 90° C. for 18 hrs. The reaction mixture was then cooled to room temperature, diluted with water (50 mL) and extracted with EtOAc (100 mL). The organic layer was separated, dried over Na₂SO₄, filtered and concentrated under reduced pressure. The crude material was then purified by reverse phase column chromatography on a 30 g C18 silica gel column (water/acetonitrile, 95:5 to 50:50 as eluent containing 0.1% formic acid) to afford an impure fraction which was further purified by chiral semi-preparative HPLC to give the title compound (43.5 mg, 0.087 mmol, 29.5% yield) as a white solid. Semi-preparative chiral HPLC: Column: Chiralpak AD-H (25×2.0 cm), 5μ. Mobile phase: n-Hexane/Ethanol, 70/30% v/v. Flow rate (ml/min): 17 ml/min. DAD detection: 220 nm. Loop: 850 μL. Total amount: 60 mg. Solubilization: 60 mg in 1.5 ml (1.0 ml 1,1,1,3,3,3-hexafluoro-2-propanol+4.0 mL EtOH/MeOH 1/1)=12 mg/mL. Injection: 10.2 mg/injection. ¹H NMR (400 MHz, DMSO-d₆) δ 13.15 (br. s., 1H), 10.30 (s, 1H), 8.51 (s, 1H), 7.56 (d, J=0.88 Hz, 2H), 7.47 (s, 1H), 7.33-7.44 (m, 2H), 7.03 (dd, J=8.03, 1.43 Hz, 1H), 3.76-3.86 (m, 4H), 3.44 (s, 3H), 3.19-3.25 (m, 4H), 2.14 (s, 3H), 1.79 (s, 6H). MS-ESI (m/z) calcd for C₂₆H₃₀N₉O₂ [M+H]+: 500.2. Found 500.3.

### Example 45. N-(3-(3-((2S,6R)-2,6-Dimethylmorpholino)phenyl)-1H-indazol-5-yl)-4,5,7,7-tetramethyl-4,7-dihydrotetrazolo[1,5-a]pyrimidine-6-carboxamide

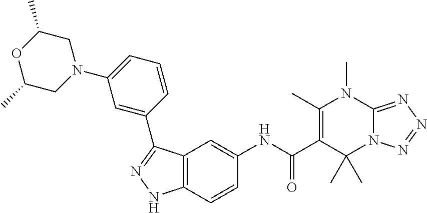 [SMILES: CC1=C(C(=O)Nc2ccc3[nH]nc(-c4cccc(N5C[C@@H](C)O[C@@H](C)C5)c4)c3c2)C(C)(C)n2nnnc2N1C]

Tetramethyl-4H,7H-[1,2,3,4]tetrazolo[1,5-a]pyrimidine-6-carboxylic acid (300 mg, 20% pure by NMR, 0.27 mmol theoretical) and 3-{3-[(2R,6S)-2,6-dimethylmorpholin-4-yl]phenyl}-1H-indazol-5-amine (130 mg, 0.4 mmol) were dissolved in dry DMF (3 mL). The solution was cooled to 0° C. with an ice-water bath and TEA (0.045 mL, 0.32 mmol) and HATU (123 mg, 0.32 mmol) were sequentially added. The mixture was stirred at 0° C. for 5 min and then at room temperature for 18 hrs. The mixture was partitioned between EtOAc (20 mL) and water (20 mL). The organic layer was separated and the aqueous phase was extracted (2×20 mL) with EtOAc. The combined organic layers were collected, dried over Na₂SO₄, filtered and concentrated under reduced pressure. The crude material was purified by preparative HPLC twice (Method A, then method B) to afford the desired product (59.9 mg, 0.114 mmol, 42.2% yield) as a beige solid. ¹H NMR (400 MHz, DMSO-d₆) δ 13.14 (s, 1H), 10.31 (s, 1H), 8.63 (s, 1H), 7.44-7.61 (m, 3H), 7.32-7.43 (m, 2H), 6.95-7.11 (m, 1H), 3.67-3.84 (m, 4H), 3.44 (s, 3H), 2.27-2.42 (m, 2H), 2.14 (s, 3H), 1.79 (s, 6H), 1.20 (d, J=6.16 Hz, 6H). MS-ESI (m/z) calcd for C₂₈H₃₄N₉O₂ [M+H]+: 528.3. Found 528.3.

### Example 46. 4,5,7,7-Tetramethyl-N-(3-phenyl-1H-indazol-5-yl)-4,7-dihydrotetrazolo[1,5-a]pyrimidine-6-carboxamide

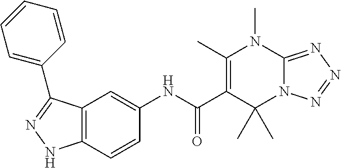 [SMILES: CC1=C(C(=O)Nc2ccc3[nH]nc(-c4ccccc4)c3c2)C(C)(C)n2nnnc2N1C]

To a solution of methyl 4,5,7,7-tetramethyl-4H,7H-[1,2,3,4]tetrazolo[1,5-a]pyrimidine-6-carboxylate (70 mg, 0.3 mmol) and 3-phenyl-1H-indazol-5-amine (Intermediate 5; 93 mg, 0.44 mmol) in dry toluene (3 mL) was added trimethylaluminum (2 M solution in toluene, 0.44 mL, 0.89 mmol). The reaction mixture was stirred for 18 hrs at 90° C. The reaction was cooled to rt, quenched with water and extracted with DCM (2×). The organic phase was passed through a phase separator and concentrated under reduced pressure. The crude material was purified by reverse phase column chromatography on a 30 g C18-silica gel column (water/acetonitrile, 95:5 to 50:50 as eluent containing 0.1% formic acid) to afford an impure fraction which was further purified by chiral semi-preparative HPLC to afford the desired product (10.5 mg, 0.025 mmol, 8.6% yield) as a white solid. Semi-preparative chiral HPLC: Column: Chiralpak AD-H (25×2.0 cm), 5μ. Mobile phase: n-hexane/EtOH, 70/30% v/v. Flow rate (ml/min): 17 ml/min. DAD detection: 220 nm. Loop: 600 μL. Total amount: 20 mg. Solubilization: 20 mg in 1.5 ml (EtOH/MeOH 1/1)=13.3 mg/mL. Injection: 8 mg/injection. LC-MS: m/z=415.21 [M+H]+, 0.89 min. ¹H NMR (400 MHz, DMSO-d₆) δ 13.22 (br. s., 1H), 10.32 (s, 1H), 8.51 (s, 1H), 7.93 (d, J=7.26 Hz, 2H), 7.52-7.63 (m, 4H), 7.38-7.47 (m, 1H), 3.44 (s, 3H), 2.14 (s, 3H), 1.79 (s, 6H). MS-ESI (m/z) calcd for C₂₂H₂₃N₈O [M+H]+: 415.2. Found 415.2.

### Example 47. (R)-4,5,7-Trimethyl-N-(3-methyl-2-oxo-2,3-dihydro-1H-benzo[d]imidazol-5-yl)-4,7-dihydrotetrazolo[1,5-a]pyrimidine-6-carboxamide

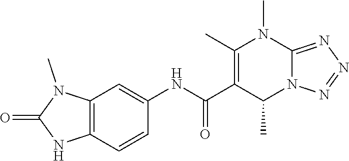 [SMILES: CC1=C(C(=O)Nc2ccc3[nH]c(=O)n(C)c3c2)[C@@H](C)n2nnnc2N1C]

5-Amino-3-methyl-1H-benzimidazol-2-one hydrochloride (200 mg, 1 mmol) was dissolved in DMSO (2 mL) and loaded onto a SCX cartridge. The compound was eluted by using as eluent MeOH and then a 1M solution of NH₃ in MeOH. Product-containing fractions were combined and concentrated under reduced pressure to give the freebase (137 mg, 0.84 mmol, 84% yield). The freebase (78. mg, 0.48 mmol) and (R)-4,5,7-trimethyl-4,7-dihydrotetrazolo[1,5-a]pyrimidine-6-carboxylic acid (Intermediate 4b; 50 mg, 0.24 mmol) were dissolved in dry DMF (2.5 mL). Then the solution was cooled to 0° C. with an ice-water bath and TEA (70 μL, 0.48 mmol) and HATU (109 mg, 0.29 mmol) were added. The mixture was stirred at 0° C. for 5 min and then at room temperature for 18 hrs. The mixture was diluted with water (20 mL) and extracted with EtOAc (2×20 mL). The combined organic layers were collected, dried over sodium sulfate, filtered and concentrated to give the crude product which was purified by reverse phase column chromatography on a 30 g C18-silica gel column (water/acetonitrile, 95:5 to 50:50, as eluent) to give the desired product (36.7 mg, 0.104 mmol, 43% yield) as a beige solid. NMR (400 MHz, DMSO-d₆) δ 10.78 (s, 1H), 10.09 (s, 1H), 7.54 (d, J=1.32 Hz, 1H), 7.13 (dd, J=8.36, 1.76 Hz, 1H), 6.93 (d, J=8.36 Hz, 1H), 5.72 (q, J=6.16 Hz, 1H), 3.43 (s, 3H), 3.26 (s, 3H), 2.18 (d, J=0.88 Hz, 3H), 1.55 (d, J=6.38 Hz, 3H). MS-ESI (m/z) calcd for C₁₆H₁₉N₈O₂ [M+H]+: 355.2. Found 355.2.

### Example 48. (R)-4,5,7-Trimethyl-N-(1-methyl-2-oxo-2,3-dihydro-1H-benzo[d]imidazol-5-yl)-4,7-dihydrotetrazolo[1,5-a]pyrimidine-6-carboxamide

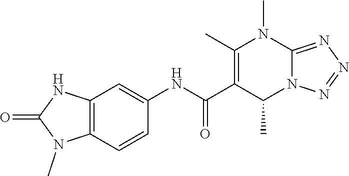 [SMILES: CC1=C(C(=O)Nc2ccc3c(c2)[nH]c(=O)n3C)[C@@H](C)n2nnnc2N1C]

(7R)-4,5,7-Trimethyl-4H,7H-[1,2,3,4]tetrazolo[1,5-a]pyrimidine-6-carboxylic acid (50 mg, 0.24 mmol) and 5-amino-1-methyl-2,3-dihydro-1H-1,3-benzodiazol-2-one (78 mg, 0.48 mmol) were dissolved in dry DMF (2.5 mL). Then the solution was cooled to 0° C. with an ice-water bath and TEA (0.07 mL, 0.48 mmol) and HATU (109 mg, 0.29 mmol) were sequentially added. The mixture was stirred at 0° C. for 5 min and then at room temperature for 18 hrs. The mixture was diluted with water (20 mL) and extracted with EtOAc (2×20 mL). The combined organic layers were collected, dried over sodium sulfate, filtered and concentrated to give a crude which was purified by reverse phase column chromatography on a 30 g C18-silica gel column (water/acetonitrile 95:5 to 40:60 as eluent containing 0.1% formic acid) to give the desired product (31.7 mg, 0.09 mmol, 37% yield) as a beige solid. ¹H NMR (400 MHz, DMSO-d₆) δ 10.82 (s, 1H), 10.07 (s, 1H), 7.53 (d, J=1.32 Hz, 1H), 7.17 (dd, J=8.36, 1.76 Hz, 1H), 7.03 (d, J=8.36 Hz, 1H), 5.71 (q, J=5.87 Hz, 1H), 3.42 (s, 3H), 3.26 (s, 3H), 2.17 (s, 3H), 1.54 (d, J=6.38 Hz, 3H). MS-ESI (m/z) calcd for C₁₆H₁₉N₈O₂ [M+H]+: 355.2. Found 355.2.

### Example 49. (R)—N-(3,3-Dimethyl-1-oxoisoindolin-5-yl)-4,5,7-trimethyl-4,7-dihydrotetrazolo[1,5-a]pyrimidine-6-carboxamide

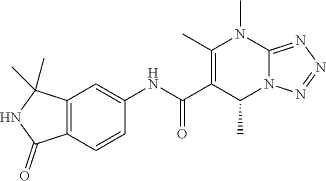 [SMILES: CC1=C(C(=O)Nc2ccc3c(c2)C(C)(C)NC3=O)[C@@H](C)n2nnnc2N1C]

### Step 1. 5-Bromo-2-(4-methoxybenzyl)isoindolin-1-one

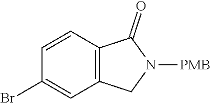

To a solution of methyl 4-bromo-2-(bromomethyl)benzoate (1.5 g, 4.87 mmol) and 4-methoxybenzylamine (800 mg, 5.84 mmol) in THF (24 mL) was added TEA (1.36 mL, 9.74 mmol). The resulting mixture was stirred at room temperature for 18 hrs. The mixture was then filtered and the residue was purified by reverse phase column chromatography on a 30 g C18-silica gel column (water/acetonitrile, 98:2 to 0:1 as eluent containing 0.1% formic acid) to give the desired product (418 mg, 1.26 mmol, 26% yield) as a white solid. ¹H NMR (400 MHz, CDCl₃) □δ 7.76 (d, J=7.92 Hz, 1H), 7.58-7.68 (m, 1H), 7.55 (d, J=0.66 Hz, 1H), 7.25 (d, J=8.58 Hz, 2H), 6.81-6.95 (m, 2H), 4.74 (s, 2H), 4.24 (s, 2H), 3.81 (s, 3H). MS-ESI (m/z) calcd for C₁₆H₁₅BrNO₂ [M+H]+: 332.0. Found 332.2.

### Step 2. 5-Bromo-2-(4-methoxybenzyl)-3,3-dimethylisoindolin-1-one

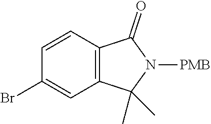

To a solution of 5-bromo-2-[(4-methoxyphenyl)methyl]-2,3-dihydro-1H-isoindol-1-one (150 mg, 0.45 mmol) in dry DMF (1.5 mL), was added NaH (36 mg, 0.9 mmol) and the reaction mixture was stirred at rt for 15 min under a nitrogen atmosphere. Iodomethane (0.17 mL, 2.7 mmol) was added and the solution was heated to 70° C. for 18 hrs. The reaction was cooled to rt, diluted with water (15 mL) and EtOAc (15 mL), the organic layer was separated, dried over sodium sulfate, filtered and concentrated. The residue was purified by column chromatography on a 10 g silica gel column (cyclohexane/EtOAc, 1:0 to 8:2, as eluent) to give the desired product (56 mg, 0.156 mmol, 35% yield) as a colorless oil. LC-MS: m/z=360.2; 362.19 [M+H]+, 1.19 min. ¹H NMR (400 MHz, CDCl₃) δ 7.76 (d, J=7.92 Hz, 1H), 7.61 (dd, J=8.03, 1.43 Hz, 1H), 7.54 (d, J=1.32 Hz, 1H), 7.31 (d, J=8.58 Hz, 2H), 6.85 (d, J=8.58 Hz, 2H), 4.69 (s, 2H), 3.80 (s, 3H), 1.38 (s, 6H). MS-ESI (m/z) calcd for C₁₈H₁₉BrNO₂ [M+H]+: 360.1. Found 360.2.

### Step 3. tert-Butyl (2-(4-methoxybenzyl)-3,3-dimethyl-1-oxoisoindolin-5-yl)carbamate

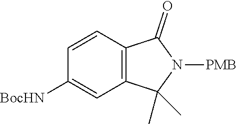

A solution of 5-bromo-2-[(4-methoxyphenyl)methyl]-3,3-dimethyl-2,3-dihydro-1H-isoindol-1-one (176 mg, 0.49 mmol), tert-butyl carbamate (86 mg, 0.74 mmol) and cesium carbonate (519 mg, 1.47 mmol) in toluene (3 mL) was degassed with nitrogen for 15 min. Then palladium acetate (11 mg, 0.05 mmol) and XantPhos (29 mg, 0.05 mmol) were added under nitrogen atmosphere and purging was continued for other 10 min. The reaction was refluxed at 110° C. for 18 hrs. The reaction mixture was filtered through a Celite pad and the filtrate was concentrated to obtain the crude product which was purified by column chromatography on a 11 g NH silica gel column (cyclohexane/EtOAc, 1:0 to 1:1 as eluent) to afford the desired product (138 mg, 0.35 mmol, 71% yield) as a colorless oil. ¹H NMR (400 MHz, CDCl₃) δ 7.77-7.79 (m, 2H), 7.29-7.35 (m, 2H), 7.10 (dd, J=8.36, 1.76 Hz, 1H), 6.81-6.88 (m, 2H), 4.69 (s, 2H), 3.80 (s, 3H), 1.55 (s, 9H), 1.37 (s, 6H). MS-ESI (m/z) calcd for C₂₃H₂₉N₂O₄ [M+H]+: 397.2. Found 397.2.

### Step 4. 5-Amino-3,3-dimethylisoindolin-1-one

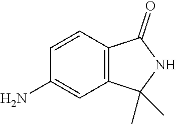

A mixture of tert-butyl N-{2-[(4-methoxyphenyl)methyl]-3,3-dimethyl-1-oxo-2,3-dihydro-1H-isoindol-5-yl}carbamate (138 mg, 0.35 mmol) in trifluoroacetic acid (1.24 mL) was heated at reflux for 18 hrs. The mixture was concentrated under reduced pressure and the residue was dissolved in MeOH (3 mL) and treated with an aqueous 2 M Na₂CO₃ solution (2 mL). The mixture was stirred at 40° C. for 2 hrs. The mixture was filtered to remove Na₂CO₃ and the filter cake was washed with MeOH (2×20 ml). The filtrate was concentrated and then the residue was dissolved in EtOAc and washed with water. The organic phase was separated, concentrated under reduced pressure and the residue was dissolved in 1 mL of DMSO and then purified on an SCX column eluted using MeOH followed by a 1M solution of NH₃ in MeOH. The compound-containing fractions were combined and concentrated to give the desired product (48 mg, 0.273 mmol, 78% yield) as a colorless oil. MS-ESI (m/z) calcd for C₁₀H₁₃N₂O [M+H]+: 177.1. Found 177.1.

### Step 5. (R)—N-(3,3-Dimethyl-1-oxoisoindolin-5-yl)-4,5,7-trimethyl-4,7-dihydrotetrazolo[1,5-a]pyrimidine-6-carboxamide

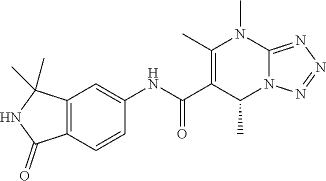 [SMILES: CC1=C(C(=O)Nc2ccc3c(c2)C(C)(C)NC3=O)[C@@H](C)n2nnnc2N1C]

To a stirred solution of (7R)-4,5,7-trimethyl-4H,7H-[1,2,3,4]tetrazolo[1,5-a]pyrimidine-6-carboxylic acid (48 mg, 0.23 mmol), 5-amino-3,3-dimethyl-2,3-dihydro-1H-isoindol-1-one (48 mg, 0.27 mmol), and TEA (95 μL, 0.68 mmol) in dry DMF (3 mL) at 0° C., was added dropwise a solution of propylphosphonic anhydride (50% solution in DMF, 166 μL, 0.27 mmol). The reaction mixture was stirred at room temperature for 18 hrs. The reaction was cooled to 0° C. and a further amount of TEA (64 μL, 0.46 mmol) and propylphosphonic anhydride (50% solution in DMF, 83 μL, 0.14 mmol) was added. The reaction was then stirred at rt for an additional 18 hrs. The mixture was diluted with water (20 mL) and extracted with EtOAc (20 mL). The organic phase was concentrated under reduced pressure to give the crude product which was purified by preparative HPLC (Method A). The product-containing fractions were combined and lyophilized to afford the desired product (13.1 mg, 0.036 mmol, 16%) as a white solid. ¹H NMR (400 MHz, DMSO-d₆) δ 10.45 (s, 1H), 8.51 (s, 1H), 7.95 (s, 1H), 7.57 (s, 2H), 5.76 (q, J=6.20 Hz, 1H), 3.44 (s, 3H), 2.19 (s, 3H), 1.55 (d, J=6.38 Hz, 3H), 1.44 (s, 6H). MS-ESI (m/z) calcd for C₁₈H₂₂N₇O₂ [M+H]+: 368.2. Found 368.3.

### Example 50. N-(1H-Indazol-5-yl)-7-isopropyl-4,5-dimethyl-4,7-dihydrotetrazolo[1,5-a]pyrimidine-6-carboxamide

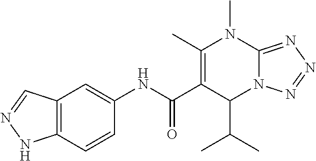 [SMILES: CC1=C(C(=O)Nc2ccc3[nH]ncc3c2)C(C(C)C)n2nnnc2N1C]

### Step 1. Ethyl 7-isopropyl-5-methyl-4,7-dihydrotetrazolo[1,5-a]pyrimidine-6-carboxylate

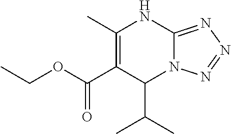 [SMILES: CCOC(=O)C1=C(C)Nc2nnnn2C1C(C)C]

A mixture of 5-aminotetrazole monohydrate (1.03 g, 10.00 mmol), EtOAc (1.26 mL, 10.00 mmol) and isobutyraldehyde (1.00 mL, 11.00 mmol) in water (45 mL) was heated at reflux for 24 hrs. The solvent was evaporated and the white residue was taken up in diethyl ether. The solid that formed was removed by filtration. The mother liquors were evaporated to afford the title compound as a white solid (0.82 g, 3.26 mmol, 33% yield). ¹H NMR (400 MHz, DMSO-d₆) δ 11.01 (s, 1H), 5.51 (d, J=2.0 Hz, 1H), 4.22-4.07 (m, 2H), 2.38 (s, 3H), 2.07-1.95 (m, 1H), 1.24 (t, 7=7.1 Hz, 3H), 1.03 (d, 7=7.0 Hz, 3H), 0.47 (d, J=6.9 Hz, 3H). MS-ESI (m/z) calculated for C₁₁H₁₈N₅O₂ [M+H]⁺: 252.14. Found 252.01.

### Step 2. Ethyl 7-isopropyl-4,5-dimethyl-4,7-dihydrotetrazolo[1,5-a]pyrimidine-6-carboxylate

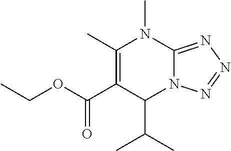 [SMILES: CCOC(=O)C1=C(C)N(C)c2nnnn2C1C(C)C]

To a solution of ethyl 5-methyl-7-(propan-2-yl)-4H,7H-[1,2,3,4]tetrazolo[1,5-a]pyrimidine-6-carboxylate (0.82 g, 3.26 mmol) in CH₃CN (25 mL) was added Mel (0.22 mL, 3.56 mmol) and Cs₂CO₃ (1.17 g, 3.56 mmol) and the mixture was stirred at 50° C. for 1 hour. The solvent was evaporated and water was added. The mixture was then stirred for 2 hours and the solid that formed was isolated by vacuum filtration to afford the title compound as a pale yellow solid (0.65 g, 2.46 mmol, 75% yield). ¹H NMR (400 MHz, DMSO-d₆) δ 5.54 (d, J=2.4 Hz, 1H), 4.26-4.07 (m, 2H), 3.47 (s, 3H), 2.54 (d, J=0.7 Hz, 3H), 1.98 (heptd, J=6.8, 2.5 Hz, 1H), 1.25 (t, J=7.1 Hz, 3H), 1.00 (d, J=6.9 Hz, 3H), 0.50 (d, J=6.9 Hz, 3H). MS-ESI (m/z) calculated for C₁₂H₂₀N₅O₂ [M+H]⁺: 266.16. Found 266.07.

### Step 3. 7-Isopropyl-4,5-dimethyl-4,7-dihydrotetrazolo[1,5-a]pyrimidine-6-carboxylic Acid

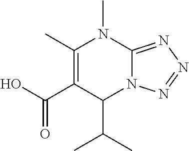 [SMILES: CC1=C(C(=O)O)C(C(C)C)n2nnnc2N1C]

To a solution of ethyl 4,5-dimethyl-7-(propan-2-yl)-4H,7H-[1,2,3,4]tetrazolo[1,5-a]pyrimidine-6-carboxylate (0.65 g, 2.46 mmol) in THF (15 mL) was added a solution of LiOH (0.31 g, 7.37 mmol) in H₂O (10 mL). The mixture was stirred at 55° C. for 24 hrs. The THF was evaporated and the aqueous solution was acidified with HCl_{(conc)}, the solid that formed was isolated by vacuum filtration and dried to afford the title compound as a white solid (375 mg, 1.58 mmol, 64% yield). ¹H NMR (400 MHz, DMSO-d₆) δ 12.62 (s, 1H), 5.50 (d, J=2.3 Hz, 1H), 3.46 (s, 3H), 2.55 (s, 3H), 2.00 (heptd, J=6.9, 2.3 Hz, 1H), 1.00 (d, J=7.0 Hz, 3H), 0.49 (d, J=6.9 Hz, 3H). MS-ESI (m/z) calculated for C₁₀H₁₆N₅O₂ [M+H]⁺: 238.13. Found 238.02.

### Step 4. N-(1H-Indazol-5-yl)-7-isopropyl-4,5-dimethyl-4,7-dihydrotetrazolo[1,5-a]pyrimidine-6-carboxamide

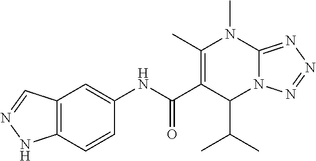 [SMILES: CC1=C(C(=O)Nc2ccc3[nH]ncc3c2)C(C(C)C)n2nnnc2N1C]

To a solution of 4,5-dimethyl-4H,7H-[1,2,4]triazolo[1,5-a]pyrimidine-6-carboxylic acid (237 mg, 1.00 mmol) in DMF (5.0 mL) was added TEA (0.14 mL, 1.00 mmol) and HATU (380 mg, 1.00 mmol). The mixture was stirred at rt for 15 min. and 5-aminoindazole (133 mg, 1.00 mmol) was added. The dark purple mixture was stirred at rt for 24 hours. Water was added and the compound was extracted with EtOAc (3×). The combined organic layers were washed with water, dried over Na₂SO₄ and evaporated to obtain a red residue which was purified by flash chromatography on a 10 g silica gel column using a 0-10% MeOH-DCM gradient as eluent to afford the title compound as a tan solid (307 mg, 0.87 mmol, 87% yield). ¹H NMR (400 MHz, DMSO-d₆) δ 13.00 (s, 1H), 10.11 (s, 1H), 8.13 (d, J=1.7 Hz, 1H), 8.03 (s, 1H), 7.51 (d, J=8.9 Hz, 1H), 7.46 (dd, J=8.9, 2.0 Hz, 1H), 5.65 (d, J=2.4 Hz, 1H), 3.42 (s, 3H), 2.24 (d, 7=1.0 Hz, 3H), 2.09 (heptd, J=6.9, 2.4 Hz, 1H), 0.98 (d, J=6.9 Hz, 3H), 0.71 (d, J=6.9 Hz, 3H). MS-ESI (m/z) calculated for C₁₇H₂₁N₈O [M+H]⁺: 353.18. Found 353.06.

### Separation of Enantiomers of N-(1H-Indazol-5-yl)-7-isopropyl-4,5-dimethyl-4,7-dihydrotetrazolo[1,5-a]pyrimidine-6-carboxamide

Racemic N-(H-indazol-5-yl)-4,5-dimethyl-7-(propan-2-yl)-4H,7H-[1,2,3,4]tetrazolo[1,5-a]pyrimidine-6-carboxamide (Intermediate 4) was subjected to semi-preparative chiral HPLC. Column: Chiralpak AS-H (25×2.0 cm), 5 μm. Mobile phase: n-hexane/EtOH 50/50% v/v. Flow rate (mL/min): 17 mL/min. DAD detection: 220 nm. Loop: 550 μL. Total amount: 50 mg. Solubilization: 50 mg in 3.5 mL (Ethanol/Methanol 1/1)=14.3 mg/mL. Injection: 7.8 mg/injection.

### Example 50a: First Eluting Enantiomer

(7S)—N-(1H-indazol-5-yl)-4,5-dimethyl-7-(propan-2-yl)-4H,7H-[1,2,3,4]tetrazolo[1,5-a]pyrimidine-6-carboxamide (21 mg, 0.06 mmol, 85% yield). ¹H NMR (400 MHz, DMSO-d₆) δ 13.00 (s, 1H), 10.11 (s, 1H), 8.13 (d, 7=1.7 Hz, 1H), 8.03 (s, 1H), 7.51 (d, 7=8.9 Hz, 1H), 7.46 (dd, J=8.9, 2.0 Hz, 1H), 5.65 (d, J=2.4 Hz, 1H), 3.42 (s, 3H), 2.24 (d, J=1.0 Hz, 3H), 2.09 (heptd, J=6.9, 2.4 Hz, 1H), 0.98 (d, J=6.9 Hz, 3H), 0.71 (d, J=6.9 Hz, 3H). MS-ESI (m/z) calculated for C₁₇H₂₁N₈O [M+H]⁺: 353.18. Found 353.22. Analytical chiral HPLC (e.e.=100%, 3.6 min). Absolute stereochemistry undetermined.

### Example 50b: Second Eluting Enantiomer

(7R)—N-(1H-indazol-5-yl)-4,5-dimethyl-7-(propan-2-yl)-4H,7H-[1,2,3,4]tetrazolo[1,5-a]pyrimidine-6-carboxamide (21 mg, 0.06 mmol, 85% yield). ¹H NMR (400 MHz, DMSO-d₆) δ 13.00 (s, 1H), 10.11 (s, 1H), 8.13 (d, 7=1.7 Hz, 1H), 8.03 (s, 1H), 7.51 (d, 7=8.9 Hz, 1H), 7.46 (dd, 7=8.9, 2.0 Hz, 1H), 5.65 (d, 7=2.4 Hz, 1H), 3.42 (s, 3H), 2.24 (d, 7=1.0 Hz, 3H), 2.09 (heptd, 7=6.9, 2.4 Hz, 1H), 0.98 (d, 7=6.9 Hz, 3H), 0.71 (d, 7=6.9 Hz, 3H). MS-ESI (m/z) calculated for C₁₇H₂₁N₈O [M+H]⁺: 353.18. Found 353.20. Analytical chiral HPLC (e.e.=100%, 5.3 min). Absolute stereochemistry undetermined.

### Example 51. 4-Acetyl-N-(2H-indazol-5-yl)-5-methyl-4,7-dihydrotetrazolo[1,5-a]pyrimidine-6-carboxamide

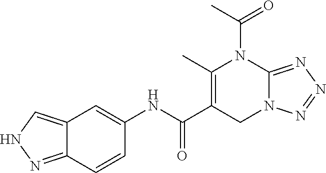 [SMILES: CC(=O)N1C(C)=C(C(=O)Nc2ccc3n[nH]cc3c2)Cn2nnnc21]

### Step 1. Ethyl 4-allyl-5-methyl-4,7-dihydrotetrazolo[1,5-a]pyrimidine-6-carboxylate

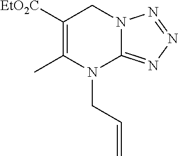 [SMILES: C=CCN1C(C)=C(C(=O)OCC)Cn2nnnc21]

To a solution of ethyl 5-methyl-4,7-dihydrotetrazolo[1,5-a]pyrimidine-6-carboxylate (2 g, 9.56 mmol) in THF (15 mL) was added NaH (573.55 mg, 14.34 mmol, 60% purity) at 0° C. The mixture was stirred at 15° C. for 0.5 h. Allyl bromide (1.50 g, 12.43 mmol, 2.20 mL) was then added to the reaction mixture at 0° C., and the mixture was stirred at 15° C. for 16 h. The reaction mixture was quenched with H₂O (100 mL) at 0° C. and extracted with EtOAc (25 mL×3). The combined organic layers were washed with brine (30 mL), dried over Na₂SO₄, filtered and concentrated under reduced pressure to give a residue. The residue was purified by column chromatography (SiO₂, petroleum ether/EtOAc=1:0 to 4:1) to give the product (820 mg, 2.57 mmol, 26.89% yield) as a yellow oil.

### Step 2. 4-Allyl-5-methyl-4,7-dihydrotetrazolo[1,5-a]pyrimidine-6-carboxylic Acid

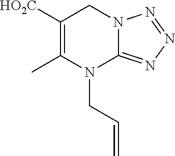 [SMILES: C=CCN1C(C)=C(C(=O)O)Cn2nnnc21]

To a solution of ethyl 4-allyl-5-methyl-4,7-dihydrotetrazolo[1,5-a]pyrimidine-6-carboxylate (820 mg, 2.57 mmol) in EtOH (10 mL) and H₂O (10 mL) was added LiOH.H₂O (323.62 mg, 7.71 mmol). The mixture was stirred at 15° C. for 16 h. The reaction mixture was concentrated under reduced pressure to remove the solvent. Then the reaction mixture was acidified with 1N HCl to pH=3. The resulting precipitate was collected by filtration to give the product (510 mg, 1.69 mmol, 65.64% yield) as a white solid.

### Step 3. 4-Allyl-N-(2H-indazol-5-yl)-5-methyl-4,7-dihydrotetrazolo[1,5-a]pyrimidine-6-carboxamide

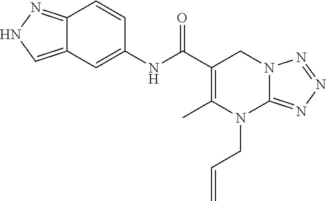 [SMILES: C=CCN1C(C)=C(C(=O)Nc2ccc3n[nH]cc3c2)Cn2nnnc21]

To a stirred solution of 4-allyl-5-methyl-4,7-dihydrotetrazolo[1,5-a]pyrimidine-6-carboxylic acid (300.00 mg, 1.36 mmol) in DCM (3 mL) was added 1H-indazol-5-amine (180.57 mg, 1.36 mmol) and T3P/EtOAc (1.29 g, 2.03 mmol, 1.21 mL, 50% purity) and the reaction mixture was stirred at 20° C. for 0.5 h. TEA (411.68 mg, 4.07 mmol, 566.28 uL) was then added and the reaction mixture was stirred at 20° C. for 12 h. The reaction mixture was concentrated and the residue was purified by prep-HPLC (basic condition) to give the product (230 mg, 683.81 umol, 50.42% yield) as a purple solid.

### Step 4. N-(2H-Indazol-5-yl)-5-methyl-4,7-dihydrotetrazolo[1,5-a]pyrimidine-6-carboxamide

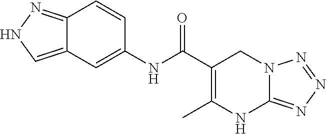 [SMILES: CC1=C(C(=O)Nc2ccc3n[nH]cc3c2)Cn2nnnc2N1]

4-Allyl-N-(2H-indazol-5-yl)-5-methyl-4,7-dihydrotetrazolo[1,5-a]pyrimidine-6-carboxamide (200 mg, 594.62 umol), 1,3-dimethylbarbituric acid (185.69 mg, 1.19 mmol) and Pd(PPh₃)₄ (68.71 mg, 59.46 umol) in DCM (10 mL) and EtOH (5 mL) was degassed and then heated to 55° C. for 12 h under N₂. After cooling to 20° C., the reaction mixture was filtered and the filtrate was concentrated. The residue was purified by prep-HPLC (basic condition) to give the product (100 mg, 337.51 umol, 56.76% yield) as a white solid.

### Step 5. 4-Acetyl-N-(2H-indazol-5-yl)-5-methyl-4,7-dihydrotetrazolo[1,5-a]pyrimidine-6-carboxamide

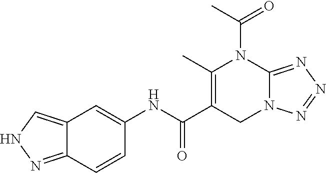 [SMILES: CC(=O)N1C(C)=C(C(=O)Nc2ccc3n[nH]cc3c2)Cn2nnnc21]

To a solution of N-(2H-Indazol-5-yl)-5-methyl-4,7-dihydrotetrazolo[1,5-a]pyrimidine-6-carboxamide (50 mg, 168.76 umol) and TEA (34.15 mg, 337.51 umol, 46.98 uL) in DCM (6 mL) was added acetyl chloride (19.87 mg, 253.13 umol, 18.06 uL) at 15° C. The reaction mixture was concentrated under reduced pressure to give a residue. The residue was purified by prep-HPLC (TFA condition) to give the product (4.13 mg, 10.74 umol, 6.36% yield, TFA salt) as a yellow gum. NMR (DMSO-d₆, 400 MHz) δ 13.01 (br s, 1H), 10.41 (s, 1H), 8.18 (s, 1H), 8.06 (s, 1H), 7.55-7.46 (m, 2H), 5.22 (d, J=1.3 Hz, 2H), 2.59 (s, 3H), 2.33-2.30 (m, 3H). MS-ESI (m/z) calcd for C₁₅H₁₅N₈O₂ [M+H]+: 339.1. Found 339.1.

### Example 52. N-(3-(2-(4-(Dimethylamino)phenyl)acetamido)-2H-indazol-5-yl)-4,5-dimethyl-4,7-dihydrotetrazolo[1,5-a]pyrimidine-6-carboxamide

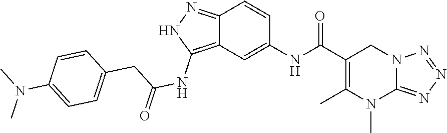 [SMILES: CC1=C(C(=O)Nc2ccc3n[nH]c(NC(=O)Cc4ccc(N(C)C)cc4)c3c2)Cn2nnnc2N1C]

### Step 1. 2-(4-(Dimethylamino)phenyl)acetyl Chloride

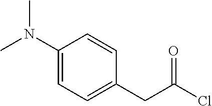

To a solution of 2-(4-(dimethylamino)phenyl)acetic acid (200 mg, 1.12 mmol) in DCM (4 mL) was added (COCl)₂ (212.47 mg, 1.67 mmol, 146.53 uL) and one drop of DMF (815.71 ug, 11.16 umol), then the mixture was stirred at 25° C. for 0.5 hr. The reaction mixture was concentrated under vacuum to afford the product (230 mg, crude) as a yellow liquid.

### Step 2. 4,5-Dimethyl-4,7-dihydrotetrazolo[1,5-a]pyrimidine-6-carbonyl Chloride

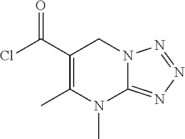

To a solution of 4,5-dimethyl-4,7-dihydrotetrazolo[1,5-a]pyrimidine-6-carboxylic acid (30 mg, 153.71 umol) in DCM (2 mL) was added (COCl)₂ (29.26 mg, 230.56 umol, 20.18 uL) and one drop of DMF (112.35 ug, 1.54 umol), then the mixture was stirred at 25° C. for 0.5 hr. The reaction mixture was concentrated under vacuum to afford the product (35 mg, crude) as a yellow solid, which was used in the next step without further purification.

### Step 3. 2-(4-(Dimethylamino)phenyl)-N-(5-nitro-2H-indazol-3-yl)acetamide

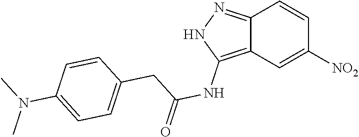 [SMILES: CN(C)c1ccc(CC(=O)Nc2[nH]nc3ccc([N+](=O)[O-])cc23)cc1]

To a solution of 5-nitro-2H-indazol-3-amine (200 mg, 1.12 mmol) in pyridine (4 mL) was added a solution of 2-(4-(dimethylamino)phenyl)acetyl chloride (230 mg, 1.16 mmol) in CH₃CN (1 mL) at 0° C. The mixture was stirred at 25° C. for 2 hrs. The reaction mixture was concentrated under vacuum. The residue was washed with MeOH (3 mL), filtered and the solid was dried under vacuum to afford the product (166 mg, crude) as a brown solid which was subsequently used without further purification.

### Step 4. N-(5-Amino-2H-indazol-3-yl)-2-(4-(dimethylamino)phenyl)acetamide

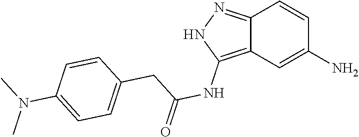 [SMILES: CN(C)c1ccc(CC(=O)Nc2[nH]nc3ccc(N)cc23)cc1]

To a solution of 2-(4-(dimethyl amino)phenyl)-N-(5-nitro-2H-indazol-3-yl)acetamide (80 mg, 235.75 umol) in EtOH (4 mL) was added 10% Pd/C (20 mg), then the mixture was stirred at 25° C. under H₂ at 15 psi for 2 hrs. The reaction mixture was filtered and the filtrate was concentrated under vacuum. The residue was purified by prep-HPLC (TFA condition) to afford the product (29 mg, 61.65 umol, 26.15% yield, TFA salt) as a yellow liquid.

### Step 5. N-(3-(2-(4-(Dimethylamino)phenyl)acetamido)-2H-indazol-5-yl)-4,5-dimethyl-4,7-dihydrotetrazolo[1,5-a]pyrimidine-6-carboxamide

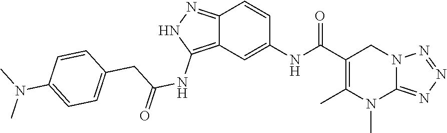 [SMILES: CC1=C(C(=O)Nc2ccc3n[nH]c(NC(=O)Cc4ccc(N(C)C)cc4)c3c2)Cn2nnnc2N1C]

To a solution of N-(5-amino-2H-indazol-3-yl)-2-(4-(dimethylamino)phenyl)acetamide (29 mg, 68.50 umol, TFA salt) in pyridine (1 mL) was added a solution of 4,5-dimethyl-4,7-dihydrotetrazolo[1,5-a]pyrimidine-6-carbonyl chloride (35 mg, 163.84 umol) in CH₃CN (0.5 mL) at 0° C. The mixture was stirred at 25° C. for 3 hrs. The mixture was concentrated under vacuum and the residue was purified by prep-HPLC (TFA condition) to afford the product (4.71 mg, 7.29 umol, 10.64% yield, TFA salt) as a pale yellow solid. ¹H NMR (DMSO-d₆, 400 MHz) δ 10.44 (s, 1H) 9.92 (s, 1H) 7.98 (s, 1H) 7.52 (d, J=8.77 Hz, 1H) 7.38 (d, J=9.21 Hz, 1H) 7.29 (br d, J=7.45 Hz, 2H) 6.96 (br s, 2H) 5.24 (s, 2H) 3.62 (s, 2H) 3.40 (s, 3H) 2.94 (s, 6H) 2.22 (s, 3H). MS-ESI (m/z) calcd for C₂₄H₂₇N₁₀O2 [M+H]+: 487.2. Found 487.2.

### Example 53. N-(4-Methoxy-1H-indazol-5-yl)-4,5-dimethyl-4,7-dihydrotetrazolo[1,5-a]pyrimidine-6-carboxamide

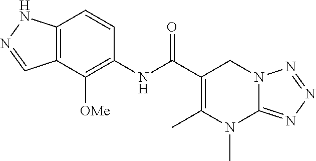 [SMILES: COc1c(NC(=O)C2=C(C)N(C)c3nnnn3C2)ccc2[nH]ncc12]

### Step 1. 4-Methoxy-5-nitro-1H-indazole

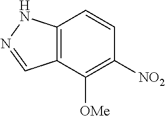 [SMILES: COc1c([N+](=O)[O-])ccc2[nH]ncc12]

H₂SO₄ (343.47 mg, 3.43 mmol, 186.67 uL, 98% purity) was added dropwise to HNO₃ (130.86 mg, 1.35 mmol, 93.47 uL, 65% purity) at 0° C. This mixture was stirred at 0° C. for min. A solution of 4-methoxy-1H-indazole (200 mg, 1.35 mmol) in H₂SO₄ (6 mL, 98% purity) was then added to the mixture of H₂SO₄ and HNO₃ at −15° C. The mixture was stirred at −15° C. for 20 min, then warmed up to −5° C. and stirred for 2 hrs. The reaction mixture was poured into cold water (10 mL) and treated with 2M NaOH to adjust the pH to 8-9, and then extracted with EtOAc (10 mL×3). The combined organic layers were washed with brine (20 mL×1), dried over Na₂SO₄, filtered and concentrated under reduced pressure to give a residue. The residue was purified by prep-TLC (SiO₂, petroleum ether/EtOAc=0:1) to afford the product (50 mg, 212.26 umol, 15.72% yield) as a yellow solid.

### Step 2. 4-Methoxy-1H-indazol-5-amine

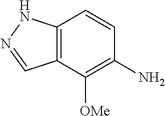 [SMILES: COc1c(N)ccc2[nH]ncc12]

To a solution of 4-methoxy-5-nitro-1H-indazole (50 mg, 258.85 umol) in EtOH (1 mL) was added 10% Pd/C (100 mg) and the mixture was stirred at 25° C. for 0.5 hr under Eh atmosphere at 15 psi. The reaction mixture was filtered and the filtrate was concentrated under reduced pressure to afford the product (50 mg) as a purple solid which was used without further purification.

### Step 3. N-(4-Methoxy-1H-indazol-5-yl)-4,5-dimethyl-4,7-dihydrotetrazolo[1,5-a]pyrimidine-6-carboxamide

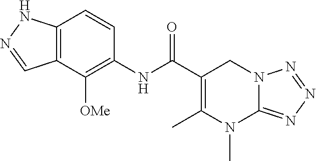 [SMILES: COc1c(NC(=O)C2=C(C)N(C)c3nnnn3C2)ccc2[nH]ncc12]

To a solution of 4-methoxy-1H-indazol-5-amine (40 mg, 245.13 umol) and 4,5-dimethyl-4,7-dihydrotetrazolo[1,5-a]pyrimidine-6-carboxylic acid (47.84 mg, 245.13 umol) in DCM (1 mL) was added TEA (99.22 mg, 980.54 umol) and T3P (467.98 mg, 735.40 umol, 50% purity in EtOAc). The mixture was stirred at 25° C. for 2 hrs. The reaction mixture was concentrated under reduced pressure to remove the solvent. The residue was purified by prep-HPLC (TFA condition) to afford the product (2.35 mg, 4.96 umol, 2.02% yield, TFA salt) as a light pink solid. NMR (DMSO-d₆, 400 MHz) δ 13.14 (br s, 1H) 9.27 (s, 1H) 8.32 (s, 1H) 7.45 (br d, J=8.60 Hz, 1H) 7.14 (br d, J=8.60 Hz, 1H) 5.25 (s, 2H) 4.14 (s, 3H) 3.42 (s, 3H) 2.31 (br s, 3H). MS-ESI (m/z) calcd for C₁₅H₁₇N₈O₂ [M+H]+: 341.1. Found 341.1.

### Example 54. 4,5-Dimethyl-N-(3-((6-methylpyridin-3-yl)carbamoyl)-2H-indazol-5-yl)-4,7-dihydrotetrazolo[1,5-a]pyrimidine-6-carboxamide

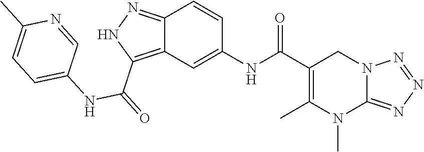 [SMILES: CC1=C(C(=O)Nc2ccc3n[nH]c(C(=O)Nc4ccc(C)nc4)c3c2)Cn2nnnc2N1C]

### Step 1. N-(6-Methylpyridin-3-yl)-5-nitro-2H-indazole-3-carboxamide

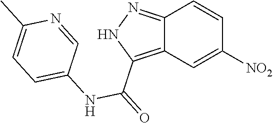 [SMILES: Cc1ccc(NC(=O)c2[nH]nc3ccc([N+](=O)[O-])cc23)cn1]

To a solution of 6-methylpyridin-3-amine (104.41 mg, 965.52 umol) in DMF (2 mL) was added 5-nitro-2H-indazole-3-carboxylic acid (200 mg, 965.52 umol) and DIPEA (249.57 mg, 1.93 mmol). The mixture was cooled to 0° C., and HATU (367.12 mg, 965.52 umol) was added. The mixture was stirred at 25° C. for 12 hrs. The reaction mixture was then filtered, and the solid was dried under reduced pressure to afford the product (150 mg, 487.19 umol, 50.46% yield) as pale yellow solid.

### Step 2. 5-Amino-N-(6-methylpyridin-3-yl)-2H-indazole-3-carboxamide

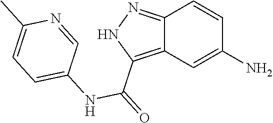 [SMILES: Cc1ccc(NC(=O)c2[nH]nc3ccc(N)cc23)cn1]

To a solution of N-(6-methylpyridin-3-yl)-5-nitro-2H-indazole-3-carboxamide (140 mg, 470.95 umol) in MeOH (2 mL) was added 10% Pd/C (0.1 g) under a nitrogen atmosphere. The mixture was degassed and purged with H₂ (3×). The mixture was stirred under H₂ (15 Psi) at 25° C. for 1 hr. The reaction mixture was filtered and the filtrate was concentrated under reduced pressure to give the product (123.4 mg, crude) as pale yellow solid, which was used in the next step without further purification.

### Step 3. 4,5-Dimethyl-N-(3-((6-methylpyridin-3-yl)carbamoyl)-2H-indazol-5-yl)-4,7-dihydrotetrazolo[1,5-a]pyrimidine-6-carboxamide

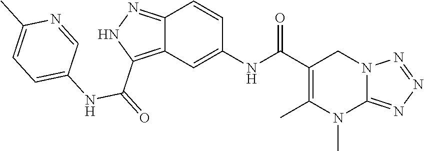 [SMILES: CC1=C(C(=O)Nc2ccc3n[nH]c(C(=O)Nc4ccc(C)nc4)c3c2)Cn2nnnc2N1C]

A mixture of 5-amino-N-(6-methylpyridin-3-yl)-2H-indazole-3-carboxamide (75 mg, 280.60 umol), 4,5-dimethyl-4,7-dihydrotetrazolo[1,5-a]pyrimidine-6-carboxylic acid (109.53 mg, 561.20 umol) and EDCI (107.58 mg, 561.20 umol) in pyridine (1 mL) was degassed and purged with N₂ (3×) and the mixture was stirred at 25° C. for 12 hrs. The reaction mixture was concentrated under reduced pressure to remove solvent. The residue was purified by prep-HPLC (TFA condition) to give the product (32.75 mg, 52.15 umol, 18.69% yield, TFA salt) as a white solid. ¹H NMR (DMSO-d₆, 400 MHz) δ 13.98 (s, 1H) 11.03 (br s, 1H) 10.12 (s, 1H) 9.23 (br s, 1H) 8.69 (s, 1H) 8.59 (br d, J=8.77 Hz, 1H) 7.63-7.74 (m, 3H) 5.30 (s, 2H) 3.44 (s, 3H) 2.61 (s, 3H) 2.27 (s, 3H). MS-ESI (m/z) calcd for C₂₁H₂₁N₁₀O₂ [M+H]+: 445.2. Found 445.1.

### Example 55. N-(3-(Furan-2-carboxamido)-2H-indazol-5-yl)-4,5-dimethyl-4,7-dihydrotetrazolo[1,5-a]pyrimidine-6-carboxamide

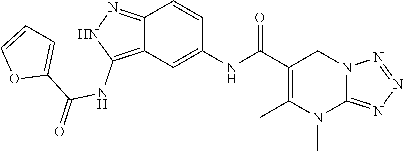 [SMILES: CC1=C(C(=O)Nc2ccc3n[nH]c(NC(=O)c4ccco4)c3c2)Cn2nnnc2N1C]

### Step 1. Furan-2-carbonyl Chloride

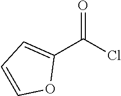

To a solution of furan-2-carboxylic acid (150 mg, 1.34 mmol) in DCM (2 mL) was added DMF (9.78 mg, 133.83 umol) and (COCl)₂ (254.80 mg, 2.01 mmol). The mixture was stirred at 20° C. for 0.5 hr. The reaction mixture was concentrated to afford the product (170 mg) as a colorless oil which was used without further purification.

### Step 2. N-(5-Nitro-2H-indazol-3-yl)furan-2-carboxamide

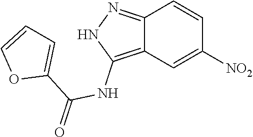 [SMILES: O=C(Nc1[nH]nc2ccc([N+](=O)[O-])cc12)c1ccco1]

To a solution of 5-nitro-2H-indazol-3-amine (210.18 mg, 1.18 mmol) in pyridine (2 mL) was added furan-2-carbonyl chloride (140 mg, 1.07 mmol) in MeCN (1 mL) at 0° C. The mixture was stirred at 20° C. for 12 hr. The reaction mixture was concentrated to give a residue. The residue was taken up in a mixture of EtOAc (3 mL) and MeOH (1 mL) and filtered. The solid was collected and dried under vacuum to afford the product (130 mg, crude) as an orange solid.

### Step 3. N-(5-amino-2H-indazol-3-yl)furan-2-carboxamide

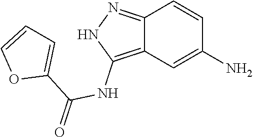 [SMILES: Nc1ccc2n[nH]c(NC(=O)c3ccco3)c2c1]

To a solution of N-(5-nitro-2H-indazol-3-yl)furan-2-carboxamide (120 mg, 440.83 umol) in EtOH (3 mL) was added 10% Pd/C (120 mg). The suspension was degassed and purged with H₂ (3×). The mixture was stirred at 20° C. for 1 hr under an Eh atmosphere (15 Psi). The reaction mixture was filtered. The filtrate was concentrated and purified by prep-TLC (SiO₂, MeOH:DCM=1:10) to afford the product (90 mg, 371.54 umol, 84.28% yield) as a white solid.

### Step 4. N-(3-(Furan-2-carboxamido)-2H-indazol-5-yl)-4,5-dimethyl-4,7-dihydrotetrazolo[1,5-a]pyrimidine-6-carboxamide

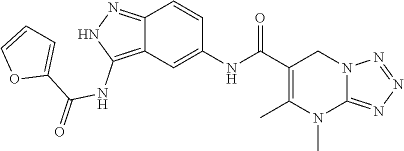 [SMILES: CC1=C(C(=O)Nc2ccc3n[nH]c(NC(=O)c4ccco4)c3c2)Cn2nnnc2N1C]

To a solution of 4,5-dimethyl-4,7-dihydrotetrazolo[1,5-a]pyrimidine-6-carboxylic acid (110 mg, 563.59 umol) in DCM (3 mL) was added T3P/EtOAc (268.98 mg, 422.69 umol, 50% purity), TEA (85.54 mg, 845.38 umol) and N-(5-amino-2H-indazol-3-yl)furan-2-carboxamide (68.26 mg, 281.79 umol). The mixture was stirred at 20° C. for 12 hr. The reaction mixture was concentrated and purified by prep-HPLC (neutral condition) to afford the product (23.86 mg, 55.77 umol, 19.79% yield) as a white solid. ¹H NMR (DMSO-d₆, 400 MHz) δ 12.79 (s, 1H) 10.66 (s, 1H) 9.97 (s, 1H) 7.98 (d, J=16.02 Hz, 2H) 7.53-7.63 (m, 1H) 7.39-7.49 (m, 2H) 6.73 (dd, J=3.42, 1.71 Hz, 1H) 5.27 (s, 2H) 3.42 (s, 3H) 2.24 (s, 3H). MS-ESI (m/z) calcd for C₁₉H₁₈N₉O₃ [M+H]+: 420.2. Found 420.1.

### Example 56. N-(3-(Cyclopropanecarboxamido)-2H-indazol-5-yl)-4,5-dimethyl-4,7-dihydrotetrazolo[1,5-a]pyrimidine-6-carboxamide

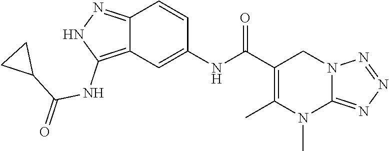 [SMILES: CC1=C(C(=O)Nc2ccc3n[nH]c(NC(=O)C4CC4)c3c2)Cn2nnnc2N1C]

### Step 1. N-(5-Nitro-2H-indazol-3-yl)cyclopropanecarboxamide

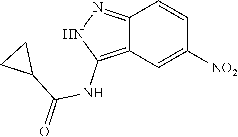 [SMILES: O=C(Nc1[nH]nc2ccc([N+](=O)[O-])cc12)C1CC1]

To a solution of 5-nitro-2H-indazol-3-amine (200 mg, 1.12 mmol) in pyridine (4 mL) was added a solution of cyclopropanecarbonyl chloride (129.09 mg, 1.23 mmol, 112.25 uL) in CH₃CN (1 mL) at 0° C. The mixture was then stirred at 0° C. for 2 hrs. The reaction mixture was concentrated under vacuum. The residue was diluted with MeOH (2 mL), filtered and the solid was dried under vacuum to afford the product (232 mg, 885.71 umol, 78.89% yield) as an orange solid.

### Step 2. N-(5-Amino-2H-indazol-3-yl)cyclopropanecarboxamide

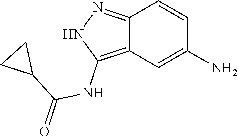 [SMILES: Nc1ccc2n[nH]c(NC(=O)C3CC3)c2c1]

To a solution of N-(5-nitro-2H-indazol-3-yl)cyclopropanecarboxamide (100 mg, 406.14 umol) in EtOH (5 mL) was added 10% Pd/C (30 mg). The mixture was then stirred at 25° C. under H₂ at 15 psi for 1 hr. The reaction mixture was filtered and the filtrate was concentrated under vacuum to give a residue which was purified by prep-HPLC (TFA condition) to afford the product (34 mg, 82.36 umol, 20.28% yield, TFA salt) as a white solid.

### Step 3. N-(3-(Cyclopropanecarboxamido)-2H-indazol-5-yl)-4,5-dimethyl-4,7-dihydrotetrazolo[1,5-a]pyrimidine-6-carboxamide

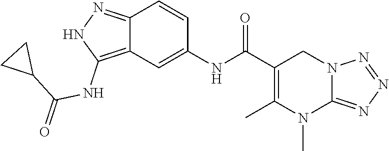 [SMILES: CC1=C(C(=O)Nc2ccc3n[nH]c(NC(=O)C4CC4)c3c2)Cn2nnnc2N1C]

To a solution of N-(5-amino-2H-indazol-3-yl)cyclopropanecarboxamide (34 mg, 102.95 umol, TFA salt) and 4,5-dimethyl-4,7-dihydrotetrazolo[1,5-a]pyrimidine-6-carboxylic acid (24.11 mg, 123.54 umol) in DCM (2 mL) was added T3P (196.54 mg, 308.85 umol, 183.68 uL, 50% purity in EtOAc) and TEA (104.17 mg, 1.03 mmol, 143.29 uL), then the mixture was stirred at 25° C. for 12 hrs. The mixture was concentrated under vacuum. The residue was purified by prep-HPLC (basic condition) to afford the product (9.36 mg, 23.79 umol, 23.11% yield) as a yellow gum. ¹H NMR (DMSO-d₆, 400 MHz) δ 12.62 (br s, 1H) 10.57 (s, 1H) 9.96 (s, 1H) 7.99 (s, 1H) 7.56 (br d, J=8.77 Hz, 1H) 7.40 (d, J=9.21 Hz, 1H) 5.27 (s, 2H) 3.42 (s, 3H) 2.24 (s, 3H) 1.88-1.96 (m, 1H) 0.83 (br d, J=4.82 Hz, 4H). MS-ESI (m/z) calcd for C₁₈H₂₀N₉O₂ [M+H]+: 394.2. Found 394.1.

### Example 57. N-(3-Butyramido-2H-indazol-5-yl)-4,5-dimethyl-4,7-dihydrotetrazolo[1,5-a]pyrimidine-6-carboxamide

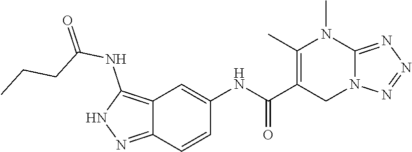 [SMILES: CCCC(=O)Nc1[nH]nc2ccc(NC(=O)C3=C(C)N(C)c4nnnn4C3)cc12]

### Step 1. N-(1-Butyryl-5-nitro-1H-indazol-3-yl)butyramide

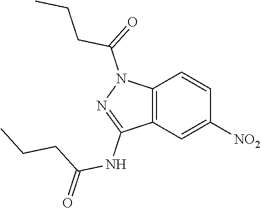 [SMILES: CCCC(=O)Nc1nn(C(=O)CCC)c2ccc([N+](=O)[O-])cc12]

To a solution of 5-nitro-1H-indazol-3-amine (200 mg, 1.12 mmol) in pyridine (3 mL) was added butyryl chloride (119.62 mg, 1.12 mmol, 117.28 uL) in ACN (0.2 mL) at 0° C. The mixture was stirred at 25° C. for 2 hrs. The mixture was concentrated and taken up in MeOH (6 mL) and filtered. The solid was dried in vacuo to afford the product (193 mg, 606.29 umol, 54.01% yield) as a yellow solid, which was used without further purification.

### Step 2. N-(5-Nitro-1H-indazol-3-yl)butyramide

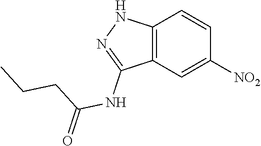 [SMILES: CCCC(=O)Nc1n[nH]c2ccc([N+](=O)[O-])cc12]

To a solution of N-(1-butyryl-5-nitro-1H-indazol-3-yl)butyramide (314.82 mg, 988.97 umol) in MeOH (3 mL) was added Na₂CO₃ (314.46 mg, 2.97 mmol). The mixture was stirred at 20° C. for 12 hrs. The mixture was concentrated and the residue was extracted with H₂O (20 mL) and ethyl acetate (15 mL×3). The organic layer was dried over Na₂SO₄ and concentrated to afford the product (210 mg, 845.96 umol, 85.54% yield) as a yellow solid, which was used without further purification.

### Step 3. N-(5-Amino-1H-indazol-3-yl)butyramide

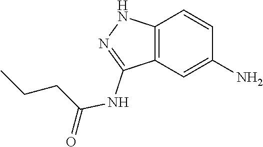 [SMILES: CCCC(=O)Nc1n[nH]c2ccc(N)cc12]

A mixture of N-(5-nitro-1H-indazol-3-yl)butyramide (100 mg, 402.84 umol), 10% Pd/C (100 mg) in MeOH (2 mL) was degassed and purged with H₂ (3×), and then the mixture was stirred at 20° C. for 2 hrs under H₂ (15 psi). The reaction mixture was filtered. The organic layer was concentrated to afford the product (81 mg, 371.13 umol, 92.13% yield) as a red oil, which was used without further purification.

### Step 4. N-(3-Butyramido-2H-indazol-5-yl)-4,5-dimethyl-4,7-dihydrotetrazolo[1,5-ci]pyrimidine-6-carboxamide

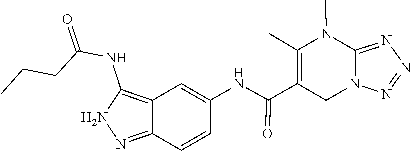

To a solution of N-(5-amino-1H-indazol-3-yl)butyramide (70 mg, 320.73 umol) in DCM (5 mL) was added T3P/EtOAc (408.20 mg, 641.45 umol, 381.49 uL, 50% purity), 4,5-dimethyl-4,7-dihydrotetrazolo[1,5-a]pyrimidine-6-carboxylic acid (68.86 mg, 352.80 umol) and TEA (97.36 mg, 962.18 umol, 133.92 uL). The mixture was stirred at 40° C. for 12 hrs. The mixture was concentrated, dissolved in DMF (2 mL) and purified by prep-HPLC (basic condition) to afford the product (14.50 mg, 11.25%) as a yellow gum. ¹H NMR (DMSO-d₆, 400 MHz) δ 12.60 (s, 1H), 10.22 (s, 1H), 9.97 (s, 1H), 7.99 (s, 1H), 7.55 (br d, J=8.68 Hz, 1H) 7.40 (d, J=8.93 Hz, 1H), 5.28 (s, 2H), 3.43 (s, 3H), 2.34-2.40 (m, 2H), 2.25 (s, 3H), 1.66 (sxt, J=7.29 Hz, 2H), 0.97 (t, J=7.34 Hz, 3H). MS-ESI (m/z) calcd for C₁₈H₂₂N₉O₂ [M+H]+: 396.2. Found 396.1.

### Example 58. 4,5-Dimethyl-N-(3-methyl-2H-indazol-5-yl)-4,7-dihydrotetrazolo[1,5-a]pyrimidine-6-carboxamide

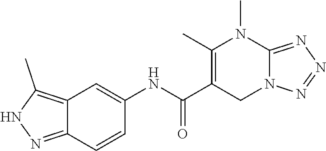 [SMILES: CC1=C(C(=O)Nc2ccc3n[nH]c(C)c3c2)Cn2nnnc2N1C]

A solution of 3-methyl-1H-indazol-5-amine (70 mg, 475.62 umol) in pyridine (1 mL) was cooled to 0° C., and then 4,5-dimethyl-4,7-dihydrotetrazolo[1,5-a]pyrimidine-6-carbonyl chloride (121.92 mg, 570.74 umol) in ACN (0.2 mL) was added dropwise to the solution. The resulting mixture was stirred at 25° C. for 12 hrs. The reaction mixture was concentrated under reduced pressure to remove the solvent. The residue was purified by prep-HPLC (TFA condition) to afford the product (36.86 mg, 78.96 umol, 16.60% yield, TFA salt) as a pale yellow solid. ¹H NMR (DMSO-d₆, 400 MHz) δ 12.58 (br s, 1H), 9.95 (s, 1H), 8.08 (s, 1H), 7.46-7.38 (m, 2H), 5.28 (s, 2H), 3.43 (s, 3H), 2.45 (s, 3H), 2.25 (s, 3H). MS-ESI (m/z) calcd for C₁₅H₁₇N₈O [M+H]+: 325.1. Found 325.1.

### Example 59. N-(3-(1H-Benzo[d]imidazol-2-yl)-1H-indazol-5-yl)-4,5-dimethyl-4,7-dihydrotetrazolo[1,5-a]pyrimidine-6-carboxamide

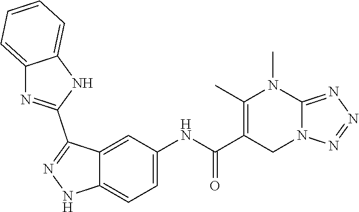

### Step 1. 3-(1H-Benzo[d]imidazol-2-yl)-5-nitro-1H-indazole

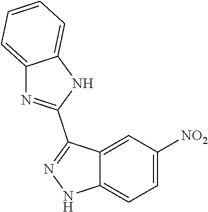

To a solution of 5-nitro-1H-indazole-3-carbaldehyde (280 mg, 1.46 mmol) in DMF (3 mL) was added 4 Å MS (500 mg) and benzene-1,2-diamine (237.62 mg, 2.20 mmol). The reaction mixture was stirred at 60° C. for 2 hrs, then heated to 80° C. for 12 hrs. After cooling to 20° C., the reaction mixture was filtered and the filtrate was concentrated. The residue was purified by prep-HPLC (neutral condition) to afford the product (130 mg, 465.53 umol, 31.78% yield) as a yellow solid.

### Step 2. 3-(1H-Benzo[d]imidazol-2-yl)-1H-indazol-5-amine

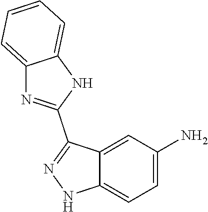

To a solution of 3-(1H-benzo[d]imidazol-2-yl)-5-nitro-1H-indazole (100 mg, 358.10 umol) in MeOH (2 mL) was added 10% Pd/C (0.1 g) under N₂. The suspension was degassed and purged with H₂ several times. The mixture was stirred under H₂ (15 psi) at 15° C. for 2 hrs. The reaction mixture was filtered and the filtrate was concentrated to afford the product (68 mg, 272.80 umol, 76.18% yield) as a yellow solid, which was used without further purification.

### Step 3. N-(3-(1H-Benzo[d]imidazol-2-yl)-1H-indazol-5-yl)-4,5-dimethyl-4,7-dihydrotetrazolo[1,5-a]pyrimidine-6-carboxamide

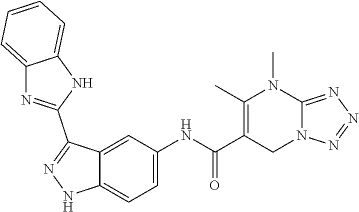

To a solution of 3-(1H-benzo[d]imidazol-2-yl)-1H-indazol-5-amine (60.00 mg, 240.70 umol) and 4,5-dimethyl-4,7-dihydrotetrazolo[1,5-a]pyrimidine-6-carboxylic acid (46.98 mg, 240.70 umol) in pyridine (1 mL) was added EDCI (69.21 mg, 361.05 umol). The reaction mixture was stirred at 30° C. for 12 hrs. The reaction mixture was concentrated. The residue was purified by prep-HPLC (neutral condition) to afford the product (27.57 mg, 59.97 umol, 24.92% yield) as a pale yellow solid. ¹H NMR (DMSO-d₆, 400 MHz) δ 13.75-13.47 (m, 1H), 13.11-12.85 (m, 1H), 10.15 (s, 1H), 8.76 (s, 1H), 7.82-7.46 (m, 4H), 7.27-7.17 (m, 2H), 5.33 (s, 2H), 3.44 (s, 3H), 2.29 (s, 3H). MS-ESI (m/z) calcd for C₂₁H₁₉N₁₀O [M+H]+: 427.2. Found 427.1.

### Example 60. 4,5,7,7-Tetramethyl-N-(3-methyl-2H-indazol-5-yl)-4,7-dihydrotetrazolo[1,5-a]pyrimidine-6-carboxamide

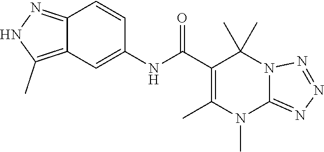 [SMILES: CC1=C(C(=O)Nc2ccc3n[nH]c(C)c3c2)C(C)(C)n2nnnc2N1C]

### Step 1. Methyl 2-acetyl-3-methylbut-2-enoate

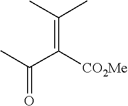

To a solution of ZnCl₂ (8.80 g, 64.59 mmol) and methyl acetoacetate (50 g, 430.61 mmol, 46.31 mL) in acetone (37.51 g, 645.91 mmol, 47.49 mL) was added Ac₂O (57.15 g, 559.79 mmol, 52.43 mL), the reaction mixture was heated at 50° C. for 48 hrs. The reaction mixture was diluted with DCM (1 L), and washed with water (300 mL), the organic phase was dried over anhydrous Na₂SO₄, filtered and the filtrate was concentrated under vacuum. The residue was purified by column chromatography (SiO₂, petroleum ether/EtOAc=1:0-10:1) to afford the product (25.6 g, 163.91 mmol, 38.07% yield) as a yellow oil.

### Step 2. Methyl 5,7,7-trimethyl-4,7-dihydrotetrazolo[1,5-a]pyrimidine-6-carboxylate

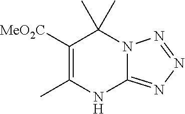

To a solution of methyl 2-acetyl-3-methylbut-2-enoate (25.6 g, 163.91 mmol) and 5-aminotetrazole (16.73 g, 196.70 mmol) in EtOH (200 mL) was added 4 Å molecular sieves (5 g), and the mixture was stirred at 80° C. for 12 hrs. The reaction mixture was cooled to room temperature and filtered. The filtrate was concentrate under vacuum to afford the product (29.46 g) as a light yellow solid which was used without further purification.

### Step 3. Methyl 4,5,7,7-tetramethyl-4,7-dihydrotetrazolo[1,5-a]pyrimidine-6-carboxylate

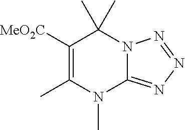

To a solution of methyl 5,7,7-trimethyl-4,7-dihydrotetrazolo[1,5-a]pyrimidine-6-carboxylate (29.46 g, 131.97 mmol) in DMF (300 mL) was added Mel (112.39 g, 791.83 mmol, 49.29 mL) and Cs₂CO₃ (257.99 g, 791.83 mmol). The reaction mixture was stirred at 50° C. for 13 hrs. The reaction mixture was concentrated under vacuum and the residue was purified by silica gel chromatography (SiO₂, petroleum ether/EtOAc=1:0-3:1) to afford the product (9.15 g, 30.85 mmol, 23.38% yield) as a light yellow solid.

### Step 4. 4,5,7,7-Tetramethyl-N-(3-methyl-2H-indazol-5-yl)-4,7-dihydrotetrazolo[1,5-a]pyrimidine-6-carboxamide

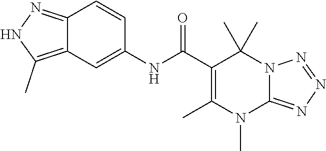 [SMILES: CC1=C(C(=O)Nc2ccc3n[nH]c(C)c3c2)C(C)(C)n2nnnc2N1C]

A solution of methyl 4,5,7,7-tetramethyl-4,7-dihydrotetrazolo[1,5-a]pyrimidine-6-carboxylate (50 mg, 339.73 umol) and 3-methyl-1H-indazol-5-amine (100 mg, 421.48 umol) in toluene (2 mL) was added A1(CH₃)₃ (2 M, 679.45 uL) and the mixture was stirred at 90° C. for 12 hrs. The reaction was quenched with MeOH (2 mL), and then the mixture was concentrated under vacuum. The residue was purified by prep-HPLC (basic condition) to afford the product (20.29 mg, 54.46 umol, 16.03% yield) as a pale yellow solid. ¹H NMR (DMSO-d₆, 400 MHz) δ 8.08 (s, 1H) 7.46 (s, 2H) 3.49 (s, 3H) 2.55 (s, 3H) 2.22 (s, 3H) 1.88 (s, 6H). MS-ESI (m/z) calcd for C₁₇H₂₁N₈O [M+H]+: 353.2. Found 353.2.

### Example 61. Methyl 5-(4,5-dimethyl-4,7-dihydrotetrazolo[1,5-a]pyrimidine-6-carboxamido)-2H-indazole-4-carboxylate

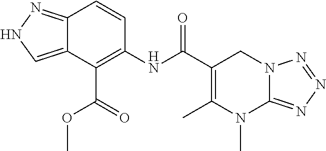

### Step 1. (E)-2-(Hydroxyimino)-N-(1H-indazol-5-yl)acetamide

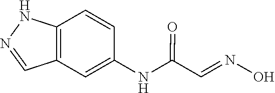 [SMILES: O=C(/C=N/O)Nc1ccc2[nH]ncc2c1]

To a solution of Na₂SO₄ (26 g, 183.05 mmol) in H₂O (15 ml) was added 1H-indazol-5-amine (1.3 g, 9.76 mmol) in 1M HCl (7 mL). Then 2,2,2-trichloroacetaldehyde (1.65 g, 11.19 mmol) was added to the mixture, after that, NH₂OH.HCl (2.2 g, 31.66 mmol) in H₂O (15 mL) was added, the resulted suspension was heated to 90° C. and stirred for 20 min. The reaction mixture was cooled to 20° C. and filtered. The solid was washed with H₂O (10 mL×5) and dried under vacuum to afford the product (1.94 g, crude) as a brown solid which was used without further purification.

### Step 2. 3,6-Dihydropyrrolo[3,2-e]indazole-7,8-dione

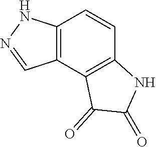 [SMILES: O=C1Nc2ccc3[nH]ncc3c2C1=O]

To H₂SO₄ (10 mL, 98% purity) was added (fs)-2-(hydroxyimino)-N-(1H-indazol-5-yl)acetamide (0.94 g, 4.60 mmol) slowly at 50° C. The reaction mixture was stirred at 75° C. for 20 min. and then poured into ice water (30 mL) and filtered. The solid that formed was collected and dried under vacuum to afford the product (800 mg, crude) as a dark purple solid which was used without further purification.

### Step 3. 5-Amino-1H-indazole-4-carboxylic acid

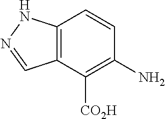 [SMILES: Nc1ccc2[nH]ncc2c1C(=O)O]

To a solution of 3,6-dihydropyrrolo[3,2-e]indazole-7,8-dione (800 mg, 4.27 mmol) in NaOH (4 mL) (2M aqueous solution) was added H₂O₂ (943.87 mg, 8.33 mmol, 30% purity) at 50° C. The mixture was cooled and stirred at 15° C. for 30 min. The reaction mixture was then acidified with 6N HCl to pH=4. The solid that formed was collected by filtration, washed with H₂O (5 mL×3), and dried under vacuum to afford the product (400 mg, crude) as a dark purple solid.

### Step 4. Methyl 5-amino-1H-indazole-4-carboxylate

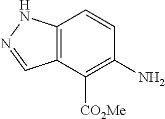 [SMILES: COC(=O)c1c(N)ccc2[nH]ncc12]

To a solution of 5-amino-1H-indazole-4-carboxylic acid (300 mg, 1.69 mmol) in MeOH (2 mL) and toluene (3 mL) was added TMSCHN₂ (2 M, 1.69 mL) (hexane solution) slowly, the resulted mixture was stirred at 20° C. for 0.5 hr. The reaction mixture was quenched with AcOH (0.5 mL) and concentrated to give a residue. The residue was purified by column chromatography (SiO₂, petroleum ether/EtOAc=l/0 to 1/4) to afford the product (250 mg, 77.22% yield) as a yellow solid.

### Step 5. Methyl 5-(4,5-dimethyl-4,7-dihydrotetrazolo[1,5-a]pyrimidine-6-carboxamido)-2H-indazole-4-carboxylate

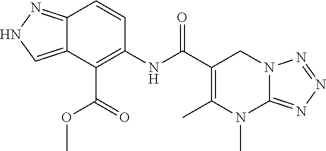 [SMILES: COC(=O)c1c(NC(=O)C2=C(C)N(C)c3nnnn3C2)ccc2n[nH]cc12]

To a solution of 4,5-dimethyl-4,7-dihydrotetrazolo[1,5-a]pyrimidine-6-carboxylic acid (250 mg, 1.28 mmol) in pyridine (5 mL) was added EDCI (368.32 mg, 1.92 mmol) and methyl 5-amino-1H-indazole-4-carboxylate (244.89 mg, 1.28 mmol). The reaction mixture was stirred at 30° C. for 12 hrs and concentrated to give a residue. The residue was taken up in H₂O (4 mL) and extracted with EtOAc (4 mL×3), the combined organic layers were washed with brine (5 mL×2), dried over Na₂SO₄, and filtered. The filtrate was concentrated to give a residue to which MeOH (10 mL) was added. A solid formed which was collected by filtration and dried in vacuo to afford crude product as a yellow solid. The material was purified by prep-HPLC (TFA condition) to afford the product (TFA salt) as a yellow solid. ¹H NMR (DMSO-d₆, 400 MHz) δ 8.48 (d, J=1 Hz, 1H) 7.66 (d, J=9 Hz, 2H) 7.07 (d, J=9 Hz, 1H) 5.46 (s, 2H) 3.86 (s, 3H) 3.50 (s, 3H) 2.19 (s, 3H). MS-ESI (m/z) calcd for C₁₆H₁₇N₈O₃ [M+H]+: 369.1. Found 369.1.

### Example 62. 4,5-Dimethyl-N-(4-methyl-2H-indazol-5-yl)-4,7-dihydrotetrazolo[1,5-a]pyrimidine-6-carboxamide

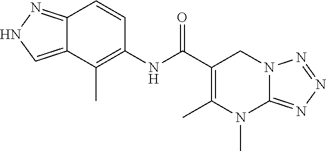 [SMILES: CC1=C(C(=O)Nc2ccc3n[nH]cc3c2C)Cn2nnnc2N1C]

To a solution of 4,5-dimethyl-4,7-dihydrotetrazolo[1,5-a]pyrimidine-6-carboxylic acid (66.31 mg, 339.73 umol) in pyridine (1 mL) was added EDCI (97.69 mg, 509.59 umol) and 4-methyl-2H-indazol-5-amine (50 mg, 339.73 umol). The reaction mixture was stirred at 30° C. for 12 hrs. The reaction mixture was concentrated to afford a residue which was purified by prep-HPLC (TFA condition) and further purified by prep-HPLC (neutral condition) to afford the product (16.75 mg, 50.70 umol, 14.92% yield) as a white solid. ¹H NMR (DMSO-d₆, 400 MHz) δ 13.02 (br s, 1H), 9.49 (s, 1H), 8.14 (s, 1H), 7.34 (d, J=8.8 Hz, 1H), 7.23 (d, J=8.6 Hz, 1H), 5.31 (br s, 2H), 3.43 (s, 3H), 2.42 (s, 3H), 2.33 (s, 3H). MS-ESI (m/z) calcd for C₁₅H₁₇N₈O [M+H]+: 325.1. Found 325.1.

### Example 63. 4,5-Dimethyl-N-(3-methyl-1H-indol-5-yl)-4,7-dihydrotetrazolo[1,5-a]pyrimidine-6-carboxamide

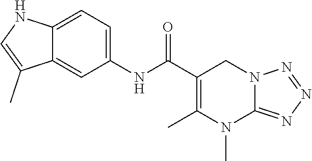 [SMILES: CC1=C(C(=O)Nc2ccc3[nH]cc(C)c3c2)Cn2nnnc2N1C]

### Step 1. I-(4-Nitrophenyl)-2-propylidenehydrazine

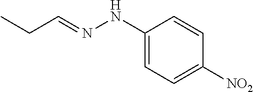

To a solution of propanaldehyde (569.75 mg, 9.81 mmol, 713.98 uL) in EtOH (6 mL) was added AcOH (196.37 mg, 3.27 mmol, 187.02 uL) and (4-nitrophenyl)hydrazine (500 mg, 3.27 mmol). The mixture was stirred at 25° C. for 12 hrs. The mixture was concentrated to afford the product (650 mg, crude) as a yellow solid, which was used without further purification.

### Step 2. 3-Methyl-5-nitro-1H-indole

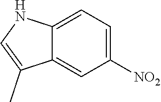

To a solution of 1-(4-nitrophenyl)-2-propylidenehydrazine (650 mg, 3.36 mmol) in toluene (12 mL) was added H₃PO₄ (5.88 g, 60.00 mmol, 3.5 mL). The biphasic reaction mixture was stirred at 95° C. for 3 hrs. after which the phases were separated. The reddish toluene phase was collected and additional fresh toluene was added to the H₃PO₄ layer. Stirring at 95° C. was continued for an additional 4 hrs after which the phases were separated and the toluene phase collected. This process was repeated an additional 2× the toluene extracts were combined, dried over Na₂CO₃ (100 mg) and the solvent was removed under reduced pressure at 60° C. to give a residue. The residue was purified by column chromatography (SiO₂, petroleum ether/EtOAc=100/0 to 85/15) to afford the product (150 mg, 851.44 umol, 25.31% yield) as an orange solid.

### Step 3. 3-Methyl-1H-indol-5-amine

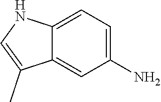

A mixture of 3-methyl-5-nitro-1H-indole (130 mg, 737.92 umol), 10% Pd/C (130 mg) in EtOH (2 mL) was degassed and purged with Eh (3×), and then the mixture was stirred at 25° C. for 2 hr under Eh (15 psi). The reaction mixture was filtered and the filtrate was concentrated to afford the product (140 mg, crude) as a black solid, which was used without further purification.

### Step 4. 4,5-Dimethyl-N-(3-methyl-1H-indol-5-yl)-4,7-dihydrotetrazolo[1,5-a]pyrimidine-6-carboxamide

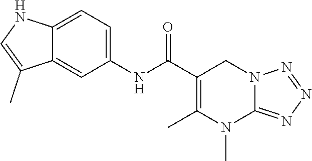 [SMILES: CC1=C(C(=O)Nc2ccc3[nH]cc(C)c3c2)Cn2nnnc2N1C]

To a solution of 3-methyl-1H-indol-5-amine (90 mg, 615.64 umol) and 4,5-dimethyl-4,7-dihydrotetrazolo[1,5-a]pyrimidine-6-carboxylic acid (120.16 mg, 615.64 umol) in pyridine (2 mL) was added EDCI (177.03 mg, 923.46 umol). The mixture was stirred at 25° C. for 4 hrs. and concentrated. The residue was purified by prep-HPLC (TFA condition) to afford the product (11.73 mg, 35.82 umol, 5.82% yield) as a pale pink solid. ¹H NMR (DMSO-d₆, 400 MHz) δ 10.68 (br s, 1H) 9.79 (s, 1H) 7.84 (s, 1H) 7.21-7.30 (m, 2H) 7.10 (s, 1H) 5.27 (s, 2H) 3.43 (s, 3H) 2.25 (s, 3H) 2.23 (s, 3H). MS-ESI (m/z) calcd for C₁₅H₁₈N₇O [M+H]+: 324.1. Found 324.1.

### Example 64. 4,5-Dimethyl-N-(3-methylimidazo[1,5-a]pyridin-6-yl)-4,7-dihydrotetrazolo[1,5-a]pyrimidine-6-carboxamide

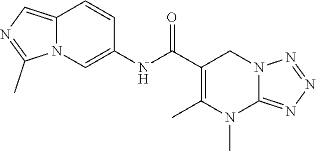 [SMILES: CC1=C(C(=O)Nc2ccc3cnc(C)n3c2)Cn2nnnc2N1C]

### Step 1. 2-(Bromomethyl)-5-nitropyridine

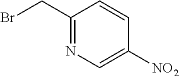 [SMILES: O=[N+]([O-])c1ccc(CBr)nc1]

To a solution of 2-methyl-5-nitropyridine (5 g, 36.20 mmol) in CCl₄ (75 mL) was added benzoyl peroxide (1.75 g, 7.24 mmol) and NBS (7.09 g, 39.82 mmol). The mixture was stirred at 80° C. for 12 hr. The reaction mixture was concentrated and purified by column chromatography (SiO₂, petroleum ether/EtOAc=1/0 to 10/1) to afford the product (2.45 g, 11.29 mmol, 31.19% yield) as a yellow oil.

### Step 2. (5-Nitropyridin-2-yl)methanamine

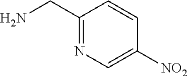 [SMILES: NCc1ccc([N+](=O)[O-])cn1]

To a mixture of NH₃.H₂O (10 mL) and dioxane (30 mL) was added 2-(bromomethyl)-5-nitropyridine (2.45 g, 11.29 mmol) in dioxane (10 mL). The resulted mixture was stirred at 25° C. for 2 hr. The reaction mixture was then concentrated to afford the product (1.7 g, crude) as a brown oil which was used without further purification.

### Step 3. 3-Methyl-6-nitroimidazo[1,5-a]pyridine

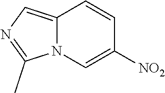

To a solution of (5-nitropyridin-2-yl)methanamine (1.7 g, 11.10 mmol) in Ac₂O (30 mL) was added PTSA (1.91 g, 11.10 mmol). The mixture was stirred at 100° C. for 2 hr. The reaction mixture was cooled to 25° C., poured into ice water (100 mL) and extracted with EtOAc (50 mL×3). The combined organic layers were dried over Na₂SO₄, filtered, and concentrated. The material was purified by column chromatography (SiO₂, petroleum ether/EtOAc=1/0 to 1/1) to afford the product (650 mg, 3.67 mmol, 33.05% yield) as a red solid.

### Step 4. 3-Methylimidazo[1,5-a]pyridin-6-amine

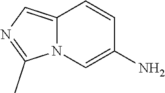

To a solution of 3-methyl-6-nitroimidazo[1,5-a]pyridine (70 mg, 395.12 umol) in MeOH (60 mL) was added 10% Pd/C (110 mg). The mixture was degassed and purged with H₂ (3×) and stirred at 25° C. for 0.5 hr under an H₂ atmosphere (15 psi). The reaction mixture was filtered and the filtrate was concentrated to afford the product (50 mg, crude) as a green oil which was used without further purification.

### Step 5. 4,5-Dimethyl-N-(3-methylimidazo[1,5-a]pyridin-6-yl)-4,7-dihydrotetrazolo[1,5-a]pyrimidine-6-carboxamide

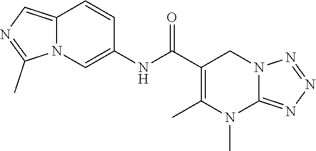 [SMILES: CC1=C(C(=O)Nc2ccc3cnc(C)n3c2)Cn2nnnc2N1C]

To a solution of 4,5-dimethyl-4,7-dihydrotetrazolo[1,5-a]pyrimidine-6-carboxylic acid (65 mg, 333.03 umol) in DMF (4 mL) was added 3-methylimidazo[1,5-a]pyridin-6-amine (49.01 mg, 333.03 umol), and DIEA (129.12 mg, 999.09 umol). A solution of HATU (189.94 mg, 499.54 umol) in DMF (1 mL) was then added to the mixture dropwise at 0° C. The mixture was stirred at 0° C. for 1 hr and then at 25° C. for 11 hrs. The reaction mixture was concentrated and purified by prep-HPLC (neutral condition) to afford the product (20.76 mg, 63.25 umol, 18.99% yield) as a gray solid. ¹H NMR (DMSO-d₆, 400 MHz) δ 9.95 (s, 1H) 8.71 (s, 1H) 7.51 (d, J=10 Hz, 1H) 7.23 (s, 1H) 6.74-6.78 (m, 1H) 5.27 (s, 2H) 3.43 (s, 3H) 2.53 (s, 3H) 2.25 (s, 3H). MS-ESI (m/z) calcd for C₁₅H₁₇N₈O [M+H]+: 325.1. Found 325.1.

### Example 65. 4,5-Dimethyl-N-(1-methyl-1H-indazol-6-yl)-4,7-dihydrotetrazolo[1,5-a]pyrimidine-6-carboxamide

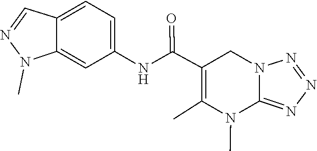

To a solution of 4,5-dimethyl-4,7-dihydrotetrazolo[1,5-a]pyrimidine-6-carboxylic acid (100 mg, 512.35 umol) in DCM (2 mL) was added 1-methyl-1H-indazol-6-amine (90.49 mg, 614.82 umol), T3P/EtOAc (489.06 mg, 768.53 umol, 457.07 uL, 50% purity) and TEA (155.53 mg, 1.54 mmol, 213.94 uL). The mixture was stirred at 25° C. for 12 hrs, and then at 60° C. for 6 hrs. The reaction mixture was concentrated under reduced pressure to give a residue. The residue was purified by prep-HPLC (TFA condition) to afford the product (18.61 mg, 52.62 umol, 10.27% yield) as a pale yellow solid. ¹H NMR (DMSO-d₆, 400 MHz) δ 10.16 (s, 1H) 8.14 (s, 1H) 7.96 (d, J=0.88 Hz, 1H) 7.68 (d, J=8.60 Hz, 1H) 7.19 (dd, 0.7=8.71, 1.65 Hz, 1H) 5.29 (s, 2H) 3.98 (s, 3H) 3.44 (s, 3H) 2.25 (s, 3H). MS-ESI (m/z) calcd for C₁₅H₁₇N₈O [M+H]+: 325.1. Found 325.1.

### Example 66. 4,5-Dimethyl-N-(3-methyl-6-(trifluoromethyl)-2H-indazol-5-yl)-4,7-dihydrotetrazolo[1,5-a]pyrimidine-6-carboxamide

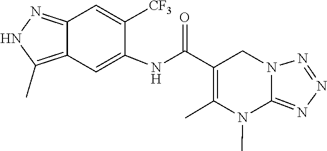

### Step 1. 3-Methyl-6-(trifluoromethyl)-2H-indazole

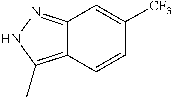

1-(2-Fluoro-4-(trifluoromethyl)phenyl)ethan-1-one (900 mg, 4.37 mmol) and N₂H₄.H₂O (334.54 mg, 6.55 mmol, 324.79 uL, 98% purity) were dissolved in ethylene glycol (11.10 g, 178.83 mmol, 10 mL) in a microwave tube. The sealed tube was heated at 200° C. for 1 hr in a microwave. The reaction mixture was diluted with water 20 mL and extracted with EtOAc (15 mL×3). The combined organic layers were dried over with Na₂SO₄, filtered and the filtrate was concentrated under reduced pressure to afford the product (1 g, crude) as a white solid which was used without further purification.

### Step 2. 3-Methyl-5-nitro-6-(trifluoromethyl)-2H-indazole

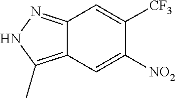 [SMILES: Cc1[nH]nc2cc(C(F)(F)F)c([N+](=O)[O-])cc12]

HNO₃ (699.97 mg, 7.22 mmol, 499.98 uL, 65% purity) was added dropwise to H₂SO₄ (1.84 g, 18.39 mmol, 1.00 mL, 98% purity) at 0° C. This was then added dropwise to a solution of 3-methyl-6-(trifluoromethyl)-2H-indazole (1 g, 5.00 mmol) in H₂SO₄ (20 mL, 98% purity) at −15° C. The mixture was then warmed up to −5° C. and stirred for 1 hr. The reaction mixture was added to ice (20 g), filtered and the solid was dried under reduced pressure to afford the product (900 mg, crude) as a light yellow solid which was used without further purification.

### Step 4. 3-Methyl-5-nitro-6-(trifluoromethyl)-1-((2-(trimethylsilyl)ethoxy)methyl)-1H-indazole

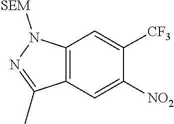 [SMILES: Cc1nn(COCC[Si](C)(C)C)c2cc(C(F)(F)F)c([N+](=O)[O-])cc12]

To a solution of 3-methyl-5-nitro-6-(trifluoromethyl)-2H-indazole (900 mg, 3.67 mmol) in THF (10 mL) was added NaH (293.66 mg, 7.34 mmol, 60% purity) at 0° C., then the mixture was stirred at 0° C. for 10 min, and SEM-Cl (734.46 mg, 4.41 mmol) was added dropwise. The reaction mixture was stirred at 25° C. for 2 hrs. and quenched by addition of water 10 mL at 25° C. The mixture was concentrated under reduced pressure to remove THF, and then extracted with EtOAc (10 mL×3). The combined organic layers were dried over Na₂SO₄, filtered and the filtrate was concentrated under reduced pressure to give a residue. The residue was purified by column chromatography (SiO₂, petroleum ether/EtOAc=1:0 to 5:1) to afford the product (700 mg, 1.86 mmol, 50.79% yield) as a light yellow oil.

### Step 5. 3-Methyl-6-(trifluoromethyl)-1-(#2-(trimethylsilyl)ethoxy)methyl)-1H-indazol-5-amine

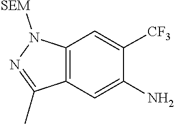 [SMILES: Cc1nn(COCC[Si](C)(C)C)c2cc(C(F)(F)F)c(N)cc12]

To a solution of 3-methyl-5-nitro-6-(trifluoromethyl)-1-((2-(trimethylsilyl)ethoxy)methyl)-1H-indazole (700 mg, 1.86 mmol) in EtOH (3 mL) and H₂O (1 mL) was added Fe (520.64 mg, 9.32 mmol) and NH₄Cl (498.70 mg, 9.32 mmol). The mixture was stirred at 80° C. for 1.5 hrs. The reaction mixture was filtered and the filtrate was concentrated under reduced pressure to remove solvent. The residue was diluted with sat. aq. NaHCO₃ (8 mL) and extracted with EtOAc (10 mL×3). The combined organic layers were dried over Na₂SO₄, filtered and the filtrate was concentrated under reduced pressure to give 752 mg crude product. 200 mg of the crude product was purified by prep-HPLC (neutral condition) to afford the product (53 mg, 153.43 umol, 8.23% yield) as a light yellow oil.

### Step 6. 4,5-Dimethyl-N-(3-methyl-6-(trifluoromethyl)-1-((2-(trimethylsilyl)ethoxy)methyl)-1H-indazol-5-yl)-4,7-dihydrotetrazolo[1,5-a]pyrimidine-6-carboxamide

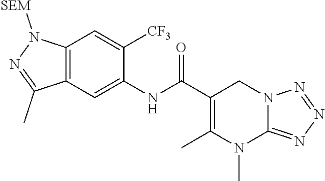 [SMILES: CC1=C(C(=O)Nc2cc3c(C)nn(COCC[Si](C)(C)C)c3cc2C(F)(F)F)Cn2nnnc2N1C]

To a solution of crude 3-methyl-6-(trifluoromethyl)-1-((2-(trimethylsilyl)ethoxy)methyl)-1H-indazol-5-amine (300 mg, crude) in DCM (1 mL), TEA (263.64 mg, 2.61 mmol, 362.64 uL) and DMAP (5.30 mg, 43.42 umol) at 0° C., was added a solution of 4,5-dimethyl-4,7-dihydrotetrazolo[1,5-a]pyrimidine-6-carbonyl chloride (371.05 mg, 1.74 mmol) in DCM (2 mL). The mixture was stirred at 25° C. for 8 hrs. The reaction mixture was diluted with water 8 mL and extracted with CH₂Cl₂ (8 mL×3) and EtOAc (8 mL×3). The combined organic layers were dried over with Na₂SO₄, filtered and concentrated under reduced pressure to afford the product (460 mg, crude) as a brown solid which was used without further purification.

### Step 7. 4,5-Dimethyl-N-(3-methyl-6-(trifluoromethyl)-2H-indazol-5-yl)-4,7-dihydrotetrazolo[1,5-a]pyrimidine-6-carboxamide

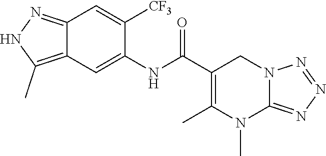 [SMILES: CC1=C(C(=O)Nc2cc3c(C)[nH]nc3cc2C(F)(F)F)Cn2nnnc2N1C]

To a solution of 4,5-dimethyl-N-(3-methyl-6-(trifluoromethyl)-1-((2-(trimethylsilyl)ethoxy)methyl)-1H-indazol-5-yl)-4,7-dihydrotetrazolo[1,5-a]pyrimidine-6-carboxamide (100 mg, 191.35 umol) in DCM (2 mL) was added TFA (616.00 mg, 5.40 mmol, 0.4 mL) at 0° C. The mixture was stirred at 0° C. for 2 hrs. The reaction mixture was concentrated under reduced pressure to give a residue. The residue was purified by prep-HPLC (neutral condition) to afford the product (4.46 mg, 11.09 umol, 5.80% yield) as a yellow solid. ¹H NMR (DMSO-d₆, 400 MHz) δ 13.16 (br s, 1H) 9.66 (s, 1H) 7.87 (d, J=9.04 Hz, 2H) 5.29 (s, 2H) 3.44 (s, 3H) 2.55 (s, 3H) 2.29-2.34 (m, 3H). MS-ESI (m/z) calcd for C₁₆H₁₆F₃N₈O [M+H]+: 393.1. Found 393.1.

### Example 67. N-(3-Benzamido-2H-indazol-5-yl)-4,5-dimethyl-4,7-dihydrotetrazolo[1,5-a]pyrimidine-6-carboxamide

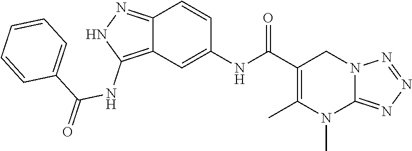 [SMILES: CC1=C(C(=O)Nc2ccc3n[nH]c(NC(=O)c4ccccc4)c3c2)Cn2nnnc2N1C]

### Step 1. N-(5-Nitro-2H-indazol-3-yl)benzamide

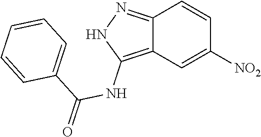 [SMILES: O=C(Nc1[nH]nc2ccc([N+](=O)[O-])cc12)c1ccccc1]

To a solution of 5-nitro-2H-indazol-3-amine (200 mg, 1.12 mmol) in pyridine (3 mL) was added a solution of benzoyl chloride (165.70 mg, 1.18 mmol, 136.94 uL) in CH₃CN (1 mL) at 0° C., then the mixture was stirred at 0° C. for 1 h. The reaction mixture was concentrated to a residue under vacuum. The residue was washed with MeOH (3 mL), filtered and the solid was dried under vacuum to afford the product (200 mg, 671.03 umol, 59.77% yield) as a yellow solid that was used without further purification.

### Step 2. N-(5-Amino-2H-indazol-3-yl)benzamide

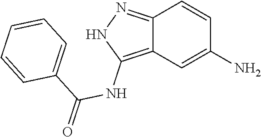 [SMILES: Nc1ccc2n[nH]c(NC(=O)c3ccccc3)c2c1]

To a solution of N-(5-nitro-2H-indazol-3-yl)benzamide (100 mg, 354.29 umol) in EtOH (2 mL) and H₂O (0.5 mL) was added Fe (98.93 mg, 1.77 mmol) and NH₄Cl (94.76 mg, 1.77 mmol), and the mixture was stirred at 80° C. for 2 hrs. The reaction mixture was filtered and the filtrate was concentrated under vacuum to afford the product (100 mg) as a yellow liquid which was used without further purification.

### Step 3. N-(3-benzamido-2H-indazol-5-yl)-4,5-dimethyl-4,7-dihydrotetrazolo[1,5-ci]pyrimidine-6-carboxamide

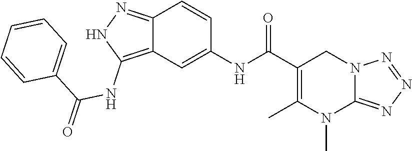 [SMILES: CC1=C(C(=O)Nc2ccc3n[nH]c(NC(=O)c4ccccc4)c3c2)Cn2nnnc2N1C]

To a stirred solution of N-(5-amino-2H-indazol-3-yl)benzamide (100 mg, 396.40 umol) and 4,5-dimethyl-4,7-dihydrotetrazolo[1,5-a]pyri mi dine-6-carboxylic acid (77.37 mg, 396.40 umol) in DCM (2 mL) was added T3P (756.76 mg, 1.19 mmol, 707.25 uL, 50% purity in EtOAc) and TEA (160.45 mg, 1.59 mmol, 220.70 uL), and then the reaction mixture was stirred at 25° C. for 12 hrs. The mixture was concentrated under vacuum. The residue was purified by prep-HPLC (neutral condition) to afford the product (11.51 mg, 24.40 umol, 6.16% yield) as a colorless gum. NMR (DMSO-d₆, 400 MHz) δ 9.97 (s, 1H) 7.94-8.09 (m, 3H) 7.43-7.62 (m, 5H) 5.24 (s, 2H) 3.39 (s, 3H) 2.21 (s, 3H). MS-ESI (m/z) calcd for C₂₁H₂₀N₉O₂ [M+H]+: 430.2. Found 430.2.

### Example A. LRRK2 Kinase Activity

LRRK2 kinase activity was measured using a LanthaScreen™ Kinase Activity Assay from ThermoFisher Scientific. Recombinant wild type or G2019S-LRRK2 protein (Life Technologies, PR8604B or PV4881, respectively), was incubated with a fluorescein-labeled peptide substrate called LRRKtide that is based upon ezrin/radixin/moesin (ERM) (Life Technologies, PV4901) in the presence of ATP and serially diluted compound. After an incubation period of 1 hr, the phosphotransferase activity was stopped and a terbium-labelled anti-pERM antibody (Life Technologies, PV4899) was added to detect the phosphorylation of LRRKtide by measuring the time resolved-Forster resonant energy transfer (TR-FRET) signal from the terbium label on the antibody to the fluorescein tag on LRRKtide, expressed as the 520 nm/495 nm emission ratio. Compound-dependent inhibition of the TR-FRET signal was used to generate a concentration-response curve for IC₅₀ determination.

The assay was carried out under the following protocol conditions: 1 mM compound in DMSO was serially diluted 1:3, 11 points in DMSO with a Biomek FX and 0.1 μL of the diluted compound was subsequently stamped into the assay plate (384-well format Lumitrac 200, Greiner, 781075) with an Echo Labcyte such that the final compound concentration in the assay was 10 μM to 169 μM. Subsequently, 5 μL of 2× kinase solution (2.9 nM final concentration) was added to the assay plate in assay buffer composed of 50 mM Tris pH 8.5 (Sigma, T6791), 5 mM MgCl₂ (Fluka, 63020), 1 mM EGTA (Sigma, E3889), 0.01% BRU-35 (Sigma, P1254) and 2 mM DTT. The reaction was started by addition of 2×ATP/LRRKtide solution in assay buffer such that the final concentration was 400 nM LRRKtide and 25 μM ATP. After 60 min incubation at room temperature, the reaction was stopped by addition of μL of 2× stop solution containing a final concentration of 2 nM anti-pERM antibody and mM EDTA. After a 30 min incubation at RT, the TR-FRET signal was measured on a Wallac 2104 EnVision® multilabel reader at an excitation wavelength of 340 nm and reading emission at 520 nm and 495 nm. The ratio of the 520 nm and 495 nm emission was used to analyze the data.

The Results of the LRRK2 kinase activity assay are shown in Table 1. Data is displayed as follows: + is IC₅₀≤100 nM; ++ is 100 nM≤IC₅₀≤1,000 nM; and +++ is 1,000 nM≤IC₅₀≤10,000 nM.

TABLE 1
LRRK2 Kinase Activity Assay

| | LRRK2 WT IC₅₀ | LRRK2 G2019S IC₅₀ |
|---|---|---|
| Example No. | (nM) | (nM) |
| 1 | ++ | + |
| 2 | ++ | + |
| 2a | + | + |
| 2b | +++ | ++ |
| 3 | +++ | +++ |
| 4 | +++ | +++ |
| 5 | ++ | ++ |
| 6 | + | + |
| 7 | + | + |
| 8 | ++ | + |
| 8a | ++ | + |
| 8b | ++ | + |
| 9 | + | + |
| 10 | + | + |
| 11 | + | + |
| 12 | ++ | + |
| 13 | + | + |
| 14 | +++ | ++ |
| 15 | ++ | + |
| 16 | +++ | ++ |
| 17 | >10000 | +++ |
| 18 | ++ | + |
| 19 | ++ | ++ |
| 20 | +++ | +++ |
| 21 | +++ | +++ |
| 22 | + | + |
| 23 | ++ | + |
| 24a | + | + |
| 24b | + | + |
| 25 | +++ | +++ |
| 26 | +++ | + |
| 27 | + | + |
| 28 | >10000 | +++ |
| 29 | >10000 | +++ |
| 30 | >10000 | +++ |
| 31 | +++ | ++ |
| 42 | ++ | + |
| 47 | +++ | + |
| 48 | >10000 | >10000 |
| 49 | >10000 | >10000 |
| 52 | +++ | ++ |
| 60 | ++ | + |
| 61 | >10000 | >10000 |
| 63 | >10000 | >10000 |

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A compound of Formula IA: 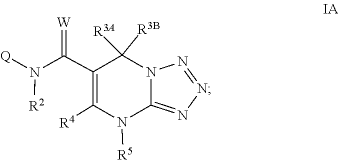
or a pharmaceutically acceptable salt thereof, wherein:
W is O or S;
Q is selected from one of the following: 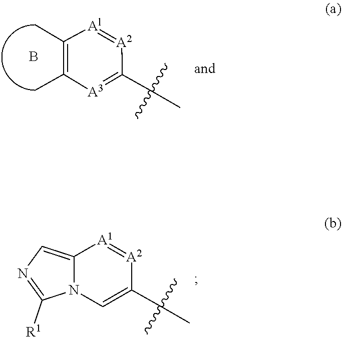
A¹, A², and A³ are each independently selected from N and CR⁶, wherein no more than two of A¹, A², and A³ in (a) are simultaneously N;
ring B is selected from: 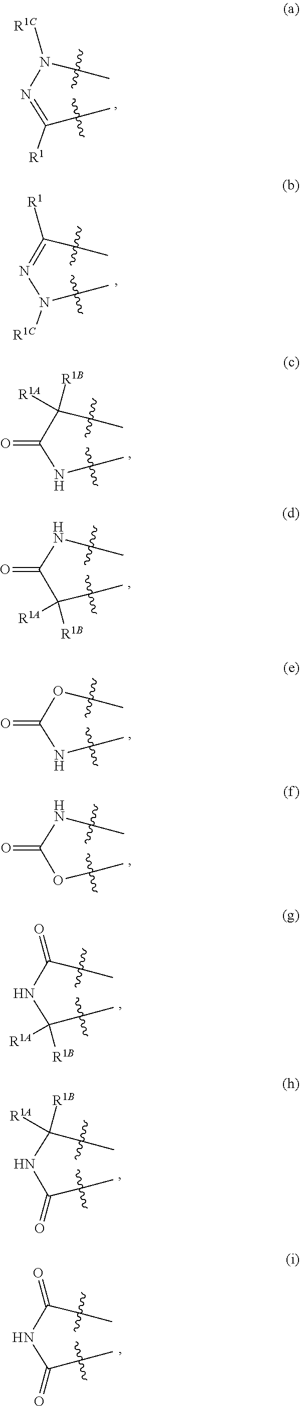 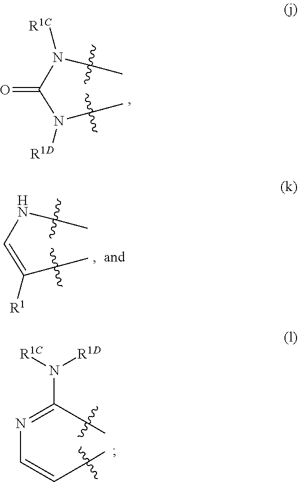
R¹, R^{1A}, and R^{1B} are each independently selected from H, halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}; wherein said Cue alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹ are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
or R^{1A} and R^{1B} together form a C₃₋₇ cycloalkyl or 4-10 membered heterocycloalkyl ring, each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
R^{1C} and R^{1D} are each independently selected from H and C₁₋₃ alkyl;
R² is H or C₁₋₄ alkyl;
R^{3A} and R^{3B} are each independently selected from H, halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹ are each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
or R^{3A} and R^{3B} together form a C₃₋₇ cycloalkyl or 4-10 membered heterocycloalkyl ring, each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
R⁴ is H, C₁₋₄ alkyl, halo, C₁₋₄ haloalkyl, or CN;
R⁵ is H, C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹ are each optionally substituted with 1, 2, 3, 4, or 5 substituents independently selected from Cy³, Cy³-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR³², C(O)R^{b2}, C(O)NR^{c2}R^{d2}, C(O)OR^{a2}, OC(O)R^{b2}, OC(O)NR^{c2}R^{d2}, NR^{c2}R^{d2} NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, or S(O)₂NR^{c2}R^{d2};
each R⁶ is independently selected from H, halo, C₁₋₆ alkyl, C₁₋₆ haloalkyl, C₆₋₁₀ aryl, C₃₋₇ cycloalkyl, 5-6 membered heteroaryl, 4-7 membered heterocycloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3}, wherein said C₁₋₆ alkyl, C₁₋₆ haloalkyl, C₆₋₁₀ aryl, C₃₋₇ cycloalkyl, 5-6 membered heteroaryl, 4-7 membered heterocycloalkyl of R⁶ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
each Cy¹ is independently selected from C₆₋₁₀ aryl, C₃₋₁₀ cycloalkyl, 5-14 membered heteroaryl, and 4-14 membered heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
each Cy² is independently selected from C₆₋₁₀ aryl, C₃₋₁₀ cycloalkyl, 5-14 membered heteroaryl, and 4-14 membered heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each Cy³ is independently selected from C₆₋₁₀ aryl, C₃₋₁₀ cycloalkyl, 5-14 membered heteroaryl, and 4-14 membered heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR³², C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each R^{a}, R^{b}, R^{c}, R^{d}, R^{a1}, R^{b1}, R^{c1}, R^{d1}, R^{a2}, R^{b2}, R^{c2}, R^{d2}, R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇cycloalkyl-C₁₋₄ alkyl, 5-10 membered heteroaryl-C₁₋₄ alkyl, and 4-10 membered heterocycloalkyl-C₁₋₄ alkyl, wherein said CM alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-CM alkyl of R^{a}, R^{b}, R^{c}, R^{d}, R^{a1}, R^{b1}, R^{c1}, R^{d1}, R^{a2}, R^{b2}, R^{c2}, R^{d2}, R^{a3}, R^{b3}, R^{c3}, or R^{d3} is optionally substituted with 1, 2, 3, 4, or 5 substituents independently selected from halo, CM alkyl, C₁₋₄ haloalkyl, CM haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
each R^{a4}, R^{b4}, R^{c4}, and R^{d4} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, 4-7 membered heterocycloalkyl, wherein said CM alkyl, CM haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl are each optionally substituted with 1, 2, or 3 substituents independently selected from OH, CN, amino, halo, C₁₋₆ alkyl, C₁₋₆alkoxy, C₁₋₆ haloalkyl, and C₁₋₆ haloalkoxy; and
each R^{e}, R^{e1}, R^{e2}, R^{e3}, and R^{e4} is independently selected from H, CM alkyl, and CN;
with the proviso that the compound is other than:

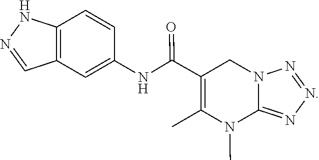 [SMILES: CC1=C(C(=O)Nc2ccc3[nH]ncc3c2)Cn2nnnc2N1C]

2. The compound of claim 1, or a pharmaceutically acceptable salt thereof, wherein Q is

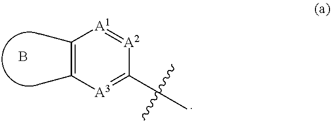

3. The compound of claim 1, or a pharmaceutically acceptable salt thereof, wherein Q is

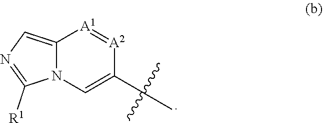

4. The compound of claim 1, or a pharmaceutically acceptable salt thereof, wherein Q is (a) and A¹, A², and A³ are each CR⁶.

5. The compound of claim 1, or a pharmaceutically acceptable salt thereof, wherein Q is (a) and A¹ is N, and A² and A³ are each CR⁶.

6. The compound of claim 1, or a pharmaceutically acceptable salt thereof, wherein Q is (a) and A¹ and A³ are each CR⁶, and A² is N.

7. The compound of claim 1, or a pharmaceutically acceptable salt thereof, wherein Q is (b) and A¹ and A² are each CR⁶.

8. The compound of claim 1, or a pharmaceutically acceptable salt thereof, wherein W is O.

9. The compound of claim 1, or a pharmaceutically acceptable salt thereof, wherein W is S.

10. The compound of claim 1, or a pharmaceutically acceptable salt thereof, wherein ring B is selected from:

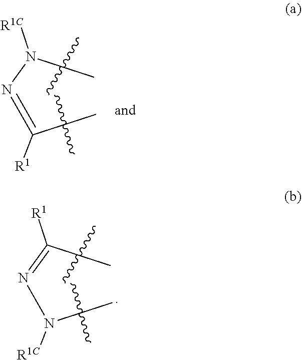

11. The compound of claim 3, or a pharmaceutically acceptable salt thereof, wherein ring B is selected from:

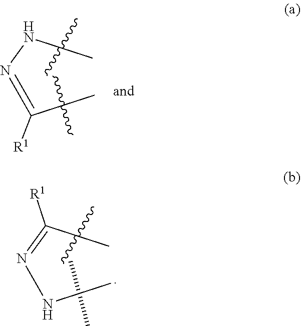

12. The compound of claim 3, or a pharmaceutically acceptable salt thereof, wherein ring B is

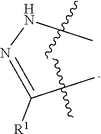

13. The compound of claim 3, or a pharmaceutically acceptable salt thereof, wherein ring B is

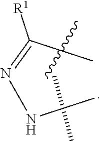

14. The compound of claim 1, or a pharmaceutically acceptable salt thereof, wherein ring B is selected from:

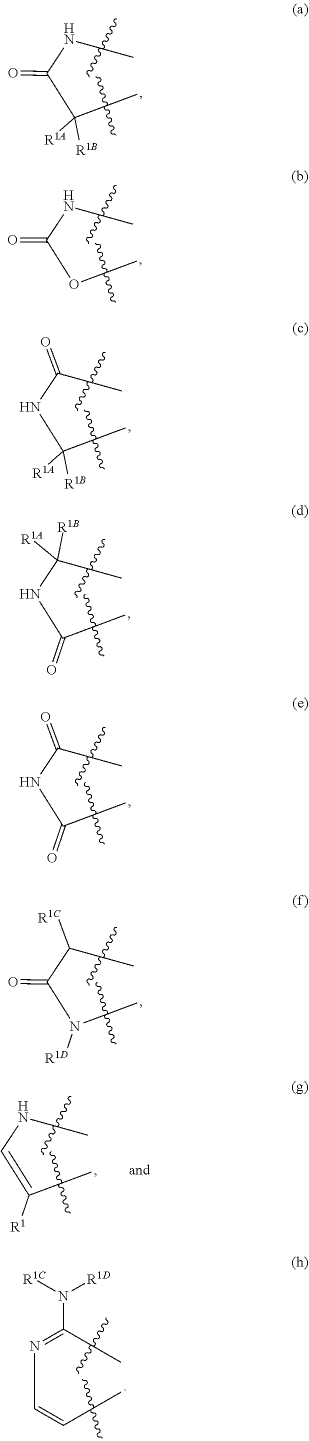

15. The compound of claim 1, or a pharmaceutically acceptable salt thereof, wherein R^{1A} and R^{1B} are each independently selected from H and C₁₋₆ alkyl.

16. The compound of claim 1, or a pharmaceutically acceptable salt thereof, wherein R^{1A} and R^{1B} are each methyl.

17. The compound of claim 1, or a pharmaceutically acceptable salt thereof, wherein R^{1A} and R^{1B} are each H.

18. The compound of claim 1, or a pharmaceutically acceptable salt thereof, wherein R^{1C} and R^{1D} are each H.

19. The compound of claim 1, or a pharmaceutically acceptable salt thereof, wherein R^{1C} is C₁₋₃ alkyl.

20. The compound of claim 1, or a pharmaceutically acceptable salt thereof, wherein R^{1C} is methyl.

21. The compound of claim 1, or a pharmaceutically acceptable salt thereof, wherein R^{1C} is H.

22. The compound of claim 1, or a pharmaceutically acceptable salt thereof, wherein R^{1D} is C₁₋₃ alkyl.

23. The compound of claim 1, or a pharmaceutically acceptable salt thereof, wherein R^{1D} is methyl.

24. The compound of claim 1, or a pharmaceutically acceptable salt thereof, wherein R^{1D} is H.

25. The compound of claim 1, or a pharmaceutically acceptable salt thereof, wherein R¹ is selected from H, halo, C₁₋₆ alkyl, C₆₋₁₀ aryl, 5-14 membered heteroaryl, C(O)R^{b}, C(O)NR^{c}R^{d}, NR^{c}R^{d}, and NR^{c}C(O)R^{b}; wherein said C₁₋₆ alkyl, C₆₋₁₀ aryl, and 5-14 membered heteroaryl are each optionally substituted with 1, 2, 3, 4, or substituents independently selected from Cy¹, Cy¹-C₁₋₄ alkyl, halo, C₁₋₆ alkyl and S(O)₂NR^{c}R^{d}.

26. The compound of claim 1, or a pharmaceutically acceptable salt thereof, wherein R¹ is selected from H, halo, C₁₋₆ alkyl, C₆₋₁₀ aryl, 4-14 membered heterocycloalkyl, 5-14 membered heteroaryl, C(O)R^{b}, C(O)OR^{a}, C(O)NR^{c}R^{d}, NR^{c}R^{d}, and NR^{c}C(O)R^{b}; wherein said C₁₋₆ alkyl, C₆₋₁₀ aryl, 4-14 membered heterocycloalkyl, and 5-14 membered heteroaryl are each optionally substituted with 1, 2, 3, 4, or 5 substituents independently selected from Cy¹, Cy¹-C₁₋₄ alkyl, halo, C₁₋₆ alkyl and S(O)₂NR^{c}R^{d}.

27. The compound of claim 1, or a pharmaceutically acceptable salt thereof, wherein R¹ is H.

28. The compound of claim 1, or a pharmaceutically acceptable salt thereof, wherein R¹ is halo.

29. The compound of claim 1, or a pharmaceutically acceptable salt thereof, wherein R¹ is Br.

30. The compound of claim 1, or a pharmaceutically acceptable salt thereof, wherein R¹ is C₁₋₆ alkyl.

31. The compound of claim 1, or a pharmaceutically acceptable salt thereof, wherein R¹ is methyl.

32. The compound of claim 1, or a pharmaceutically acceptable salt thereof, wherein R¹ is methyl or isopropyl.

33. The compound of claim 1, or a pharmaceutically acceptable salt thereof, wherein R¹ is C₆₋₁₀ aryl, optionally substituted with Cy¹ or SO₂NH₂.

34. The compound of claim 1, or a pharmaceutically acceptable salt thereof, wherein R¹ is phenyl.

35. The compound of claim 1, or a pharmaceutically acceptable salt thereof, wherein R¹ is 5-10 membered heteroaryl, optionally substituted with Cy¹.

36. The compound of claim 1, or a pharmaceutically acceptable salt thereof, wherein R¹ is pyridinyl or pyrimidinyl.

37. The compound of claim 1, or a pharmaceutically acceptable salt thereof, wherein R¹ is pyridinyl, pyrimidinyl, or 1H-benzo[d]imidazolyl, each optionally substituted with Cy¹.

38. The compound of claim 1, or a pharmaceutically acceptable salt thereof, wherein R¹ is 4-14 membered heterocycloalkyl optionally substituted with 1, 2, 3, 4, or 5 substituents independently selected from Cy¹, Cy¹-C₁₋₄ alkyl, halo, C₁₋₆ alkyl and S(O)₂NR^{c}R^{d}.

39. The compound of claim 1, or a pharmaceutically acceptable salt thereof, wherein R¹ is pyrrolidinyl.

40. The compound of claim 1, or a pharmaceutically acceptable salt thereof, wherein R¹ is NH₂.

41. The compound of claim 1, or a pharmaceutically acceptable salt thereof, wherein R¹ is CONH₂.

42. The compound of claim 1, or a pharmaceutically acceptable salt thereof, wherein R¹ is C(O)OR^{a}.

43. The compound of claim 1, or a pharmaceutically acceptable salt thereof, wherein R¹ is NR^{c}C(O)R^{b}.

44. The compound of claim 1, or a pharmaceutically acceptable salt thereof, wherein R¹ is C(O)NR^{c}R^{d}.

45. The compound of claim 1, or a pharmaceutically acceptable salt thereof, wherein R² is H.

46. The compound of claim 1, or a pharmaceutically acceptable salt thereof, wherein R² is C₁₋₄ alkyl.

47. The compound of claim 1, or a pharmaceutically acceptable salt thereof, wherein R² is methyl.

48. The compound of claim 1, or a pharmaceutically acceptable salt thereof, wherein R^{3A} and R^{3B} are each independently selected from H, C₁₋₆ alkyl, C₆₋₁₀ aryl, and 5-14 membered heteroaryl, wherein said C₁₋₆ alkyl and 5-14 membered heteroaryl are each optionally substituted with 1, 2, 3, 4, or 5 substituents independently selected from Cy², Cy²-C₁₋₄ alkyl, halo, C₁₋₆ alkyl, and OR^{a1}.

49. The compound of claim 1, or a pharmaceutically acceptable salt thereof, wherein R^{3A} and R^{3B} are each independently selected from H, methyl, ethyl, isopropyl, phenyl, and OH.

50. The compound of claim 1, or a pharmaceutically acceptable salt thereof, wherein R^{3A} is C₁₋₆ alkyl optionally substituted with OR^{a1}.

51. The compound of claim 1, or a pharmaceutically acceptable salt thereof, wherein R^{3A} and R^{3B} are each H.

52. The compound of claim 1, or a pharmaceutically acceptable salt thereof, wherein R^{3A} and R^{3B} are each methyl.

53. The compound of claim 1, or a pharmaceutically acceptable salt thereof, wherein R^{3A} is methyl and R^{3B} is H.

54. The compound of claim 1, or a pharmaceutically acceptable salt thereof, wherein R^{3A} and R^{3B} together form a C₃₋₇ cycloalkyl.

55. The compound of claim 1, or a pharmaceutically acceptable salt thereof, wherein R^{3A} and R^{3B} together form a cyclopentyl group.

56. The compound of claim 1, or a pharmaceutically acceptable salt thereof, wherein R⁴ is H.

57. The compound of claim 1, or a pharmaceutically acceptable salt thereof, wherein R⁴ is C₁₋₄ alkyl.

58. The compound of claim 1, or a pharmaceutically acceptable salt thereof, wherein R⁴ is methyl.

59. The compound of claim 1, or a pharmaceutically acceptable salt thereof, wherein R⁴ is ethyl.

60. The compound of claim 1, or a pharmaceutically acceptable salt thereof, wherein R⁵ is H, C₁₋₆ alkyl, C₆₋₁₀ aryl, C₃₋₁₀ cycloalkyl, 5-14 membered heteroaryl, 4-14 membered heterocycloalkyl, 4-10 membered heterocycloalkyl-C₁₋₄ alkyl, CN, NO₂, or C(O)NR^{c2}R^{d2}.

61. The compound of claim 1, or a pharmaceutically acceptable salt thereof, wherein R⁵ is H, C₁₋₆ alkyl, C₆₋₁₀ aryl, C₃₋₁₀ cycloalkyl, 5-14 membered heteroaryl, 4-14 membered heterocycloalkyl, 4-10 membered heterocycloalkyl-C₁₋₄ alkyl, CN, NO₂, C(O)NR^{c2}R^{d2}, or C(O)R^{b2}, wherein said C₁₋₆ alkyl, C₆₋₁₀ aryl, C₃₋₁₀ cycloalkyl, 5-14 membered heteroaryl, 4-14 membered heterocycloalkyl, and 4-10 membered heterocycloalkyl-C₁₋₄ alkyl are each optionally substituted with 1, 2, 3, 4, or 5 substituents independently selected from Cy³, Cy³-C₁₋₄ alkyl, halo, C₁₋₆ alkyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, and NR^{c2}R^{d2}.

62. The compound of claim 1, or a pharmaceutically acceptable salt thereof, wherein R⁵ is H.

63. The compound of claim 1, or a pharmaceutically acceptable salt thereof, wherein R⁵ is C₁₋₆ alkyl.

64. The compound of claim 1, or a pharmaceutically acceptable salt thereof, wherein R⁵ is methyl.

65. The compound of claim 1, or a pharmaceutically acceptable salt thereof, wherein R⁵ is ethyl.

66. The compound of claim 1, or a pharmaceutically acceptable salt thereof, wherein R⁵ is C₆₋₁₀ aryl.

67. The compound of claim 1, or a pharmaceutically acceptable salt thereof, wherein R⁵ is phenyl.

68. The compound of claim 1, or a pharmaceutically acceptable salt thereof, wherein R⁵ is 4-10 membered heterocycloalkyl-C₁₋₄ alkyl.

69. The compound of claim 1, or a pharmaceutically acceptable salt thereof, wherein R⁵ is morpholino-C₁₋₄ alkyl.

70. The compound of claim 1, or a pharmaceutically acceptable salt thereof, wherein R⁵ is C(O)NR^{c2}R^{d2}.

71. The compound of claim 1, or a pharmaceutically acceptable salt thereof, wherein R⁶ is H, halo, OR^{a3}, C(O)NR^{c3}R^{d3}, C(O)OR^{a3}, or NR^{c3}R^{d3}.

72. The compound of claim 1, or a pharmaceutically acceptable salt thereof, wherein each R⁶ is independently selected from H, halo, OR^{a3}, C(O)NR^{c3}R^{d3}, C(O)OR^{a3}, and NR^{c3}R^{d3}.

73. The compound of claim 1, or a pharmaceutically acceptable salt thereof, wherein each R⁶ is independently selected from H, halo, OR^{a3}, C₁₋₆ alkyl, C₁₋₆ haloalkyl, C(O)NR^{c3}R^{d3}, C(O)OR^{a3}, and NR^{c3}R^{d3}.

74. The compound of claim 1, or a pharmaceutically acceptable salt thereof, wherein each R⁶ is independently selected from H, F, methyl, methoxy, and CF₃.

75. The compound of claim 1, or a pharmaceutically acceptable salt thereof, wherein R⁶ is H.

76. The compound of claim 1, or a pharmaceutically acceptable salt thereof, wherein R⁶ is halo.

77. The compound of claim 1, or a pharmaceutically acceptable salt thereof, wherein each R⁶ is independently selected from H and halo.

78. The compound of claim 1, or a pharmaceutically acceptable salt thereof, wherein R⁶ is F.

79. The compound of claim 1, or a pharmaceutically acceptable salt thereof, wherein each R⁶ is independently selected from H and F.

80. The compound of claim 1, or a pharmaceutically acceptable salt thereof, wherein R⁶ is methoxy.

81. The compound of claim 1, or a pharmaceutically acceptable salt thereof, wherein each R⁶ is independently selected from H and methoxy.

82. The compound of claim 1, or a pharmaceutically acceptable salt thereof, wherein each R⁶ is independently selected from H, C(O)NR^{c3}R^{d3}, and NR^{c3}R^{d3}.

83. The compound of claim 1, having Formula II: 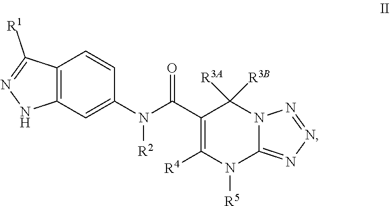
or a pharmaceutically acceptable salt thereof.

84. The compound of claim 1, having Formula III: 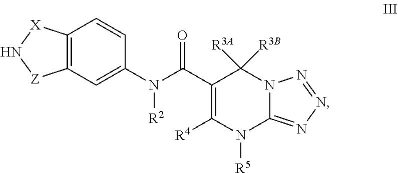
or a pharmaceutically acceptable salt thereof, wherein
X is oxo (═O) or CR^{1A}R^{1B}; and
Z is oxo (═O) or CR^{1A}R^{1B},
wherein if X is CR^{1A}R^{1B} then Z is not CR^{1A}R^{1B}.

85. The compound of claim 1, having Formula IV: 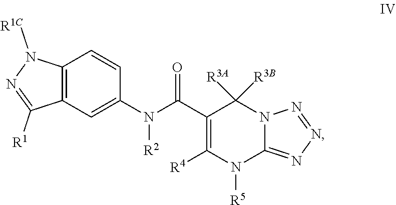
or a pharmaceutically acceptable salt thereof.

86. The compound of claim 1, or a pharmaceutically acceptable salt thereof, wherein:
W is O or S;
Q is selected from one of the following: 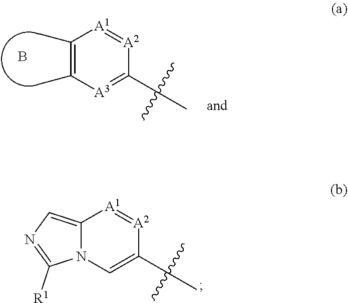
A¹, A², and A³ are each independently selected from N and CR⁶, wherein no more than two of A¹, A², and A³ in (a) are simultaneously N;
ring B is selected from: 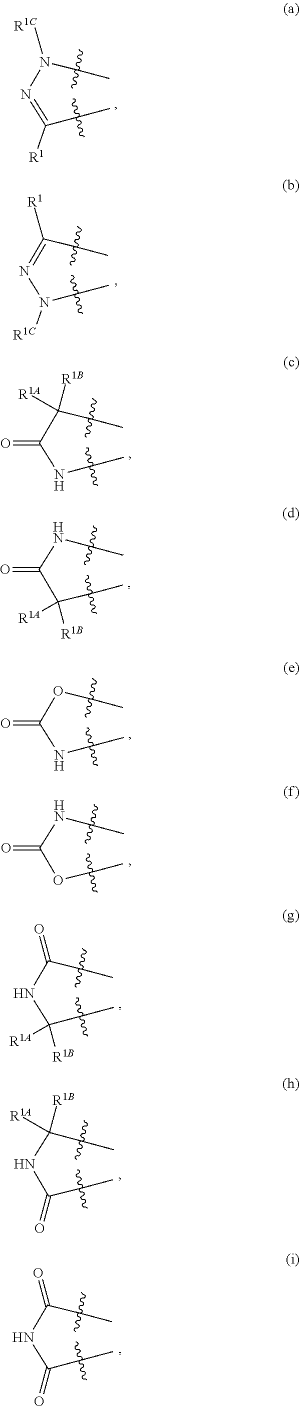 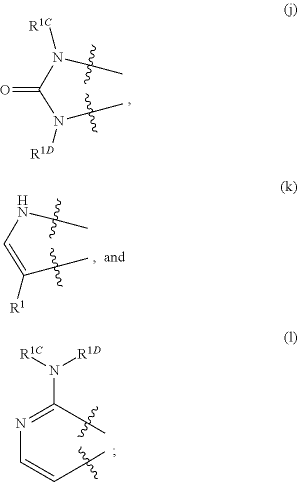
R¹, R^{1A}, and R^{1B} are each independently selected from H, halo, C₁₋₆ alkyl, C₆₋₁₀ aryl, 5-14 membered heteroaryl, C(O)R^{b}, C(O)NR^{c}R^{d}, NR^{c}R^{d}, and NR^{c}C(O)R^{b}; wherein said C₁₋₆ alkyl, C₆₋₁₀ aryl, and 5-14 membered heteroaryl are each optionally substituted with 1, 2, 3, 4, or 5 substituents independently selected from Cy¹, Cy¹-C₁₋₄ alkyl, halo, C₁₋₆ alkyl and S(O)₂NR^{c}R^{d};
R^{1C} and R^{1D} are each independently selected from H and C₁₋₃ alkyl;
R² is H or C₁₋₄ alkyl;
R^{3A} and R^{3B} are each independently selected from H, C₁₋₆ alkyl, C₆₋₁₀ aryl, 5-14 membered heteroaryl, wherein said C₁₋₆ alkyl and 5-14 membered heteroaryl are each optionally substituted with 1, 2, 3, 4, or 5 substituents independently selected from Cy², Cy²-C₁₋₄ alkyl, halo, C₁₋₆ alkyl, and OR^{a1};
or R^{3A} and R^{3B} together form a C₃₋₇ cycloalkyl optionally substituted with 1, 2, 3, 4, or 5 substituents independently selected from Cy², Cy²-C₁₋₄ alkyl, halo, C₁₋₆ alkyl, and OR^{a1};
R⁴ is H or C₁₋₄ alkyl;
R⁵ is H, C₁₋₆ alkyl, C₆₋₁₀ aryl, C₃₋₁₀ cycloalkyl, 5-14 membered heteroaryl, 4-14 membered heterocycloalkyl, 4-10 membered heterocycloalkyl-C₁₋₄ alkyl, CN, NO₂, or C(O)NR^{c2}R^{d2};
R⁶ is H, halo, C₁₋₆ alkyl, C₁₋₆ haloalkyl, OR^{a3}, C(O)NR^{c3}R^{d3}, C(O)OR^{a3}, or NR^{c3}R^{d3};
each Cy¹ is independently selected from 5-14 membered heteroaryl and 4-14 membered heterocycloalkyl;
each Cy² is independently selected from C₆₋₁₀ aryl, C₃₋₁₀ cycloalkyl, 5-14 membered heteroaryl, and 4-14 membered heterocycloalkyl;
each R^{b}, R^{c}, R^{d}, R^{a1}, R^{c2}, R^{d2}, R^{a3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-CM alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{b}, R^{c}, R^{d}, R^{a1}, R^{c2}, R^{d2}, R^{a3}, R^{c3}, or R^{d3} is optionally substituted with 1, 2, 3, 4, or 5 substituents independently selected from halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
each R^{a4}, R^{b4}, R^{c4}, and R^{d4} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, 4-7 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy; and
each R^{e4} is independently selected from H, C₁₋₄ alkyl, and CN;
with the proviso that the compound is other than:

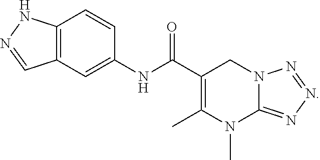 [SMILES: CC1=C(C(=O)Nc2ccc3[nH]ncc3c2)Cn2nnnc2N1C]

87. The compound of claim 1, having Formula I: 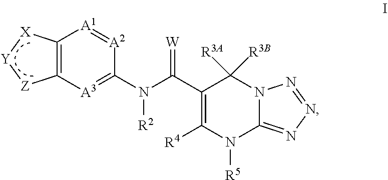
or a pharmaceutically acceptable salt thereof, wherein:
A¹, A², and A³ are each independently selected from N and CR⁶, wherein no more than two of A¹, A², and A³ are simultaneously N;
W is O or S;
the moiety 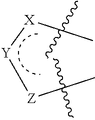
is selected from: 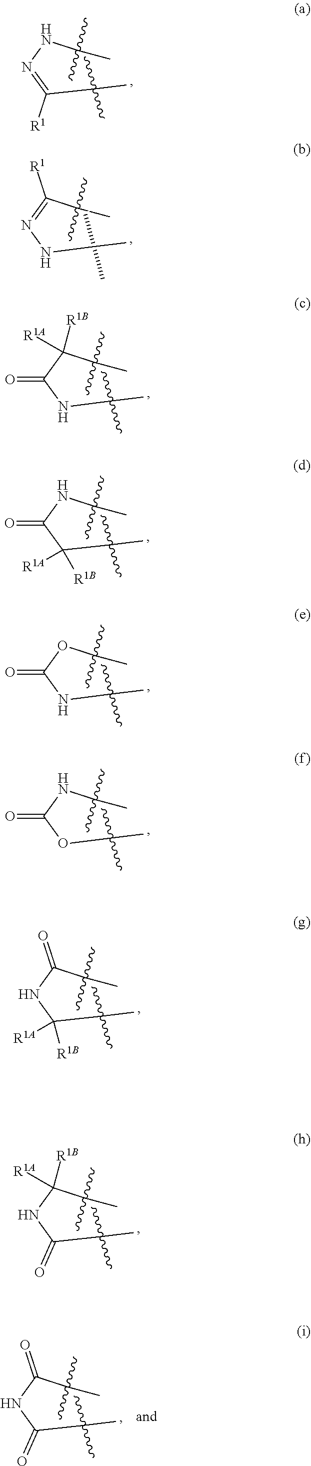 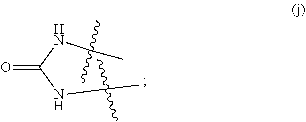 [SMILES: *NC(=O)N*]
R¹, R^{1A}, and R^{1B} are each independently selected from H, halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}; wherein said C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹ are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
or R^{1A} and R^{1B} together form a C₃₋₇ cycloalkyl or 4-10 membered heterocycloalkyl ring, each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
R² is H or C₁₋₄ alkyl;
R^{3A} and R^{3B} are each independently selected from H, halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹ are each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
or R^{3A} and R^{3B} together form a C₃₋₇ cycloalkyl or 4-10 membered heterocycloalkyl ring, each optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
R⁴ is H, C₁₋₄ alkyl, halo, C₁₋₄ haloalkyl, or CN;
R⁵ is H, C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹ are each optionally substituted with 1, 2, 3, 4, or 5 substituents independently selected from Cy³, Cy³-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR³², C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, or S(O)₂NR^{c2}R^{d2};
each R⁶ is independently selected from H, halo, C₁₋₆ alkyl, C₁₋₆ haloalkyl, C₆₋₁₀ aryl, C₃₋₇ cycloalkyl, 5-6 membered heteroaryl, 4-7 membered heterocycloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3}, wherein said C₁₋₆ alkyl, C₁₋₆ haloalkyl, C₆₋₁₀ aryl, C₃₋₇ cycloalkyl, 5-6 membered heteroaryl, 4-7 membered heterocycloalkyl of R⁶ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
each Cy¹ is independently selected from C₆₋₁₀ aryl, C₃₋₁₀ cycloalkyl, 5-14 membered heteroaryl, and 4-14 membered heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
each Cy² is independently selected from C₆₋₁₀ aryl, C₃₋₁₀ cycloalkyl, 5-14 membered heteroaryl, and 4-14 membered heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each Cy³ is independently selected from C₆₋₁₀ aryl, C₃₋₁₀ cycloalkyl, 5-14 membered heteroaryl, and 4-14 membered heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR³², C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each R^{a}, R^{b}, R^{c}, R^{d}, R^{a1}, R^{b1}, R^{c1}, R^{d1}, R^{a2}, R^{b2}, R^{c2}, R^{d2}, R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇cycloalkyl-C₁₋₄ alkyl, 5-10 membered heteroaryl-C₁₋₄ alkyl, and 4-10 membered heterocycloalkyl-C₁₋₄ alkyl, wherein said CM alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-CM alkyl of R^{a}, R^{b}, R^{c}, R^{d}, R^{a1}, R^{b1}, R^{c1}, R^{d1}, R^{a2}, R^{b2}, R^{c2}, R^{d2}, R^{a3}, R^{b3}, R^{c3}, or R^{d3} is optionally substituted with 1, 2, 3, 4, or 5 substituents independently selected halo, C₁₋₄ alkyl, CM haloalkyl, CM haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
each R^{a4}, R^{b4}, R^{c4}, and R^{d4} are independently selected from H, CM alkyl, CM haloalkyl, C₂₋₆ alkenyl, C₂₋₆ alkynyl, C₆₋₁₀ aryl, C₃₋₇ cycloalkyl, 5-6 membered heteroaryl, 4-7 membered heterocycloalkyl, wherein said CM alkyl, CM haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl are each optionally substituted with 1, 2, or 3 substituents independently selected from OH, CN, amino, halo, C₁₋₆ alkyl, C₁₋₆alkoxy, C₁₋₆ haloalkyl, and C₁₋₆ haloalkoxy; and
each R^{e}, R^{e1}, R^{e2}, R^{e3}, and R^{e4} is independently selected from H, C₁₋₄ alkyl, and CN;
with the proviso that the compound is other than:

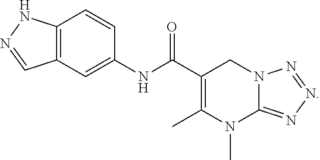 [SMILES: CC1=C(C(=O)Nc2ccc3[nH]ncc3c2)Cn2nnnc2N1C]

88. The compound of claim 1 selected from:
N-(1H-indazol-5-yl)-4,5,7-trimethyl-4H,7H-[1,2,3,4]tetrazolo[1,5-a]pyrimidine-6-carboxamide;
N-(1H-indazol-5-yl)-4,5,7-trimethyl-4H,7H-[1,2,3,4]tetrazolo[1,5-a]pyrimidine-6-carboxamide;
N-(1H-indazol-6-yl)-4,5-dimethyl-4H,7H-[1,2,3,4]tetrazolo[1,5-a]pyrimidine-6-carboxamide;
N-(1H-Indazol-5-yl)-5-methyl-4-[2-(morpholin-4-yl)ethyl]-4H,7H-[1,2,3,4]tetrazolo[1,5-a]pyrimidine-6-carboxamide;
5-Ethyl-N-(1H-indazol-5-yl)-4-methyl-4H,7H-[1,2,3,4]tetrazolo[1,5-a]pyrimidine-6-carb oxamide;
4,5-dimethyl-N-(3-(pyridin-4-yl)-1H-indazol-5-yl)-4,7-dihydrotetrazolo[1,5-a]pyrimidine-6-carboxamide;
4,5-Dimethyl-N-(3-(2-morpholinopyridin-4-yl)-1H-indazol-5-yl)-4,7-dihydrotetrazolo[1,5-a]pyrimidine-6-carboxamide;
7-Ethyl-N-(1H-indazol-5-yl)-4,5-dimethyl-4H,7H-[1,2,3,4]tetrazolo[1,5-a]pyrimidine-6-carboxamide;
4,5-Dimethyl-N-(3-(6-morpholinopyrimidin-4-yl)-1H-indazol-5-yl)-4,7-dihydrotetrazolo[1,5-a]pyrimidine-6-carboxamide;
N-(3-Bromo-1H-indazol-5-yl)-4,5-dimethyl-4,7-dihydrotetrazolo[1,5-a]pyrimidine-6-carboxamide;
4,5-Dimethyl-N-(3-(4-sulfamoylphenyl)-1H-indazol-5-yl)-4,7-dihydrotetrazolo[1,5-a]pyrimidine-6-carboxamide;
N⁶-(1H-indazol-5-yl)-N⁴,N⁴,5-trimethyltetrazolo[1,5-a]pyrimidine-4,6(7H)-dicarboxamide;
N-(1H-indazol-5-yl)-5-methyl-4-phenyl-4,7-dihydrotetrazolo[1,5-a]pyrimidine-6-carboxamide;
N-(4-fluoro-1H-indazol-5-yl)-4,5-dimethyl-4,7-dihydrotetrazolo[1,5-a]pyrimidine-6-carboxamide;
N-(1H-indazol-5-yl)-4,5-dimethyl-4,7-dihydrotetrazolo[1,5-a]pyrimidine-6-carbothioamide;
4,5,7-trimethyl-N-(2H-pyrazolo[3,4-b]pyridin-5-yl)-4,7-dihydrotetrazolo[1,5-a]pyrimidine-6-carboxamide;
N-(6-methoxy-2H-indazol-5-yl)-4,5-dimethyl-4,7-dihydrotetrazolo[1,5-a]pyrimidine-6-carboxamide;
N-(3-carbamoyl-2H-indazol-5-yl)-4,5-dimethyl-4,7-dihydrotetrazolo[1,5-a]pyrimidine-6-carboxamide;
N-(6-fluoro-2H-indazol-5-yl)-4,5-dimethyl-4,7-dihydrotetrazolo[1,5-a]pyrimidine-6-carboxamide;
N-(6-carbamoyl-2H-indazol-5-yl)-4,5-dimethyl-4,7-dihydrotetrazolo[1,5-a]pyrimidine-6-carboxamide;
N-(6-amino-2H-indazol-5-yl)-4,5-dimethyl-4,7-dihydrotetrazolo[1,5-a]pyrimidine-6-carboxamide;
(7R)—N-(3-bromo-2H-indazol-5-yl)-4,5,7-trimethyl-4,7-dihydrotetrazolo[1,5-a]pyrimidine-6-carboxamide;
4,5,7-trimethyl-N-(1H-pyrazolo[3,4-c]pyridin-5-yl)-4,7-dihydrotetrazolo[1,5-a]pyrimidine-6-carboxamide;
4,5,7-trimethyl-N-(3-(2-morpholinopyridin-4-yl)-1H-indazol-5-yl)-4,7-dihydrotetrazolo[1,5-a]pyrimidine-6-carboxamide;
N-(6-amino-2H-indazol-5-yl)-4,5,7-trimethyl-4,7-dihydrotetrazolo[1,5-a]pyrimidine-6-carboxamide;
N-(2H-indazol-5-yl)-4,5,7,7-tetramethyl-4,7-dihydrotetrazolo[1,5-a]pyrimidine-6-carboxamide;
(R)-4,5,7-trimethyl-N-(3-(pyridin-4-yl)-1H-indazol-5-yl)-4,7-dihydrotetrazolo[1,5-a]pyrimidine-6-carboxamide;
N-(3-acetamido-2H-indazol-5-yl)-4,5-dimethyl-4,7-dihydrotetrazolo[1,5-a]pyrimidine-6-carboxamide;
4,5-dimethyl-N-(2-oxoindolin-5-yl)-4,7-dihydrotetrazolo[1,5-a]pyrimidine-6-carboxamide;
4,5-dimethyl-N-(2-oxo-2,3-dihydrobenzo[d]oxazol-6-yl)-4,7-dihydrotetrazolo[1,5-a]pyrimidine-6-carboxamide; and
4,5-dimethyl-N-(2-oxo-2,3-dihydro-1H-benzo[d]imidazol-5-yl)-4,7-dihydrotetrazolo[1,5-a]pyrimidine-6-carboxamide;
or a pharmaceutically acceptable salt thereof.

89. The compound of claim 1 selected from:
4′,5′-dimethyl-N-(3-methyl-2H-indazol-5-yl)-4′H-spiro[cyclopentane-1,7′-tetrazolo[1,5-a]pyrimidine]-6′-carboxamide;
4,5-dimethyl-N-{3-[3-(morpholin-4-yl)phenyl]-1H-indazol-5-yl}-4H-spiro[[1,2,3,4]tetrazolo[1,5-a]pyrimidine-7,1′-cyclopentane]-6-carboxamide;
(R)—N-(3-(2-((2S,6R)-2,6-dimethylmorpholino)pyridin-4-yl)-1H-indazol-5-yl)-4,5,7-trimethyl-4,7-dihydrotetrazolo[1,5-a]pyrimidine-6-carboxamide;
(R)-4,5,7-trimethyl-N-(3-(pyrrolidin-1-yl)-1H-indazol-5-yl)-4,7-dihydrotetrazolo[1,5-a]pyrimidine-6-carboxamide;
(R)—N-(3-isopropyl-1H-indazol-5-yl)-4,5,7-trimethyl-4,7-dihydrotetrazolo[1,5-a]pyrimidine-6-carboxamide;
trans-(7R)—N-(3-(2-(2,6-dimethyl morpholino)pyridin-4-yl)-1H-indazol-5-yl)-4,5,7-trimethyl-4,7-dihydrotetrazolo[1,5-a]pyrimidine-6-carboxamide;
(7R)—N-(3-{2-[(2S,6S)-2,6-dimethylmorpholin-4-yl]pyridin-4-yl}-1H-indazol-5-yl)-4,5,7-trimethyl-4H,7H-[1,2,3,4]tetrazolo[1,5-a]pyrimidine-6-carboxamide;
(7R)—N-(3-{2-[(2R,6R)-2,6-dimethylmorpholin-4-yl]pyridin-4-yl}-1H-indazol-5-yl)-4,5,7-trimethyl-4H,7H-[1,2,3,4]tetrazolo[1,5-a]pyrimidine-6-carboxamide;
(R)—N-(3-(2-((3R,5S)-3,5-dimethylpiperidin-1-yl)pyridin-4-yl)-1H-indazol-5-yl)-4,5,7-trimethyl-4,7-dihydrotetrazolo[1,5-a]pyrimidine-6-carboxamide;
(R)-4,5,7-trimethyl-N-(3-phenyl-1H-indazol-5-yl)-4,7-dihydrotetrazolo[1,5-a]pyrimidine-6-carboxamide;
(R)—N-(3-(3-((2S,6R)-2,6-dimethylmorpholino)phenyl)-1H-indazol-5-yl)-4,5,7-trimethyl-4,7-dihydrotetrazolo[1,5-a]pyrimidine-6-carboxamide;
4,5,7-trimethyl-N-(3-methyl-1H-indazol-5-yl)-4,7-dihydrotetrazolo[1,5-a]pyrimidine-6-carboxamide;
(R)—N-(1-aminoisoquinolin-6-yl)-4,5,7-trimethyl-4,7-dihydrotetrazolo[1,5-a]pyrimidine-6-carboxamide;
4,5,7,7-tetramethyl-N-(3-(2-morpholinopyridin-4-yl)-1H-indazol-5-yl)-4,7-dihydrotetrazolo[1,5-a]pyrimidine-6-carboxamide;
4,5,7,7-tetramethyl-N-(3-(3-morpholinophenyl)-1H-indazol-5-yl)-4,7-dihydrotetrazolo[1,5-a]pyrimidine-6-carboxamide;
N-(3-(3-((2S,6R)-2,6-dDimethylmorpholino)phenyl)-1H-indazol-5-yl)-4,5,7,7-tetramethyl-4,7-dihydrotetrazolo[1,5-a]pyrimidine-6-carboxamide;
4,5,7,7-tetramethyl-N-(3-phenyl-1H-indazol-5-yl)-4,7-dihydrotetrazolo[1,5-a]pyrimidine-6-carboxamide;
(R)-4,5,7-trimethyl-N-(3-methyl-2-oxo-2,3-dihydro-1H-benzo[d]imidazol-5-yl)-4,7-dihydrotetrazolo[1,5-a]pyrimidine-6-carboxamide;
(R)-4,5,7-trimethyl-N-(1-methyl-2-oxo-2,3-dihydro-1H-benzo[d]imidazol-5-yl)-4,7-dihydrotetrazolo[1,5-a]pyrimidine-6-carboxamide;
(R)—N-(3,3-dimethyl-1-oxoisoindolin-5-yl)-4,5,7-trimethyl-4,7-dihydrotetrazolo[1,5-a]pyrimidine-6-carboxamide;
N-(1H-indazol-5-yl)-7-isopropyl-4,5-dimethyl-4,7-dihydrotetrazolo[1,5-a]pyrimidine-6-carboxamide;
4-acetyl-N-(2H-indazol-5-yl)-5-methyl-4,7-dihydrotetrazolo[1,5-a]pyrimidine-6-carboxamide;
N-(3-(2-(4-(dimethylamino)phenyl)acetamido)-2H-indazol-5-yl)-4,5-dimethyl-4,7-dihydrotetrazolo[1,5-a]pyrimidine-6-carboxamide;
N-(4-methoxy-1H-indazol-5-yl)-4,5-dimethyl-4,7-dihydrotetrazolo[1,5-a]pyrimidine-6-carboxamide;
4,5-dimethyl-N-(3-((6-methylpyridin-3-yl)carbamoyl)-2H-indazol-5-yl)-4,7-dihydrotetrazolo[1,5-a]pyrimidine-6-carboxamide;
N-(3-(furan-2-carboxamido)-2H-indazol-5-yl)-4,5-dimethyl-4,7-dihydrotetrazolo[1,5-a]pyrimidine-6-carboxamide;
N-(3-(cyclopropanecarboxamido)-2H-indazol-5-yl)-4,5-dimethyl-4,7-dihydrotetrazolo[1,5-a]pyrimidine-6-carboxamide;
N-(3-butyramido-2H-indazol-5-yl)-4,5-dimethyl-4,7-dihydrotetrazolo[1,5-a]pyrimidine-b-carboxamide;
4,5-dimethyl-N-(3-methyl-2H-indazol-5-yl)-4,7-dihydrotetrazolo[1,5-a]pyrimidine-6-carboxamide;
N-(3-(1H-benzo[d]imidazol-2-yl)-1H-indazol-5-yl)-4,5-dimethyl-4,7-dihydrotetrazolo[1,5-a]pyrimidine-6-carboxamide;
4,5,7,7-tetramethyl-N-(3-methyl-2H-indazol-5-yl)-4,7-dihydrotetrazolo[1,5-a]pyrimidine-6-carboxamide;
methyl 5-(4,5-dimethyl-4,7-dihydrotetrazolo[1,5-a]pyrimidine-6-carboxamido)-2H-indazole-4-carboxylate;
4,5-dimethyl-N-(4-methyl-2H-indazol-5-yl)-4,7-dihydrotetrazolo[1,5-a]pyrimidine-6-carboxamide;
4,5-dimethyl-N-(3-methyl-1H-indol-5-yl)-4,7-dihydrotetrazolo[1,5-a]pyrimidine-6-carboxamide;
4,5-dimethyl-N-(1-methyl-1H-indazol-6-yl)-4,7-dihydrotetrazolo[1,5-a]pyrimidine-6-carboxamide;
4,5-dimethyl-N-(3-methyl-6-(trifluoromethyl)-2H-indazol-5-yl)-4,7-dihydrotetrazolo[1,5-a]pyrimidine-6-carboxamide; and
N-(3-benzamido-2H-indazol-5-yl)-4,5-dimethyl-4,7-dihydrotetrazolo[1,5-a]pyrimidine-6-carboxamide;
or a pharmaceutically acceptable salt thereof.

90. A pharmaceutical composition comprising a compound of claim 1, or a pharmaceutically acceptable salt thereof, and at least one pharmaceutically acceptable carrier.

91. A method of inhibiting LRRK2 activity, said method comprising contacting a compound of claim 1, or a pharmaceutically acceptable salt thereof with LRRK2.

92. The method of claim 91, wherein the LRRK2 is characterized by a G2019S mutation.

93. The method of claim 91, wherein the contacting comprises administering the compound to a patient.

94. A method of treating a disease or disorder associated with elevated expression or activity of LRRK2, or functional variants thereof, said method comprising administering to a patient in need thereof a therapeutically effective amount of a compound of claim 1, or a pharmaceutically acceptable salt thereof.

95. The method of claim 94, wherein the LRRK2 is characterized by a G2019S mutation.

96. A method for treating a neurodegenerative disease in a patient, said method comprising: administering to the patient a therapeutically effective amount of the compound of claim 1, or a pharmaceutically acceptable salt thereof.

97. The method of claim 96, wherein said neurodegenerative disease is selected from Parkinson's disease, Parkinson disease with dementia, Parkinson's disease at risk syndrome, dementia with Lewy bodies, Lewy body variant of Alzheimer's disease, combined Parkinson's disease and Alzheimer's disease, multiple system atrophy, striatonigral degeneration, olivopontocerebellar atrophy, and Shy-Drager syndrome.

98. The method of claim 96, wherein said neurodegenerative disease is Parkinson's disease.

99. The method of claim 96, wherein the Parkinson's disease is characterized by a G2019S mutation in LRRK2.
